(19) United States
(12) Patent Application Publication
(10) Pub. No.: US 2025/0297234 A1
(43) Pub. Date: Sep. 25, 2025

(54) ENGINEERED RNA DEMETHYLASES FOR IMPROVED PLANT GROWTH

(71) Applicant: The University of Chicago, Chicago, IL (US)

(72) Inventors: Chuan HE, Chicago, IL (US); Liudan JIANG, Chicago, IL (US); Jiangbo WEI, Singapore (SG)

(73) Assignee: The University of Chicago, Chicago, IL (US)

(21) Appl. No.: 19/086,929

(22) Filed: Mar. 21, 2025

Related U.S. Application Data
(60) Provisional application No. 63/569,000, filed on Mar. 22, 2024.

(51) Int. Cl. C12N 9/02; C12N 15/82; C12Q 1/6895

(52) U.S. Cl. CPC C12N 9/0071; C12N 15/8262; C12Q 1/6895; C12Q 2600/13

(57) ABSTRACT

The present disclosure relates to engineered RNA m⁶A demethylases with disrupted low complexity regions (LCRs) for expression in plants, as well as recombinant DNA encoding the engineered RNA m⁶A demethylases, vectors encoding the recombinant DNA, plants including the engineered RNA m⁶A demethylases, and method of improving the growth of plants using the engineered RNA m⁶A demethylases, including increased root growth and elevated photosynthesis. The present disclosure further relates to methods of improving growth of a plant, including increased root growth and elevated photosynthesis, by modifying endogenous ALKBH5 homolog genes to include disrupted LCRs, and plants produced by those methods.

### CROSS-REFERENCE TO RELATED APPLICATIONS

This application claims priority to U.S. Provisional Patent Application No. 63/569,000, filed Mar. 22, 2024, which is incorporated herein by reference in its entirety.

### REFERENCE TO AN ELECTRONIC SEQUENCE LISTING

The content of the electronic sequence listing (076482000100subseqlist.xml; Size: 229,245 bytes; and Date of Creation: Mar. 30, 2025) is herein incorporated by reference in its entirety.

### FIELD

The present disclosure relates generally to improvements of plant growth, and more specifically to improvements of plant growth through engineered RNA demethylases.

### BACKGROUND

N⁶-methyladenosine (m⁶A), an abundant RNA modification in mRNA and chromatin-associated RNA in higher eukaryotes, undergoes dynamic regulation orchestrated by writer, eraser, and reader proteins (PMID: 22575960, 22608085, 30262497). While playing crucial roles in regulating mRNA metabolism and translation that impacts diverse biological processes, m⁶A modifications on chromatin-associated regulatory RNA (carRNA) have been reported to control local and global chromatin states as well as the transcription of downstream genes in mammals (PMID: 35511947, 31949099). FTO and ALKBH5 are two demethylases that can reverse m⁶A methylation (PMID: 22002720, 23177736). The knockout of FTO in mouse embryonic stem cells (mESCs) revealed an increase in m⁶A levels, particularly in LINE1 and other repeat RNAs, compared to wild-type control cells. This led to a reduction in LINE1 RNA abundance and a more closed chromatin (PMID: 22002720). More recently, overexpression of human FTO in rice and potato led to a remarkable yield and biomass increases of approximately 50% in field trials (PMID: 34294912).

With the exponential rise of population, it is estimated that in 2050 70% more food will be required to be available for human consumption than is consumed today (PMID: 31304264). To address the food security and climate change challenge, scientists have employed diverse genetic approaches aimed at enhancing crop yields and increase crop tolerance to environmental stresses while also maximizing yield. Although genes responsible for important agricultural traits have been identified, but the process of repeated cycles of selection is time-consuming and results only in slow change. A universal and disruptive technology has not been identified since the application of hybridization to increase crop yields. As such, a need exists for additional techniques to improve plant yield.

### BRIEF SUMMARY

An aspect of the disclosure includes a recombinant DNA encoding an engineered RNA m⁶A demethylase, wherein the engineered RNA m⁶A demethylase includes at least one disrupted endogenous low complexity region (LCR). In a further embodiment of this aspect, the RNA m⁶A demethylase includes a disrupted endogenous N-terminal LCR. In an additional embodiment of the aspect, the RNA m⁶A demethylase includes a disrupted endogenous C-terminal LCR. In some embodiments of this aspect, which may be combined with any of the preceding embodiments, the RNA m⁶A demethylase includes a disrupted endogenous N-terminal LCR and a disrupted C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the RNA m⁶A demethylase includes a deletion of at least about 10%, 20%, 30%, 40%, 50%, 60%, 70%, 80%, 90%, or 100% deletion of at least one endogenous LCR. In additional embodiments of this aspect, the RNA m⁶A demethylase lacks at least one endogenous LCR. In some further embodiments of this aspect, the RNA m⁶A demethylase lacks an endogenous N-terminal LCR. In some further embodiments of this aspect, the RNA m⁶A demethylase lacks an endogenous C-terminal LCR. In yet another embodiment of this aspect, the RNA m⁶A demethylase lacks both an endogenous N-terminal LCR and an endogenous C-terminal LCR.

In some embodiments of this aspect, which may be combined with any of the preceding embodiments, the RNA m⁶A demethylase is an engineered ALKBH5 or an engineered ALKBH5 homolog. In certain embodiments of this aspect, which may be combined with any of the preceding embodiments, the RNA m⁶A demethylase includes an amino acid sequence with at least about 70%, 75%, 80%, 85%, 90%, 91%, 92%, 93%, 94%, 95%, 96%, 97%, 98%, 99%, or 100% sequence identity to SEQ ID NO: 42. In another embodiment of this aspect, which may be combined with any of the preceding embodiments, the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 43. In a separate embodiment of this aspect, which may be combined with any of the preceding embodiments, the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 44. In an additional embodiment of this aspect, which may be combined with any of the preceding embodiments, the endogenous LCR leads to assembly of the endogenous RNA m⁶A demethylase in foci or condensates within the cell. In a certain embodiment of this aspect, which may be combined with any of the preceding embodiments, the engineered RNA m⁶A demethylase is an engineered ALKBH5. In one embodiment of this aspect, the ALKBH5 includes a deletion of at least about 10%, 20%, 30%, 40%, 50%, 60%, 70%, 80%, 90%, or 100% of (i) amino acids 30-81 and/or (ii) amino acids 298-394 of endogenous ALKBH5 (SEQ ID NO: 1). In some embodiments of this aspect, the ALKBH5 includes a deletion of at least about 10%, 20%, 30%, 40%, 50%, 60%, 70%, 80%, 90%, or 100% of amino acids 30-81 of endogenous ALKBH5 (SEQ ID NO: 1). In one embodiment of this aspect, the ALKBH5 includes a deletion of at least about 10%, 20%, 30%, 40%, 50%, 60%, 70%, 80%, 90%, or 100% of amino acids 298-394 of endogenous ALKBH5 (SEQ ID NO: 1). In an additional embodiment of this aspect, the ALKBH5 includes a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of endogenous ALKBH5 (SEQ ID NO: 1). In yet another embodiment of this aspect, the RNA m⁶A demethylase is selected from ALKBH5_{Δ30-81}, ALKBH5_{Δ298-394}, and ALKBH5_{Δ30-81 and 298-394}. In one embodiment of this aspect, the RNA m⁶A demethylase is ALKBH5_{Δ298-394}. In some embodiments of this aspect, which may be combined with any of the preceding embodiments, the RNA m⁶A demethylase is an engineered ALKBH5 homolog. In a further embodiment of this aspect, the RNA m⁶A demethylase is an animal ALKBH5 homolog. In some embodiments of this aspect, the RNA m⁶A demethylase is a mammalian ALKBH5 homolog. In other embodiments of this aspect, the RNA m⁶A demethylase is a non-animal ALKBH5 homolog. In an additional embodiment of this aspect, the RNA m⁶A demethylase is a plant ALKBH5 homolog. In a further embodiment of this aspect, the RNA m⁶A demethylase is an ALKBH5 homolog from Arabidopsis, rice, rapeseed, tobacco, alfalfa, sorghum, maize, wheat, or soy. In an embodiment of this aspect, the RNA m⁶A demethylase is an Arabidopsis ALKBH5 homolog. In a further embodiment of this aspect, the RNA m⁶A demethylase is selected from ALKBH9B and ALKBH10B. In yet another embodiment of this aspect, the RNA m⁶A demethylase is ALKBH9B. In some embodiments of this aspect, the ALKBH9B includes a deletion of at least about 10%, 20%, 30%, 40%, 50%, 60%, 70%, 80%, 90%, or 100% of (i) amino acids 76-102, (ii) amino acids 145-183, and/or (iii) amino acids 432-507 of endogenous Arabidopsis ALKBH9B (SEQ ID NO: 3). In one embodiment of this aspect, the ALKBH9B includes a deletion of at least about 10%, 20%, 30%, 40%, 50%, 60%, 70%, 80%, 90%, or 100% of amino acids 76-102 of endogenous Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 145-183 of endogenous Arabidopsis ALKBH9B (SEQ ID NO: 3). In yet another embodiment of this aspect, the ALKBH9B includes a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In an additional embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of endogenous Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In still another embodiment of this aspect, the ALKBH9B includes a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In a further embodiment of this aspect, the ALKBH9B includes a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In yet another embodiment of this aspect, the ALKBH9B is selected from ALKBH9B_{Δ176-102}, ALKBH9B_{Δ145-183}, and ALKBH9B_{Δ432-507}. In some embodiments of this aspect, the RNA m⁶A demethylase is ALKBH10B. In certain embodiments of this aspect, the ALKBH10B includes a deletion of at least about 10%, 20%, 30%, 40%, 50%, 60%, 70%, 80%, 90%, or 100% of (i) amino acids 131-190 and/or (ii) amino acids 501-569 of endogenous Arabidopsis ALKBH10B (SEQ ID NO: 5). In one embodiment of this aspect, the ALKBH10B includes a deletion of at least about 10%, 20%, 30%, 40%, 50%, 60%, 70%, 80%, 90%, or 100% of amino acids 131-190 of endogenous Arabidopsis ALKBH10B (SEQ ID NO: 5). In another embodiment of this aspect, the ALKBH10B includes a deletion of at least about 10%, 20%, 30%, 40%, 50%, 60%, 70%, 80%, 90%, or 100% of amino acids 501-569 of endogenous Arabidopsis ALKBH10B (SEQ ID NO: 5). In yet another embodiment of this aspect, the ALKBH10B includes a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of endogenous Arabidopsis ALKBH10B (SEQ ID NO: 5). In some embodiments of this aspect, the RNA m⁶A demethylase is a rice ALKBH5 homolog. In a further embodiment of this aspect, the RNA m⁶A demethylase is selected from Os9B and Os10B. In yet another embodiment of this aspect, the RNA m⁶A demethylase is Os9B. In an additional embodiment of this aspect, the Os9B includes a deletion of at least about 10%, 20%, 30%, 40%, 50%, 60%, 70%, 80%, 90%, or 100% of (i) amino acids 60-170 and/or (ii) amino acids 428-616 of endogenous Os9B (SEQ ID NO: 6). In a further embodiment of this aspect, the Os9B includes a deletion of at least about 10%, 20%, 30%, 40%, 50%, 60%, 70%, 80%, 90%, or 100% of amino acids 60-170 of endogenous Os9B (SEQ ID NO: 6). In another embodiment of this aspect, the Os9B includes a deletion of at least about 10%, 20%, 30%, 40%, 50%, 60%, 70%, 80%, 90%, or 100% of amino acids 428-616 of endogenous Os9B (SEQ ID NO: 6). In an additional embodiment of this aspect, the Os9B includes a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of endogenous Os9B (SEQ ID NO: 6). In still another embodiment of this aspect, the Os9B is selected from Os9B. 160-170 and 428-616, and Os9B_{Δ428-616}. In yet another embodiment of this aspect, the RNA m⁶A demethylase is Os10B. In some embodiments of this aspect, the Os10B includes a deletion of at least about 10%, 20%, 30%, 40%, 50%, 60%, 70%, 80%, 90%, or 100% of (i) amino acids 2-30, (ii) amino acids 99-126, and/or (iii) amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 of endogenous Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 99-126 of endogenous Os10B (SEQ ID NO: 8). In one embodiment of this aspect, the Os10B includes a deletion of at least about 10%, 20%, 30%, 40%, 50%, 60%, 70%, 80%, 90%, or 100% of amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of endogenous Os10B (SEQ ID NO: 8). In a further embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of endogenous Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of endogenous Os10B (SEQ ID NO: 8). In a further embodiment of this aspect, the Os10B is selected from Os10B_{Δ2-30, 99-126, and 491-595}, and Os10B_{Δ2-30, 99-126, and 389-595}. In yet another embodiment of this aspect, the RNA m⁶A demethylase is rapeseed ALKBH9B. In some embodiments of this aspect, the ALKBH9B includes a deletion of at least about 10%, 20%, 30%, 40%, 50%, 60%, 70%, 80%, 90%, or 100% of (i) amino acids 55-137 and/or (ii) amino acids 401-467 of endogenous rapeseed ALKBH9B (SEQ ID NO: 10). In a further embodiment of this aspect, the ALKBH9B includes a deletion of at least about 10%, 20%, 30%, 40%, 50%, 60%, 70%, 80%, 90%, or 100% of amino acids 55-137 of endogenous rapeseed ALKBH9B (SEQ ID NO: 10). In one embodiment of this aspect, the ALKBH9B includes a deletion of at least about 10%, 20%, 30%, 40%, 50%, 60%, 70%, 80%, 90%, or 100% of amino acids 401-467 of endogenous rapeseed ALKBH9B (SEQ ID NO: 10). In an additional embodiment of this aspect, the ALKBH9B includes a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of endogenous rapeseed ALKBH9B (SEQ ID NO: 10). In yet another embodiment of this aspect, the RNA m⁶A demethylase is tobacco ALKBH9B. In still another embodiment of this aspect, the ALKBH9B includes a deletion of at least about 10%, 20%, 30%, 40%, 50%, 60%, 70%, 80%, 90%, or 100% of (i) amino acids 121-243 and/or (ii) amino acids 447-541 of endogenous tobacco ALKBH9B (SEQ ID NO: 12). In an additional embodiment of this aspect, the ALKBH9B includes a deletion of at least about 10%, 20%, 30%, 40%, 50%, 60%, 70%, 80%, 90%, or 100% of amino acids 121-243 of endogenous tobacco ALKBH9B (SEQ ID NO: 12). In one embodiment of this aspect, the ALKBH9B includes a deletion of at least about 10%, 20%, 30%, 40%, 50%, 60%, 70%, 80%, 90%, or 100% of amino acids 447-541 of endogenous tobacco ALKBH9B (SEQ ID NO: 12). In a further embodiment of this aspect, the ALKBH9B includes a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of endogenous tobacco ALKBH9B (SEQ ID NO: 12). In an additional embodiment of this aspect, the RNA m⁶A demethylase is tobacco ALKBH10B. In some embodiments of this aspect, the ALKBH10B includes a deletion of at least about 10%, 20%, 30%, 40%, 50%, 60%, 70%, 80%, 90%, or 100% of (i) amino acids 181-235 and/or (ii) amino acids 504-640 of endogenous tobacco ALKBH10B (SEQ ID NO: 14). In one embodiment of this aspect, the ALKBH10B includes a deletion of at least about 10%, 20%, 30%, 40%, 50%, 60%, 70%, 80%, 90%, or 100% of amino acids 181-235 of endogenous tobacco ALKBH10B (SEQ ID NO: 14). In another embodiment of this aspect, the ALKBH10B includes a deletion of at least about 10%, 20%, 30%, 40%, 50%, 60%, 70%, 80%, 90%, or 100% of amino acids 504-640 of endogenous tobacco ALKBH10B (SEQ ID NO: 14). In an additional embodiment of this aspect, the ALKBH10B includes a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of endogenous tobacco ALKBH10B (SEQ ID NO: 14). In a further embodiment of this aspect, the RNA m⁶A demethylase is alfalfa ALKBH9B. In one embodiment of this aspect, the ALKBH9B includes a deletion of at least about 10%, 20%, 30%, 40%, 50%, 60%, 70%, 80%, 90%, or 100% of amino acids 442-508 of endogenous alfalfa ALKBH9B (SEQ ID NO: 16). In a different embodiment of this aspect, the RNA m⁶A demethylase is sorghum ALKBH9B. In a further embodiment of this aspect, the ALKBH9B includes a deletion of at least about 10%, 20%, 30%, 40%, 50%, 60%, 70%, 80%, 90%, or 100% of (i) amino acids 66-179 and/or (ii) amino acids 423-611 of endogenous sorghum ALKBH9B (SEQ ID NO: 18). In another embodiment of this aspect, the ALKBH9B includes a deletion of at least about 10%, 20%, 30%, 40%, 50%, 60%, 70%, 80%, 90%, or 100% of amino acids 66-179 of endogenous sorghum ALKBH9B (SEQ ID NO: 18). In yet another embodiment of this aspect, the ALKBH9B includes a deletion of at least about 10%, 20%, 30%, 40%, 50%, 60%, 70%, 80%, 90%, or 100% of amino acids 423-611 of endogenous sorghum ALKBH9B (SEQ ID NO: 18). In still another embodiment of this aspect, the ALKBH9B includes a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of endogenous sorghum ALKBH9B (SEQ ID NO: 18). In some embodiments of this aspect, the RNA m⁶A demethylase is maize ALKBH9B. In certain embodiments of this aspect, the ALKBH9B includes a deletion of at least about 10%, 20%, 30%, 40%, 50%, 60%, 70%, 80%, 90%, or 100% of (i) amino acids 72-165 and/or (ii) amino acids 423-615 of endogenous maize ALKBH9B (SEQ ID NO: 20). In a further embodiment of this aspect, the ALKBH9B includes a deletion of at least about 10%, 20%, 30%, 40%, 50%, 60%, 70%, 80%, 90%, or 100% of amino acids 72-165 of endogenous maize ALKBH9B (SEQ ID NO: 20). In one embodiment of this aspect, the ALKBH9B includes a deletion of at least about 10%, 20%, 30%, 40%, 50%, 60%, 70%, 80%, 90%, or 100% of amino acids 423-615 of endogenous maize ALKBH9B (SEQ ID NO: 20). In another embodiment of this aspect, the ALKBH9B includes a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of endogenous maize ALKBH9B (SEQ ID NO: 20). In yet another embodiment of this aspect, the RNA m⁶A demethylase is maize ALKBH10B. In one embodiment of this aspect, the ALKBH10B includes a deletion of at least about 10%, 20%, 30%, 40%, 50%, 60%, 70%, 80%, 90%, or 100% of (i) amino acids 109-210 and/or (ii) amino acids 437-573 of endogenous maize ALKBH10B (SEQ ID NO: 22). In another embodiment of this aspect, the ALKBH10B includes a deletion of at least about 10%, 20%, 30%, 40%, 50%, 60%, 70%, 80%, 90%, or 100% of amino acids 109-210 of endogenous maize ALKBH10B (SEQ ID NO: 22). In still another embodiment of this aspect, the ALKBH10B includes a deletion of at least about 10%, 20%, 30%, 40%, 50%, 60%, 70%, 80%, 90%, or 100% of amino acids 437-573 of endogenous maize ALKBH10B (SEQ ID NO: 22). In a further embodiment of this aspect, the ALKBH10B includes a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of endogenous maize ALKBH10B (SEQ ID NO: 22). In an additional embodiment of this aspect, the RNA m⁶A demethylase is wheat ALKBH9B. In some embodiments of this aspect, the ALKBH9B includes a deletion of at least about 10%, 20%, 30%, 40%, 50%, 60%, 70%, 80%, 90%, or 100% of (i) amino acids 57-163 and/or (ii) amino acids 417-612 of endogenous wheat ALKBH9B (SEQ ID NO: 24). In an embodiment of this aspect, the ALKBH9B includes a deletion of at least about 10%, 20%, 30%, 40%, 50%, 60%, 70%, 80%, 90%, or 100% of amino acids 57-163 of endogenous wheat ALKBH9B (SEQ ID NO: 24). In yet another embodiment of this aspect, the ALKBH9B includes a deletion of at least about 10%, 20%, 30%, 40%, 50%, 60%, 70%, 80%, 90%, or 100% of amino acids 417-612 of endogenous wheat ALKBH9B (SEQ ID NO: 24). In an additional embodiment of this aspect, the ALKBH9B includes a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of endogenous wheat ALKBH9B (SEQ ID NO: 24). In still another embodiment of this aspect, the RNA m⁶A demethylase is wheat ALKBH10B. In some embodiments of this aspect, the ALKBH10B includes a deletion of at least about 10%, 20%, 30%, 40%, 50%, 60%, 70%, 80%, 90%, or 100% of (i) amino acids 91-211 and/or (ii) amino acids 440-566 of endogenous wheat ALKBH10B (SEQ ID NO: 26). In another embodiment of this aspect, the ALKBH10B includes a deletion of at least about 10%, 20%, 30%, 40%, 50%, 60%, 70%, 80%, 90%, or 100% of amino acids 91-211 of endogenous wheat ALKBH10B (SEQ ID NO: 26). In a further embodiment of this aspect, the ALKBH10B includes a deletion of at least about 10%, 20%, 30%, 40%, 50%, 60%, 70%, 80%, 90%, or 100% of amino acids 440-566 of endogenous wheat ALKBH10B (SEQ ID NO: 26). In still another embodiment of this aspect, the ALKBH10B includes a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of endogenous wheat ALKBH10B (SEQ ID NO: 26). In a further embodiment of this aspect, the RNA m⁶A demethylase is cassava ALKBH9B. In another embodiment of this aspect, the RNA m⁶A demethylase is cassava ALKBH10B. In an additional embodiment of this aspect, the RNA m⁶A demethylase is cowpea ALKBH9B. In another embodiment of this aspect, the RNA m⁶A demethylase is cowpea ALKBH10B. In a further embodiment of this aspect, the RNA m⁶A demethylase is cotton ALKBH9B. In another embodiment of this aspect, the RNA m⁶A demethylase is cotton ALKBH10B.

In one embodiment of this aspect, which may be combined with any of the previous embodiments, the RNA m⁶A demethylase is operably linked to at least one nuclear localization signal (NLS). In a further embodiment of this aspect, the NLS is encoded by SEQ ID NO: 40. In yet another embodiment of this aspect, the amino acid sequence of the NLS is SEQ ID NO: 41. In still another embodiment of this aspect, the RNA m⁶A demethylase is operably linked to at least one heterologous nuclear localization signal (NLS). In an additional embodiment of this aspect, which may be combined with any of the previous embodiments, the RNA m⁶A demethylase is operably linked to a promoter for expression in a plant. In some embodiments of this aspect, the promoter is a constitutive promoter.

Some aspects of the disclosure include an expression vector including the recombinant DNA of any of the preceding embodiments. An additional aspect of the disclosure provides an expression vector including a nucleic acid encoding an engineered RNA m⁶A demethylase, wherein the engineered RNA m⁶A demethylase includes at least one disrupted endogenous low complexity region (LCR), and wherein the expression vector is for expression in a plant or plant cell. Some aspects of the disclosure further include a transformation vector including the recombinant DNA of any of the preceding embodiments, or the expression vector of any of the preceding embodiments.

A further aspect of the disclosure provides a plant or plant cell including the recombinant DNA of any of the preceding embodiments, or the expression vector of any of the preceding embodiments. In some embodiments of this aspect, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant or a plant including the plant cell of any of the preceding embodiments. An additional aspect of the disclosure provides a commercial product derived from the plant or a plant including the plant cell of any of the preceding embodiments, or the seed of any of the preceding embodiments.

An additional aspect of the disclosure provides a method of improving growth of a plant including a) engineering a plant to include the recombinant DNA of any of the preceding recombinant DNA embodiments, the expression vector of any of the preceding expression vector embodiments, or the transformation vector of any of the preceding transformation vector embodiments, and b) growing the plant, wherein the plant has improved growth compared to a control plant. In another embodiment of this aspect, the plant has improved growth under abiotic stress conditions. In an additional embodiment of this aspect, the plant has improved growth under drought stress, salt stress, heat stress, or cold stress. In yet another embodiment of this aspect, the plant has improved growth under biotic stress conditions. In still another embodiment of this aspect, the plant is improved in a characteristic selected from biomass, yield, root growth, root expansion, stem cell expansion of the root, stem cell expansion of the shoot, drought resistance, and pest resistance. In a further embodiment of this aspect, the plant has elevated photosynthesis. In a further embodiment of this aspect, which may be combined with any of the preceding embodiments, the plant is selected from Arabidopsis, rice, corn, soybean, potato, cassava, cowpea, cotton, turf grass, strawberry, blueberry, blackberry, raspberry, and wheat.

Further aspects of the disclosure include a plant including nucleic acid encoding an engineered RNA m⁶A demethylase, wherein the engineered RNA m⁶A demethylase includes at least one disrupted endogenous low complexity region (LCR). In an embodiment of this aspect, the RNA m⁶A demethylase includes a disrupted endogenous N-terminal LCR. In a further embodiment of this aspect, the RNA m⁶A demethylase includes a disrupted endogenous C-terminal LCR. In an additional embodiment of this aspect, the RNA m⁶A demethylase includes a disrupted endogenous N-terminal LCR and a disrupted C-terminal LCR. In yet another embodiment of this aspect, which may be combined with any of the preceding plant embodiments, the disruption of the endogenous LCR is selected from a partial truncation, a full truncation, a deletion, and a replacement. In another embodiment of this aspect, which may be combined with any of the preceding plant embodiments, the RNA m⁶A demethylase includes a deletion of at least about 10%, 20%, 30%, 40%, 50%, 60%, 70%, 80%, 90%, or 100% deletion of at least one endogenous LCR. In additional embodiments of this aspect, the RNA m⁶A demethylase lacks at least one endogenous LCR. In some further embodiments of this aspect, the RNA m⁶A demethylase lacks an endogenous N-terminal LCR. In some further embodiments of this aspect, the RNA m⁶A demethylase lacks an endogenous C-terminal LCR. In yet another embodiment of this aspect, the RNA m⁶A demethylase lacks both an endogenous N-terminal LCR and an endogenous C-terminal LCR.

In some embodiments of this aspect, which may be combined with any of the preceding plant embodiments, the RNA m⁶A demethylase is an engineered ALKBH5 or an engineered ALKBH5 homolog. In certain embodiments of this aspect, which may be combined with any of the preceding embodiments, the RNA m⁶A demethylase includes an amino acid sequence with at least about 70%, 75%, 80%, 85%, 90%, 91%, 92%, 93%, 94%, 95%, 96%, 97%, 98%, 99%, or 100% sequence identity to SEQ ID NO: 42. In an additional embodiment of this aspect, which may be combined with any of the preceding plant embodiments, the endogenous LCR leads to assembly of the endogenous RNA m⁶A demethylase in foci or condensates within the cell. In still another embodiment of this aspect, which may be combined with any of the preceding plant embodiments, the engineered RNA m⁶A demethylase is a heterologous RNA m⁶A demethylase. In an additional embodiment of this aspect, which may be combined with any of the preceding plant embodiments, the engineered RNA m⁶A demethylase is an endogenous RNA m⁶A demethylase. In a certain embodiment of this aspect, which may be combined with any of the preceding plant embodiments, the engineered RNA m⁶A demethylase is an engineered ALKBH5. In one embodiment of this aspect, the ALKBH5 includes a deletion of at least about 10%, 20%, 30%, 40%, 50%, 60%, 70%, 80%, 90%, or 100% of (i) amino acids 30-81 and/or (ii) amino acids 298-394 of ALKBH5 (SEQ ID NO: 1). In some embodiments of this aspect, the ALKBH5 includes a deletion of at least about 10%, 20%, 30%, 40%, 50%, 60%, 70%, 80%, 90%, or 100% of amino acids 30-81 of ALKBH5 (SEQ ID NO: 1). In one embodiment of this aspect, the ALKBH5 includes a deletion of at least about 10%, 20%, 30%, 40%, 50%, 60%, 70%, 80%, 90%, or 100% of amino acids 298-394 of ALKBH5 (SEQ ID NO: 1). In an additional embodiment of this aspect, the ALKBH5 includes a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of ALKBH5 (SEQ ID NO: 1). In yet another embodiment of this aspect, the RNA m⁶A demethylase is selected from ALKBH5_{Δ30-81}, ALKBH5_{Δ298-394}, and ALKBH5_{Δ30-81 and 298-394}. In one embodiment of this aspect, the RNA m⁶A demethylase is ALKBH5_{Δ298-394}. In some embodiments of this aspect, which may be combined with any of the preceding plant embodiments, the RNA m⁶A demethylase is an engineered ALKBH5 homolog. In a further embodiment of this aspect, the RNA m⁶A demethylase is an animal ALKBH5 homolog. In some embodiments of this aspect, the RNA m⁶A demethylase is a mammalian ALKBH5 homolog. In other embodiments of this aspect, the RNA m⁶A demethylase is a non-animal ALKBH5 homolog. In an additional embodiment of this aspect, the RNA m⁶A demethylase is a plant ALKBH5 homolog. In a further embodiment of this aspect, the RNA m⁶A demethylase is an ALKBH5 homolog from Arabidopsis, rice, rapeseed, tobacco, alfalfa, sorghum, maize, wheat, or soy. In an embodiment of this aspect, the RNA m⁶A demethylase is an Arabidopsis ALKBH5 homolog. In a further embodiment of this aspect, the RNA m⁶A demethylase is selected from ALKBH9B and ALKBH10B. In yet another embodiment of this aspect, the RNA m⁶A demethylase is ALKBH9B. In some embodiments of this aspect, the ALKBH9B includes a deletion of at least about 10%, 20%, 30%, 40%, 50%, 60%, 70%, 80%, 90%, or 100% of (i) amino acids 76-102, (ii) amino acids 145-183, and/or (iii) amino acids 432-507 of Arabidopsis ALKBH9B (SEQ ID NO: 3). In one embodiment of this aspect, the ALKBH9B includes a deletion of at least about 10%, 20%, 30%, 40%, 50%, 60%, 70%, 80%, 90%, or 100% of amino acids 76-102 of Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 145-183 of Arabidopsis ALKBH9B (SEQ ID NO: 3). In yet another embodiment of this aspect, the ALKBH9B includes a deletion of at least about 10%, 20%, 30%, 40%, 50%, 60%, 70%, 80%, 90%, or 100% of amino acids 432-507 of Arabidopsis ALKBH9B (SEQ ID NO: 3). In an additional embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of Arabidopsis ALKBH9B (SEQ ID NO: 3). In still another embodiment of this aspect, the ALKBH9B includes a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of Arabidopsis ALKBH9B (SEQ ID NO: 3). In a further embodiment of this aspect, the ALKBH9B includes a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of Arabidopsis ALKBH9B (SEQ ID NO: 3). In yet another embodiment of this aspect, the ALKBH9B is selected from ALKBH9B_{Δ176-102}, ALKBH9B_{Δ145-183}, and ALKBH9B_{Δ432-507}. In some embodiments of this aspect, the RNA m⁶A demethylase is ALKBH10B. In certain embodiments of this aspect, the ALKBH10B includes a deletion of at least about 10%, 20%, 30%, 40%, 50%, 60%, 70%, 80%, 90%, or 100% of (i) amino acids 131-190 and/or (ii) amino acids 501-569 of Arabidopsis ALKBH10B (SEQ ID NO: 5). In one embodiment of this aspect, the ALKBH10B includes a deletion of at least about 10%, 20%, 30%, 40%, 50%, 60%, 70%, 80%, 90%, or 100% of amino acids 131-190 of Arabidopsis ALKBH10B (SEQ ID NO: 5). In another embodiment of this aspect, the ALKBH10B includes a deletion of at least about 10%, 20%, 30%, 40%, 50%, 60%, 70%, 80%, 90%, or 100% of amino acids 501-569 of Arabidopsis ALKBH10B (SEQ ID NO: 5). In yet another embodiment of this aspect, the ALKBH10B includes a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of Arabidopsis ALKBH10B (SEQ ID NO: 5). In some embodiments of this aspect, the RNA m⁶A demethylase is a rice ALKBH5 homolog. In a further embodiment of this aspect, the RNA m⁶A demethylase is selected from Os9B and Os10B. In yet another embodiment of this aspect, the RNA m⁶A demethylase is Os9B. In an additional embodiment of this aspect, the Os9B includes a deletion of at least about 10%, 20%, 30%, 40%, 50%, 60%, 70%, 80%, 90%, or 100% of (i) amino acids 60-170 and/or (ii) amino acids 428-616 of Os9B (SEQ ID NO: 6). In a further embodiment of this aspect, the Os9B includes a deletion of at least about 10%, 20%, 30%, 40%, 50%, 60%, 70%, 80%, 90%, or 100% of amino acids 60-170 of Os9B (SEQ ID NO: 6). In another embodiment of this aspect, the Os9B includes a deletion of at least about 10%, 20%, 30%, 40%, 50%, 60%, 70%, 80%, 90%, or 100% of amino acids 428-616 of Os9B (SEQ ID NO: 6). In an additional embodiment of this aspect, the Os9B includes a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of Os9B (SEQ ID NO: 6). In still another embodiment of this aspect, the Os9B is selected from Os9B_{Δ60-170 and 428-616}, and Os9B_{Δ428-616}. In yet another embodiment of this aspect, the RNA m⁶A demethylase is Os10B. In some embodiments of this aspect, the Os10B includes a deletion of at least about 10%, 20%, 30%, 40%, 50%, 60%, 70%, 80%, 90%, or 100% of (i) amino acids 2-30, (ii) amino acids 99-126, and/or (iii) amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 of Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 99-126 of Os10B (SEQ ID NO: 8). In one embodiment of this aspect, the Os10B includes a deletion of at least about 10%, 20%, 30%, 40%, 50%, 60%, 70%, 80%, 90%, or 100% of amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of Os10B (SEQ ID NO: 8). In a further embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of Os10B (SEQ ID NO: 8). In a further embodiment of this aspect, the Os10B is selected from Os10B_{Δ2-30, 99-126, and 491-595}, and Os10B_{Δ2-30, 99-126, and 389-595}. In yet another embodiment of this aspect, the RNA m⁶A demethylase is rapeseed ALKBH9B. In some embodiments of this aspect, the ALKBH9B includes a deletion of at least about 10%, 20%, 30%, 40%, 50%, 60%, 70%, 80%, 90%, or 100% of (i) amino acids 55-137 and/or (ii) amino acids 401-467 of rapeseed ALKBH9B (SEQ ID NO: 10). In a further embodiment of this aspect, the ALKBH9B includes a deletion of at least about 10%, 20%, 30%, 40%, 50%, 60%, 70%, 80%, 90%, or 100% of amino acids 55-137 of rapeseed ALKBH9B (SEQ ID NO: 10). In one embodiment of this aspect, the ALKBH9B includes a deletion of at least about 10%, 20%, 30%, 40%, 50%, 60%, 70%, 80%, 90%, or 100% of amino acids 401-467 of rapeseed ALKBH9B (SEQ ID NO: 10). In an additional embodiment of this aspect, the ALKBH9B includes a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of rapeseed ALKBH9B (SEQ ID NO: 10). In yet another embodiment of this aspect, the RNA m⁶A demethylase is tobacco ALKBH9B. In still another embodiment of this aspect, the ALKBH9B includes a deletion of at least about 10%, 20%, 30%, 40%, 50%, 60%, 70%, 80%, 90%, or 100% of (i) amino acids 121-243 and/or (ii) amino acids 447-541 of tobacco ALKBH9B (SEQ ID NO: 12). In an additional embodiment of this aspect, the ALKBH9B includes a deletion of at least about 10%, 20%, 30%, 40%, 50%, 60%, 70%, 80%, 90%, or 100% of amino acids 121-243 of tobacco ALKBH9B (SEQ ID NO: 12). In one embodiment of this aspect, the ALKBH9B includes a deletion of at least about 10%, 20%, 30%, 40%, 50%, 60%, 70%, 80%, 90%, or 100% of amino acids 447-541 of tobacco ALKBH9B (SEQ ID NO: 12). In a further embodiment of this aspect, the ALKBH9B includes a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of tobacco ALKBH9B (SEQ ID NO: 12). In an additional embodiment of this aspect, the RNA m⁶A demethylase is tobacco ALKBH10B. In some embodiments of this aspect, the ALKBH10B includes a deletion of at least about 10%, 20%, 30%, 40%, 50%, 60%, 70%, 80%, 90%, or 100% of (i) amino acids 181-235 and/or (ii) amino acids 504-640 of tobacco ALKBH10B (SEQ ID NO: 14). In one embodiment of this aspect, the ALKBH10B includes a deletion of at least about 10%, 20%, 30%, 40%, 50%, 60%, 70%, 80%, 90%, or 100% of amino acids 181-235 of tobacco ALKBH10B (SEQ ID NO: 14). In another embodiment of this aspect, the ALKBH10B includes a deletion of at least about 10%, 20%, 30%, 40%, 50%, 60%, 70%, 80%, 90%, or 100% of amino acids 504-640 of tobacco ALKBH10B (SEQ ID NO: 14). In an additional embodiment of this aspect, the ALKBH10B includes a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of tobacco ALKBH10B (SEQ ID NO: 14). In a further embodiment of this aspect, the RNA m⁶A demethylase is alfalfa ALKBH9B. In one embodiment of this aspect, the ALKBH9B includes a deletion of at least about 10%, 20%, 30%, 40%, 50%, 60%, 70%, 80%, 90%, or 100% of amino acids 442-508 of alfalfa ALKBH9B (SEQ ID NO: 16). In a different embodiment of this aspect, the RNA m⁶A demethylase is sorghum ALKBH9B. In a further embodiment of this aspect, the ALKBH9B includes a deletion of at least about 10%, 20%, 30%, 40%, 50%, 60%, 70%, 80%, 90%, or 100% of (i) amino acids 66-179 and/or (ii) amino acids 423-611 of sorghum ALKBH9B (SEQ ID NO: 18). In another embodiment of this aspect, the ALKBH9B includes a deletion of at least about 10%, 20%, 30%, 40%, 50%, 60%, 70%, 80%, 90%, or 100% of amino acids 66-179 of sorghum ALKBH9B (SEQ ID NO: 18). In yet another embodiment of this aspect, the ALKBH9B includes a deletion of at least about 10%, 20%, 30%, 40%, 50%, 60%, 70%, 80%, 90%, or 100% of amino acids 423-611 of sorghum ALKBH9B (SEQ ID NO: 18). In still another embodiment of this aspect, the ALKBH9B includes a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of sorghum ALKBH9B (SEQ ID NO: 18). In some embodiments of this aspect, the RNA m⁶A demethylase is maize ALKBH9B. In certain embodiments of this aspect, the ALKBH9B includes a deletion of at least about 10%, 20%, 30%, 40%, 50%, 60%, 70%, 80%, 90%, or 100% of (i) amino acids 72-165 and/or (ii) amino acids 423-615 of maize ALKBH9B (SEQ ID NO: 20). In a further embodiment of this aspect, the ALKBH9B includes a deletion of at least about 10%, 20%, 30%, 40%, 50%, 60%, 70%, 80%, 90%, or 100% of amino acids 72-165 of maize ALKBH9B (SEQ ID NO: 20). In one embodiment of this aspect, the ALKBH9B includes a deletion of at least about 10%, 20%, 30%, 40%, 50%, 60%, 70%, 80%, 90%, or 100% of amino acids 423-615 of maize ALKBH9B (SEQ ID NO: 20). In another embodiment of this aspect, the ALKBH9B includes a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of maize ALKBH9B (SEQ ID NO: 20). In yet another embodiment of this aspect, the RNA m⁶A demethylase is maize ALKBH10B. In one embodiment of this aspect, the ALKBH10B includes a deletion of at least about 10%, 20%, 30%, 40%, 50%, 60%, 70%, 80%, 90%, or 100% of (i) amino acids 109-210 and/or (ii) amino acids 437-573 of maize ALKBH10B (SEQ ID NO: 22). In another embodiment of this aspect, the ALKBH10B includes a deletion of at least about 10%, 20%, 30%, 40%, 50%, 60%, 70%, 80%, 90%, or 100% of amino acids 109-210 of maize ALKBH10B (SEQ ID NO: 22). In still another embodiment of this aspect, the ALKBH10B includes a deletion of at least about 10%, 20%, 30%, 40%, 50%, 60%, 70%, 80%, 90%, or 100% of amino acids 437-573 of maize ALKBH10B (SEQ ID NO: 22). In a further embodiment of this aspect, the ALKBH10B includes a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of maize ALKBH10B (SEQ ID NO: 22). In an additional embodiment of this aspect, the RNA m⁶A demethylase is wheat ALKBH9B. In some embodiments of this aspect, the ALKBH9B includes a deletion of at least about 10%, 20%, 30%, 40%, 50%, 60%, 70%, 80%, 90%, or 100% of (i) amino acids 57-163 and/or (ii) amino acids 417-612 of wheat ALKBH9B (SEQ ID NO: 24). In an embodiment of this aspect, the ALKBH9B includes a deletion of at least about 10%, 20%, 30%, 40%, 50%, 60%, 70%, 80%, 90%, or 100% of amino acids 57-163 of wheat ALKBH9B (SEQ ID NO: 24). In yet another embodiment of this aspect, the ALKBH9B includes a deletion of at least about 10%, 20%, 30%, 40%, 50%, 60%, 70%, 80%, 90%, or 100% of amino acids 417-612 of wheat ALKBH9B (SEQ ID NO: 24). In an additional embodiment of this aspect, the ALKBH9B includes a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of wheat ALKBH9B (SEQ ID NO: 24). In still another embodiment of this aspect, the RNA m⁶A demethylase is wheat ALKBH10B. In some embodiments of this aspect, the ALKBH10B includes a deletion of at least about 10%, 20%, 30%, 40%, 50%, 60%, 70%, 80%, 90%, or 100% of (i) amino acids 91-211 and/or (ii) amino acids 440-566 of wheat ALKBH10B (SEQ ID NO: 26). In another embodiment of this aspect, the ALKBH10B includes a deletion of at least about 10%, 20%, 30%, 40%, 50%, 60%, 70%, 80%, 90%, or 100% of amino acids 91-211 of wheat ALKBH10B (SEQ ID NO: 26). In a further embodiment of this aspect, the ALKBH10B includes a deletion of at least about 10%, 20%, 30%, 40%, 50%, 60%, 70%, 80%, 90%, or 100% of amino acids 440-566 of wheat ALKBH10B (SEQ ID NO: 26). In still another embodiment of this aspect, the ALKBH10B includes a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of wheat ALKBH10B (SEQ ID NO: 26). In a further embodiment of this aspect, the RNA m⁶A demethylase is cassava ALKBH9B. In another embodiment of this aspect, the RNA m⁶A demethylase is cassava ALKBH10B. In an additional embodiment of this aspect, the RNA m⁶A demethylase is cowpea ALKBH9B. In another embodiment of this aspect, the RNA m⁶A demethylase is cowpea ALKBH10B. In a further embodiment of this aspect, the RNA m⁶A demethylase is cotton ALKBH9B. In another embodiment of this aspect, the RNA m⁶A demethylase is cotton ALKBH10B.

In one embodiment of this aspect, which may be combined with any of the previous plant embodiments, the RNA m⁶A demethylase is operably linked to at least one nuclear localization signal (NLS). In a further embodiment of this aspect, the NLS is encoded by SEQ ID NO: 40. In yet another embodiment of this aspect, the amino acid sequence of the NLS is SEQ ID NO: 41. In still another embodiment of this aspect, the RNA m⁶A demethylase is operably linked to at least one heterologous nuclear localization signal (NLS). In one embodiment of this aspect, the RNA m⁶A demethylase is operably linked to a promoter for expression in a plant. In an additional embodiment of this aspect, which may be combined with any of the previous plant embodiments, the promoter is a constitutive promoter.

A further aspect of the disclosure provides a plant part, tissue, or cell of the plant of any of the preceding plant embodiments. In some embodiments of this aspect, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant of any of the preceding plant embodiments. An additional aspect of the disclosure provides a commercial product derived from the plant of any of the preceding plant embodiments, or the seed of any of the preceding embodiments.

Some aspects of the disclosure include a plant or plant cell including a modified endogenous ALKBH5 homolog gene, wherein the modified endogenous ALKBH5 homolog gene encodes a protein including at least one disrupted endogenous low complexity region (LCR). In a further embodiment of this aspect, the modified endogenous ALKBH5 homolog gene encodes a protein including a disrupted N-terminal LCR. In an additional embodiment of this aspect, the modified endogenous ALKBH5 homolog gene encodes a protein including a disrupted C-terminal LCR. In some embodiments of this aspect, which may be combined with any of the preceding embodiments, the modified endogenous ALKBH5 homolog gene includes a disrupted endogenous N-terminal LCR and a disrupted C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the modified endogenous ALKBH5 homolog gene includes a deletion of at least about 10%, 20%, 30%, 40%, 50%, 60%, 70%, 80%, 90%, or 100% deletion of at least one endogenous LCR. In additional embodiments of this aspect, the modified endogenous ALKBH5 homolog gene encodes a protein that lacks at least one endogenous LCR. In some further embodiments of this aspect, the modified endogenous ALKBH5 homolog gene encodes a protein that lacks an endogenous C-terminal LCR. In an additional embodiment of this aspect, the modified endogenous ALKBH5 homolog gene was modified to encode an mRNA that includes a stop codon before the C-terminal LCR. In a further embodiment of this aspect, the modified endogenous ALKBH5 homolog gene includes a deletion of at least about 10%, 20%, 30%, 40%, 50%, 60%, 70%, 80%, 90%, or 100% of the nucleotides encoding the C-terminal LCR. In some further embodiments of this aspect, the modified endogenous ALKBH5 homolog gene encodes a protein that lacks an endogenous N-terminal LCR. In a further embodiment of this aspect, the modified endogenous ALKBH5 homolog gene includes a deletion of at least about 10%, 20%, 30%, 40%, 50%, 60%, 70%, 80%, 90%, or 100% of the nucleotides encoding the N-terminal LCR. In yet another embodiment of this aspect, the plant or plant cell was modified by a genome editing technique selected from TALEN editing, zinc finger nuclease editing, RNA-guided endonuclease-mediated editing, prime editing, and base editing. In still another embodiment of this aspect, the modified endogenous ALKBH5 homolog gene was modified to add a nuclear localization signal (NLS) to the encoded protein. In a further embodiment of this aspect, the NLS is encoded by SEQ ID NO: 40. In yet another embodiment of this aspect, the amino acid sequence of the NLS is SEQ ID NO: 41. In one embodiment of this aspect, the NLS is a heterologous NLS. In another embodiment of this aspect, the NLS is an endogenous NLS.

In a further embodiment of this aspect, which may be combined with any of the preceding embodiments, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant or a plant including the plant cell of any of the preceding embodiments. An additional aspect of the disclosure provides a commercial product derived from the plant or a plant including the plant cell of any of the preceding embodiments, or the seed of any of the preceding embodiments.

Further aspects of the disclosure include a method of improving growth of a plant, including a) genetically modifying the plant by transforming the plant with one or more gene editing components that target an endogenous ALKBH5 homolog gene, wherein the endogenous ALKBH5 homolog gene is modified to encode a protein including at least one disrupted endogenous low complexity region (LCR), and b) growing the plant, wherein the plant has improved growth compared to a control plant. In a further embodiment of this aspect, the endogenous ALKBH5 homolog gene is modified to encode a protein including a disrupted endogenous N-terminal LCR. In yet another embodiment of this aspect, the endogenous ALKBH5 homolog gene is modified to encode a protein including a disrupted endogenous C-terminal LCR. In an additional embodiment of this aspect, the endogenous ALKBH5 homolog gene is modified to encode a protein including a disrupted endogenous N-terminal LCR and a disrupted endogenous C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the endogenous ALKBH5 homolog gene is modified to include a deletion of at least about 10%, 20%, 30%, 40%, 50%, 60%, 70%, 80%, 90%, or 100% deletion of at least one endogenous LCR. In a further embodiment of this aspect, the endogenous ALKBH5 homolog gene is modified to encode a protein that lacks an endogenous C-terminal LCR. In yet another embodiment of this aspect, the endogenous ALKBH5 homolog gene is modified to encode an mRNA that includes a stop codon before the C-terminal LCR. In still another embodiment of this aspect, the endogenous ALKBH5 homolog gene is modified to include a deletion of at least about 10%, 20%, 30%, 40%, 50%, 60%, 70%, 80%, 90%, or 100% of the nucleotides encoding the C-terminal LCR. In another embodiment of this aspect, the endogenous ALKBH5 homolog gene is modified to encode a protein that lacks an endogenous N-terminal LCR. In another embodiment of this aspect, the endogenous ALKBH5 homolog gene is modified to include a deletion of at least about 10%, 20%, 30%, 40%, 50%, 60%, 70%, 80%, 90%, or 100% of the nucleotides encoding the N-terminal LCR. In a further embodiment of this aspect, which may be combined with any of the preceding embodiments, the one or more gene editing components include a TALEN, a ZFN, an RNA-guided endonuclease, a fusion protein including an RNA-guided endonuclease, a fusion protein including a reverse transcriptase, a fusion protein including an RNA-guided endonuclease fused to a reverse transcriptase, a guide RNA, a template RNA, and/or a donor oligonucleotide. In some embodiments of this aspect, which may be combined with any of the preceding embodiments, the endogenous ALKBH5 homolog gene is modified to add a nuclear localization signal (NLS) to the encoded protein. In a further embodiment of this aspect, the NLS is encoded by SEQ ID NO: 40. In yet another embodiment of this aspect, the amino acid sequence of the NLS is SEQ ID NO: 41. In one embodiment of this aspect, a heterologous NLS is added.

In another embodiment of this aspect, an endogenous NLS is added. In an additional embodiment of this aspect, the plant is improved in a characteristic selected from biomass, yield, root growth, root expansion, stem cell expansion of the root, stem cell expansion of the shoot, drought resistance, and pest resistance. In another embodiment of this aspect, the plant has improved growth under abiotic stress conditions. In an additional embodiment of this aspect, the plant has improved growth under drought stress, salt stress, heat stress, or cold stress. In yet another embodiment of this aspect, the plant has improved growth under biotic stress conditions. In still another embodiment of this aspect, the plant is improved in a characteristic selected from biomass, yield, root growth, root expansion, stem cell expansion of the root, stem cell expansion of the shoot, drought resistance, and pest resistance. In a further embodiment of this aspect, the plant has elevated photosynthesis.

A further aspect of the disclosure provides a plant produced by the method of any of the preceding embodiments. In one embodiment of this aspect, which may be combined with any of the previous plant embodiments, the plant is selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant of any of the preceding embodiments. An additional aspect of the disclosure provides a commercial product derived from the plant of any of the preceding embodiments, or the seed of any of the preceding embodiments.

### BRIEF DESCRIPTION OF THE DRAWINGS

The present application can be understood by reference to the following description taken in conjunction with the accompanying figures.

The patent or application file contains at least one drawing executed in color. Copies of this patent or patent application publication with color drawing(s) will be provided by the Office upon request and payment of the necessary fee.

FIGS. 1A-1G show analysis of FTO and ALKBH5 demethylases. FIG. 1A shows the three major clades of FTO gene distribution shown by maximum likelihood tree. Adapted from J Mol Evol 66, 80-84 (2008). FIG. 1B shows UHPLC-QQQ-MS/MS results of in vitro biochemistry assay of Micromonas commoda FTO (XP_002502764.1; SEQ ID NO: 32) purified in HEK293T cells. m⁶A-containing ssRNA oligo is used as the substrate. FIG. 1C shows the disordered protein regions in Arabidopsis thaliana ALKBH10B and Micromonas commoda FTO (XP_002502764.1; SEQ ID NO: 32) predicted using IUPred2A and ANCHOR, respectively. The design of low-complexity region (LCR) truncated mutants are shown. FIG. 1D shows a sequence alignment of Homo sapiens FTO (SEQ ID NO: 28), Homo sapiens ALKBH5 (SEQ ID NO: 29), Arabidopsis thaliana ALKBH9B (SEQ ID NO: 30), Arabidopsis thaliana ALKBH10B (SEQ ID NO: 31), Micromonas commoda FTO (XP_002502764.1; SEQ ID NO: 32), Ostreococcus tauri FTO (OUS43030.1; SEQ ID NO:33), Micromonas pusilla FTO (EEH54525.1; SEQ ID NO: 34), and Ostreococcus lucimarinus FTO (ABO99101.1; SEQ ID NO: 35). FIG. 1E shows a cladogram analysis of intrinsically disordered regions (IDRs) in ALKBH5 homologs across plant species. The trait of ALKBH5 lacking an IDR was present in the ancestral land plant A. trichopoda (marked by the asterisk), lost during evolution, and reemerged in select species (highlighted in red). FIG. 1F shows a phylogeny of ALKBH5 and ALKBH5 homologs in soybean, tomato, Arabidopsis, tomato, and human. Variants lacking an IDR are highlighted and labeled. FIG. 1G shows the disordered protein regions in human hALKBH5 (top), soybean GmALKBH5-ΔLCD-1 (second), soybean GmALKBH5-ΔLCD-2 (third), and tomato SIALKBH5-ΔLCD (bottom) predicted using IUPred. A score greater than 0.5 indicates an LCD/IDR.

FIGS. 2A-2D show the effects of overexpression of human FTO on whole-cell LINE1 RNA level in mouse embryonic stem cells (mESCs) and on global chromatin accessibility in Tobacco leaves. FIG. 2A shows a bar graph of the relative expression levels of cellular total LINE1 RNA and indicated young LINE1 subfamily RNAs measured by RT-qPCR with overexpression of human FTO and control empty vector (Ctrl) in mESCs. FIG. 2B shows a bar graph of relative m⁶A fold enrichment of LINE1 RNA and indicated young LINE1 subfamily RNAs from the chromatin-associated fraction of mESCs with overexpression of human FTO and control empty vector (Ctrl) quantified by MeRIP-RT-qPCR. P values were determined using unpaired two-tailed t tests. Ns=no significance; *=P<0.05; **=P<0.01. FIG. 2C shows the results of a DNase I-treated TUNEL assay showing no visible change of chromatin state in mESCs with overexpression of human FTO; the control (Ctrl) was mESCs overexpressed with empty vector. FIG. 2D shows on the left images of a DNase I-treated TUNEL assay showing increased global chromatin accessibility (or more open chromatin) upon transient overexpression of human FTO in the leaves of N. benthamiana using Agrobacterium infiltration. A human FTO catalytically inactive mutant (R316Q/R322Q) was used as Ctrl. On the right, a box-and-whiskers plot shows quantification of the fluorescence intensity from the images on the left. P values were determined using unpaired two-tailed/tests. ****, P<0.0001.

FIGS. 3A-3F show the effects of overexpression of truncated human ALKBH5 variants on whole-cell LINE1 RNA level in mESCs and on global chromatin accessibility in Tobacco leaves. FIG. 3A shows a bar graph of the relative expression levels of cellular total LINE1 RNA and indicated young LINE1 subfamily RNAs measured by RT-qPCR with overexpression of wild-type (WT) and truncated forms of human ALKBH5. mESCs overexpressed with empty vector were used as Ctrl. FIG. 3B shows a bar graph of relative m⁶A fold enrichment of LINE1 RNA and indicated young LINE1 subfamily RNAs from the chromatin-associated fraction of mESCs with overexpression of wild-type (WT) and truncated forms of ALKBH5 variants measured by MeRIP-RT-qPCR. mESCs overexpressed with empty vector were used as Ctrl. P values were determined using unpaired two-tailed/tests. Ns=no significance; *=P<0.05; **=P<0.01. FIG. 3C shows images from a DNase I-treated TUNEL assay showing increased global chromatin accessibility (or more open chromatin) upon transient overexpression of truncated ALKBH5 in the leaves of N. benthamiana using Agrobacterium infiltration. Nuclear localization signal (NLS) was added to ensure nuclear localization. Catalytically inactive mutants (H204A) in all truncated forms (ALKBH5WT^{mut}, ALKBHΔ1^{mut}, ALKBHΔ2^{mut} and ALKBHΔ3^{mut}) were used as controls. Scale bars, 10 μm. P values were determined using unpaired two-tailed t tests. ****=P<0.0001; ns=no significance. FIG. 3D shows prediction of disordered protein regions in the human ALKBH5 sequence using IUPred3, with the design of LCR-truncated constructs illustrated. FIG. 3E shows representative confocal images of mESCs expressing Flag-tagged human FTO, full-length human ALKBH5, or truncated forms of human ALKBH5 protein. Scale bars, 10 μm. FIG. 3F shows root growth phenotype of the wild-type (Col-0) Arabidopsis and three independent human ALKBH5Δ2 overexpressing Arabidopsis lines. Seeds were germinated and grown on MS medium for 10 days. Data are means±SEM (n>10). P values were determined using unpaired two-tailed/tests. ****=P<0.0001. For FIGS. 3A-3F, ALKBH5Δ1 indicated deletion of residues 30-81, ALKBH5Δ2 indicated deletion of residues 298-394, and ALKBH5Δ3 indicated deletion of residues 30-81 and 298-394.

FIGS. 4A-4I show engineering of Arabidopsis and rice ALKBH5 orthologs. FIG. 4A shows amino acid sequence alignments of human ALKBH5 (SEQ ID NO: 36), Arabidopsis ALKBH9B (SEQ ID NO: 37), and rice AlkB family proteins (ALKBH5 orthologs; Os9B=SEQ ID NO: 38; Os10B=SEQ ID NO: 39). Red boxes indicate amino acid identity, red characters show similarity within the highlighted group, and blue frames highlight similarity across groups. Alignment was performed using CLUSTALW and assembly was performed by EsPript 3.0 (PMID: 24753421). The predicted conserved catalytic residues are indicated with asterisks (*). FIG. 4B shows m⁶A/A ratio of non-ribosomal RNA from soluble nuclear fraction (neRNA) quantified by UHPLC-MS/MS from mESCs overexpressed with empty vector, Os9B, and Os10B. P values were determined using unpaired two-tailed/tests. ****=P<0.0001. FIG. 4C shows a prediction of disordered regions in ALKBH9B, ALKBH10B, Os9B, and Os10B using IUPred3. FIG. 4D shows a schematic diagram of the full-length and truncated forms of ALKBH9B, ALKBH10B, Os9B, and Os10B proteins. FIG. 4E shows a bar graph of relative expression levels of cellular total LINE1 RNA and indicated young LINE1 subfamily RNAs measured by RT-qPCR with overexpression of full-length and truncated ALKBH9B, ALKBH10B, Os9B, and Os10B variants. FIG. 4F shows images on the left from a DNase I-treated TUNEL assay showing increased global chromatin accessibility (or more open chromatin) upon transient overexpression of ALKBH9B variants in the leaves of N. benthamiana using agrobacterium infiltration. Wild-type (WT) and catalytical inactive mutants (ALKBH9BWT^{mut}, ALKBH9BΔ1^{mut}, ALKBH9BΔ2^{mut} and ALKBH9BΔ3^{mut}: mutant H335A/D337A) were used as controls. Scale bars, 10 μm. On the right, a box-and-whiskers plot shows quantification of the fluorescence intensity from the images on the left. P values were determined using unpaired two-tailed/tests. ****=P<0.0001; ns=no significance. FIG. 4G shows images on the left from a DNase I-treated TUNEL assay showing increased global chromatin accessibility (or more open chromatin) upon transient overexpression of Os9B and Os10B variants in the leaves of N. benthamiana using agrobacterium infiltration. Wild-type (WT) and catalytical inactive mutants (ALKBH9BWT^{mut}, ALKBH9BΔ1^{mut}, ALKBH9BΔ2^{mut} and ALKBH9BΔ3^{mut}: mutant H335A/D337A; Os9BWT^{mut}, Os9BΔ1^{mut} and Os9BΔ2^{mut}: mutant H324A/D326A; Os10BWT^{mut}, OsTOBΔ1^{mut} and Os10BΔ2^{mut}: mutant H308A/H310A) were used as controls. Scale bars, 10 μm. On the right, a box-and-whiskers plot shows quantification of the fluorescence intensity from the images on the left. P values were determined using unpaired two-tailed t tests. ****=P<0.0001; ns=no significance. FIG. 4H shows on the left root growth phenotype of the wild-type (Col-0) Arabidopsis compared to ALKBH9BΔ2. On the right are box-and-whisker plots of root length. Error bars represent SEM (n>10). P values were determined using unpaired two-tailed/tests. *=P<0.05 FIG. 4I shows root growth phenotype of the wild-type (Col-0) Arabidopsis compared to three independent Os9BΔ1 overexpressing Arabidopsis lines (top row), and three independent Os9BΔ2 overexpressing Arabidopsis lines (bottom row). Seeds were germinated and grown on MS medium for 10 days. Data are means±SEM (n>10). P values were determined using unpaired two-tailed t tests. ****=P<0.0001. For FIGS. 4D-4I, ALKBH9BΔ1 indicated deletion of amino acids 76-102; ALKBH9BΔ2 indicated deletion of amino acids 145-183; ALKBH9BΔ3 indicated deletion of amino acids 432-507; ALKBH10BΔ1 indicated deletion of amino acids 131-190; ALKBH10BΔ2 indicated deletion of residues 501-569; Os9BΔ1 indicated deletion of residues 60-170 and 428-616; Os9BΔ2 indicated deletion of residues 428-616; Os10BΔ1 indicated deletion of residues 2-30, 99-126 and 491-595; Os10BΔ2 indicated deletion of residues 2-30, 99-126 and 389-595.

FIG. 5 shows that full length ALKBH5 does not promote root expansion in Arabidopsis. On the left is a representative image showing seedlings growth on MS medium 10 days after germination for wild-type Col-0 and ALKBH5 overexpression plants. On the right is a bar graph of measurements of root lengths. Error bars represent SEM (n>10). P values were determined using unpaired two-tailed t tests. ns=no significance.

FIGS. 6A-6T show that ALKBH5 cIDR deletion induces open chromatin and transcription upregulation in mESCs. FIG. 6A shows a Western blot analysis confirming the knockout of Alkbh5 in mESCs for Alkbh5 KO-1 and Alkbh5 KO-2 clones, with GAPDH used as a loading control. FIG. 6B shows bar graphs of quantification of the m⁶A/A ratio by LC-MS/MS in (left) whole-cell polyadenylated RNA and (right) non-ribosomal caRNA extracted from WT and Alkbh5^{-/-} mESCs. Data are shown as mean±SEM from three independent experiments. Unpaired two-tailed t tests; n.s., not significant; **p<0.01; ****p<0.0001. FIG. 6C shows the IUPred3 score of human ALKBH5 (top) and FTO (bottom). A score greater than 0.5 indicates an IDR. FIG. 6D shows schematics of Flag-tagged human ALKBH5 variants (top), and representative images of mESCs expressing the indicated Flag-tagged variants (bottom; scale bars=10 μm). The mESC images are identical to those in FIG. 3E, but the labels read “hFTO” for FLAG-tagged human FTO instead of “Flag-FTO” for the same FLAG-tagged human FTO. Similar label changes are made for ALKBH5, ALKBH5Δ1, and ALKBH5Δ2. FIG. 6E shows the generation of HA-tagged truncated mouse ALKBH5. Top: IUPred3 score of mouse ALKBH5. A score greater than 0.5 indicates an IDR. The IDR distribution in human and mouse ALKBH5 are similar. Middle: Schematic diagram illustrating the CRISPR-Cas9 strategy used to delete the cIDR of endogenous ALKBH5 in mESCs. Bottom: Representative Sanger sequencing chromatogram confirming the desired HA-tagged stop codon knock-in at the targeted Alkbh5 locus in mESCs. FIG. 6F shows a Western blot analysis of full-length mALKBH5 and mALKBH5-ΔcIDR proteins from WT and Alkbh5-ΔcIDR mESCs, respectively. Left: detection of full-length mALKBH5 in WT mESCs and mALKBH5-ΔcIDR in Alkbh5-ΔcIDR mESCs. Right: confirmation of HA-tag knock-in at the endogenous Alkbh5 locus in Alkbh5-ΔcIDR mESCs. GAPDH was used as a loading control. FIG. 6G shows nascent RNA synthesis in WT, Alkbh5 KO and Alkbh5-ΔcIDR mESCs measured by 5-ethynyl uridine (EU) incorporation. Left: representative images. Right: boxplots representing relative EU fluorescence intensity (n>550 cells per condition). Unpaired two-tailed t tests; n.s., not significant; ****p<0.0001. Scale bars, 50 μm. FIG. 6H shows chromatin accessibility analysis of WT, Alkbh5 KO and Alkbh5-ΔcIDR mESCs revealed by TUNEL signals. FIG. 6I shows Venn diagrams of the overlaps of eCLIP-seq peaks acquired from two biological replicates with high reproducibility. FIG. 6J shows scatter plots showing the correlation between two biological replicates of eCLIP-seq for full-length mALKBH5 (left) and mALKBH5-ΔcIDR (right). FIG. 6K shows a Venn diagram illustrating the overlap of eCLIP-seq peaks between full-length mALKBH5 and mALKBH5-ΔcIDR. FIG. 6L shows aggregation profiles showing the meta distributions of eCLIP-seq peaks for full-length mALKBH5 and mALKBH5-ΔcIDR, along with m⁶A signals in WT mESCs across protein-coding genes. FIG. 6M shows heatmaps showing m⁶A levels on mRNA, transcriptional activity, and histone modifications across different eCLIP peak categories. FIG. 6N shows a genome browser visualization of chromatin states at differential eCLIP-seq peaks between full-length mALKBH5 and mALKBH5-ΔcIDR. Regions enriched in mALKBH5-ΔcIDR are highlighted in red, while regions enriched in full-length mALKBH5 are highlighted in blue. FIG. 6O shows bar graphs of the proportion of eCLIP-seq peak of full-length mALKBH5 and mALKBH5-ΔcIDR overlapping with m⁶A peaks on mRNA. FIG. 6P shows bar graphs of the percentage of eCLIP-seq peaks overlapping with different RNA types. FIG. 6Q shows differential transcription factor motif enrichment analysis in FL-unique and ΔcIDR-unique peaks. FIG. 6R shows a comparison of m⁶A levels (top) and eCLIP-seq signals (bottom) between full-length mALKBH5 and mALKBH5-ΔcIDR at FTO regulatory regions. FIG. 6S shows chromatin accessibility and transcriptional changes across different eCLIP peak categories in Alkbh5-ΔcIDR mESCs expressing mALKBH5-ΔcIDR compared to WT mESCs expressing full-length mALKBH5 (ΔcIDR/FL). Points indicate medians, and error bars represent SEM. Different letters denote statistically significant differences (Fisher's LSD test). FIG. 6T shows frequency distribution of upregulated ATAC, caRNA, and mRNA signals in Alkbh5-ΔcIDR mESCs relative to the nearest ΔcIDR-unique peaks. For FIGS. 6F and 61-6T, FL represents full-length mALKBH5 and ΔcIDR represents mALKBH5-ΔcIDR.

FIGS. 7A-7AD show that cIDR deletion redirects mALKBH5 to chromatin-associated repeat RNAs in mESCs. FIG. 7A shows a Venn diagram showing the overlap of differentially methylated m6A regions (DMRs) between conditions. Significance was assessed using a one-sided Fisher's exact test. Expected overlap is indicated. n.s., not significant. FIG. 7B shows enrichment analysis of DMRs across RNA categories, highlighting over-representation within specific RNA types. FIG. 7C shows the genomic distribution of hypermethylated m⁶A peaks for FTO (Fto^{-/-} vs WT mESCs) and ALKBH5 (Alkbh5^{-/-} vs WT mESCs), and hypomethylated m⁶A peaks for ALKBH5-ΔcIDR (Alkbh5-ΔcIDR vs WT mESCs) across distinct genomic regions. FIG. 7D shows boxplots showing m⁶A methylation levels of exons and introns in camRNA from WT and Alkbh5^{-/-} mESCs. Mann-Whitney U test, ****p<0.0001. FIG. 7E shows aggregation plots showing eCLIP-seq signals for full-length mALKBH5 (FL) and mALKBH5-ΔcIDR across hypermethylated regions upon Alkbh5 KO (left) and hypomethylated regions upon Alkbh5-ΔcIDR (right). FIG. 7F shows fold changes in expression levels of m⁶A-marked and non-m⁶A-marked camRNA in Alkbh5^{-/-} versus WT mESCs. Mann-Whitney U test, ****p<0.0001. FIG. 7G shows fold changes in expression levels of m⁶A-marked and non-m⁶A-marked carRNA in Alkbh5-ΔcIDR versus WT mESCs. Mann-Whitney U test, ****p<0.0001. FIG. 7H shows scatter plots showing the negative correlation of fold changes between m⁶A levels and expression levels for camRNA upon Alkbh5 KO (left) and for carRNA upon Alkbh5-ΔcIDR (right). Pearson's correlation coefficients and p values are shown. FIG. 7I shows a summary of repeat RNAs upon Alkbh5-ΔcIDR. Top: number of hypomethylated peaks across repeat subfamilies. Middle: proportion of hypomethylated peaks overlapping specific repeat subfamilies, normalized to the total hypomethylated peaks. Bottom: fold changes in m⁶A level for the indicated subfamilies. FIG. 7J shows fold changes in expression levels for the indicated repeat subfamilies in Alkbh5-ΔcIDR versus WT mESCs. FIG. 7K shows gene set enrichment analysis (GSEA) illustrating the enrichment of DMRs within repeat subfamilies in Alkbh5-ΔcIDR versus WT mESCs. Significant repeat subfamilies are highlighted in red. FIG. 7L shows a scatter plot showing the negative correlation of fold changes between m⁶A and expression levels for enriched LINE1 subfamilies in Alkbh5-ΔcIDR versus WT mESCs. Pearson's correlation coefficients and p values are shown. FIG. 7M shows fold changes in m⁶A levels on young and old LINE1 RNAs in Alkbh5-ΔcIDR versus WT mESCs. ‘Random’ represents repeat RNAs randomly selected across the genome and matched in number to LINE1 RNAs. Mann-Whitney U test, n.s., not significant; ****p<0.0001. FIG. 7N shows aggregation plots showing changes in m⁶A levels (left) and expression levels (right) of repeat RNAs within mFTO regulatory regions in Alkbh5-ΔcIDR and WT mESCs. FIG. 7O shows the half lifetime of nuclear LINE1 RNA in WT and Alkbh5-ΔcIDR mESCs, measured by RT-qPCR after actinomycin D (ActD) treatment. Data are shown as mean±SEM from three independent experiments. FIG. 7P shows bar graphs of CLIP-qPCR showing relative enrichment of LINE1 RNA by YTHDC1 in WT and Alkbh5-ΔcIDR mESCs. Data are represented as mean±SEM from three independent experiments. Unpaired two-tailed t test, *p<0.05, **p<0.01. FIG. 7Q shows a heatmap showing nascent repeat RNAs transcription levels over time in WT, Alkbh5-ΔcIDR, and Alkbh5 KO mESCs. FIG. 7R shows line graphs of nascent repeat RNA transcription levels over time in WT, Alkbh5 KO and Alkbh5-ΔcIDR mESCs. Data represent mean transcription levels normalized to the 10-minute time point. Statistical differences between groups were determined using Fisher's LSD test. FIG. 7S shows transcription rate changes of repeat RNAs at hypomethylated regions upon Alkbh5-ΔcIDR (ΔcIDR/WT) and hypermethylated regions upon Alkbh5 KO (Alkbh5^{-/-}/WT) (left), and comparison of transcription rate changes between LINE1 repeats and other repeats in Alkbh5-ΔcIDR and Alkbh5/mESCs relative to WT mESCs (right). Points represent the medians; error bars represent SEM. Mann-Whitney U test; n.s., not significant; **p<0.01; ****p<0.0001. FIG. 7T shows the proportion of hypomethylated LINE1 RNAs located in intergenic versus intragenic regions upon Alkbh5-ΔcIDR (left); and fold changes in m⁶A levels of LINE1 RNAs in intergenic versus intragenic regions upon Alkbh5-ΔcIDR (right). Mann-Whitney U test, ****p<0.0001. FIG. 7U shows a schematic model illustrating distinct substrate specificities of ALKBH5, FTO, and ALKBH5-ΔcIDR on chromatin. FIG. 7V shows gel images of co-immunoprecipitation assays in WT and Alkbh5-ΔcIDR mESCs demonstrating that mALKBH5 interacts with components of EJC, while mFTO and HA-tagged mALKBH5-ΔcIDR don't show such interactions. FIG. 7W shows on top: a schematic of human ALKBH5 variants ectopically expressed in Fto^{-/-} mESCs, and on bottom: growth curves of WT and Fto^{-/-} mESCs expressing the indicated variants. Fto^{-/-} mESCs transfected with empty vectors served as controls (Ctrl). FIG. 7X shows a heatmap showing expression levels of differentiation markers in WT mESCs and Fto^{-/-} mESCs ectopically expressing empty vector (Ctrl), human ALKBH5, ALKBH5Δ1 or ALKBH5Δ2 variants. WT mESCs, Fto^{-/-} mESCs rescued by human FTO, and Fto^{-/-} mESCs rescued by human ALKBH5Δ2 were clustered together. FIG. 7Y shows on top: a schematic of the FTO-cIDR fusion variant, with the cIDR of human ALKBH5 fused to the C-terminus of human FTO; on bottom: representative immunofluorescence images of mESCs expressing Flag-tagged FTO-cIDR. FIG. 7Z shows LC-MS/MS quantification of the m⁶A/A ratio in (left) whole cell polyadenylated RNA and (right) non-ribosomal caRNA isolated from mESCs ectopically expressing empty vector (Ctrl), human FTO, or FTO-CIDR. Data are shown as mean±SEM from three independent experiments. Unpaired two-tailed t tests; n.s., not significant; **p<0.01; ***p<0.001. FIG. 7AA shows growth curves showing reduced proliferation rate of Alkbh5^{-/-} mESCs compared to WT mESCs. FIG. 7AB shows bar graphs of relative expression levels of differentiation markers in embryoid bodies (EBs) derived from WT and Alkbh5^{-/-} mESCs. Data are shown as mean±SEM from three independent experiments. WT mESCs served as control, and p values represent comparisons to the WT group, determined using unpaired two-tailed t tests. *p<0.05; **p<0.01. FIG. 7AC shows growth curves of WT and Alkbh5^{-/-} mESCs ectopically expressing the indicated variants. Alkbh5^{-/-} mESCs expressing empty vector served as Ctrl. FIG. 7AD shows a heatmap showing expression levels of differentiation markers in WT mESCs and Alkbh5^{-/-} mESCs ectopically expressing empty vector (Ctrl), human FTO and FTO-cIDR. WT mESCs and Alkbh5^{-/-} mESCs rescued by FTO-cIDR were clustered together. In FIGS. 7W, 7AA, and 7AC, cell numbers at each time point were normalized to cell numbers on Day 1. Statistical significance was assessed based on cell numbers on the last day. Data are shown as mean±SEM from three independent experiments. Unpaired two-tailed t tests; n.s., not significant; ****p<0.0001.

FIGS. 8A-8P show that m⁶A-hypomethylated LINE1 acts as enhancer-like element to modulate chromatin state. FIG. 8A shows a Jaccard index showing the overlap between hypomethylated m⁶A peaks upon Alkbh5-ΔcIDR and ChIP-seq peaks of chromatin modifiers and histone modifications. FIG. 8B shows profiles of H3K27ac and H3K4me3 in WT mESCs and Alkbh5-ΔcIDR mESCs across the genome (top) and at LINE1 RNA-targeted genomic sites (bottom). FIG. 8C shows genome-wide profiles of H3K9me3 in WT and Alkbh5-ΔcIDR mESCs. FIG. 8D shows scatter plots showing the lack of significant correlation between fold changes in m⁶A levels and H3K9me3 levels in Alkbh5-ΔcIDR versus WT mESCs. FIG. 8E shows scatter plots showing the negative correlation between changes in m⁶A levels and H3K27ac (left) or H3K4me3 (right) levels in Alkbh5-ΔcIDR versus WT mESCs. Pearson's correlation coefficient and p values shown. FIG. 8F shows profiles of H3K27ac (left) and H3K4me3 (right) in WT and Alkbh5-ΔcIDR mESCs at hypomethylated m⁶A regions, respectively. FIG. 8G shows aggregation plot showing chromatin accessibility in WT and Alkbh5-ΔcIDR mESCs within LINE1 RNA-targeted genomic sites. FIG. 8H shows profiles of YY1 and P300 binding in WT and Alkbh5-ΔcIDR mESCs at upregulated H3K27ac regions (top) and upregulated H3K4me3 regions (bottom). Upregulated H3K27ac and H3K4me3 regions were identified by comparing Alkbh5-ΔcIDR mESCs to WT mESCs. FIG. 8I shows scatter plots showing the positive correlation between changes in H3K27ac levels and changes in P300 binding (left) or YY1 binding (right) in Alkbh5-ΔcIDR versus WT mESCs. FIG. 8J shows aggregation plots showing H3K27ac levels across LINE1 elements with ±2 kb flanking regions in WT and Alkbh5-ΔcIDR mESCs. FIG. 8K shows the proportion of loop-related m⁶A-hypomethylated LINE1 regions located within enhancers in Alkbh5-ΔcIDR mESCs. FIG. 8L shows on the left, a schematic illustrating the concepts of “loop-related peaks” and “random peaks”, and on the right: fold changes of chromatin accessibility (ATAC-seq), H3K27ac and H3K4me3 occupancy, and mRNA abundance at loop-related peaks and random peaks in Alkbh5-ΔcIDR versus WT mESCs. Mann-Whitney U test; n.s., not significant; **p<0.01; ****p<0.0001. FIG. 8M shows a genome browser visualization of histone modification changes at m⁶A-hypomethylated LINE1-related enhancer-promoter loops. m⁶A-hypomethylated LINE1 regions are highlighted in red, and promoter regions are highlighted in gray. FIG. 8N shows GO analysis of genes upregulated through m⁶A-hypomethylated LINE1-related loops in Alkbh5-ΔcIDR mESCs. BP, Biological Process; MF, Molecular Function; CC, Cellular Component. FIG. 8O shows GO analysis of upregulated genes in Alkbh5-ΔcIDR mESCs versus WT mESCs. Enriched GO terms are categorized into Biological Process (BP, blue), Molecular Function (MF, green), and Cellular Component (CC, purple). FIG. 8P shows a schematic model illustrating how ALKBH5-ΔcIDR-mediated hypomethylation of intergenic LINE1 elements modulates chromatin states.

FIGS. 9A-9V shows a systematic workflow for engineering ALKBH5 and its plant homologs for carRNA m⁶A demethylation. FIG. 9A shows a schematic of the workflow for engineering ALKBH5 homologs. FIG. 9B shows RT-qPCR analysis of whole-cell total LINE1 RNA and young LINE1 subfamilies RNAs in mESCs ectopically expressing human FTO. Empty vector-transfected mESCs served as controls (Ctrl). Data are shown as mean±SEM from three independent experiments. Unpaired two-tailed t tests; n.s., not significant; *p<0.05; **p<0.01; ***p<0.001; ****p<0.0001. Data presented include the data in FIG. 2A and two additional biological replicates. FIG. 9C shows RT-qPCR analysis of whole-cell total LINE1 RNA and young LINE1 subfamilies RNAs in mESCs ectopically expressing human ALKBH5. Empty vector-transfected mESCs served as controls (Ctrl). Data are shown as mean±SEM from three independent experiments. Unpaired two-tailed t tests; n.s., not significant; *p<0.05; **p<0.01; ***p<0.001; ****p<0.0001. Data presented include the data in FIG. 3A and two additional biological replicates. FIG. 9D shows schematics of human ALKBH5 variants ectopically expressed in mESCs, transiently infiltrated into tobacco leaves, and stably transformed into Arabidopsis. FIG. 9E shows relative expression levels of whole-cell total LINE1 RNA and young LINE1 subfamilies RNAs measured by RT-qPCR in mESCs ectopically expressing catalytically inactive human ALKBH5 variants. Data represent means±SEM from three independent experiments. Unpaired two-tailed t tests; n.s., not significant. FIG. 9F shows relative m⁶A enrichment on chromatin-associated total LINE1 RNA and young LINE1 subfamilies RNAs measured by MeRIP-qPCR in mESCs ectopically expressing human FTO variants. Empty vector-transfected mESCs served as controls (Ctrl). Data represent means±SEM from three independent experiments. Unpaired two-tailed t tests; n.s., not significant; *p<0.05; **p<0.01; ***p<0.001. Data presented include the data in FIG. 2B and two additional biological replicates. FIG. 9G shows relative m⁶A enrichment on chromatin-associated total LINE1 RNA and young LINE1 subfamilies RNAs measured by MeRIP-qPCR in mESCs ectopically expressing ALKBH5 variants. Empty vector-transfected mESCs served as controls (Ctrl). Data represent means±SEM from three independent experiments. Unpaired two-tailed t tests; n.s., not significant; *p<0.05; **p<0.01; ***p<0.001. Data presented include the data in FIG. 3B and two additional biological replicates. FIG. 9H shows DNase I-treated TUNEL assays showing increased global chromatin accessibility in tobacco leaves transiently overexpressing human FTO. Boxplots represent relative TUNEL fluorescence intensity. Unpaired two-tailed t tests; ****p<0.0001. Scale bars, 50 μm. Leaves infiltrated with empty vector served as controls (Ctrl). FIG. 9I shows DNase I-treated TUNEL assays showing increased global chromatin accessibility in tobacco leaves transiently overexpressing IDR-deleted human ALKBH5 variants. Boxplots represent relative TUNEL fluorescence intensity. Unpaired two-tailed t tests; ****p<0.0001. Scale bars, 50 μm. Data presented include the data in FIG. 3C and two additional biological replicates. FIG. 9J shows DNase I-treated TUNEL assays showing that chromatin opening induced by IDR-deleted human ALKBH5 variants in tobacco leaves is m⁶A-dependent. TUNEL fluorescence intensity data for ALKBH5, ALKBH5Δ1, ALKBH5Δ2 and ALKBH5Δ3 is derived from FIG. 9I. Unpaired two-tailed t tests; n.s., not significant; ****p<0.0001. FIG. 9K shows a Western blot analysis showing the expression of Flag-tagged human ALKBH5 variants and FTO in Arabidopsis transgenic lines. β-Actin was used as a loading control. FIG. 9L shows root growth phenotypes of 10-day-old Arabidopsis transgenic lines expressing human FTO. Representative images are shown on the left, and primary root length measurements are shown on the right. Data are shown as mean±SEM. Unpaired two-tailed t tests; n.s., not significant; *p<0.05; ***p<0.001; ****p<0.0001. Scale bars, 1 cm. FTO^{mut} indicates R316Q/R322Q mutation. FIG. 9M shows root growth phenotypes of 10-day-old Arabidopsis transgenic lines expressing human ALKBH5 variants as indicated in FIG. 9D. Representative images are shown on the left, and primary root length measurements are shown on the right. Data are shown as mean±SEM. Unpaired two-tailed t tests; n.s., not significant; *p <0.05; ***p<0.001; ****p<0.0001. Scale bars, 1 cm. FIG. 9N shows root growth phenotypes of 10-day-old Arabidopsis transgenic lines expressing human ALKBH5 variants as indicated in FIG. 9D. Representative images are shown on the left, and primary root length measurements are shown on the right. Data are shown as mean±SEM. Unpaired two-tailed t tests; n.s., not significant; *p<0.05; ***p<0.001; ****p<0.0001. Scale bars, 1 cm. FIG. 9O shows root growth phenotypes of 10-day-old Arabidopsis transgenic lines expressing human ALKBH5 variants as indicated in FIG. 9D. Representative images are shown on the left, and primary root length measurements are shown on the right. Data are shown as mean±SEM. Unpaired two-tailed t tests; n.s., not significant; *p<0.05; ***p<0.001; ****p<0.0001. Scale bars, 1 cm. FIG. 9P shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. FIG. 9Q shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. FIG. 9R shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. FIG. 9S shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. FIG. 9T shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. FIG. 9U shows primary root length measurements of 10-day-old Arabidopsis lines expressing indicated ALKBH5 variants. Data are shown as means±SEM. Unpaired two-tailed t tests; n.s., not significant; *p<0.05; **p <0.01; ****p<0.0001. FIG. 9V shows primary root length measurements of 10-day-old Arabidopsis lines expressing indicated ALKBH5 variants. Data are shown as means±SEM. Unpaired two-tailed t tests; n.s., not significant; *p<0.05; **p<0.01; ****p<0.0001. For FIGS. 9A-9V, truncation variants of ALKBH5 correspond to specific residue deletions as detailed in FIG. 9D; FL represents full-length; ALKBH5^{mut}, ALKBH5Δ1^{mut}, ALKBH5Δ2^{mut} and ALKBH5Δ3^{mut} indicate variants with H204A mutation.

FIGS. 10A-10P show that IDR-deleted ALBKH5 mediates caRNA m⁶A demethylation and chromatin activation in Arabidopsis. FIG. 10A shows on top: Venn diagram showing overlap of Flag CUT&RUN peaks between Flag-tagged ALKBH5 and ALKBH5-ΔcIDR in Arabidopsis; and on bottom: Profiles of Flag CUT&RUN signals for ALKBH5 and ALKBH5-ΔcIDR. FIG. 10B shows profiles of H3K27ac (top) and H3K4me3 (bottom) signals in ALKBH5- and ALKBH5-ΔcIDR-Arabidopsis. FIG. 10C shows a Volcano plot showing differential changes in H3K4mc3 signal in ALKBH5-ΔcIDR-Arabidopsis versus ALKBH5-Arabidopsis. FIG. 10D shows a Volcano plot showing differential changes in H3K27ac signal in ALKBH5-ΔcIDR-Arabidopsis versus ALKBH5-Arabidopsis. FIG. 10E shows profiles of ATAC signals in ALKBH5- and ALKBH5-ΔcIDR-Arabidopsis. FIG. 10F shows Venn diagrams of the overlap between Flag CUT&RUN peaks and H3K27ac-marked regions (left) or H3K4me3-marked regions (right) in Flag-tagged ALKBH5-ΔcIDR-Arabidopsis. FIG. 10G shows scatter plots showing the positive correlations between fold changes in Flag CUT&RUN signals and H3K27ac (left) or H3K4me3 (right) levels in ALKBH5-ΔcIDR-versus ALKBH5-Arabidopsis. Pearson's correlation test. FIG. 10H shows fold changes in m⁶A levels (left) and expression levels of m⁶A-marked caRNAs (right) in ALKBH5- and ALKBH5-ΔcIDR-Arabidopsis relative to catalytically inactive mutant lines. Mann-Whitney U test, ****p<0.0001. FIG. 10I shows boxplots showing H3K27ac (left) and H3K4me3 (right) signals at hypomethylated m⁶A regions in ALKBH5-ΔcIDR- and ALKBH5-Arabidopsis, respectively. Hypomethylated m⁶A regions were identified by comparing ALKBH5-ΔcIDR-to ALKBH5-Arabidopsis. Mann-Whitney U test, n.s., not significant; ****p<0.0001. FIG. 10J shows comparative analysis of m⁶A levels and expression of caRNAs between ALKBH5-ΔcIDR- and ALKBH5-Arabidopsis. Left: percentage of hypomethylated m⁶A peaks across RNA categories in ALKBH5-ΔcIDR-Arabidopsis relative to ALKBH5-Arabidopsis. Middle: fold changes in m⁶A levels across RNA categories in ALKBH5-ΔcIDR-versus ALKBH5-Arabidopsis. Right: fold changes in expression levels across RNA categories in ALKBH5-ΔcIDR-versus ALKBH5-Arabidopsis. FIG. 10K shows a heatmap of gene expression changes in ALKBH5- and ALKBH5-ΔcIDR-Arabidopsis compared to catalytically inactive mutant lines. FIG. 10L shows GO enrichment analysis of upregulated genes in ALKBH5-ΔcIDR-Arabidopsis relative to ALKBH5-Arabidopsis. FIG. 10M shows chromatin states analysis of At3g56825 (U2.4) interacted sites in ALKBH5- and ALKBH5-ΔcIDR-Arabidopsis. Left: proportion of At3g56825 interacted sites marked with H3K27ac. Right: fold changes of H3K27ac signal on At3g56825 interacted sites in ALKBH5-ΔcIDR-versus ALKBH5-Arabidopsis. FIG. 10N shows chromatin states analysis of At3g56825 (U2.4) interacted sites in ALKBH5- and ALKBH5-ΔcIDR-Arabidopsis. Left: proportion of At3g56825 interacted sites marked with H3K4me3. Right: fold changes of H3K4me3 signals on At3g56825 interacted sites in ALKBH5-ΔcIDR-versus ALKBH5-Arabidopsis. FIG. 10O shows chromatin states analysis of At3g56705 (U2.6) interacted sites in ALKBH5- and ALKBH5-ΔcIDR-Arabidopsis. Pie charts show the proportion of sites marked with H3K27ac and H3K4me3 across At3g56705 interacted regions. Violin plots show fold changes of H3K27ac (left) or H3K4me3 (right) signals on At3g56705 interacted sites in ALKBH5-ΔcIDR-versus ALKBH5-Arabidopsis. FIG. 10P shows chromatin states analysis of At5g61455 (U2.7) interacted sites in ALKBH5- and ALKBH5-ΔcIDR-Arabidopsis. Pie charts show the proportion of sites marked with H3K27ac and H3K4me3 across At5g61455 interacted regions. Violin plots show fold changes of H3K27ac (left) or H3K4me3 (right) signals on At5g61455 interacted sites in ALKBH5-ΔcIDR-versus ALKBH5-Arabidopsis.

FIGS. 11A-11AI show engineering Arabidopsis and rice ALKBH5 orthologs to promote plant growth. FIG. 11A shows the m⁶A/A ratio of non-ribosomal RNA from the soluble nuclear fraction (nuRNA) quantified by LC-MS/MS in mESCs ectopically expressing empty vector (Ctrl), Os9B, and Os10B. Data are shown as means±SEM from three independent experiments. Unpaired two-tailed t tests; ****p<0.0001. Data are comparable to those in FIG. 4B, with minor changes in the y-axis labeling. FIG. 11B shows on top: a prediction of IDRs in ALKBH9B, Os9B and Os10B using IUPred3. A score greater than 0.5 indicates the presence of an IDR. IUPred3 analyses are comparable to those in FIG. 4C; on bottom: schematics of the different variants of ALKBH9B, Os9B, and Os10B that were ectopically expressed in mESCs, transiently infiltrated into tobacco leaves, and stably transformed into Arabidopsis. FIG. 11C shows RT-qPCR analysis of whole-cell total LINE1 RNA and young LINE1 subfamilies RNAs in mESCs ectopically expressing Arabidopsis ALKBH9B variants as shown in FIG. 11B. Empty vector-transfected mESCs served as controls (Ctrl). Data are shown as mean±SEM from three independent experiments. Unpaired two-tailed t tests; n.s., not significant; *p<0.05; **p<0.01; ***p<0.001; ****p<0.0001. FIG. 11D shows RT-qPCR analysis of whole-cell total LINE1 RNA and young LINE1 subfamilies RNAs in mESCs ectopically expressing rice Os9B variants as shown in FIG. 11B. Empty vector-transfected mESCs served as controls (Ctrl). Data are shown as mean±SEM from three independent experiments. Unpaired two-tailed t tests; n.s., not significant; *p<0.05; **p<0.01; ***p<0.001; ****p<0.0001. Data presented include the data from FIG. 4E and two additional biological replicates; Os9BΔ1 here is equivalent to Os9BΔ2 in FIG. 4E, and Os9BΔ2 here is equivalent to Os9BΔ1 in FIG. 4E. FIG. 11E shows relative expression levels of whole-cell total LINE1 RNA and young LINE1 subfamilies RNAs measured by RT-qPCR in mESCs ectopically expressing catalytically inactive variants of Arabidopsis ALKBH9B. Data represent mean±SEM from three independent experiments. Unpaired two-tailed t tests; n.s., not significant. FIG. 11F shows relative expression levels of whole-cell total LINE1 RNA and young LINE1 subfamilies RNAs measured by RT-qPCR in mESCs ectopically expressing catalytically inactive variants of rice Os9B. Data represent mean±SEM from three independent experiments. Unpaired two-tailed t tests; n.s., not significant. FIG. 11G shows relative expression levels of whole-cell total LINE1 RNA and young LINE1 subfamilies RNAs measured by RT-qPCR in mESCs ectopically expressing catalytically active variants of rice Os10B. Data represent mean±SEM from three independent experiments. Unpaired two-tailed t tests; n.s., not significant. FIG. 11H shows relative expression levels of whole-cell total LINE1 RNA and young LINE1 subfamilies RNAs measured by RT-qPCR in mESCs ectopically expressing catalytically inactive variants of rice Os10B. Data represent mean±SEM from three independent experiments. Unpaired two-tailed t tests; n.s., not significant. FIG. 11I shows RT-qPCR analysis of whole cell total LINE1 RNA and young LINE1 subfamilies RNAs in mESCs ectopically expressing Arabidopsis ALKBH10B variants. ALKBH10BΔ1 refers to deletion of residues 131-190, ALKBH10BA2 refers to deletion of residues 501-569. FIG. 11J shows images from DNase I-treated TUNEL assays showing increased global chromatin accessibility in tobacco leaves transiently overexpressing IDR-deleted variants of Arabidopsis ALKBH9B. Boxplots represent relative TUNEL fluorescence intensity. Unpaired two-tailed t tests; ****p<0.0001. Scale bars, 50 μm. Data are comparable to those in FIG. 4F. FIG. 11K shows images from DNase I-treated TUNEL assays showing increased global chromatin accessibility in tobacco leaves transiently overexpressing IDR-deleted variants of rice Os9B. Boxplots represent relative TUNEL fluorescence intensity. Unpaired two-tailed t tests; ****p<0.0001. Scale bars, 50 μm. Data shows are comparable to data in FIG. 4G. FIG. 11L shows analysis from DNase I-treated TUNEL assays showing that chromatin opening induced by IDR-deleted ALKBH9B, Os9B or Os10B variants in tobacco leaves is m⁶A-dependent. TUNEL fluorescence intensity data for ALKBH9B, ALKBH9BΔ1, ALKBH9BΔ2, ALKBH9BΔ3, Os9B, Os9BΔ1 and Os9BΔ2 derived from FIGS. 11J-11K. Unpaired two-tailed t tests; n.s., not significant; ****p<0.0001. FIG. 11M shows Western blot analysis showing the expression of GFP-Flag-tagged ALKBH9B variants, and Flag-tagged Os9B and Os10B variants in Arabidopsis transgenic lines. GFP tag was added to enhance expression levels of ALKBH9B. β-Actin was used as a loading control. FIG. 11N shows root growth phenotypes of 10-day-old Arabidopsis transgenic lines expressing variants of Arabidopsis ALKBH9B. Representative images are shown on the left, and primary root length measurements are shown on the right. Data are shown as mean±SEM. Unpaired two-tailed t tests; n.s., not significant; ****p<0.0001. Scale bars, 1 cm. ALKBH9BΔ3^{mut} indicates the variant with H335A/D337A mutation. FIG. 11O shows root growth phenotypes of 10-day-old Arabidopsis transgenic lines expressing variants of rice Os9B. Representative images are shown on the left, and primary root length measurements are shown on the right. Data are shown as mean±SEM. Unpaired two-tailed t tests; n.s., not significant; ****p <0.0001. Scale bars, 1 cm. ALKBH9BΔ3^{mut} indicates the variant with H335A/D337A mutation; Os9BΔ1^{mut} and Os9BΔ2^{mut} indicate variants with H324A/D326A mutation. FIG. 11P shows root growth phenotypes of 10-day-old Arabidopsis transgenic lines expressing variants of rice Os9B. Representative images are shown on the left, and primary root length measurements are shown on the right. Data are shown as mean±SEM. Unpaired two-tailed t tests; n.s., not significant; ****p <0.0001. Scale bars, 1 cm. ALKBH9BΔ3^{mut} indicates the variant with H335A/D337A mutation; Os9BΔ1^{mut} and Os9BΔ2^{mut} indicate variants with H324A/D326A mutation. FIG. 11Q shows primary root length measurements of 10-day-old Arabidopsis lines expressing indicated ALKBH9B, Os9B, or Os10B variants. Data are shown as mean±SEM. Unpaired two-tailed t tests; n.s., not significant; *p<0.05; ****p<0.0001. FIG. 11R shows MeRIP-qPCR showing relative m⁶A methylation levels of chromatin-associated non-coding RNAs in transgenic Arabidopsis lines expressing full-length or cIDR-deleted variants of ALKBH9B, Os9B and Os10B. Data are shown as means±SEM from three independent experiments. Unpaired two-tailed t tests; n.s., not significant; *p<0.05; **p<0.01; ***p<0.001; ****p<0.0001. FIG. 11S shows relative expression levels of chromatin-associated non-coding RNAs in transgenic Arabidopsis lines expressing full-length or cIDR-deleted variants of ALKBH9B, Os9B and Os10B. Data are shown as means±SEM from three independent experiments. Unpaired two-tailed t tests; n.s., not significant; *p<0.05; **p<0.01; ***p<0.001; ****p<0.0001. FIG. 11T shows Western blot analysis showing H3K4me3 and H3K27ac levels in transgenic Arabidopsis lines expressing full-length or cIDR-deleted variants of ALKBH9B, Os9B and Os10B. Histone H3 was used as a loading control. Data are shown as means±SEM from three independent experiments. Unpaired two-tailed t tests; n.s., not significant; *p<0.05; **p<0.01; ***p<0.001; ****p<0.0001. FIG. 11U shows relative expression levels of human ALKBH5 variants in three independent transgenic rice lines, measured by RT-qPCR. WT ZH11 served as the control. FIG. 11V shows root length of 15-day-old WT (ZH11) and transgenic rice seedlings expressing human ALKBH5 variants grown under hydroponic culture conditions. Representative images are shown. Scale bars, 10 cm. FIG. 11W shows root length of 15-day-old WT (ZH11) and transgenic rice seedlings expressing human ALKBH5 variants grown under hydroponic culture conditions. FIG. 11X shows tiller number per plant of WT (ZH11) and transgenic rice lines expressing human ALKBH5 variants grown in the greenhouse. Representative images are shown. Scale bars, 10 cm. FIG. 11Y shows total grain number per plant of WT (ZH11) and transgenic rice lines expressing human ALKBH5 variants grown in the greenhouse. Representative images are shown. Scale bars, 4.5 cm (J). FIG. 11Z shows dry biomass per plant of WT (ZH11) and transgenic rice lines expressing human ALKBH5 variants grown in the greenhouse. FIG. 11AA shows photosynthetic rate measurement of WT (ZH11) and transgenic rice lines expressing human ALKBH5 variants grown in the greenhouse. Representative images are shown. Scale bars, 10 cm. FIG. 11AB shows photosynthetic rate measurement of WT (ZH11) and transgenic rice lines expressing human ALKBH5 variants grown in the field. Representative images are shown. Scale bars, 10 cm. FIG. 11AC shows stomatal conductance rate measurements of WT (ZH11) and transgenic rice lines expressing human ALKBH5 variants grown in the field. Representative images are shown. Scale bars, 10 cm. FIG. 11AD shows tiller number per plant of WT (ZH11) and transgenic rice lines expressing human ALKBH5 variants grown in the field. Representative images are shown. Scale bars, 10 cm. FIG. 11AE shows tiller number per plant of WT (ZH11) and transgenic rice lines expressing human ALKBH5 variants grown in the greenhouse. Representative images are shown. Scale bars, 10 cm. FIG. 11AF shows total grain number per plant of WT (ZH11) and transgenic rice lines expressing human ALKBH5 variants grown in the greenhouse. Representative images are shown. Scale bars, 4 cm. FIG. 11AG shows total grain number per plant of WT (ZH11) and transgenic rice lines expressing human ALKBH5 variants grown in the field. Representative images are shown. Scale bars, 4 cm. FIG. 11AH shows dry biomass per plant of WT (ZH11) and transgenic rice lines expressing human ALKBH5 variants grown in the field. FIG. 11AI shows dry biomass per plant of WT (ZH11) and transgenic rice lines expressing human ALKBH5 variants grown in the greenhouse. For FIGS. 11W-11AI, data are shown as means±SEM. Unpaired two-tailed t tests against ZH11 group; n.s., not significant; *p<0.05; **p<0.01; ***p<0.001. For FIGS. 11W, 11AE-11AF, 11AI, data for ZH11, ALKBH5-, ALKBH5Δ2- and ALKBH5Δ2^{mut}-rice was derived from FIGS. 11V and 11X-11Z.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

As used herein, the terms “a,” “an,” and “the” are intended to include the plural forms as well, unless the context clearly indicates otherwise. Where a term is provided in the singular, the inventors also contemplate aspects of the invention described by the plural of that term.

As used here, the term “about” refers to the usual error range for the respective value readily known to the skilled person in this technical field. Reference to “about” a value or parameter herein includes (and describes) embodiments that are directed to that value or parameter per se.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or B; A or C; B or C; A and B; A and C; B and C; A (alone); B (alone); and C (alone).

As used herein, a modifier to the initial item in a list is taken to apply to each item in the list. Thus, the phrase “at least about 10%, 20%, or 30%” herein is intended to have the same meaning as “at least about 10%, at least about 20%, or at least about 30%.”

### Recombinant DNA Molecules, Expression Vectors, Transformation Vectors, Plant Cells, and Plants Including an Engineered RNA m⁶A Demethylase; and Related Methods

RNA m⁶A demethylases are proteins that mediate oxidative removal (demethylation) of the N⁶-methly group of N⁶-methyladenosine (m⁶A) in RNA. Two different mammalian RNA m⁶A demethylases have been discovered: FTO (PMID: 22002720) and ALKBH5 (PMID: 23177736). FTO has homologs in animals and other organisms but not in most plants. ALKBH5 has homologs widely distributed in different plant species that also mediate demethylation of RNA m⁶A (PMID: 29180595)

In some variations, the disrupted region is a low complexity region. Low complexity regions (LCRs), or low complexity domains (LCDs) are segments within protein sequences characterized by a biased composition of a few amino acids, typically including, e.g., glycine, arginine, lysine, and serine, or by repetitive sequences. Due to their repetitive nature and the presence of a limited set of amino acids, low complexity regions often lack well-defined secondary or tertiary structures. However, they can play important functional roles in various cellular processes such as protein-protein interactions, RNA binding, and phase separation. Calculating LCRs can be performed as described previously, as in Conrad et al. (2016) (PMID: 27040163) or as in Beckmann et al. (2015) (PMID: 26632259). In some embodiments, the LCR has an IUPred score of greater than 0.5. In some embodiments, the LCR has a predicted local distance difference test (pLDDT) of less than 70. In some embodiments, the LCR has a pLDDT of less than 70 and greater than or equal to 50. In some embodiments, the LCR has a pLDDT of less than 50. In some embodiments, the LCR has less than 70%, less than 65%, less than 60%, less than 55%, less than 50%, less than 45%, less than 40%, less than 35%, less than 30%, less than 25%, less than 20%, less than 15%, less than 10%, or less than 5% identity with the catalytic domain of ALKBH5. In some variations, the disrupted region is an intrinsically disordered region (IDR) or an intrinsically disordered domain (IDD). In one variation, the intrinsically disordered region is an LCR. In one variation, the low complexity region is an IDR. Intrinsically disordered regions (IDRs) are compositionally biased regions, unstructured and flexible linkers, unstructured and flexible regions, etc. Calculating IDRs can be performed as described previously, as in Conrad et al. (2016) (PMID: 27040163) or as in Beckmann et al. (2015) (PMID: 26632259), or by using a tool such as AlphaFold (Jumper et al. 2021). In some embodiments, the IDR has an IUPred score of greater than 0.5. In some embodiments, the IDR has a predicted local distance difference test (pLDDT) of less than 70. In some embodiments, the IDR has a pLDDT of less than 70 and greater than or equal to 50. In some embodiments, the IDR has a pLDDT of less than 50. In some embodiments, the IDR has less than 70%, less than 65%, less than 60%, less than 55%, less than 50%, less than 45%, less than 40%, less than 35%, less than 30%, less than 25%, less than 20%, less than 15%, less than 10%, or less than 5% identity with the catalytic domain of ALKBH5. An aspect of the disclosure includes a method of identifying an intrinsically disordered region (IDR) or intrinsically disordered domain (IDD) in an ALKBH5 gene or ALKBH5 homolog, the method comprising (i) providing a polypeptide that encodes an ALKBH5 gene or ALKBH5 homolog, (ii) identifying a region of the polypeptide for which (a) the IUPred score of the region exceeds 0.5, (b) in an AlphaFold-predicted structure of the polypeptide the region has a pLDDT of less than 70, and (c) the region has less than 70%, less than 65%, less than 60%, less than 55%, less than 50%, less than 45%, less than 40%, less than 35%, less than 30%, less than 25%, less than 20%, less than 15%, less than 10%, or less than 5% identity with the catalytic domain of ALKBH5, wherein the region is therefore identified as an IDR or IDD. In some embodiments, the region has a pLDDT of less than 70 but greater than or equal to 50 in the AlphaFold-predicted structure. In some embodiments, the region has a pLDDT of less than 50 in the AlphaFold-predicted structure. In some embodiments, the region has less than 70%, less than 65%, less than 60%, less than 55%, less than 50%, less than 45%, less than 40%, less than 35%, less than 30%, less than 25%, less than 20%, less than 15%, less than 10%, or less than 5% identity with the amino acids 74-292 of human ALKBH5. In some embodiments, the method further comprises a) genetically modifying a plant by transforming the plant with one or more gene editing components that target an endogenous ALKBH5 homolog gene, wherein the endogenous ALKBH5 homolog gene is modified to encode a protein in which the identified IDR is disrupted, and b) growing the plant, wherein the plant has improved growth compared to a control plant. In some embodiments, the method further comprises a) engineering a plant to include a recombinant DNA encoding an engineered RNA m⁶A demethylase, wherein the identified IDR is disrupted in the engineered RNA m⁶A demethylase, and b) growing the plant, wherein the plant has improved growth compared to a control plant.

An aspect of the disclosure includes a recombinant DNA encoding an engineered RNA m⁶A demethylase, wherein the engineered RNA m⁶A demethylase includes at least one disrupted endogenous region, wherein the region is a low complexity region (LCR) and/or an intrinsically disordered region (IDR). An aspect of the disclosure includes a recombinant DNA encoding an engineered RNA m⁶A demethylase, wherein the engineered RNA m⁶A demethylase includes at least one disrupted endogenous low complexity region (LCR). In a further embodiment of this aspect, the RNA m⁶A demethylase includes a disrupted endogenous N-terminal LCR. In an additional embodiment of the aspect, the RNA m⁶A demethylase includes a disrupted endogenous C-terminal LCR. In some embodiments of this aspect, which may be combined with any of the preceding embodiments, the RNA m⁶A demethylase includes a disrupted endogenous N-terminal LCR and a disrupted C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the RNA m⁶A demethylase includes a deletion of at least about 10%, 20%, 30%, 40%, 50%, 60%, 70%, 80%, 90%, or 100% deletion of at least one endogenous LCR. In additional embodiments of this aspect, the RNA m⁶A demethylase lacks at least one endogenous LCR. In some further embodiments of this aspect, the RNA m⁶A demethylase lacks an endogenous N-terminal LCR. In some further embodiments of this aspect, the RNA m⁶A demethylase lacks an endogenous C-terminal LCR. In yet another embodiment of this aspect, the RNA m⁶A demethylase lacks both an endogenous N-terminal LCR and an endogenous C-terminal LCR.

In some embodiments of this aspect, which may be combined with any of the preceding embodiments, the RNA m⁶A demethylase is an engineered ALKBH5 or an engineered ALKBH5 homolog. In certain embodiments of this aspect, which may be combined with any of the preceding embodiments, the RNA m⁶A demethylase includes an amino acid sequence with at least about 70%, 75%, 80%, 85%, 90%, 91%, 92%, 93%, 94%, 95%, 96%, 97%, 98%, 99%, or 100% sequence identity to SEQ ID NO: 42. In another embodiment of this aspect, which may be combined with any of the preceding embodiments, the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 43. In a separate embodiment of this aspect, which may be combined with any of the preceding embodiments, the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 44. In an additional embodiment of this aspect, which may be combined with any of the preceding embodiments, the endogenous LCR leads to assembly of the endogenous RNA m⁶A demethylase in foci or condensates within the cell. In a certain embodiment of this aspect, which may be combined with any of the preceding embodiments, the engineered RNA m⁶A demethylase is an engineered ALKBH5. In one embodiment of this aspect, the ALKBH5 includes a deletion of at least about 10%, 20%, 30%, 40%, 50%, 60%, 70%, 80%, 90%, or 100% of (i) amino acids 30-81 and/or (ii) amino acids 298-394 of endogenous ALKBH5 (SEQ ID NO: 1). In some embodiments of this aspect, the ALKBH5 includes a deletion of at least about 10%, 20%, 30%, 40%, 50%, 60%, 70%, 80%, 90%, or 100% of amino acids 30-81 of endogenous ALKBH5 (SEQ ID NO: 1). In one embodiment of this aspect, the ALKBH5 includes a deletion of at least about 10%, 20%, 30%, 40%, 50%, 60%, 70%, 80%, 90%, or 100% of amino acids 298-394 of endogenous ALKBH5 (SEQ ID NO: 1). In an additional embodiment of this aspect, the ALKBH5 includes a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of endogenous ALKBH5 (SEQ ID NO: 1). In yet another embodiment of this aspect, the RNA m⁶A demethylase is selected from ALKBH5_{Δ30-81}, ALKBH5_{Δ298-394}, and ALKBH5_{Δ30-81 and 298-394}. In one embodiment of this aspect, the RNA m⁶A demethylase is ALKBH5_{Δ298-394}. In some embodiments of this aspect, which may be combined with any of the preceding embodiments, the RNA m⁶A demethylase is an engineered ALKBH5 homolog. In a further embodiment of this aspect, the RNA m⁶A demethylase is an animal ALKBH5 homolog. In some embodiments of this aspect, the RNA m⁶A demethylase is a mammalian ALKBH5 homolog. In other embodiments of this aspect, the RNA m⁶A demethylase is a non-animal ALKBH5 homolog. In an additional embodiment of this aspect, the RNA m⁶A demethylase is a plant ALKBH5 homolog. In a further embodiment of this aspect, the RNA m⁶A demethylase is an ALKBH5 homolog from Arabidopsis, rice, rapeseed, tobacco, alfalfa, sorghum, maize, wheat, or soy. In an embodiment of this aspect, the RNA m⁶A demethylase is an Arabidopsis ALKBH5 homolog. In a further embodiment of this aspect, the RNA m⁶A demethylase is selected from ALKBH9B and ALKBH10B. In yet another embodiment of this aspect, the RNA m⁶A demethylase is ALKBH9B. In some embodiments of this aspect, the ALKBH9B includes a deletion of at least about 10%, 20%, 30%, 40%, 50%, 60%, 70%, 80%, 90%, or 100% of (i) amino acids 76-102, (ii) amino acids 145-183, and/or (iii) amino acids 432-507 of endogenous Arabidopsis ALKBH9B (SEQ ID NO: 3). In one embodiment of this aspect, the ALKBH9B includes a deletion of at least about 10%, 20%, 30%, 40%, 50%, 60%, 70%, 80%, 90%, or 100% of amino acids 76-102 of endogenous Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 145-183 of endogenous Arabidopsis ALKBH9B (SEQ ID NO: 3). In yet another embodiment of this aspect, the ALKBH9B includes a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In an additional embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of endogenous Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In still another embodiment of this aspect, the ALKBH9B includes a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In a further embodiment of this aspect, the ALKBH9B includes a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of endogenous Arabidopsis ALKBH9B (SEQ ID NO: 3). In yet another embodiment of this aspect, the ALKBH9B is selected from ALKBH9B_{Δ176-102}, ALKBH9B_{Δ145-183}, and ALKBH9B_{Δ432-507}. In some embodiments of this aspect, the RNA m⁶A demethylase is ALKBH10B. In certain embodiments of this aspect, the ALKBH10B includes a deletion of at least about 10%, 20%, 30%, 40%, 50%, 60%, 70%, 80%, 90%, or 100% of (i) amino acids 131-190 and/or (ii) amino acids 501-569 of endogenous Arabidopsis ALKBH10B (SEQ ID NO: 5). In one embodiment of this aspect, the ALKBH10B includes a deletion of at least about 10%, 20%, 30%, 40%, 50%, 60%, 70%, 80%, 90%, or 100% of amino acids 131-190 of endogenous Arabidopsis ALKBH10B (SEQ ID NO: 5). In another embodiment of this aspect, the ALKBH10B includes a deletion of at least about 10%, 20%, 30%, 40%, 50%, 60%, 70%, 80%, 90%, or 100% of amino acids 501-569 of endogenous Arabidopsis ALKBH10B (SEQ ID NO: 5). In yet another embodiment of this aspect, the ALKBH10B includes a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of endogenous Arabidopsis ALKBH10B (SEQ ID NO: 5). In some embodiments of this aspect, the RNA m⁶A demethylase is a rice ALKBH5 homolog. In a further embodiment of this aspect, the RNA m⁶A demethylase is selected from Os9B and Os10B. In yet another embodiment of this aspect, the RNA m⁶A demethylase is Os9B. In an additional embodiment of this aspect, the Os9B includes a deletion of at least about 10%, 20%, 30%, 40%, 50%, 60%, 70%, 80%, 90%, or 100% of (i) amino acids 60-170 and/or (ii) amino acids 428-616 of endogenous Os9B (SEQ ID NO: 6). In a further embodiment of this aspect, the Os9B includes a deletion of at least about 10%, 20%, 30%, 40%, 50%, 60%, 70%, 80%, 90%, or 100% of amino acids 60-170 of endogenous Os9B (SEQ ID NO: 6). In another embodiment of this aspect, the Os9B includes a deletion of at least about 10%, 20%, 30%, 40%, 50%, 60%, 70%, 80%, 90%, or 100% of amino acids 428-616 of endogenous Os9B (SEQ ID NO: 6). In an additional embodiment of this aspect, the Os9B includes a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of endogenous Os9B (SEQ ID NO: 6). In still another embodiment of this aspect, the Os9B is selected from Os9B_{Δ60-170 and 428-616}, and Os9B_{Δ428-616}. In yet another embodiment of this aspect, the RNA m⁶A demethylase is Os10B. In some embodiments of this aspect, the Os10B includes a deletion of at least about 10%, 20%, 30%, 40%, 50%, 60%, 70%, 80%, 90%, or 100% of (i) amino acids 2-30, (ii) amino acids 99-126, and/or (iii) amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 of endogenous Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 99-126 of endogenous Os10B (SEQ ID NO: 8). In one embodiment of this aspect, the Os10B includes a deletion of at least about 10%, 20%, 30%, 40%, 50%, 60%, 70%, 80%, 90%, or 100% of amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of endogenous Os10B (SEQ ID NO: 8). In a further embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of endogenous Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of endogenous Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of endogenous Os10B (SEQ ID NO: 8). In a further embodiment of this aspect, the Os10B is selected from Os10B_{Δ2-30, 99-126, and 491-595}, and Os10B_{Δ2-30, 99-126, and 389-595}. In yet another embodiment of this aspect, the RNA m⁶A demethylase is rapeseed ALKBH9B. In some embodiments of this aspect, the ALKBH9B includes a deletion of at least about 10%, 20%, 30%, 40%, 50%, 60%, 70%, 80%, 90%, or 100% of (i) amino acids 55-137 and/or (ii) amino acids 401-467 of endogenous rapeseed ALKBH9B (SEQ ID NO: 10). In a further embodiment of this aspect, the ALKBH9B includes a deletion of at least about 10%, 20%, 30%, 40%, 50%, 60%, 70%, 80%, 90%, or 100% of amino acids 55-137 of endogenous rapeseed ALKBH9B (SEQ ID NO: 10). In one embodiment of this aspect, the ALKBH9B includes a deletion of at least about 10%, 20%, 30%, 40%, 50%, 60%, 70%, 80%, 90%, or 100% of amino acids 401-467 of endogenous rapeseed ALKBH9B (SEQ ID NO: 10). In an additional embodiment of this aspect, the ALKBH9B includes a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of endogenous rapeseed ALKBH9B (SEQ ID NO: 10). In yet another embodiment of this aspect, the RNA m⁶A demethylase is tobacco ALKBH9B. In still another embodiment of this aspect, the ALKBH9B includes a deletion of at least about 10%, 20%, 30%, 40%, 50%, 60%, 70%, 80%, 90%, or 100% of (i) amino acids 121-243 and/or (ii) amino acids 447-541 of endogenous tobacco ALKBH9B (SEQ ID NO: 12). In an additional embodiment of this aspect, the ALKBH9B includes a deletion of at least about 10%, 20%, 30%, 40%, 50%, 60%, 70%, 80%, 90%, or 100% of amino acids 121-243 of endogenous tobacco ALKBH9B (SEQ ID NO: 12). In one embodiment of this aspect, the ALKBH9B includes a deletion of at least about 10%, 20%, 30%, 40%, 50%, 60%, 70%, 80%, 90%, or 100% of amino acids 447-541 of endogenous tobacco ALKBH9B (SEQ ID NO: 12). In a further embodiment of this aspect, the ALKBH9B includes a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of endogenous tobacco ALKBH9B (SEQ ID NO: 12). In an additional embodiment of this aspect, the RNA m⁶A demethylase is tobacco ALKBH10B. In some embodiments of this aspect, the ALKBH10B includes a deletion of at least about 10%, 20%, 30%, 40%, 50%, 60%, 70%, 80%, 90%, or 100% of (i) amino acids 181-235 and/or (ii) amino acids 504-640 of endogenous tobacco ALKBH10B (SEQ ID NO: 14). In one embodiment of this aspect, the ALKBH10B includes a deletion of at least about 10%, 20%, 30%, 40%, 50%, 60%, 70%, 80%, 90%, or 100% of amino acids 181-235 of endogenous tobacco ALKBH10B (SEQ ID NO: 14). In another embodiment of this aspect, the ALKBH10B includes a deletion of at least about 10%, 20%, 30%, 40%, 50%, 60%, 70%, 80%, 90%, or 100% of amino acids 504-640 of endogenous tobacco ALKBH10B (SEQ ID NO: 14). In an additional embodiment of this aspect, the ALKBH10B includes a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of endogenous tobacco ALKBH10B (SEQ ID NO: 14). In a further embodiment of this aspect, the RNA m⁶A demethylase is alfalfa ALKBH9B. In one embodiment of this aspect, the ALKBH9B includes a deletion of at least about 10%, 20%, 30%, 40%, 50%, 60%, 70%, 80%, 90%, or 100% of amino acids 442-508 of endogenous alfalfa ALKBH9B (SEQ ID NO: 16). In a different embodiment of this aspect, the RNA m⁶A demethylase is sorghum ALKBH9B. In a further embodiment of this aspect, the ALKBH9B includes a deletion of at least about 10%, 20%, 30%, 40%, 50%, 60%, 70%, 80%, 90%, or 100% of (i) amino acids 66-179 and/or (ii) amino acids 423-611 of endogenous sorghum ALKBH9B (SEQ ID NO: 18). In another embodiment of this aspect, the ALKBH9B includes a deletion of at least about 10%, 20%, 30%, 40%, 50%, 60%, 70%, 80%, 90%, or 100% of amino acids 66-179 of endogenous sorghum ALKBH9B (SEQ ID NO: 18). In yet another embodiment of this aspect, the ALKBH9B includes a deletion of at least about 10%, 20%, 30%, 40%, 50%, 60%, 70%, 80%, 90%, or 100% of amino acids 423-611 of endogenous sorghum ALKBH9B (SEQ ID NO: 18). In still another embodiment of this aspect, the ALKBH9B includes a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of endogenous sorghum ALKBH9B (SEQ ID NO: 18). In some embodiments of this aspect, the RNA m⁶A demethylase is maize ALKBH9B. In certain embodiments of this aspect, the ALKBH9B includes a deletion of at least about 10%, 20%, 30%, 40%, 50%, 60%, 70%, 80%, 90%, or 100% of (i) amino acids 72-165 and/or (ii) amino acids 423-615 of endogenous maize ALKBH9B (SEQ ID NO: 20). In a further embodiment of this aspect, the ALKBH9B includes a deletion of at least about 10%, 20%, 30%, 40%, 50%, 60%, 70%, 80%, 90%, or 100% of amino acids 72-165 of endogenous maize ALKBH9B (SEQ ID NO: 20). In one embodiment of this aspect, the ALKBH9B includes a deletion of at least about 10%, 20%, 30%, 40%, 50%, 60%, 70%, 80%, 90%, or 100% of amino acids 423-615 of endogenous maize ALKBH9B (SEQ ID NO: 20). In another embodiment of this aspect, the ALKBH9B includes a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of endogenous maize ALKBH9B (SEQ ID NO: 20). In yet another embodiment of this aspect, the RNA m⁶A demethylase is maize ALKBH10B. In one embodiment of this aspect, the ALKBH10B includes a deletion of at least about 10%, 20%, 30%, 40%, 50%, 60%, 70%, 80%, 90%, or 100% of (i) amino acids 109-210 and/or (ii) amino acids 437-573 of endogenous maize ALKBH10B (SEQ ID NO: 22). In another embodiment of this aspect, the ALKBH10B includes a deletion of at least about 10%, 20%, 30%, 40%, 50%, 60%, 70%, 80%, 90%, or 100% of amino acids 109-210 of endogenous maize ALKBH10B (SEQ ID NO: 22). In still another embodiment of this aspect, the ALKBH10B includes a deletion of at least about 10%, 20%, 30%, 40%, 50%, 60%, 70%, 80%, 90%, or 100% of amino acids 437-573 of endogenous maize ALKBH10B (SEQ ID NO: 22). In a further embodiment of this aspect, the ALKBH10B includes a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of endogenous maize ALKBH10B (SEQ ID NO: 22). In an additional embodiment of this aspect, the RNA m⁶A demethylase is wheat ALKBH9B. In some embodiments of this aspect, the ALKBH9B includes a deletion of at least about 10%, 20%, 30%, 40%, 50%, 60%, 70%, 80%, 90%, or 100% of (i) amino acids 57-163 and/or (ii) amino acids 417-612 of endogenous wheat ALKBH9B (SEQ ID NO: 24). In an embodiment of this aspect, the ALKBH9B includes a deletion of at least about 10%, 20%, 30%, 40%, 50%, 60%, 70%, 80%, 90%, or 100% of amino acids 57-163 of endogenous wheat ALKBH9B (SEQ ID NO: 24). In yet another embodiment of this aspect, the ALKBH9B includes a deletion of at least about 10%, 20%, 30%, 40%, 50%, 60%, 70%, 80%, 90%, or 100% of amino acids 417-612 of endogenous wheat ALKBH9B (SEQ ID NO: 24). In an additional embodiment of this aspect, the ALKBH9B includes a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of endogenous wheat ALKBH9B (SEQ ID NO: 24). In still another embodiment of this aspect, the RNA m⁶A demethylase is wheat ALKBH10B. In some embodiments of this aspect, the ALKBH10B includes a deletion of at least about 10%, 20%, 30%, 40%, 50%, 60%, 70%, 80%, 90%, or 100% of (i) amino acids 91-211 and/or (ii) amino acids 440-566 of endogenous wheat ALKBH10B (SEQ ID NO: 26). In another embodiment of this aspect, the ALKBH10B includes a deletion of at least about 10%, 20%, 30%, 40%, 50%, 60%, 70%, 80%, 90%, or 100% of amino acids 91-211 of endogenous wheat ALKBH10B (SEQ ID NO: 26). In a further embodiment of this aspect, the ALKBH10B includes a deletion of at least about 10%, 20%, 30%, 40%, 50%, 60%, 70%, 80%, 90%, or 100% of amino acids 440-566 of endogenous wheat ALKBH10B (SEQ ID NO: 26). In still another embodiment of this aspect, the ALKBH10B includes a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of endogenous wheat ALKBH10B (SEQ ID NO: 26). In a further embodiment of this aspect, the RNA m⁶A demethylase is cassava ALKBH9B. In another embodiment of this aspect, the RNA m⁶A demethylase is cassava ALKBH10B. In an additional embodiment of this aspect, the RNA m⁶A demethylase is cowpea ALKBH9B. In another embodiment of this aspect, the RNA m⁶A demethylase is cowpea ALKBH10B. In a further embodiment of this aspect, the RNA m⁶A demethylase is cotton ALKBH9B. In another embodiment of this aspect, the RNA m⁶A demethylase is cotton ALKBH10B. ALKBH5 and ALKBH5 homolog sequences are provided in Table 1.

TABLE 1
Sequences of ALKBH5 and ALKBH5 homologs

| Name | Type | SEQ ID NO |
|---|---|---|
| ALKBH5 | Protein | 1 |
| ALKBH5 | DNA | 2 |
| Arabidopsis ALKBH9B | Protein | 3 |
| Arabidopsis ALKBH9B | DNA | 4 |
| Arabidopsis ALKBH10B | Protein | 5 |
| Arabidopsis ALKBH10B | DNA | 61 |
| Arabidopsis ALKBH10B - mESC | DNA | 62 |
| codon optimized | | |
| Rice Os9B | Protein | 6 |
| Rice Os9B | DNA | 7 |
| Rice Os10B | Protein | 8 |
| Rice Os10B | DNA | 9 |
| Rapeseed ALKBH9B | Protein | 10 |
| Rapeseed ALKBH9B | DNA | 11 |
| Tobacco ALKBH9B | Protein | 12 |
| Tobacco ALKBH9B | DNA | 13 |
| Tobacco ALKBH10B | Protein | 14 |
| Tobacco ALKBH10B | DNA | 15 |
| Alfalfa ALKBH9B | Protein | 16 |
| Alfalfa ALKBH9B | DNA | 17 |
| Sorghum ALKBH9B | Protein | 18 |
| Sorghum ALKBH9B | DNA | 19 |
| Maize ALKBH9B | Protein | 20 |
| Maize ALKBH9B | DNA | 21 |
| Maize ALKBH10B | Protein | 22 |
| Maize ALKBH10B | DNA | 23 |
| Wheat ALKBH9B | Protein | 24 |
| Wheat ALKBH9B | DNA | 25 |
| Wheat ALKBH10B | Protein | 26 |
| Wheat ALKBH10B | DNA | 27 |
| Cassava ALKBH9B | Protein | 49 |
| Cassava ALKBH9B | DNA | 50 |
| Cassava ALKBH10B | Protein | 51 |
| Cassava ALKBH10B | DNA | 52 |
| Cowpea ALKBH9B | Protein | 53 |
| Cowpea ALKBH9B | DNA | 54 |
| Cowpea ALKBH10B | Protein | 55 |
| Cowpea ALKBH10B | DNA | 56 |
| Cotton ALKBH9B | Protein | 57 |
| Cotton ALKBH9B | DNA | 58 |
| Cotton ALKBH10B | Protein | 59 |
| Cotton ALKBH10B | DNA | 60 |
| Soybean G.max_Fiskeby | Protein | 186 |
| v1.1\|GlymaFiskIII.17G220300.1.p | | |
| Soybean G.max_Lee | Protein | 187 |
| v1.1\|GlymaLee.08G186500.1.p | | |
| Tomato Solyc02g062180.3.1 | Protein | 188 |
| Tomato Solyc02g083960.4.1 | Protein | 189 |
| Tomato Solyc01g104130.4.1 | Protein | 190 |
| Arabidopsis ALKBH9C | Protein | 191 |
| Arabidopsis ALKBH9A | Protein | 192 |

In one embodiment of this aspect, which may be combined with any of the previous embodiments, the RNA m⁶A demethylase is operably linked to at least one nuclear localization signal (NLS). In a further embodiment of this aspect, the NLS is encoded by SEQ ID NO: 40. In yet another embodiment of this aspect, the amino acid sequence of the NLS is SEQ ID NO: 41. In still another embodiment of this aspect, the RNA m⁶A demethylase is operably linked to at least one heterologous nuclear localization signal (NLS). In an additional embodiment of this aspect, which may be combined with any of the previous embodiments, the RNA m⁶A demethylase is operably linked to a promoter for expression in a plant. In some embodiments of this aspect, the promoter is a constitutive promoter.

Some aspects of the disclosure include an expression vector including the recombinant DNA of any of the preceding embodiments. An additional aspect of the disclosure provides an expression vector including a nucleic acid encoding an engineered RNA m⁶A demethylase, wherein the engineered RNA m⁶A demethylase includes at least one disrupted endogenous low complexity region (LCR), and wherein the expression vector is for expression in a plant or plant cell. Some aspects of the disclosure further include a transformation vector including the recombinant DNA of any of the preceding embodiments, or the expression vector of any of the preceding embodiments.

A further aspect of the disclosure provides a plant or plant cell including the recombinant DNA of any of the preceding embodiments, or the expression vector of any of the preceding embodiments. In some embodiments of this aspect, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. Photosynthesis can be measured using Leaf Gas Exchange Measurements, such as those obtained using an LI-6400XT Portable Photosynthesis System (LI-COR Inc., NE, USA) on attached leaves of target plants to accurately assess leaf-level gas exchange parameters that can reflect the intensity of photosynthesis. These measurements include the CO₂ assimilation rate, stomatal conductance, and transpiration rate. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant or a plant including the plant cell of any of the preceding embodiments. An additional aspect of the disclosure provides a commercial product derived from the plant or a plant including the plant cell of any of the preceding embodiments, or the seed of any of the preceding embodiments.

An additional aspect of the disclosure provides a method of improving growth of a plant including a) engineering a plant to include the recombinant DNA of any of the preceding recombinant DNA embodiments, the expression vector of any of the preceding expression vector embodiments, or the transformation vector of any of the preceding transformation vector embodiments, and b) growing the plant, wherein the plant has improved growth compared to a control plant. In another embodiment of this aspect, the plant has improved growth under abiotic stress conditions. In an additional embodiment of this aspect, the plant has improved growth under drought stress, salt stress, heat stress, or cold stress. In yet another embodiment of this aspect, the plant has improved growth under biotic stress conditions. In still another embodiment of this aspect, the plant is improved in a characteristic selected from biomass, yield, root growth, root expansion, stem cell expansion of the root, stem cell expansion of the shoot, drought resistance, and pest resistance. In a further embodiment of this aspect, the plant has elevated photosynthesis. Photosynthesis can be measured using Leaf Gas Exchange Measurements, such as those obtained using an LI-6400XT Portable Photosynthesis System (LI-COR Inc., NE, USA) on attached leaves of target plants to accurately assess leaf-level gas exchange parameters that can reflect the intensity of photosynthesis. These measurements include the CO₂ assimilation rate, stomatal conductance, and transpiration rate. In a further embodiment of this aspect, which may be combined with any of the preceding embodiments, the plant is selected from Arabidopsis, rice, corn, soybean, potato, cassava, cowpea, cotton, turf grass, strawberry, blueberry, blackberry, raspberry, and wheat.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

Plants with Engineered RNA m⁶A Demethylases

An aspect of the disclosure includes a plant including nucleic acid encoding an engineered RNA m⁶A demethylase, wherein the engineered RNA m⁶A demethylase includes at least one disrupted endogenous region, wherein the region is a low complexity region (LCR) and/or an intrinsically disordered region (IDR). Further aspects of the disclosure include a plant including nucleic acid encoding an engineered RNA m⁶A demethylase, wherein the engineered RNA m⁶A demethylase includes at least one disrupted endogenous low complexity region (LCR). In an embodiment of this aspect, the RNA m⁶A demethylase includes a disrupted endogenous N-terminal LCR. In a further embodiment of this aspect, the RNA m⁶A demethylase includes a disrupted endogenous C-terminal LCR. In an additional embodiment of this aspect, the RNA m⁶A demethylase includes a disrupted endogenous N-terminal LCR and a disrupted C-terminal LCR. In yet another embodiment of this aspect, which may be combined with any of the preceding plant embodiments, the disruption of the endogenous LCR is selected from a partial truncation, a full truncation, a deletion, and a replacement. In another embodiment of this aspect, which may be combined with any of the preceding plant embodiments, the RNA m⁶A demethylase includes a deletion of at least about 10%, 20%, 30%, 40%, 50%, 60%, 70%, 80%, 90%, or 100% deletion of at least one endogenous LCR. In additional embodiments of this aspect, the RNA m⁶A demethylase lacks at least one endogenous LCR. In some further embodiments of this aspect, the RNA m⁶A demethylase lacks an endogenous N-terminal LCR. In some further embodiments of this aspect, the RNA m⁶A demethylase lacks an endogenous C-terminal LCR. In yet another embodiment of this aspect, the RNA m⁶A demethylase lacks both an endogenous N-terminal LCR and an endogenous C-terminal LCR.

In some embodiments of this aspect, which may be combined with any of the preceding plant embodiments, the RNA m⁶A demethylase is an engineered ALKBH5 or an engineered ALKBH5 homolog. In certain embodiments of this aspect, which may be combined with any of the preceding embodiments, the RNA m⁶A demethylase includes an amino acid sequence with at least about 70%, 75%, 80%, 85%, 90%, 91%, 92%, 93%, 94%, 95%, 96%, 97%, 98%, 99%, or 100% sequence identity to SEQ ID NO: 42. In an additional embodiment of this aspect, which may be combined with any of the preceding plant embodiments, the endogenous LCR leads to assembly of the endogenous RNA m⁶A demethylase in foci or condensates within the cell. In still another embodiment of this aspect, which may be combined with any of the preceding plant embodiments, the engineered RNA m⁶A demethylase is a heterologous RNA m⁶A demethylase. In an additional embodiment of this aspect, which may be combined with any of the preceding plant embodiments, the engineered RNA m⁶A demethylase is an endogenous RNA m⁶A demethylase. In a certain embodiment of this aspect, which may be combined with any of the preceding plant embodiments, the engineered RNA m⁶A demethylase is an engineered ALKBH5. In one embodiment of this aspect, the ALKBH5 includes a deletion of at least about 10%, 20%, 30%, 40%, 50%, 60%, 70%, 80%, 90%, or 100% of (i) amino acids 30-81 and/or (ii) amino acids 298-394 of ALKBH5 (SEQ ID NO: 1). In some embodiments of this aspect, the ALKBH5 includes a deletion of at least about 10%, 20%, 30%, 40%, 50%, 60%, 70%, 80%, 90%, or 100% of amino acids 30-81 of ALKBH5 (SEQ ID NO: 1). In one embodiment of this aspect, the ALKBH5 includes a deletion of at least about 10%, 20%, 30%, 40%, 50%, 60%, 70%, 80%, 90%, or 100% of amino acids 298-394 of ALKBH5 (SEQ ID NO: 1). In an additional embodiment of this aspect, the ALKBH5 includes a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of ALKBH5 (SEQ ID NO: 1). In yet another embodiment of this aspect, the RNA m⁶A demethylase is selected from ALKBH5_{Δ30-81}, ALKBH5_{Δ298-394}, and ALKBH5_{Δ30-81 and 298-394}. In one embodiment of this aspect, the RNA m⁶A demethylase is ALKBH5_{Δ298-394}. In some embodiments of this aspect, which may be combined with any of the preceding plant embodiments, the RNA m⁶A demethylase is an engineered ALKBH5 homolog. In a further embodiment of this aspect, the RNA m⁶A demethylase is an animal ALKBH5 homolog. In some embodiments of this aspect, the RNA m⁶A demethylase is a mammalian ALKBH5 homolog. In other embodiments of this aspect, the RNA m⁶A demethylase is a non-animal ALKBH5 homolog. In an additional embodiment of this aspect, the RNA m⁶A demethylase is a plant ALKBH5 homolog. In a further embodiment of this aspect, the RNA m⁶A demethylase is an ALKBH5 homolog from Arabidopsis, rice, rapeseed, tobacco, alfalfa, sorghum, maize, wheat, or soy. In an embodiment of this aspect, the RNA m⁶A demethylase is an Arabidopsis ALKBH5 homolog. In a further embodiment of this aspect, the RNA m⁶A demethylase is selected from ALKBH9B and ALKBH10B. In yet another embodiment of this aspect, the RNA m⁶A demethylase is ALKBH9B. In some embodiments of this aspect, the ALKBH9B includes a deletion of at least about 10%, 20%, 30%, 40%, 50%, 60%, 70%, 80%, 90%, or 100% of (i) amino acids 76-102, (ii) amino acids 145-183, and/or (iii) amino acids 432-507 of Arabidopsis ALKBH9B (SEQ ID NO: 3). In one embodiment of this aspect, the ALKBH9B includes a deletion of at least about 10%, 20%, 30%, 40%, 50%, 60%, 70%, 80%, 90%, or 100% of amino acids 76-102 of Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 145-183 of Arabidopsis ALKBH9B (SEQ ID NO: 3). In yet another embodiment of this aspect, the ALKBH9B includes a deletion of at least about 10%, 20%, 30%, 40%, 50%, 60%, 70%, 80%, 90%, or 100% of amino acids 432-507 of Arabidopsis ALKBH9B (SEQ ID NO: 3). In an additional embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of Arabidopsis ALKBH9B (SEQ ID NO: 3). In another embodiment of this aspect, the ALKBH9B includes a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of Arabidopsis ALKBH9B (SEQ ID NO: 3). In still another embodiment of this aspect, the ALKBH9B includes a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of Arabidopsis ALKBH9B (SEQ ID NO: 3). In a further embodiment of this aspect, the ALKBH9B includes a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of Arabidopsis ALKBH9B (SEQ ID NO: 3). In yet another embodiment of this aspect, the ALKBH9B is selected from ALKBH9B_{Δ176-102}, ALKBH9B_{Δ145-183}, and ALKBH9B_{Δ432-507}. In some embodiments of this aspect, the RNA m⁶A demethylase is ALKBH10B. In certain embodiments of this aspect, the ALKBH10B includes a deletion of at least about 10%, 20%, 30%, 40%, 50%, 60%, 70%, 80%, 90%, or 100% of (i) amino acids 131-190 and/or (ii) amino acids 501-569 of Arabidopsis ALKBH10B (SEQ ID NO: 5). In one embodiment of this aspect, the ALKBH10B includes a deletion of at least about 10%, 20%, 30%, 40%, 50%, 60%, 70%, 80%, 90%, or 100% of amino acids 131-190 of Arabidopsis ALKBH10B (SEQ ID NO: 5). In another embodiment of this aspect, the ALKBH10B includes a deletion of at least about 10%, 20%, 30%, 40%, 50%, 60%, 70%, 80%, 90%, or 100% of amino acids 501-569 of Arabidopsis ALKBH10B (SEQ ID NO: 5). In yet another embodiment of this aspect, the ALKBH10B includes a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of Arabidopsis ALKBH10B (SEQ ID NO: 5). In some embodiments of this aspect, the RNA m⁶A demethylase is a rice ALKBH5 homolog. In a further embodiment of this aspect, the RNA m⁶A demethylase is selected from Os9B and Os10B. In yet another embodiment of this aspect, the RNA m⁶A demethylase is Os9B. In an additional embodiment of this aspect, the Os9B includes a deletion of at least about 10%, 20%, 30%, 40%, 50%, 60%, 70%, 80%, 90%, or 100% of (i) amino acids 60-170 and/or (ii) amino acids 428-616 of Os9B (SEQ ID NO: 6). In a further embodiment of this aspect, the Os9B includes a deletion of at least about 10%, 20%, 30%, 40%, 50%, 60%, 70%, 80%, 90%, or 100% of amino acids 60-170 of Os9B (SEQ ID NO: 6). In another embodiment of this aspect, the Os9B includes a deletion of at least about 10%, 20%, 30%, 40%, 50%, 60%, 70%, 80%, 90%, or 100% of amino acids 428-616 of Os9B (SEQ ID NO: 6). In an additional embodiment of this aspect, the Os9B includes a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of Os9B (SEQ ID NO: 6). In still another embodiment of this aspect, the Os9B is selected from Os9B_{Δ60-170 and 428-616}, and Os9B_{Δ428-616}. In yet another embodiment of this aspect, the RNA m⁶A demethylase is Os10B. In some embodiments of this aspect, the Os10B includes a deletion of at least about 10%, 20%, 30%, 40%, 50%, 60%, 70%, 80%, 90%, or 100% of (i) amino acids 2-30, (ii) amino acids 99-126, and/or (iii) amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 of Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 99-126 of Os10B (SEQ ID NO: 8). In one embodiment of this aspect, the Os10B includes a deletion of at least about 10%, 20%, 30%, 40%, 50%, 60%, 70%, 80%, 90%, or 100% of amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of Os10B (SEQ ID NO: 8). In a further embodiment of this aspect, the Os10B includes a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of Os10B (SEQ ID NO: 8). In another embodiment of this aspect, the Os10B includes a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of Os10B (SEQ ID NO: 8). In an additional embodiment of this aspect, the Os10B includes a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of Os10B (SEQ ID NO: 8). In a further embodiment of this aspect, the Os10B is selected from Os10B_{Δ2-30, 99-126, and 491-595}, and Os10B_{Δ2-30, 99-126, and 389-595}. In yet another embodiment of this aspect, the RNA m⁶A demethylase is rapeseed ALKBH9B. In some embodiments of this aspect, the ALKBH9B includes a deletion of at least about 10%, 20%, 30%, 40%, 50%, 60%, 70%, 80%, 90%, or 100% of (i) amino acids 55-137 and/or (ii) amino acids 401-467 of rapeseed ALKBH9B (SEQ ID NO: 10). In a further embodiment of this aspect, the ALKBH9B includes a deletion of at least about 10%, 20%, 30%, 40%, 50%, 60%, 70%, 80%, 90%, or 100% of amino acids 55-137 of rapeseed ALKBH9B (SEQ ID NO: 10). In one embodiment of this aspect, the ALKBH9B includes a deletion of at least about 10%, 20%, 30%, 40%, 50%, 60%, 70%, 80%, 90%, or 100% of amino acids 401-467 of rapeseed ALKBH9B (SEQ ID NO: 10). In an additional embodiment of this aspect, the ALKBH9B includes a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of rapeseed ALKBH9B (SEQ ID NO: 10). In yet another embodiment of this aspect, the RNA m⁶A demethylase is tobacco ALKBH9B. In still another embodiment of this aspect, the ALKBH9B includes a deletion of at least about 10%, 20%, 30%, 40%, 50%, 60%, 70%, 80%, 90%, or 100% of (i) amino acids 121-243 and/or (ii) amino acids 447-541 of tobacco ALKBH9B (SEQ ID NO: 12). In an additional embodiment of this aspect, the ALKBH9B includes a deletion of at least about 10%, 20%, 30%, 40%, 50%, 60%, 70%, 80%, 90%, or 100% of amino acids 121-243 of tobacco ALKBH9B (SEQ ID NO: 12). In one embodiment of this aspect, the ALKBH9B includes a deletion of at least about 10%, 20%, 30%, 40%, 50%, 60%, 70%, 80%, 90%, or 100% of amino acids 447-541 of tobacco ALKBH9B (SEQ ID NO: 12). In a further embodiment of this aspect, the ALKBH9B includes a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of tobacco ALKBH9B (SEQ ID NO: 12). In an additional embodiment of this aspect, the RNA m⁶A demethylase is tobacco ALKBH10B. In some embodiments of this aspect, the ALKBH10B includes a deletion of at least about 10%, 20%, 30%, 40%, 50%, 60%, 70%, 80%, 90%, or 100% of (i) amino acids 181-235 and/or (ii) amino acids 504-640 of tobacco ALKBH10B (SEQ ID NO: 14). In one embodiment of this aspect, the ALKBH10B includes a deletion of at least about 10%, 20%, 30%, 40%, 50%, 60%, 70%, 80%, 90%, or 100% of amino acids 181-235 of tobacco ALKBH10B (SEQ ID NO: 14). In another embodiment of this aspect, the ALKBH10B includes a deletion of at least about 10%, 20%, 30%, 40%, 50%, 60%, 70%, 80%, 90%, or 100% of amino acids 504-640 of tobacco ALKBH10B (SEQ ID NO: 14). In an additional embodiment of this aspect, the ALKBH10B includes a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of tobacco ALKBH10B (SEQ ID NO: 14). In a further embodiment of this aspect, the RNA m⁶A demethylase is alfalfa ALKBH9B. In one embodiment of this aspect, the ALKBH9B includes a deletion of at least about 10%, 20%, 30%, 40%, 50%, 60%, 70%, 80%, 90%, or 100% of amino acids 442-508 of alfalfa ALKBH9B (SEQ ID NO: 16). In a different embodiment of this aspect, the RNA m⁶A demethylase is sorghum ALKBH9B. In a further embodiment of this aspect, the ALKBH9B includes a deletion of at least about 10%, 20%, 30%, 40%, 50%, 60%, 70%, 80%, 90%, or 100% of (i) amino acids 66-179 and/or (ii) amino acids 423-611 of sorghum ALKBH9B (SEQ ID NO: 18). In another embodiment of this aspect, the ALKBH9B includes a deletion of at least about 10%, 20%, 30%, 40%, 50%, 60%, 70%, 80%, 90%, or 100% of amino acids 66-179 of sorghum ALKBH9B (SEQ ID NO: 18). In yet another embodiment of this aspect, the ALKBH9B includes a deletion of at least about 10%, 20%, 30%, 40%, 50%, 60%, 70%, 80%, 90%, or 100% of amino acids 423-611 of sorghum ALKBH9B (SEQ ID NO: 18). In still another embodiment of this aspect, the ALKBH9B includes a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of sorghum ALKBH9B (SEQ ID NO: 18). In some embodiments of this aspect, the RNA m⁶A demethylase is maize ALKBH9B. In certain embodiments of this aspect, the ALKBH9B includes a deletion of at least about 10%, 20%, 30%, 40%, 50%, 60%, 70%, 80%, 90%, or 100% of (i) amino acids 72-165 and/or (ii) amino acids 423-615 of maize ALKBH9B (SEQ ID NO: 20). In a further embodiment of this aspect, the ALKBH9B includes a deletion of at least about 10%, 20%, 30%, 40%, 50%, 60%, 70%, 80%, 90%, or 100% of amino acids 72-165 of maize ALKBH9B (SEQ ID NO: 20). In one embodiment of this aspect, the ALKBH9B includes a deletion of at least about 10%, 20%, 30%, 40%, 50%, 60%, 70%, 80%, 90%, or 100% of amino acids 423-615 of maize ALKBH9B (SEQ ID NO: 20). In another embodiment of this aspect, the ALKBH9B includes a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of maize ALKBH9B (SEQ ID NO: 20). In yet another embodiment of this aspect, the RNA m⁶A demethylase is maize ALKBH10B. In one embodiment of this aspect, the ALKBH10B includes a deletion of at least about 10%, 20%, 30%, 40%, 50%, 60%, 70%, 80%, 90%, or 100% of (i) amino acids 109-210 and/or (ii) amino acids 437-573 of maize ALKBH10B (SEQ ID NO: 22). In another embodiment of this aspect, the ALKBH10B includes a deletion of at least about 10%, 20%, 30%, 40%, 50%, 60%, 70%, 80%, 90%, or 100% of amino acids 109-210 of maize ALKBH10B (SEQ ID NO: 22). In still another embodiment of this aspect, the ALKBH10B includes a deletion of at least about 10%, 20%, 30%, 40%, 50%, 60%, 70%, 80%, 90%, or 100% of amino acids 437-573 of maize ALKBH10B (SEQ ID NO: 22). In a further embodiment of this aspect, the ALKBH10B includes a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of maize ALKBH10B (SEQ ID NO: 22). In an additional embodiment of this aspect, the RNA m⁶A demethylase is wheat ALKBH9B. In some embodiments of this aspect, the ALKBH9B includes a deletion of at least about 10%, 20%, 30%, 40%, 50%, 60%, 70%, 80%, 90%, or 100% of (i) amino acids 57-163 and/or (ii) amino acids 417-612 of wheat ALKBH9B (SEQ ID NO: 24). In an embodiment of this aspect, the ALKBH9B includes a deletion of at least about 10%, 20%, 30%, 40%, 50%, 60%, 70%, 80%, 90%, or 100% of amino acids 57-163 of wheat ALKBH9B (SEQ ID NO: 24). In yet another embodiment of this aspect, the ALKBH9B includes a deletion of at least about 10%, 20%, 30%, 40%, 50%, 60%, 70%, 80%, 90%, or 100% of amino acids 417-612 of wheat ALKBH9B (SEQ ID NO: 24). In an additional embodiment of this aspect, the ALKBH9B includes a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of wheat ALKBH9B (SEQ ID NO: 24). In still another embodiment of this aspect, the RNA m⁶A demethylase is wheat ALKBH10B. In some embodiments of this aspect, the ALKBH10B includes a deletion of at least about 10%, 20%, 30%, 40%, 50%, 60%, 70%, 80%, 90%, or 100% of (i) amino acids 91-211 and/or (ii) amino acids 440-566 of wheat ALKBH10B (SEQ ID NO: 26). In another embodiment of this aspect, the ALKBH10B includes a deletion of at least about 10%, 20%, 30%, 40%, 50%, 60%, 70%, 80%, 90%, or 100% of amino acids 91-211 of wheat ALKBH10B (SEQ ID NO: 26). In a further embodiment of this aspect, the ALKBH10B includes a deletion of at least about 10%, 20%, 30%, 40%, 50%, 60%, 70%, 80%, 90%, or 100% of amino acids 440-566 of wheat ALKBH10B (SEQ ID NO: 26). In still another embodiment of this aspect, the ALKBH10B includes a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of wheat ALKBH10B (SEQ ID NO: 26). In a further embodiment of this aspect, the RNA m⁶A demethylase is cassava ALKBH9B. In another embodiment of this aspect, the RNA m⁶A demethylase is cassava ALKBH10B. In an additional embodiment of this aspect, the RNA m⁶A demethylase is cowpea ALKBH9B. In another embodiment of this aspect, the RNA m⁶A demethylase is cowpea ALKBH10B. In a further embodiment of this aspect, the RNA m⁶A demethylase is cotton ALKBH9B. In another embodiment of this aspect, the RNA m⁶A demethylase is cotton ALKBH10B.

In one embodiment of this aspect, which may be combined with any of the previous plant embodiments, the RNA m⁶A demethylase is operably linked to at least one nuclear localization signal (NLS). In a further embodiment of this aspect, the NLS is encoded by SEQ ID NO: 40. In yet another embodiment of this aspect, the amino acid sequence of the NLS is SEQ ID NO: 41. In still another embodiment of this aspect, the RNA m⁶A demethylase is operably linked to at least one heterologous nuclear localization signal (NLS). In one embodiment of this aspect, the RNA m⁶A demethylase is operably linked to a promoter for expression in a plant. In an additional embodiment of this aspect, which may be combined with any of the previous plant embodiments, the promoter is a constitutive promoter.

A further aspect of the disclosure provides a plant part, tissue, or cell of the plant of any of the preceding plant embodiments. In some embodiments of this aspect, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. Photosynthesis can be measured using Leaf Gas Exchange Measurements, such as those obtained using an LI-6400XT Portable Photosynthesis System (LI-COR Inc., NE, USA) on attached leaves of target plants to accurately assess leaf-level gas exchange parameters that can reflect the intensity of photosynthesis. These measurements include the CO₂ assimilation rate, stomatal conductance, and transpiration rate. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant of any of the preceding plant embodiments. An additional aspect of the disclosure provides a commercial product derived from the plant of any of the preceding plant embodiments, or the seed of any of the preceding embodiments.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

Plants with Modified ALKBH5 Homolog Genes

Some aspects of the disclosure include a plant or plant cell including a modified endogenous ALKBH5 homolog gene, wherein the modified endogenous ALKBH5 homolog gene encodes a protein including at least one disrupted endogenous region, wherein the region is a low complexity region (LCR) and/or an intrinsically disordered region (IDR). Some aspects of the disclosure include a plant or plant cell including a modified endogenous ALKBH5 homolog gene, wherein the modified endogenous ALKBH5 homolog gene encodes a protein including at least one disrupted endogenous low complexity region (LCR). In a further embodiment of this aspect, the modified endogenous ALKBH5 homolog gene encodes a protein including a disrupted N-terminal LCR. In an additional embodiment of this aspect, the modified endogenous ALKBH5 homolog gene encodes a protein including a disrupted C-terminal LCR. In some embodiments of this aspect, which may be combined with any of the preceding embodiments, the modified endogenous ALKBH5 homolog gene includes a disrupted endogenous N-terminal LCR and a disrupted C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the modified endogenous ALKBH5 homolog gene includes a deletion of at least about 10%, 20%, 30%, 40%, 50%, 60%, 70%, 80%, 90%, or 100% deletion of at least one endogenous LCR. In additional embodiments of this aspect, the modified endogenous ALKBH5 homolog gene encodes a protein that lacks at least one endogenous LCR. In some further embodiments of this aspect, the modified endogenous ALKBH5 homolog gene encodes a protein that lacks an endogenous C-terminal LCR. In an additional embodiment of this aspect, the modified endogenous ALKBH5 homolog gene was modified to encode an mRNA that includes a stop codon before the C-terminal LCR. In a further embodiment of this aspect, the modified endogenous ALKBH5 homolog gene includes a deletion of at least about 10%, 20%, 30%, 40%, 50%, 60%, 70%, 80%, 90%, or 100% of the nucleotides encoding the C-terminal LCR. In some further embodiments of this aspect, the modified endogenous ALKBH5 homolog gene encodes a protein that lacks an endogenous N-terminal LCR. In a further embodiment of this aspect, the modified endogenous ALKBH5 homolog gene includes a deletion of at least about 10%, 20%, 30%, 40%, 50%, 60%, 70%, 80%, 90%, or 100% of the nucleotides encoding the N-terminal LCR. In yet another embodiment of this aspect, the plant or plant cell was modified by a genome editing technique selected from TALEN editing, zinc finger nuclease editing, RNA-guided endonuclease-mediated editing, prime editing, and base editing. In still another embodiment of this aspect, the modified endogenous ALKBH5 homolog gene was modified to add a nuclear localization signal (NLS) to the encoded protein. In a further embodiment of this aspect, the NLS is encoded by SEQ ID NO: 40. In yet another embodiment of this aspect, the amino acid sequence of the NLS is SEQ ID NO: 41. In one embodiment of this aspect, the NLS is a heterologous NLS. In another embodiment of this aspect, the NLS is an endogenous NLS.

In a further embodiment of this aspect, which may be combined with any of the preceding embodiments, the plant or a plant including the plant cell has improved growth compared to a control plant. In another embodiment of this aspect, the plant or a plant including the plant cell has improved growth under abiotic stress conditions. In an additional embodiment of this aspect, the plant or a plant including the plant cell has improved growth under drought stress, salt stress, heat stress, or cold stress. In yet another embodiment of this aspect, the plant or a plant including the plant cell has improved growth under biotic stress conditions. In still another embodiment of this aspect, the plant or a plant including the plant cell is improved in a characteristic selected from biomass, yield, root growth, root expansion, stem cell expansion of the root, stem cell expansion of the shoot, drought resistance, and pest resistance. In a further embodiment of this aspect, the plant or a plant including the plant cell has elevated photosynthesis. Photosynthesis can be measured using Leaf Gas Exchange Measurements, such as those obtained using an LI-6400XT Portable Photosynthesis System (LI-COR Inc., NE, USA) on attached leaves of target plants to accurately assess leaf-level gas exchange parameters that can reflect the intensity of photosynthesis. These measurements include the CO₂ assimilation rate, stomatal conductance, and transpiration rate. In another embodiment of this aspect, which may be combined with any of the previous plant embodiments, the plant or plant cell is a plant selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant or a plant including the plant cell of any of the preceding embodiments. An additional aspect of the disclosure provides a commercial product derived from the plant or a plant including the plant cell of any of the preceding embodiments, or the seed of any of the preceding embodiments.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Methods of Improving Growth of a Plant

Further aspects of the disclosure include a method of improving growth of a plant, including a) genetically modifying the plant by transforming the plant with one or more gene editing components that target an endogenous ALKBH5 homolog gene, wherein the endogenous ALKBH5 homolog gene is modified to encode a protein including at least one disrupted endogenous region, wherein the region is a low complexity region (LCR) and/or an intrinsically disordered region (IDR), and b) growing the plant, wherein the plant has improved growth compared to a control plant. Further aspects of the disclosure include a method of improving growth of a plant, including a) genetically modifying the plant by transforming the plant with one or more gene editing components that target an endogenous ALKBH5 homolog gene, wherein the endogenous ALKBH5 homolog gene is modified to encode a protein including at least one disrupted endogenous low complexity region (LCR), and b) growing the plant, wherein the plant has improved growth compared to a control plant. In a further embodiment of this aspect, the endogenous ALKBH5 homolog gene is modified to encode a protein including a disrupted endogenous N-terminal LCR. In yet another embodiment of this aspect, the endogenous ALKBH5 homolog gene is modified to encode a protein including a disrupted endogenous C-terminal LCR. In an additional embodiment of this aspect, the endogenous ALKBH5 homolog gene is modified to encode a protein including a disrupted endogenous N-terminal LCR and a disrupted endogenous C-terminal LCR. In some embodiments, which may be combined with any of the preceding embodiments, the disruption of the endogenous LCR is selected from a partial truncation, a full truncation, a deletion, and a replacement. In another embodiment of this aspect, which may be combined with any of the preceding embodiments, the endogenous ALKBH5 homolog gene is modified to include a deletion of at least about 10%, 20%, 30%, 40%, 50%, 60%, 70%, 80%, 90%, or 100% deletion of at least one endogenous LCR. In a further embodiment of this aspect, the endogenous ALKBH5 homolog gene is modified to encode a protein that lacks an endogenous C-terminal LCR. In yet another embodiment of this aspect, the endogenous ALKBH5 homolog gene is modified to encode an mRNA that includes a stop codon before the C-terminal LCR. In still another embodiment of this aspect, the endogenous ALKBH5 homolog gene is modified to include a deletion of at least about 10%, 20%, 30%, 40%, 50%, 60%, 70%, 80%, 90%, or 100% of the nucleotides encoding the C-terminal LCR. In another embodiment of this aspect, the endogenous ALKBH5 homolog gene is modified to encode a protein that lacks an endogenous N-terminal LCR. In another embodiment of this aspect, the endogenous ALKBH5 homolog gene is modified to include a deletion of at least about 10%, 20%, 30%, 40%, 50%, 60%, 70%, 80%, 90%, or 100% of the nucleotides encoding the N-terminal LCR. In a further embodiment of this aspect, which may be combined with any of the preceding embodiments, the one or more gene editing components include a TALEN, a ZFN, an RNA-guided endonuclease, a fusion protein including an RNA-guided endonuclease, a fusion protein including a reverse transcriptase, a fusion protein including an RNA-guided endonuclease fused to a reverse transcriptase, a guide RNA, a template RNA, and/or a donor oligonucleotide. In some embodiments of this aspect, which may be combined with any of the preceding embodiments, the endogenous ALKBH5 homolog gene is modified to add a nuclear localization signal (NLS) to the encoded protein. In a further embodiment of this aspect, the NLS is encoded by SEQ ID NO: 40. In yet another embodiment of this aspect, the amino acid sequence of the NLS is SEQ ID NO: 41. In one embodiment of this aspect, a heterologous NLS is added.

In another embodiment of this aspect, an endogenous NLS is added. In an additional embodiment of this aspect, the plant is improved in a characteristic selected from biomass, yield, root growth, root expansion, stem cell expansion of the root, stem cell expansion of the shoot, drought resistance, and pest resistance. In another embodiment of this aspect, the plant has improved growth under abiotic stress conditions. In an additional embodiment of this aspect, the plant has improved growth under drought stress, salt stress, heat stress, or cold stress. In yet another embodiment of this aspect, the plant has improved growth under biotic stress conditions. In still another embodiment of this aspect, the plant is improved in a characteristic selected from biomass, yield, root growth, root expansion, stem cell expansion of the root, stem cell expansion of the shoot, drought resistance, and pest resistance. In a further embodiment of this aspect, the plant has elevated photosynthesis.

A further aspect of the disclosure provides a plant produced by the method of any of the preceding embodiments. In one embodiment of this aspect, which may be combined with any of the previous plant embodiments, the plant is selected from Arabidopsis, rice, corn, soybean, potato, cassava, cowpea, cotton, turf grass, strawberry, blueberry, blackberry, raspberry, and wheat, or a plant cell therefrom. Another aspect of the disclosure provides a seed produced by the plant of any of the preceding embodiments. An additional aspect of the disclosure provides a commercial product derived from the plant of any of the preceding embodiments, or the seed of any of the preceding embodiments.

### ENUMERATED EMBODIMENTS

The following enumerated embodiments are representative of some aspects of the invention.

1. A recombinant DNA encoding an engineered RNA m⁶A demethylase, wherein the engineered RNA m⁶A demethylase comprises at least one disrupted endogenous region, wherein the region is a low complexity region (LCR) and/or an intrinsically disordered region.

1a. A recombinant DNA encoding an engineered RNA m⁶A demethylase, wherein the engineered RNA m⁶A demethylase comprises at least one disrupted endogenous low complexity region (LCR).

2. The recombinant DNA of embodiment 1, wherein the RNA m⁶A demethylase comprises a disrupted endogenous N-terminal LCR.

3. The recombinant DNA of embodiment 1, wherein the RNA m⁶A demethylase comprises a disrupted endogenous C-terminal LCR.

4. The recombinant DNA of any one of embodiments 1-3, wherein the RNA m⁶A demethylase comprises a disrupted endogenous N-terminal LCR and a disrupted C-terminal LCR.

5. The recombinant DNA of any one of embodiments 1-4, wherein the disruption of the endogenous LCR is selected from the group consisting of a partial truncation, a full truncation, a deletion, and a replacement.

6. The recombinant DNA of any one of embodiments 1-4, wherein the RNA m⁶A demethylase comprises a deletion of at least about 10%, 20%, 30%, 40%, 50%, 60%, 70%, 80%, 90%, or 100% deletion of at least one endogenous LCR.

7. The recombinant DNA of embodiment 1, wherein the RNA m⁶A demethylase lacks at least one endogenous LCR.

8. The recombinant DNA of any one of embodiments 1, 2, 4, and 7, wherein the RNA m⁶A demethylase lacks an endogenous N-terminal LCR.

9. The recombinant DNA of any one of embodiments 1, 3, 4, and 7, wherein the RNA m⁶A demethylase lacks an endogenous C-terminal LCR.

10. The recombinant DNA of any one of embodiments 1-9, wherein the RNA m⁶A demethylase lacks both an endogenous N-terminal LCR and an endogenous C-terminal LCR.

11. The recombinant DNA of any one of embodiments 1-10, wherein the RNA m⁶A demethylase is an engineered ALKBH5 or an engineered ALKBH5 homolog.

12. The recombinant DNA of any one of embodiments 1-11, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 42.

13. The recombinant DNA of any one of embodiments 1-11, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 43.

14. The recombinant DNA of any one of embodiments 1-11, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 44.

15. The recombinant DNA of any one of embodiments 1-14, wherein the endogenous LCR leads to assembly of the endogenous RNA m⁶A demethylase in foci or condensates within the cell.

16. The recombinant DNA of any one of embodiments 1-15, wherein the engineered RNA m⁶A demethylase is an engineered ALKBH5.

17. The recombinant DNA of embodiment 16, wherein the ALKBH5 comprises a deletion of at least about 10%, 20%, 30%, 40%, 50%, 60%, 70%, 80%, 90%, or 100% of:
(i) amino acids 30-81 and/or
(ii) amino acids 298-394 of endogenous ALKBH5 (SEQ ID NO: 1).

18. The recombinant DNA of embodiment 17, wherein the ALKBH5 comprises a deletion of at least about 10%, 20%, 30%, 40%, 50%, 60%, 70%, 80%, 90%, or 100% of amino acids 30-81 of endogenous ALKBH5 (SEQ ID NO: 1).

19. The recombinant DNA of embodiment 17, wherein the ALKBH5 comprises a deletion of at least about 10%, 20%, 30%, 40%, 50%, 60%, 70%, 80%, 90%, or 100% of amino acids 298-394 of endogenous ALKBH5 (SEQ ID NO: 1).

20. The recombinant DNA of embodiment 17, wherein the ALKBH5 comprises a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of endogenous ALKBH5 (SEQ ID NO: 1).

21. The recombinant DNA of embodiment 16, wherein the RNA m⁶A demethylase is selected from the group consisting of ALKBH5_{Δ30-81}, ALKBH5_{Δ298-394}, and ALKBH5_{Δ30-81 and 298-394}.

22. The recombinant DNA of embodiment 21, wherein the RNA m⁶A demethylase is ALKBH5_{Δ298-394}.

23. The recombinant DNA of any one of embodiments 1-14, wherein the RNA m⁶A demethylase is an engineered ALKBH5 homolog.

24. The recombinant DNA of embodiment 23, wherein the RNA m⁶A demethylase is an animal ALKBH5 homolog.

25. The recombinant DNA of embodiment 24, wherein the RNA m⁶A demethylase is a mammalian ALKBH5 homolog.

26. The recombinant DNA of embodiment 23, wherein the RNA m⁶A demethylase is a non-animal ALKBH5 homolog.

27. The recombinant DNA of embodiment 26, wherein the RNA m⁶A demethylase is a plant ALKBH5 homolog.

28. The recombinant DNA of embodiment 27, wherein the RNA m⁶A demethylase is an Arabidopsis ALKBH5 homolog.

29. The recombinant DNA of embodiment 28, wherein the RNA m⁶A demethylase is selected from the group consisting of ALKBH9B and ALKBH10B.

30. The recombinant DNA of embodiment 29, wherein the RNA m⁶A demethylase is ALKBH9B.

31. The recombinant DNA of embodiment 30, wherein the ALKBH9B comprises a deletion of at least about 10%, 20%, 30%, 40%, 50%, 60%, 70%, 80%, 90%, or 100% of:
(i) amino acids 76-102,
(ii) amino acids 145-183, and/or
(iii) amino acids 432-507 of endogenous ALKBH9B (SEQ ID NO: 3).

32. The recombinant DNA of embodiment 31, wherein the ALKBH9B comprises a deletion of at least about 10%, 20%, 30%, 40%, 50%, 60%, 70%, 80%, 90%, or 100% of amino acids 76-102 of endogenous ALKBH9B (SEQ ID NO: 3).

33. The recombinant DNA of embodiment 31, wherein the ALKBH9B comprises a deletion of at least about 10%, 20%, 30%, 40%, 50%, 60%, 70%, 80%, 90%, or 100% of amino acids 145-183 of endogenous ALKBH9B (SEQ ID NO: 3).

34. The recombinant DNA of embodiment 31, wherein the ALKBH9B comprises a deletion of at least about 10%, 20%, 30%, 40%, 50%, 60%, 70%, 80%, 90%, or 100% of amino acids 432-507 of endogenous ALKBH9B (SEQ ID NO: 3).

35. The recombinant DNA of embodiment 31, wherein the ALKBH9B comprises a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of endogenous ALKBH9B (SEQ ID NO: 3).

36. The recombinant DNA of embodiment 31, wherein the ALKBH9B comprises a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of endogenous ALKBH9B (SEQ ID NO: 3).

37. The recombinant DNA of embodiment 31, wherein the ALKBH9B comprises a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of endogenous ALKBH9B (SEQ ID NO: 3).

38. The recombinant DNA of embodiment 31, wherein the ALKBH9B comprises a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of endogenous ALKBH9B (SEQ ID NO: 3).

39. The recombinant DNA of embodiment 30, wherein the ALKBH9B is selected from the group consisting of ALKBH9B 176-102, ALKBH9B 1145-183, and ALKBH9B 1432-507.

40. The recombinant DNA of embodiment 26, wherein the RNA m⁶A demethylase is ALKBH10B.

41. The recombinant DNA of embodiment 40, wherein the ALKBH10B comprises a deletion of at least about 10%, 20%, 30%, 40%, 50%, 60%, 70%, 80%, 90%, or 100% of:
(i) amino acids 131-190 and/or
(ii) amino acids 501-569
of endogenous ALKBH10B (SEQ ID NO: 5).

42. The recombinant DNA of embodiment 40, wherein the ALKBH10B comprises a deletion of at least about 10%, 20%, 30%, 40%, 50%, 60%, 70%, 80%, 90%, or 100% of amino acids 131-190 of endogenous ALKBH10B (SEQ ID NO: 5).

43. The recombinant DNA of embodiment 40, wherein the ALKBH10B comprises a deletion of at least about 10%, 20%, 30%, 40%, 50%, 60%, 70%, 80%, 90%, or 100% of amino acids 501-569 of endogenous ALKBH10B (SEQ ID NO: 5).

44. The recombinant DNA of embodiment 40, wherein the ALKBH10B comprises a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of endogenous ALKBH10B (SEQ ID NO: 5).

45. The recombinant DNA of embodiment 27, wherein the RNA m⁶A demethylase is a rice ALKBH5 homolog.

46. The recombinant DNA of embodiment 45, wherein the RNA m⁶A demethylase is selected from the group consisting of Os9B and Os10B.

47. The recombinant DNA of embodiment 46, wherein the RNA m⁶A demethylase is Os9B.

48. The recombinant DNA of embodiment 47, wherein the Os9B comprises a deletion of at least about 10%, 20%, 30%, 40%, 50%, 60%, 70%, 80%, 90%, or 100% of:
(i) amino acids 60-170 and/or
(ii) amino acids 428-616
of endogenous Os9B (SEQ ID NO: 6).

49. The recombinant DNA of embodiment 47, wherein the Os9B comprises a deletion of at least about 10%, 20%, 30%, 40%, 50%, 60%, 70%, 80%, 90%, or 100% of amino acids 60-170 of endogenous Os9B (SEQ ID NO: 6).

50. The recombinant DNA of embodiment 47, wherein the Os9B comprises a deletion of at least about 10%, 20%, 30%, 40%, 50%, 60%, 70%, 80%, 90%, or 100% of amino acids 428-616 of endogenous Os9B (SEQ ID NO: 6).

51. The recombinant DNA of embodiment 47, wherein the Os9B comprises a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of endogenous Os9B (SEQ ID NO: 6).

52. The recombinant DNA of embodiment 47, wherein the Os9B is selected from the group consisting of Os9B 360-170 and 428-616, and Os9B 1428-616.

53. The recombinant DNA of embodiment 46, wherein the RNA m⁶A demethylase is Os10B.

54. The recombinant DNA of embodiment 53, wherein the Os10B comprises a deletion of at least about 10%, 20%, 30%, 40%, 50%, 60%, 70%, 80%, 90%, or 100% of:
(i) amino acids 2-30,
(ii) amino acids 99-126, and/or
(iii) amino acids 491-595
of endogenous Os10B (SEQ ID NO: 8).

55. The recombinant DNA of embodiment 54, wherein the Os10B comprises a deletion of at least about 10%, 20%, 30%, 40%, 50%, 60%, 70%, 80%, 90%, or 100% of amino acids 2-30 of endogenous Os10B (SEQ ID NO: 8).

56. The recombinant DNA of embodiment 54, wherein the Os10B comprises a deletion of at least about 10%, 20%, 30%, 40%, 50%, 60%, 70%, 80%, 90%, or 100% of amino acids 99-126 of endogenous Os10B (SEQ ID NO: 8).

57. The recombinant DNA of embodiment 54, wherein the Os10B comprises a deletion of at least about 10%, 20%, 30%, 40%, 50%, 60%, 70%, 80%, 90%, or 100% of amino acids 491-595 of endogenous Os10B (SEQ ID NO: 8).

58. The recombinant DNA of embodiment 54, wherein the Os10B comprises a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of endogenous Os10B (SEQ ID NO: 8).

59. The recombinant DNA of embodiment 54, wherein the Os10B comprises a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of endogenous Os10B (SEQ ID NO: 8).

60. The recombinant DNA of embodiment 54, wherein the Os10B comprises a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of endogenous Os10B (SEQ ID NO: 8).

61. The recombinant DNA of embodiment 54, wherein the Os10B comprises a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of endogenous Os10B (SEQ ID NO: 8).

62. The recombinant DNA of embodiment 53, wherein the Os10B is selected from the group consisting of Os10B 12-30, 99-126, and 491-595, and Os10B 12-30, 99-126, and 389-595.

63. The recombinant DNA of embodiment 27, wherein the RNA m⁶A demethylase is rapeseed ALKBH9B.

64. The recombinant DNA of embodiment 63, wherein the ALKBH9B comprises a deletion of at least about 10%, 20%, 30%, 40%, 50%, 60%, 70%, 80%, 90%, or 100% of:
(i) amino acids 55-137 and/or
(ii) amino acids 401-467
of endogenous rapeseed ALKBH9B (SEQ ID NO: 10).

65. The recombinant DNA of embodiment 63, wherein the ALKBH9B comprises a deletion of at least about 10%, 20%, 30%, 40%, 50%, 60%, 70%, 80%, 90%, or 100% of amino acids 55-137 of endogenous rapeseed ALKBH9B (SEQ ID NO: 10).

66. The recombinant DNA of embodiment 63, wherein the ALKBH9B comprises a deletion of at least about 10%, 20%, 30%, 40%, 50%, 60%, 70%, 80%, 90%, or 100% of amino acids 401-467 of endogenous rapeseed ALKBH9B (SEQ ID NO: 10).

67. The recombinant DNA of embodiment 63, wherein the ALKBH9B comprises a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of endogenous rapeseed ALKBH9B (SEQ ID NO: 10).

68. The recombinant DNA of embodiment 27, wherein the RNA m⁶A demethylase is tobacco ALKBH9B.

69. The recombinant DNA of embodiment 68, wherein the ALKBH9B comprises a deletion of at least about 10%, 20%, 30%, 40%, 50%, 60%, 70%, 80%, 90%, or 100% of:
(i) amino acids 121-243 and/or
(ii) amino acids 447-541
of endogenous tobacco ALKBH9B (SEQ ID NO: 12).

70. The recombinant DNA of embodiment 68, wherein the ALKBH9B comprises a deletion of at least about 10%, 20%, 30%, 40%, 50%, 60%, 70%, 80%, 90%, or 100% of amino acids 121-243 of endogenous tobacco ALKBH9B (SEQ ID NO: 12).

71. The recombinant DNA of embodiment 68, wherein the ALKBH9B comprises a deletion of at least about 10%, 20%, 30%, 40%, 50%, 60%, 70%, 80%, 90%, or 100% of amino acids 447-541 of endogenous tobacco ALKBH9B (SEQ ID NO: 12).

72. The recombinant DNA of embodiment 68, wherein the ALKBH9B comprises a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of endogenous tobacco ALKBH9B (SEQ ID NO: 12).

73. The recombinant DNA of embodiment 27, wherein the RNA m⁶A demethylase is tobacco ALKBH10B.

74. The recombinant DNA of embodiment 73, wherein the ALKBH10B comprises a deletion of at least about 10%, 20%, 30%, 40%, 50%, 60%, 70%, 80%, 90%, or 100% of:
(i) amino acids 181-235 and/or
(ii) amino acids 504-640
of endogenous tobacco ALKBH10B (SEQ ID NO: 14).

75. The recombinant DNA of embodiment 73, wherein the ALKBH10B comprises a deletion of at least about 10%, 20%, 30%, 40%, 50%, 60%, 70%, 80%, 90%, or 100% of amino acids 181-235 of endogenous tobacco ALKBH10B (SEQ ID NO: 14).

76. The recombinant DNA of embodiment 73, wherein the ALKBH10B comprises a deletion of at least about 10%, 20%, 30%, 40%, 50%, 60%, 70%, 80%, 90%, or 100% of amino acids 504-640 of endogenous tobacco ALKBH10B (SEQ ID NO: 14).

77. The recombinant DNA of embodiment 73, wherein the ALKBH10B comprises a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of endogenous tobacco ALKBH10B (SEQ ID NO: 14).

78. The recombinant DNA of embodiment 27, wherein the RNA m⁶A demethylase is alfalfa ALKBH9B.

79. The recombinant DNA of embodiment 78, wherein the ALKBH9B comprises a deletion of at least about 10%, 20%, 30%, 40%, 50%, 60%, 70%, 80%, 90%, or 100% of amino acids 442-508 of endogenous alfalfa ALKBH9B (SEQ ID NO: 16).

80. The recombinant DNA of embodiment 27, wherein the RNA m⁶A demethylase is sorghum ALKBH9B.

81. The recombinant DNA of embodiment 80, wherein the ALKBH9B comprises a deletion of at least about 10%, 20%, 30%, 40%, 50%, 60%, 70%, 80%, 90%, or 100% of:
(i) amino acids 66-179 and/or
(ii) amino acids 423-611
of endogenous sorghum ALKBH9B (SEQ ID NO: 18).

82. The recombinant DNA of embodiment 80, wherein the ALKBH9B comprises a deletion of at least about 10%, 20%, 30%, 40%, 50%, 60%, 70%, 80%, 90%, or 100% of amino acids 66-179 of endogenous sorghum ALKBH9B (SEQ ID NO: 18).

83. The recombinant DNA of embodiment 80, wherein the ALKBH9B comprises a deletion of at least about 10%, 20%, 30%, 40%, 50%, 60%, 70%, 80%, 90%, or 100% of amino acids 423-611 of endogenous sorghum ALKBH9B (SEQ ID NO: 18).

84. The recombinant DNA of embodiment 80, wherein the ALKBH9B comprises a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of endogenous sorghum ALKBH9B (SEQ ID NO: 18).

85. The recombinant DNA of embodiment 27, wherein the RNA m⁶A demethylase is maize ALKBH9B.

86. The recombinant DNA of embodiment 85, wherein the ALKBH9B comprises a deletion of at least about 10%, 20%, 30%, 40%, 50%, 60%, 70%, 80%, 90%, or 100% of:
(i) amino acids 72-165 and/or
(ii) amino acids 423-615
of endogenous maize ALKBH9B (SEQ ID NO: 20).

87. The recombinant DNA of embodiment 85, wherein the ALKBH9B comprises a deletion of at least about 10%, 20%, 30%, 40%, 50%, 60%, 70%, 80%, 90%, or 100% of amino acids 72-165 of endogenous maize ALKBH9B (SEQ ID NO: 20).

88. The recombinant DNA of embodiment 85, wherein the ALKBH9B comprises a deletion of at least about 10%, 20%, 30%, 40%, 50%, 60%, 70%, 80%, 90%, or 100% of amino acids 423-615 of endogenous maize ALKBH9B (SEQ ID NO: 20).

89. The recombinant DNA of embodiment 85, wherein the ALKBH9B comprises a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of endogenous maize ALKBH9B (SEQ ID NO: 20).

90. The recombinant DNA of embodiment 27, wherein the RNA m⁶A demethylase is maize ALKBH10B.

91. The recombinant DNA of embodiment 90, wherein the ALKBH10B comprises a deletion of at least about 10%, 20%, 30%, 40%, 50%, 60%, 70%, 80%, 90%, or 100% of:
(i) amino acids 109-210 and/or
(ii) amino acids 437-573
of endogenous maize ALKBH10B (SEQ ID NO: 22).

92. The recombinant DNA of embodiment 90, wherein the ALKBH10B comprises a deletion of at least about 10%, 20%, 30%, 40%, 50%, 60%, 70%, 80%, 90%, or 100% of amino acids 109-210 of endogenous maize ALKBH10B (SEQ ID NO: 22).

93. The recombinant DNA of embodiment 90, wherein the ALKBH10B comprises a deletion of at least about 10%, 20%, 30%, 40%, 50%, 60%, 70%, 80%, 90%, or 100% of amino acids 437-573 of endogenous maize ALKBH10B (SEQ ID NO: 22).

94. The recombinant DNA of embodiment 90, wherein the ALKBH10B comprises a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of endogenous maize ALKBH10B (SEQ ID NO: 22).

95. The recombinant DNA of embodiment 27, wherein the RNA m⁶A demethylase is wheat ALKBH9B.

96. The recombinant DNA of embodiment 95, wherein the ALKBH9B comprises a deletion of at least about 10%, 20%, 30%, 40%, 50%, 60%, 70%, 80%, 90%, or 100% of:
(i) amino acids 57-163 and/or
(ii) amino acids 417-612
of endogenous wheat ALKBH9B (SEQ ID NO: 24).

97. The recombinant DNA of embodiment 95, wherein the ALKBH9B comprises a deletion of at least about 10%, 20%, 30%, 40%, 50%, 60%, 70%, 80%, 90%, or 100% of amino acids 57-163 of endogenous wheat ALKBH9B (SEQ ID NO: 24).

98. The recombinant DNA of embodiment 95, wherein the ALKBH9B comprises a deletion of at least about 10%, 20%, 30%, 40%, 50%, 60%, 70%, 80%, 90%, or 100% of amino acids 417-612 of endogenous wheat ALKBH9B (SEQ ID NO: 24).

99. The recombinant DNA of embodiment 95, wherein the ALKBH9B comprises a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of endogenous wheat ALKBH9B (SEQ ID NO: 24).

100. The recombinant DNA of embodiment 27, wherein the RNA m⁶A demethylase is wheat ALKBH10B.

101. The recombinant DNA of embodiment 100, wherein the ALKBH10B comprises a deletion of at least about 10%, 20%, 30%, 40%, 50%, 60%, 70%, 80%, 90%, or 100% of:
(i) amino acids 91-211 and/or
(ii) amino acids 440-566
of endogenous wheat ALKBH10B (SEQ ID NO: 26).

102. The recombinant DNA of embodiment 100, wherein the ALKBH10B comprises a deletion of at least about 10%, 20%, 30%, 40%, 50%, 60%, 70%, 80%, 90%, or 100% of amino acids 91-211 of endogenous wheat ALKBH10B (SEQ ID NO: 26).

103. The recombinant DNA of embodiment 100, wherein the ALKBH10B comprises a deletion of at least about 10%, 20%, 30%, 40%, 50%, 60%, 70%, 80%, 90%, or 100% of amino acids 440-566 of endogenous wheat ALKBH10B (SEQ ID NO: 26).

104. The recombinant DNA of embodiment 100, wherein the ALKBH10B comprises a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of endogenous wheat ALKBH10B (SEQ ID NO: 26).

105. The recombinant DNA of any one of embodiments 1-104, wherein the RNA m⁶A demethylase is operably linked to at least one nuclear localization signal (NLS).

106. The recombinant DNA of embodiment 105, wherein the RNA m⁶A demethylase is operably linked to at least one heterologous nuclear localization signal (NLS).

107. The recombinant DNA of any one of embodiments 1-106, wherein the engineered RNA m⁶A demethylase is operably linked to a promoter for expression in a plant.

108. The recombinant DNA of embodiment 107, wherein the promoter is a constitutive promoter.

109. An expression vector comprising the recombinant DNA of any one of embodiments 1-108.

110. An expression vector comprising a nucleic acid encoding an engineered RNA m⁶A demethylase, wherein the engineered RNA m⁶A demethylase comprises at least one disrupted endogenous low complexity region (LCR), and wherein the expression vector is for expression in a plant or plant cell.

111. A transformation vector comprising the recombinant DNA of any one of embodiments 1-108 or the expression vector of embodiment 109 or embodiment 110.

112. A plant or plant cell comprising the recombinant DNA of any one of embodiments 1-108, or the expression vector of embodiment 109 or embodiment 110.

113. The plant or plant cell of embodiment 112, wherein the plant or a plant comprising the plant cell has improved growth compared to a control plant.

114. The plant or plant cell of embodiment 113, wherein the plant or a plant comprising the plant cell has improved growth under abiotic stress conditions.

115. The plant or plant cell of embodiment 114, wherein the plant or a plant comprising the plant cell has improved growth under drought stress, salt stress, heat stress, or cold stress.

116. The plant or plant cell of embodiment 113, wherein the plant or a plant comprising the plant cell has improved growth under biotic stress conditions.

117. The plant or plant cell of embodiment 113, wherein the plant or a plant comprising the plant cell is improved in a characteristic selected from the group consisting of biomass, yield, root growth, root expansion, stem cell expansion of the root, stem cell expansion of the shoot, drought resistance, and pest resistance.

118. The plant or plant cell of embodiment 113, wherein the plant or a plant comprising the plant cell has elevated photosynthesis.

119. The plant or plant cell of any one of embodiments 112-118, wherein the plant or plant cell is a plant selected from the group consisting of Arabidopsis, rice, corn, soybean, potato, cassava, cowpea, cotton, turf grass, strawberry, blueberry, blackberry, raspberry, and wheat, or a plant cell therefrom.

120. A seed produced by the plant or a plant comprising the plant cell of any one of embodiments 112-119.

121. A commercial product derived from the plant or a plant comprising the plant cell of any one of embodiments 112-119 or the seed of embodiment 120.

122. A plant comprising nucleic acid encoding an engineered RNA m⁶A demethylase, wherein the engineered RNA m⁶A demethylase comprises at least one disrupted endogenous low complexity region (LCR).

123. The plant of embodiment 122, wherein the RNA m⁶A demethylase comprises a disrupted endogenous N-terminal LCR.

124. The plant of embodiment 122, wherein the RNA m⁶A demethylase comprises a disrupted endogenous C-terminal LCR.

125. The plant of any one of embodiments 122-125, wherein the RNA m⁶A demethylase comprises a disrupted endogenous N-terminal LCR and a disrupted C-terminal LCR.

126. The plant of any one of embodiments 122-126, wherein the disruption of the endogenous LCR is selected from the group consisting of a partial truncation, a full truncation, a deletion, and a replacement.

127. The plant of any one of embodiments 122-125, wherein the RNA m⁶A demethylase comprises a deletion of at least about 10%, 20%, 30%, 40%, 50%, 60%, 70%, 80%, 90%, or 100% deletion of at least one endogenous LCR.

128. The plant of embodiment 122, wherein the RNA m⁶A demethylase lacks at least one endogenous LCR.

129. The plant of any one of embodiments 122, 123, 125, and 128, wherein the RNA m⁶A demethylase lacks an endogenous N-terminal LCR.

130. The plant of any one of embodiments 122, 124, 125, and 128, wherein the RNA m⁶A demethylase lacks an endogenous C-terminal LCR.

131. The plant of any one of embodiments 122-130, wherein the RNA m⁶A demethylase lacks both an endogenous N-terminal LCR and an endogenous C-terminal LCR.

132. The plant of any one of embodiments 122-131, wherein the RNA m⁶A demethylase is an engineered ALKBH5 or an engineered ALKBH5 homolog.

133. The plant of any one of embodiments 122-132, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO:

134. The plant of any one of embodiments 122-132, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 43.

135. The plant of any one of embodiments 122-132, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to SEQ ID NO: 44.

136. The plant of any one of embodiments 122-135, wherein the endogenous LCR leads to assembly of the endogenous RNA m⁶A demethylase in foci or condensates within the cell.

137. The plant of any one of embodiments 122-136, wherein the engineered RNA m⁶A demethylase is a heterologous RNA m⁶A demethylase.

138. The plant of any one of embodiments 122-136, wherein the engineered RNA m⁶A demethylase is an endogenous RNA m⁶A demethylase.

139. The plant of any one of embodiments 122-136, wherein the engineered RNA m⁶A demethylase is an engineered ALKBH5.

140. The plant of embodiment 139, wherein the ALKBH5 comprises a deletion of at least about 10%, 20%, 30%, 40%, 50%, 60%, 70%, 80%, 90%, or 100% of:
(i) amino acids 30-81 and/or
(ii) amino acids 298-394
of endogenous ALKBH5 (SEQ ID NO: 1).

141. The plant of embodiment 139, wherein the ALKBH5 comprises a deletion of at least about 10%, 20%, 30%, 40%, 50%, 60%, 70%, 80%, 90%, or 100% of amino acids 30-81 of endogenous ALKBH5 (SEQ ID NO: 1).

142. The plant of embodiment 139, wherein the ALKBH5 comprises a deletion of at least about 10%, 20%, 30%, 40%, 50%, 60%, 70%, 80%, 90%, or 100% of amino acids 298-394 of endogenous ALKBH5 (SEQ ID NO: 1).

143. The plant of embodiment 139, wherein the ALKBH5 comprises a deletion of at least about 10%, 20%, 30%, 40%, 50%, 60%, 70%, 80%, 90%, or 100% of amino acids 30-81 and a deletion of at least about 10%, 20%, 30%, 40%, 50%, 60%, 70%, 80%, 90%, or 100% of amino acids 298-394 of endogenous ALKBH5 (SEQ ID NO: 1).

144. The plant of embodiment 139, wherein the RNA m⁶A demethylase is selected from the group consisting of ALKBH5_{Δ30-81}, ALKBH5_{Δ298-394}, and ALKBH5_{Δ30-81 and 298-394}.

145. The plant of embodiment 144, wherein the RNA m⁶A demethylase is ALKBH5_{Δ298-394}.

146. The plant of any one of embodiments 122-136, wherein the RNA m⁶A demethylase is an engineered ALKBH5 homolog.

147. The plant of embodiment 146, wherein the RNA m⁶A demethylase is an animal ALKBH5 homolog.

148. The plant of embodiment 147, wherein the RNA m⁶A demethylase is a mammalian ALKBH5 homolog.

149. The plant of embodiment 146, wherein the RNA m⁶A demethylase is a non-animal ALKBH5 homolog.

150. The plant of embodiment 146, wherein the RNA m⁶A demethylase is a plant ALKBH5 homolog.

151. The plant of embodiment 150, wherein the RNA m⁶A demethylase is an Arabidopsis ALKBH5 homolog.

152. The plant of embodiment 151, wherein the RNA m⁶A demethylase is selected from the group consisting of ALKBH9B and ALKBH10B.

153. The plant of embodiment 152, wherein the RNA m⁶A demethylase is ALKBH9B.

154. The plant of embodiment 153, wherein the ALKBH9B comprises a deletion of at least about 10%, 20%, 30%, 40%, 50%, 60%, 70%, 80%, 90%, or 100% of:
(i) amino acids 76-102,
(ii) amino acids 145-183, and/or
(iii) amino acids 432-507
of Arabidopsis ALKBH9B (SEQ ID NO: 3).

155. The plant of embodiment 154, wherein the ALKBH9B comprises a deletion of at least about 10%, 20%, 30%, 40%, 50%, 60%, 70%, 80%, 90%, or 100% of amino acids 76-102 of Arabidopsis ALKBH9B (SEQ ID NO: 3).

156. The plant of embodiment 154, wherein the ALKBH9B comprises a deletion of at least about 10%, 20%, 30%, 40%, 50%, 60%, 70%, 80%, 90%, or 100% of amino acids 145-183 of Arabidopsis ALKBH9B (SEQ ID NO: 3).

157. The plant of embodiment 154, wherein the ALKBH9B comprises a deletion of at least about 10%, 20%, 30%, 40%, 50%, 60%, 70%, 80%, 90%, or 100% of amino acids 432-507 of Arabidopsis ALKBH9B (SEQ ID NO: 3).

158. The plant of embodiment 154, wherein the ALKBH9B comprises a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 145-183 of Arabidopsis ALKBH9B (SEQ ID NO: 3).

159. The plant of embodiment 154, wherein the ALKBH9B comprises a deletion of at least about 10%, 20%, 30%, 40%, 50%, 60%, 70%, 80%, 90%, or 100% of amino acids 76-102 and a deletion of at least about 10%, 20%, 30%, 40%, 50%, 60%, 70%, 80%, 90%, or 100% of amino acids 432-507 of Arabidopsis ALKBH9B (SEQ ID NO: 3).

160. The plant of embodiment 154, wherein the ALKBH9B comprises a deletion of at least about 10%, 20%, 30%, 40%, 50%, 60%, 70%, 80%, 90%, or 100% of amino acids 145-183 and a deletion of at least about 10%, 20%, 30%, 40%, 50%, 60%, 70%, 80%, 90%, or 100% of amino acids 432-507 of Arabidopsis ALKBH9B (SEQ ID NO: 3).

161. The plant of embodiment 154, wherein the ALKBH9B comprises a deletion of at least about 10%, 20%, 30%, 40%, 50%, 60%, 70%, 80%, 90%, or 100% of amino acids 76-102, a deletion of at least about 10%, 20%, 30%, 40%, 50%, 60%, 70%, 80%, 90%, or 100% of amino acids 145-183, and a deletion of at least about 10%, 20%, 30%, 40%, 50%, 60%, 70%, 80%, 90%, or 100% of amino acids 432-507 of Arabidopsis ALKBH9B (SEQ ID NO: 3).

162. The plant of embodiment 153, wherein the ALKBH9B is selected from the group consisting of ALKBH9B 176-102, ALKBH9B 1145-183, and ALKBH9B 1432-507.

163. The plant of embodiment 152, wherein the RNA m⁶A demethylase is ALKBH10B.

164. The plant of embodiment 163, wherein the ALKBH10B comprises a deletion of at least about 10%, 20%, 30%, 40%, 50%, 60%, 70%, 80%, 90%, or 100% of:
(i) amino acids 131-190 and/or
(ii) amino acids 501-569
of Arabidopsis ALKBH10B (SEQ ID NO: 5).

165. The plant of embodiment 164, wherein the ALKBH10B comprises a deletion of at least about 10%, 20%, 30%, 40%, 50%, 60%, 70%, 80%, 90%, or 100% of amino acids 131-190 of Arabidopsis ALKBH10B (SEQ ID NO: 5).

166. The plant of embodiment 164, wherein the ALKBH10B comprises a deletion of at least about 10%, 20%, 30%, 40%, 50%, 60%, 70%, 80%, 90%, or 100% of amino acids 501-569 of Arabidopsis ALKBH10B (SEQ ID NO: 5).

167. The plant of embodiment 164, wherein the ALKBH10B comprises a deletion of at least about 10%, 20%, 30%, 40%, 50%, 60%, 70%, 80%, 90%, or 100% of amino acids 131-190 and a deletion of at least about 10%, 20%, 30%, 40%, 50%, 60%, 70%, 80%, 90%, or 100% of amino acids 501-569 of Arabidopsis ALKBH10B (SEQ ID NO: 5).

168. The plant of embodiment 150, wherein the RNA m⁶A demethylase is a rice ALKBH5 homolog.

169. The plant of embodiment 168, wherein the RNA m⁶A demethylase is selected from the group consisting of Os9B and Os10B.

170. The plant of embodiment 169, wherein the RNA m⁶A demethylase is Os9B.

171. The plant of embodiment 170, wherein the Os9B comprises a deletion of at least about 10%, 20%, 30%, 40%, 50%, 60%, 70%, 80%, 90%, or 100% of:
(i) amino acids 60-170 and/or
(ii) amino acids 428-616
of Os9B (SEQ ID NO: 6).

172. The plant of embodiment 171, wherein the Os9B comprises a deletion of at least about 10%, 20%, 30%, 40%, 50%, 60%, 70%, 80%, 90%, or 100% of amino acids 60-170 of Os9B (SEQ ID NO: 6).

173. The plant of embodiment 171, wherein the Os9B comprises a deletion of at least about 10%, 20%, 30%, 40%, 50%, 60%, 70%, 80%, 90%, or 100% of amino acids 428-616 of Os9B (SEQ ID NO: 6).

174. The plant of embodiment 171, wherein the Os9B comprises a deletion of at least about 10%, 20%, 30%, 40%, 50%, 60%, 70%, 80%, 90%, or 100% of amino acids 60-170 and a deletion of at least about 10%, 20%, 30%, 40%, 50%, 60%, 70%, 80%, 90%, or 100% of amino acids 428-616 of Os9B (SEQ ID NO: 6).

175. The plant of embodiment 170, wherein the Os9B is selected from the group consisting of Os9B 360-170 and 428-616, and Os9B 1428-616.

176. The plant of embodiment 169, wherein the RNA m⁶A demethylase is Os10B.

177. The plant of embodiment 176, wherein the Os10B comprises a deletion of at least about 10%, 20%, 30%, 40%, 50%, 60%, 70%, 80%, 90%, or 100% of:
(i) amino acids 2-30,
(ii) amino acids 99-126, and/or
(iii) amino acids 491-595
of Os10B (SEQ ID NO: 8).

178. The plant of embodiment 177, wherein the Os10B comprises a deletion of at least about 10%, 20%, 30%, 40%, 50%, 60%, 70%, 80%, 90%, or 100% of amino acids 2-30 of Os10B (SEQ ID NO: 8).

179. The plant of embodiment 177, wherein the Os10B comprises a deletion of at least about 10%, 20%, 30%, 40%, 50%, 60%, 70%, 80%, 90%, or 100% of amino acids 99-126 of Os10B (SEQ ID NO: 8).

180. The plant of embodiment 177, wherein the Os10B comprises a deletion of at least about 10%, 20%, 30%, 40%, 50%, 60%, 70%, 80%, 90%, or 100% of amino acids 491-595 of Os10B (SEQ ID NO: 8).

181. The plant of embodiment 177, wherein the Os10B comprises a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 99-126 of Os10B (SEQ ID NO: 8).

182. The plant of embodiment 177, wherein the Os10B comprises a deletion of at least about 10%, 20%, 30%, 40%, 50%, 60%, 70%, 80%, 90%, or 100% of amino acids 2-30 and a deletion of at least about 10%, 20%, 30%, 40%, 50%, 60%, 70%, 80%, 90%, or 100% of amino acids 491-595 of Os10B (SEQ ID NO: 8).

183. The plant of embodiment 177, wherein the Os10B comprises a deletion of at least about 10%, 20%, 30%, 40%, 50%, 60%, 70%, 80%, 90%, or 100% of amino acids 99-126 and a deletion of at least about 10%, 20%, 30%, 40%, 50%, 60%, 70%, 80%, 90%, or 100% of amino acids 491-595 of Os10B (SEQ ID NO: 8).

184. The plant of embodiment 177, wherein the Os10B comprises a deletion of at least about 10%, 20%, 30%, 40%, 50%, 60%, 70%, 80%, 90%, or 100% of amino acids 2-30, a deletion of at least about 10%, 20%, 30%, 40%, 50%, 60%, 70%, 80%, 90%, or 100% of amino acids 99-126, and a deletion of at least about 10%, 20%, 30%, 40%, 50%, 60%, 70%, 80%, 90%, or 100% of amino acids 491-595 of Os10B (SEQ ID NO: 8).

185. The plant of embodiment 176, wherein the Os10B is selected from the group consisting of Os10B 12-30, 99-126, and 491-595, and Os10B 12-30, 99-126, and 389-595.

186. The plant of embodiment 150, wherein the RNA m⁶A demethylase is rapeseed ALKBH9B.

187. The plant of embodiment 186, wherein the ALKBH9B comprises a deletion of at least about 10%, 20%, 30%, 40%, 50%, 60%, 70%, 80%, 90%, or 100% of:
(i) amino acids 55-137 and/or
(ii) amino acids 401-467
of rapeseed ALKBH9B (SEQ ID NO: 10).

188. The plant of embodiment 187, wherein the ALKBH9B comprises a deletion of at least about 10%, 20%, 30%, 40%, 50%, 60%, 70%, 80%, 90%, or 100% of amino acids 55-137 of rapeseed ALKBH9B (SEQ ID NO: 10).

189. The plant of embodiment 187, wherein the ALKBH9B comprises a deletion of at least about 10%, 20%, 30%, 40%, 50%, 60%, 70%, 80%, 90%, or 100% of amino acids 401-467 of rapeseed ALKBH9B (SEQ ID NO: 10).

190. The plant of embodiment 187, wherein the ALKBH9B comprises a deletion of at least about 10%, 20%, 30%, 40%, 50%, 60%, 70%, 80%, 90%, or 100% of amino acids 55-137 and a deletion of at least about 10%, 20%, 30%, 40%, 50%, 60%, 70%, 80%, 90%, or 100% of amino acids 401-467 of rapeseed ALKBH9B (SEQ ID NO: 10).

191. The plant of embodiment 150, wherein the RNA m⁶A demethylase is tobacco ALKBH9B.

192. The plant of embodiment 191, wherein the ALKBH9B comprises a deletion of at least about 10%, 20%, 30%, 40%, 50%, 60%, 70%, 80%, 90%, or 100% of:
(i) amino acids 121-243 and/or
(ii) amino acids 447-541
of tobacco ALKBH9B (SEQ ID NO: 12).

193. The plant of embodiment 192, wherein the ALKBH9B comprises a deletion of at least about 10%, 20%, 30%, 40%, 50%, 60%, 70%, 80%, 90%, or 100% of amino acids 121-243 of tobacco ALKBH9B (SEQ ID NO: 12).

194. The plant of embodiment 192, wherein the ALKBH9B comprises a deletion of at least about 10%, 20%, 30%, 40%, 50%, 60%, 70%, 80%, 90%, or 100% of amino acids 447-541 of tobacco ALKBH9B (SEQ ID NO: 12).

195. The plant of embodiment 192, wherein the ALKBH9B comprises a deletion of at least about 10%, 20%, 30%, 40%, 50%, 60%, 70%, 80%, 90%, or 100% of amino acids 121-243 and a deletion of at least about 10%, 20%, 30%, 40%, 50%, 60%, 70%, 80%, 90%, or 100% of amino acids 447-541 of tobacco ALKBH9B (SEQ ID NO: 12).

196. The plant of embodiment 150, wherein the RNA m⁶A demethylase is tobacco ALKBH10B.

197. The plant of embodiment 196, wherein the ALKBH10B comprises a deletion of at least about 10%, 20%, 30%, 40%, 50%, 60%, 70%, 80%, 90%, or 100% of:
(i) amino acids 181-235 and/or
(ii) amino acids 504-640
of tobacco ALKBH10B (SEQ ID NO: 14).

198. The plant of embodiment 197, wherein the ALKBH10B comprises a deletion of at least about 10%, 20%, 30%, 40%, 50%, 60%, 70%, 80%, 90%, or 100% of amino acids 181-235 of tobacco ALKBH10B (SEQ ID NO: 14).

199. The plant of embodiment 197, wherein the ALKBH10B comprises a deletion of at least about 10%, 20%, 30%, 40%, 50%, 60%, 70%, 80%, 90%, or 100% of amino acids 504-640 of tobacco ALKBH10B (SEQ ID NO: 14).

200. The plant of embodiment 197, wherein the ALKBH10B comprises a deletion of at least about 10%, 20%, 30%, 40%, 50%, 60%, 70%, 80%, 90%, or 100% of amino acids 181-235 and a deletion of at least about 10%, 20%, 30%, 40%, 50%, 60%, 70%, 80%, 90%, or 100% of amino acids 504-640 of tobacco ALKBH10B (SEQ ID NO: 14).

201. The plant of embodiment 150, wherein the RNA m⁶A demethylase is alfalfa ALKBH9B.

202. The plant of embodiment 201, wherein the ALKBH9B comprises a deletion of at least about 10%, 20%, 30%, 40%, 50%, 60%, 70%, 80%, 90%, or 100% of amino acids 442-508 of alfalfa ALKBH9B (SEQ ID NO: 16).

203. The plant of embodiment 27, wherein the RNA m⁶A demethylase is sorghum ALKBH9B.

204. The plant of embodiment 203, wherein the ALKBH9B comprises a deletion of at least about 10%, 20%, 30%, 40%, 50%, 60%, 70%, 80%, 90%, or 100% of:
(i) amino acids 66-179 and/or
(ii) amino acids 423-611
of sorghum ALKBH9B (SEQ ID NO: 18).

205. The plant of embodiment 204, wherein the ALKBH9B comprises a deletion of at least about 10%, 20%, 30%, 40%, 50%, 60%, 70%, 80%, 90%, or 100% of amino acids 66-179 of sorghum ALKBH9B (SEQ ID NO: 18).

206. The plant of embodiment 204, wherein the ALKBH9B comprises a deletion of at least about 10%, 20%, 30%, 40%, 50%, 60%, 70%, 80%, 90%, or 100% of amino acids 423-611 of sorghum ALKBH9B (SEQ ID NO: 18).

207. The plant of embodiment 204, wherein the ALKBH9B comprises a deletion of at least about 10%, 20%, 30%, 40%, 50%, 60%, 70%, 80%, 90%, or 100% of amino acids 66-179 and a deletion of at least about 10%, 20%, 30%, 40%, 50%, 60%, 70%, 80%, 90%, or 100% of amino acids 423-611 of sorghum ALKBH9B (SEQ ID NO: 18).

208. The plant of embodiment 150, wherein the RNA m⁶A demethylase is maize ALKBH9B.

209. The plant of embodiment 208, wherein the ALKBH9B comprises a deletion of at least about 10%, 20%, 30%, 40%, 50%, 60%, 70%, 80%, 90%, or 100% of:
(i) amino acids 72-165 and/or
(ii) amino acids 423-615 of maize ALKBH9B (SEQ ID NO: 20).

210. The plant of embodiment 209, wherein the ALKBH9B comprises a deletion of at least about 10%, 20%, 30%, 40%, 50%, 60%, 70%, 80%, 90%, or 100% of amino acids 72-165 of maize ALKBH9B (SEQ ID NO: 20).

211. The plant of embodiment 209, wherein the ALKBH9B comprises a deletion of at least about 10%, 20%, 30%, 40%, 50%, 60%, 70%, 80%, 90%, or 100% of amino acids 423-615 of maize ALKBH9B (SEQ ID NO: 20).

212. The plant of embodiment 209, wherein the ALKBH9B comprises a deletion of at least about 10%, 20%, 30%, 40%, 50%, 60%, 70%, 80%, 90%, or 100% of amino acids 72-165 and a deletion of at least about 10%, 20%, 30%, 40%, 50%, 60%, 70%, 80%, 90%, or 100% of amino acids 423-615 of maize ALKBH9B (SEQ ID NO: 20).

213. The plant of embodiment 150, wherein the RNA m⁶A demethylase is maize ALKBH10B.

214. The plant of embodiment 213, wherein the ALKBH10B comprises a deletion of at least about 10%, 20%, 30%, 40%, 50%, 60%, 70%, 80%, 90%, or 100% of:
(i) amino acids 109-210 and/or
(ii) amino acids 437-573
of maize ALKBH10B (SEQ ID NO: 22).

215. The plant of embodiment 214, wherein the ALKBH10B comprises a deletion of at least about 10%, 20%, 30%, 40%, 50%, 60%, 70%, 80%, 90%, or 100% of amino acids 109-210 of maize ALKBH10B (SEQ ID NO: 22).

216. The plant of embodiment 214, wherein the ALKBH10B comprises a deletion of at least about 10%, 20%, 30%, 40%, 50%, 60%, 70%, 80%, 90%, or 100% of amino acids 437-573 of maize ALKBH10B (SEQ ID NO: 22).

217. The plant of embodiment 214, wherein the ALKBH10B comprises a deletion of at least about 10%, 20%, 30%, 40%, 50%, 60%, 70%, 80%, 90%, or 100% of amino acids 109-210 and a deletion of at least about 10%, 20%, 30%, 40%, 50%, 60%, 70%, 80%, 90%, or 100% of amino acids 437-573 of maize ALKBH10B (SEQ ID NO: 22).

218. The plant of embodiment 150, wherein the RNA m⁶A demethylase is wheat ALKBH9B.

219. The plant of embodiment 218, wherein the ALKBH9B comprises a deletion of at least about 10%, 20%, 30%, 40%, 50%, 60%, 70%, 80%, 90%, or 100% of:
(i) amino acids 57-163 and/or
(ii) amino acids 417-612
of wheat ALKBH9B (SEQ ID NO: 24).

220. The plant of embodiment 219, wherein the ALKBH9B comprises a deletion of at least about 10%, 20%, 30%, 40%, 50%, 60%, 70%, 80%, 90%, or 100% of amino acids 57-163 of wheat ALKBH9B (SEQ ID NO: 24).

221. The plant of embodiment 219, wherein the ALKBH9B comprises a deletion of at least about 10%, 20%, 30%, 40%, 50%, 60%, 70%, 80%, 90%, or 100% of amino acids 417-612 of wheat ALKBH9B (SEQ ID NO: 24).

222. The plant of embodiment 219, wherein the ALKBH9B comprises a deletion of at least about 10%, 20%, 30%, 40%, 50%, 60%, 70%, 80%, 90%, or 100% of amino acids 57-163 and a deletion of at least about 10%, 20%, 30%, 40%, 50%, 60%, 70%, 80%, 90%, or 100% of amino acids 417-612 of wheat ALKBH9B (SEQ ID NO: 24).

223. The plant of embodiment 150, wherein the RNA m⁶A demethylase is wheat ALKBH10B.

224. The plant of embodiment 223, wherein the ALKBH10B comprises a deletion of at least about 10%, 20%, 30%, 40%, 50%, 60%, 70%, 80%, 90%, or 100% of:
(i) amino acids 91-211 and/or
(ii) amino acids 440-566
of wheat ALKBH10B (SEQ ID NO: 26).

225. The plant of embodiment 224, wherein the ALKBH10B comprises a deletion of at least about 10%, 20%, 30%, 40%, 50%, 60%, 70%, 80%, 90%, or 100% of amino acids 91-211 of wheat ALKBH10B (SEQ ID NO: 26).

226. The plant of embodiment 224, wherein the ALKBH10B comprises a deletion of at least about 10%, 20%, 30%, 40%, 50%, 60%, 70%, 80%, 90%, or 100% of amino acids 440-566 of wheat ALKBH10B (SEQ ID NO: 26).

227. The plant of embodiment 224, wherein the ALKBH10B comprises a deletion of at least about 10%, 20%, 30%, 40%, 50%, 60%, 70%, 80%, 90%, or 100% of amino acids 91-211 and a deletion of at least about 10%, 20%, 30%, 40%, 50%, 60%, 70%, 80%, 90%, or 100% of amino acids 440-566 of wheat ALKBH10B (SEQ ID NO: 26).

228. The plant of any one of embodiments 122-227, wherein the RNA m⁶A demethylase is operably linked to at least one nuclear localization signal (NLS).

229. The plant of embodiment 228, wherein the RNA m⁶A demethylase is operably linked to at least one heterologous nuclear localization signal (NLS).

230. The plant of any one of embodiments 122-229, wherein the RNA m⁶A demethylase is operably linked to a promoter for expression in a plant.

231. The plant of embodiment 230, wherein the promoter is a constitutive promoter.

232. A plant part, tissue, or cell of the plant of any one of embodiments 122-231.

233. The plant of any one of embodiments 122-231, wherein the plant has improved growth compared to a control plant.

234. The plant of embodiment 233, wherein the plant has improved growth under abiotic stress conditions.

235. The plant of embodiment 233, wherein the plant has improved growth under drought stress, salt stress, heat stress, or cold stress.

236. The plant of embodiment 233, wherein the plant has improved growth under biotic stress conditions.

237. The plant of embodiment 233, wherein the plant is improved in a characteristic selected from the group consisting of biomass, yield, root growth, root expansion, stem cell expansion of the root, stem cell expansion of the shoot, drought resistance, and pest resistance.

238. The plant of embodiment 233, wherein the plant has elevated photosynthesis.

239. The plant of any one of embodiments 122-238, wherein the plant is selected from the group consisting of Arabidopsis, rice, corn, soybean, potato, cassava, cowpea, cotton, turf grass, strawberry, blueberry, blackberry, raspberry, and wheat.

240. A seed produced by the plant of any one of embodiments 122-239.

241. A commercial product derived from the plant of any one of embodiments 122-239 or the seed of embodiment 240.

242. A plant or plant cell comprising a modified endogenous ALKBH5 homolog gene, wherein the modified endogenous ALKBH5 homolog gene encodes a protein comprising at least one disrupted endogenous low complexity region (LCR).

243. The plant or plant cell of embodiment 242, wherein the modified endogenous ALKBH5 homolog gene encodes a protein comprising a disrupted N-terminal LCR.

244. The plant or plant cell of embodiment 242, wherein the modified endogenous ALKBH5 homolog gene encodes a protein comprising a disrupted C-terminal LCR.

245. The plant or plant cell of embodiment 242, wherein the modified endogenous ALKBH5 homolog gene encodes a protein comprising a disrupted N-terminal and a disrupted C-terminal LCR.

246. The plant or plant cell of any one of embodiments 242-245, wherein the disruption of the endogenous LCR is selected from the group consisting of a partial truncation, a full truncation, a deletion, and a replacement.

247. The plant or plant cell of any one of embodiments 242-245, wherein the modified endogenous ALKBH5 homolog gene comprises a deletion of at least about 10%, 20%, 30%, 40%, 50%, 60%, 70%, 80%, 90%, or 100% deletion of at least one endogenous LCR.

248. The plant or plant cell of embodiment 242, wherein the modified endogenous ALKBH5 homolog gene encodes a protein that lacks at least one endogenous LCR.

249. The plant or plant cell of embodiment 248, wherein the modified endogenous ALKBH5 homolog gene encodes a protein that lacks an endogenous C-terminal LCR.

250. The plant or plant cell of embodiment 249, wherein the modified endogenous ALKBH5 homolog gene was modified to encode an mRNA that comprises a stop codon before the C-terminal LCR.

251. The plant or plant cell of embodiment 249, wherein the modified endogenous ALKBH5 homolog gene comprises a deletion of at least about 10%, 20%, 30%, 40%, 50%, 60%, 70%, 80%, 90%, or 100% of the nucleotides encoding the C-terminal LCR.

252. The plant or plant cell of embodiment 248, wherein the modified endogenous ALKBH5 homolog gene encodes a protein that lacks an endogenous N-terminal LCR.

253. The plant or plant cell of embodiment 252, wherein the modified endogenous ALKBH5 homolog gene comprises a deletion of at least about 10%, 20%, 30%, 40%, 50%, 60%, 70%, 80%, 90%, or 100% of the nucleotides encoding the N-terminal LCR.

254. The plant or plant cell of any one of embodiments 248-253, wherein the plant or plant cell was modified by a genome editing technique selected from the group consisting of TALEN editing, zinc finger nuclease editing, RNA-guided endonuclease-mediated editing, prime editing, and base editing.

255. The plant or plant cell of any one of embodiments 248-254, wherein the modified endogenous ALKBH5 homolog gene was modified to add a nuclear localization signal (NLS) to the encoded protein.

256. The plant or plant cell of embodiment 255, wherein the NLS is a heterologous NLS.

257. The plant or plant cell of embodiment 255, wherein the NLS is an endogenous NLS.

258. The plant or plant cell of any one of embodiments 248-257, wherein the plant or a plant comprising the plant cell has improved growth compared to a control plant.

259. The plant or plant cell of embodiment 258, wherein the plant or a plant comprising the plant cell has improved growth under abiotic stress conditions.

260. The plant or plant cell of embodiment 259, wherein the plant or a plant comprising the plant cell has improved growth under drought stress, salt stress, heat stress, or cold stress.

261. The plant or plant cell of embodiment 258, wherein the plant or a plant comprising the plant cell has improved growth under biotic stress conditions.

262. The plant or plant cell of embodiment 258, wherein the plant or a plant comprising the plant cell is improved in a characteristic selected from the group consisting of biomass, yield, root growth, root expansion, stem cell expansion of the root, stem cell expansion of the shoot, drought resistance, and pest resistance.

263. The plant or plant cell of embodiment 258, wherein the plant or a plant comprising the plant cell has elevated photosynthesis.

264. A seed produced by the plant or a plant comprising the plant cell of any one of embodiments 122-263.

265. A commercial product derived from the plant or a plant comprising the plant cell of any one of embodiments 122-263 or the seed of embodiment 264.

266. A method of improving growth of a plant, comprising:
a) engineering a plant to comprise the recombinant DNA of any one of embodiments 1-108, the expression vector of embodiment 109 or embodiment 110, or the transformation vector of embodiment 111, and
b) growing the plant,
wherein the plant has improved growth compared to a control plant.

267. The method of embodiment 266, wherein the plant has improved growth under abiotic stress conditions.

268. The method of embodiment 266, wherein the plant has improved growth under drought stress, salt stress, heat stress, or cold stress.

269. The method of embodiment 266, wherein the plant has improved growth under biotic stress conditions.

270. The method of embodiment 266, wherein the plant is improved in a characteristic selected from the group consisting of biomass, yield, root growth, root expansion, stem cell expansion of the root, stem cell expansion of the shoot, drought resistance, and pest resistance.

271. The method of embodiment 266, wherein the plant has elevated photosynthesis.

272. The method of any one of embodiments 266-271, wherein the plant is selected from the group consisting of Arabidopsis, rice, corn, soybean, potato, cassava, cowpea, cotton, turf grass, strawberry, blueberry, blackberry, raspberry, and wheat.

273. A method of improving growth of a plant, comprising:
a) genetically modifying the plant by transforming the plant with one or more gene editing components that target an endogenous ALKBH5 homolog gene, wherein the endogenous ALKBH5 homolog gene is modified to encode a protein comprising at least one disrupted endogenous low complexity region (LCR), and
b) growing the plant,
wherein the plant has improved growth compared to a control plant.

274. The method of embodiment 273, wherein the endogenous ALKBH5 homolog gene is modified to encode a protein comprising a disrupted endogenous N-terminal LCR.

275. The method of embodiment 273, wherein the endogenous ALKBH5 homolog gene is modified to encode a protein comprising a disrupted endogenous C-terminal LCR.

276. The method of embodiment 273, wherein the endogenous ALKBH5 homolog gene is modified to encode a protein comprising a disrupted endogenous N-terminal LCR and a disrupted endogenous C-terminal LCR.

277. The method of any one of embodiments 273-276, wherein the disruption of the endogenous LCR is selected from the group consisting of a partial truncation, a full truncation, a deletion, and a replacement.

278. The method of any one of embodiments 273-276, wherein the endogenous ALKBH5 homolog gene is modified to comprise a deletion of at least about 10%, 20%, 30%, 40%, 50%, 60%, 70%, 80%, 90%, or 100% deletion of at least one endogenous LCR.

279. The method of embodiment 273, wherein the endogenous ALKBH5 homolog gene is modified to encode a protein that lacks an endogenous C-terminal LCR.

280. The method of embodiment 279, wherein the endogenous ALKBH5 homolog gene is modified to encode an mRNA that comprises a stop codon before the C-terminal LCR.

281. The method of embodiment 279, wherein the endogenous ALKBH5 homolog gene is modified to comprise a deletion of at least about 10%, 20%, 30%, 40%, 50%, 60%, 70%, 80%, 90%, or 100% of the nucleotides encoding the C-terminal LCR.

282. The method of embodiment 273, wherein the endogenous ALKBH5 homolog gene is modified to encode a protein that lacks an endogenous N-terminal LCR.

283. The method of embodiment 282, wherein the endogenous ALKBH5 homolog gene is modified to comprise a deletion of at least about 10%, 20%, 30%, 40%, 50%, 60%, 70%, 80%, 90%, or 100% of the nucleotides encoding the N-terminal LCR.

284. The method of any one of embodiments 273-283, wherein the one or more gene editing components comprise a TALEN, a ZFN, an RNA-guided endonuclease, a fusion protein comprising an RNA-guided endonuclease, a fusion protein comprising a reverse transcriptase, a fusion protein comprising an RNA-guided endonuclease fused to a reverse transcriptase, a guide RNA, a template RNA, and/or a donor oligonucleotide.

285. The method of any one of embodiments 273-284, wherein the endogenous ALKBH5 homolog gene is modified to add a nuclear localization signal (NLS) to the encoded protein.

286. The method of embodiment 285, wherein a heterologous NLS is added.

287. The method of embodiment 285, wherein an endogenous NLS is added.

288. The method of any one of embodiments 273-287, wherein the plant is improved in a characteristic selected from the group consisting of biomass, yield, root growth, root expansion, stem cell expansion of the root, stem cell expansion of the shoot, drought resistance, and pest resistance.

289. The method of any one of embodiments 273-288, wherein the plant has improved growth under abiotic stress conditions.

290. The method of any one of embodiments 273-289, wherein the plant has improved growth under drought stress, salt stress, heat stress, or cold stress.

291. The method of any one of embodiments 273-290, wherein the plant has improved growth under biotic stress conditions.

292. The method of any one of embodiments 273-291, wherein the plant is selected from the group consisting of Arabidopsis, rice, rapeseed, tobacco, alfalfa, sorghum, maize, wheat, cassava, cowpea, and cotton.

293. A plant produced by the method of any one of embodiments 266-292.

294. A seed produced by the plant of embodiment 293.

295. A commercial product derived from the plant of embodiment 293 or the seed of embodiment 294.

### EXAMPLES

The presently disclosed subject matter will be better understood by reference to the following Examples, which are provided as exemplary of the invention, and not by way of limitation.

### Example 1a: Development of a Pipeline for Evaluating RNA Demethylases in mESCs and Tobacco Leaves

This example shows the development and validation of a model pipeline for determining the efficacy of constructs of various RNA demethylases in plants.

### Methods

### Biochemistry Assay of the RNA m⁶A Demethylase Activity In Vitro

The activity assay was conducted as reported (PMID: 22002720). In brief, the demethylation activity assay was carried out in a 50 μl reaction mixture containing m⁶A-modified RNA (SEQ ID NO: 63 sequence: CGCUUGUGUGUGCUGCUGGACUUGGm⁶ACUCGCGC), Algae FTO or other demethylase protein, 283 μM (NH₄)₂ Fe(SO₄)₂·6H₂O, 300 μM α-KG, 2 mM L-ascorbic acid, 50 μg/ml BSA, SUPERase·In™ RNase Inhibitor (0.2 U/ml, life technology), and 50 mM HEPES buffer (pH 7.0). The reactions were incubated at 37° C. for 1 hour, and quenched by the addition of 5 mM EDTA followed by heating for 10 min at 95° C. Control samples were treated with an excess amount of EDTA. Finally, RNA purification was performed using the Oligo Clean & Concentrator™ kit (Zymo Research) according to the manufacture's manual.

### Quantitative Analysis of m⁶A Level Using UHPLC-QQQ-MS/MS

The purified RNA was digested with 1 μl of Nuclease P1 (NEB) in 20 μl of 1× Nuclease P1 buffer (NEB) for 2 hours at 37° C. Subsequently, FastAP Thermosensitive Alkaline Phosphatase (1U, Thermo Scientific™) and 2.5 μl of FastAP buffer (Thermo Scientific™) were added, and the solution was further incubated at 37° C. for 4 hours. The resulting solution was then filtered through a 0.22 μm syringe filter and diluted to a final volume of 60 μl. Subsequently, 10 μl of the solution was injected into an LC-MS/MS system. Nucleosides were separated using reverse-phase ultra-performance liquid chromatography on a C18 column, coupled with online mass spectrometry detection using an Agilent 6410 Triple Quadrupole LC mass spectrometer in positive electrospray ionization mode. The nucleosides were quantified using the nucleoside to base ion mass transitions of 282 to 150 (m⁶A), and 268 to 136 (A). Quantification was performed by comparison with the standard curve obtained from pure nucleoside standards running at the same batch of samples. The ratio of m⁶A to A was calculated based on the calculated concentrations.

### Low Complexity Domain Prediction

To predict protein disordered regions, two criteria were utilized to define disordered regions: firstly, the prediction algorithm ‘IUPred’ (PMID: 29860432) was used with default settings, selecting regions with an IUPred score threshold exceeding 0.5. Additionally, structural annotations from AlphaFold were incorporated. Regions with low and very low confidence were identified to avoid potential overlap with functional domains. Based on these criteria, various truncations were designed.

### Results

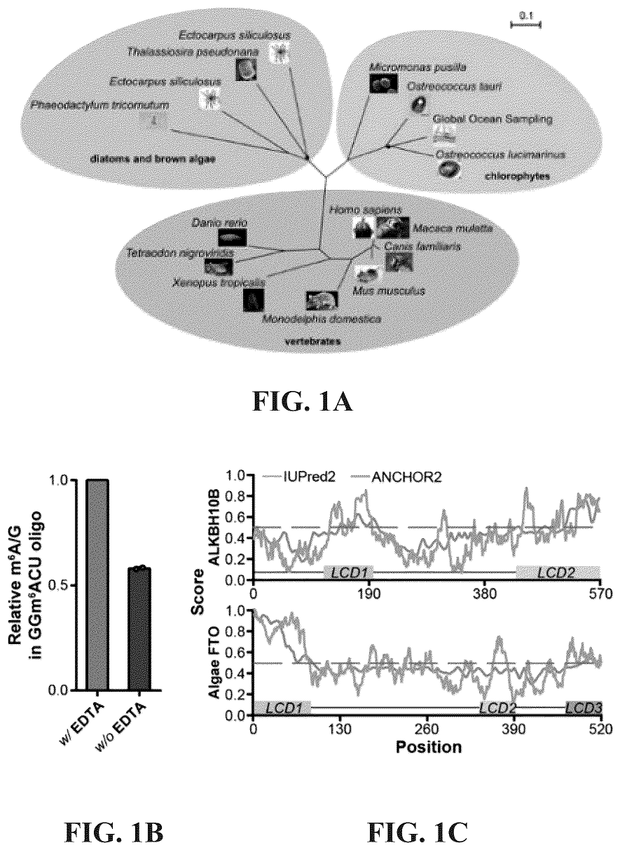

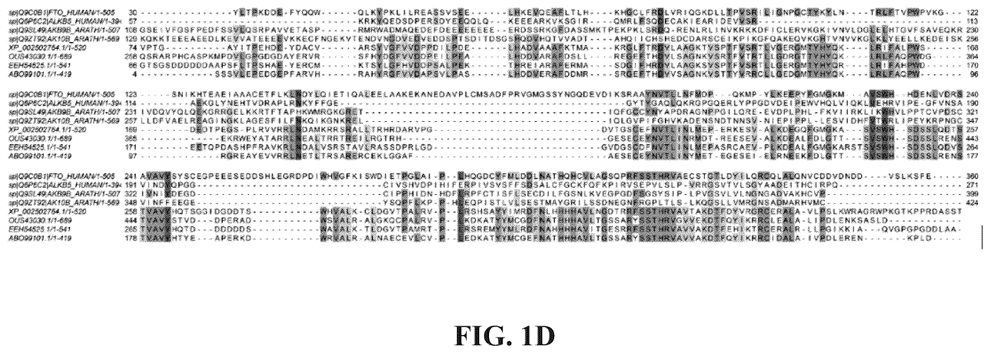

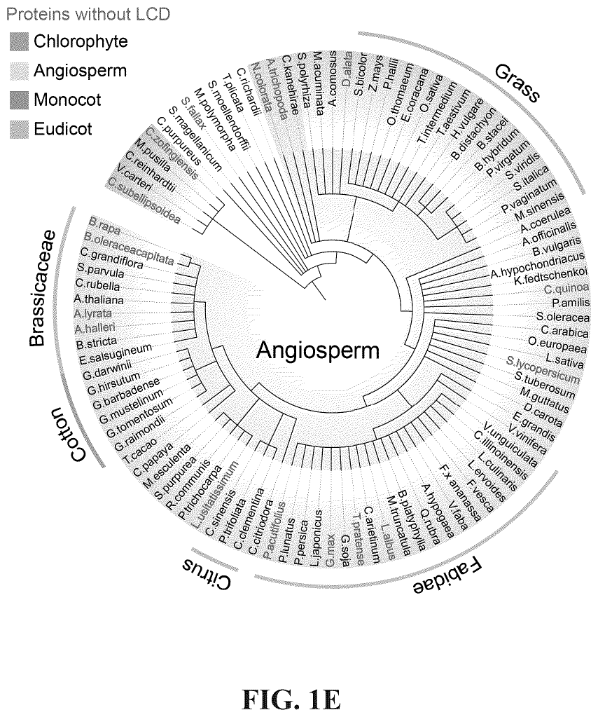

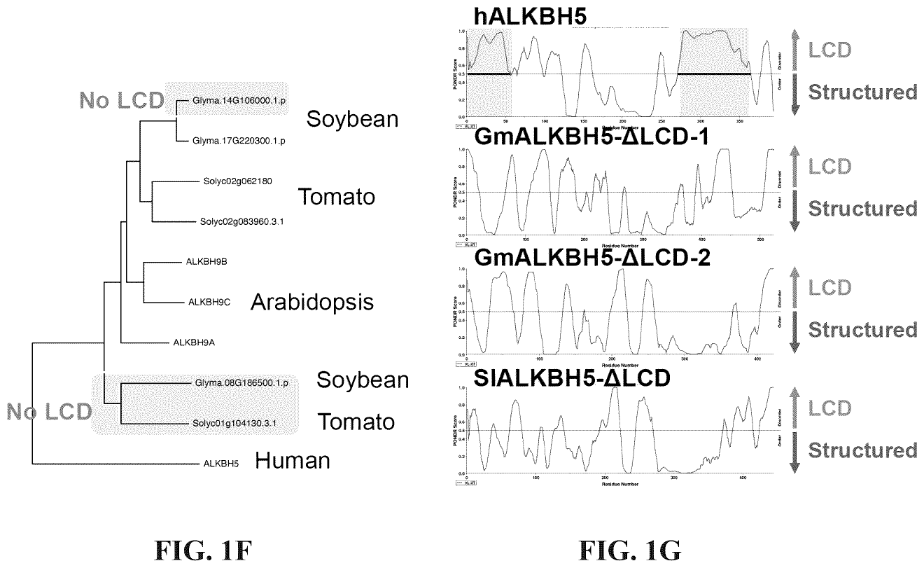

In mammals, both FTO and ALKBH5 function as m⁶A demethylases. Notably, plants lack an FTO homolog; only ALKBH5 homologs exist as m⁶A demethylases in plants. Plant ALKBH5 homologs ALKBH9B and ALKBH10B have been shown to function as RNA m⁶A demethylases that could actively remove m⁶A on RNA (PMID: 28923956, 29180595). There are a number (>25) of non-animal FTO homologs identified bioinformatically in other non-animal species such as green algae, brown algae, diatoms, xanthophyceae and oomycetes species (FIG. 1A), with several algae FTO homologs exhibiting high protein sequence similarity to human FTO in the catalytic domain (FIG. 1D). Accordingly, exemplified by wildtype Micromonas commoda FTO, moderate RNA m⁶A demethylation activity of this enzyme was observed based on an in vitro biochemistry assay (FIG. 1B). Notably, these non-animal FTO or plant m⁶A demethylases showed major differences outside the catalytic domain, with most of them possessing additional low complexity regions (LCRs) (FIG. 1C), suggesting potential regulatory roles.

These low complexity regions (LCRs) may restrict ALKBH5 and its homologues from accessing different chromatin sites for demethylation of m⁶A on chromatin-associated RNAs (caRNAs); this is in comparison to the caRNA demethylation activity by human and mouse FTO which is critical to the chromatin state regulation (PMID: 35511947). FTO does not possess these LCRs and thus may broadly access different chromatin-associated RNAs.

In mESCs, long-interspersed element-1 (LINE1) is a major substrate of FTO in chromatin-associated fraction, and LINE1 RNA abundance was markedly reduced upon FTO KO in a m⁶A dependent manner (PMID: 35511947). The LINE1 RNA level (and LINE1 RNA m⁶A level) could therefore be used in mESCs to screen engineered demethylase constructs overexpressed in mESCs. Demethylase constructs capable of accessing LINE1 RNA for m⁶A demethylation resulted in elevated LINE1 RNA level. Direct measurement of LINE1 RNA levels served as a quick way to identify active constructs in mESCs.

Once demethylases were identified that could elevate LINE1 RNA level in mESCs (through reducing m⁶A on LINE1 RNA), plasmids for expression of the demethylases in Tobacco were designed and developed, and transient infection was then performed of the corresponding constructs (using Agrobacterium) to Tobacco leaves. Active m⁶A demethylation by transiently expressed FTO was shown to cause more open chromatin in Tobacco leaves. This served as a model system to confirm that m⁶A demethylation by a demethylase construct could indeed induce global chromatin state change in plants.

Demethylase constructs that showed activity in driving a more open chromatin in Tobacco leaves were then moved into Arabidopsis and rice. A transgenic incorporation of the FTO-type demethylase drove longer root growth in Arabidopsis compared with an inactive mutant control and a control with empty plasmid.

Transgenic rice are also used to monitor root growth as well as biomass and seed yield increases as phenotypes to confirm the effect of engineered demethylase.

### Example 1B: Development of a Pipeline for Evaluating RNA Demethylases in mESCs and Tobacco Leaves

### Methods

Methods are provided after Example 6.

### Results

Previous research discovered that transgenic expression of human FTO in rice and potato markedly increased yield and biomass by facilitating chromatin opening and transcriptional activation through carRNA m⁶A demethylation in plant cells (Yu et al., 2021). These findings suggest a conservation of m⁶A-mediated chromatin regulation across mammals and plants (Wei et al., 2022). All plant m⁶A demethylases are homologous to ALKBH5, but not FTO, and these plant ALKBH5 homologs mostly act on plant mRNA to impact post-transcriptional regulation instead of chromatin regulation (Martinez-Perez et al., 2017; Xue et al., 2024; Tang et al., 2024; Duan et al., 2017). It was therefore hypothesized that a similar IDR-deletion strategy might release the catalytic domain of the ALKBH5 family demethylases to engage in chromatin-associated regulatory RNA (carRNA) m⁶A demethylation and chromatin regulation in plants. If successful, it could be possible to bypass animal FTO and use engineered plant ALKBH proteins for promoting plant biomass and yield increases, providing a much more favorable approach of crop engineering.

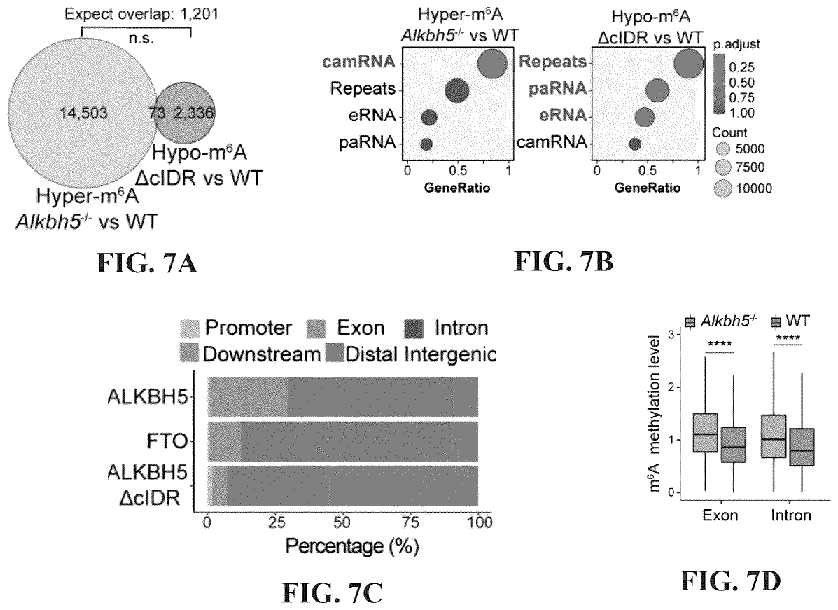

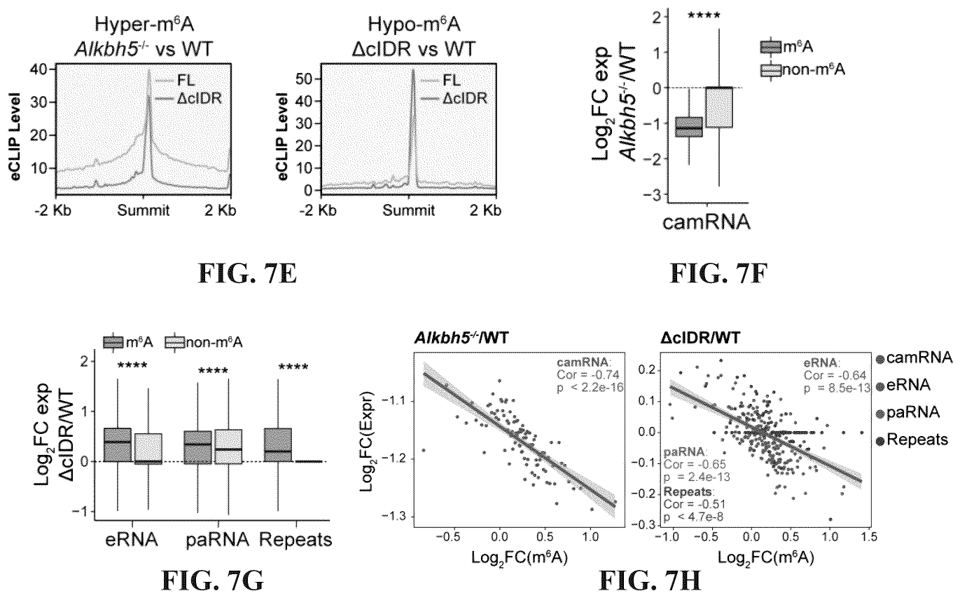

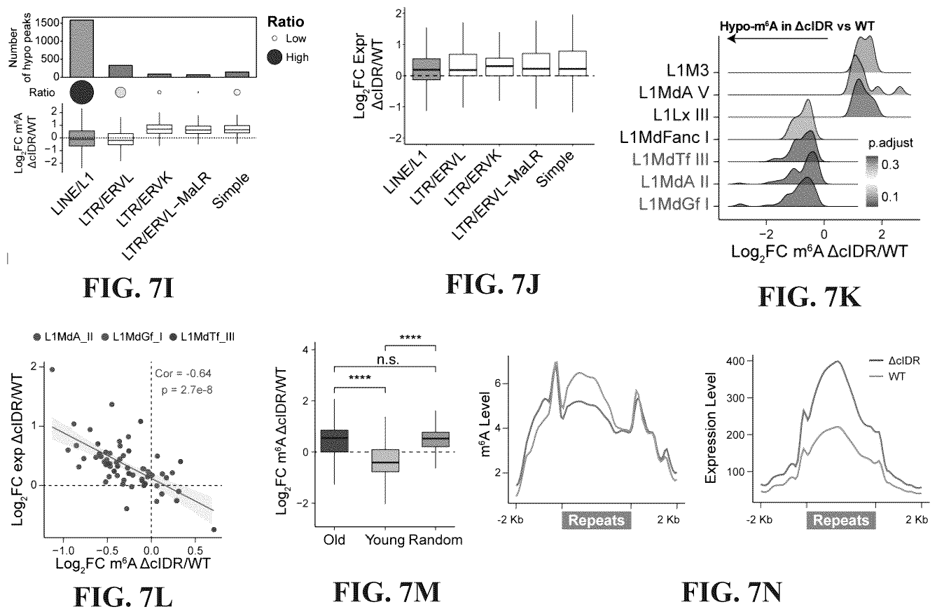

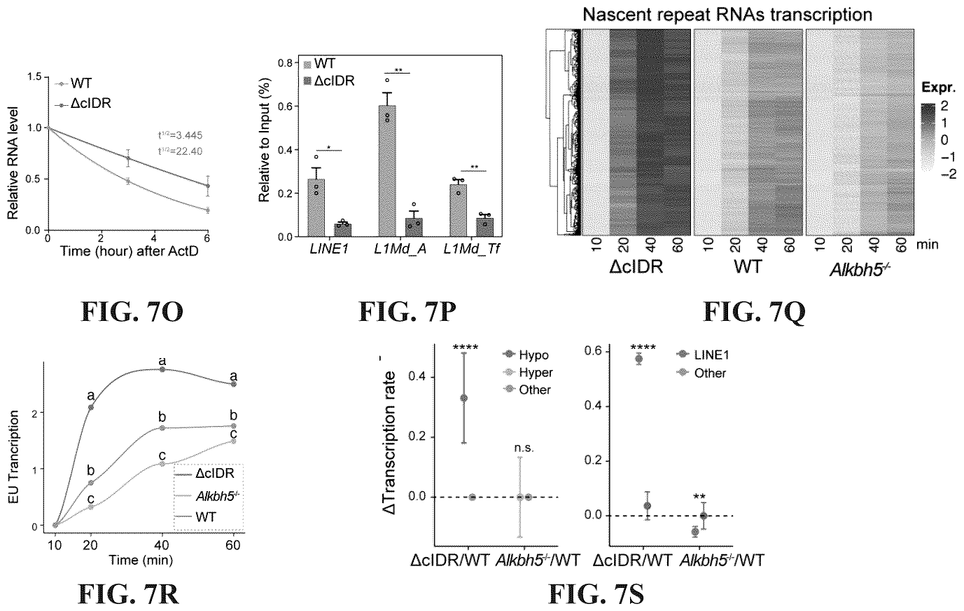

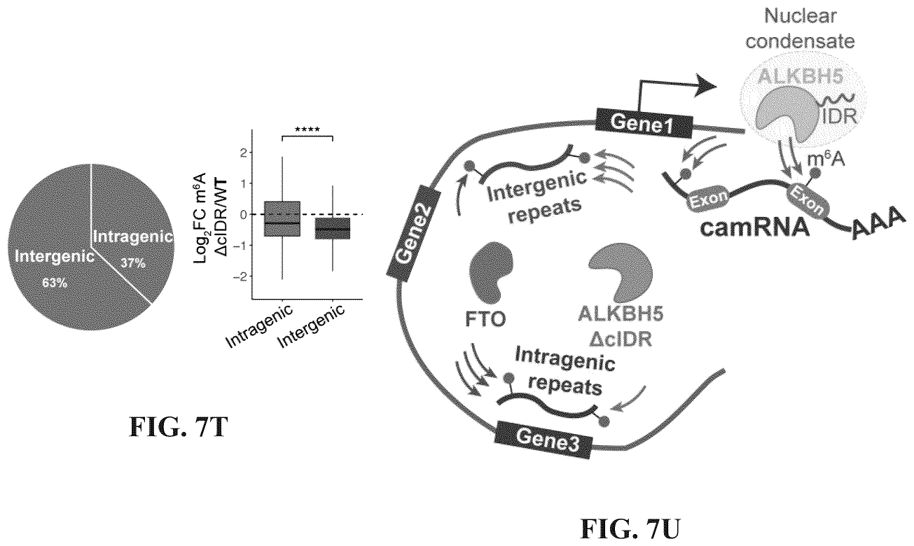

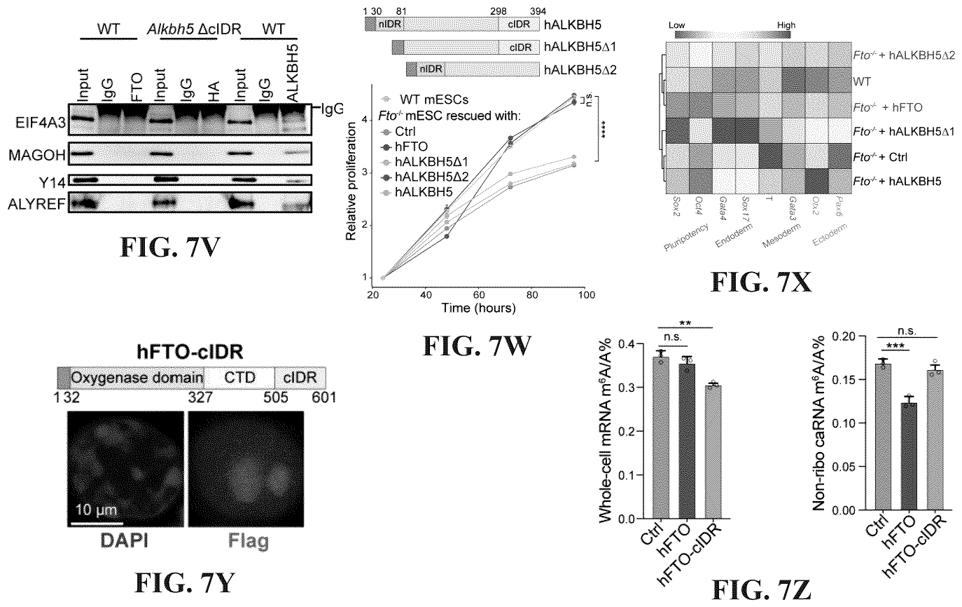

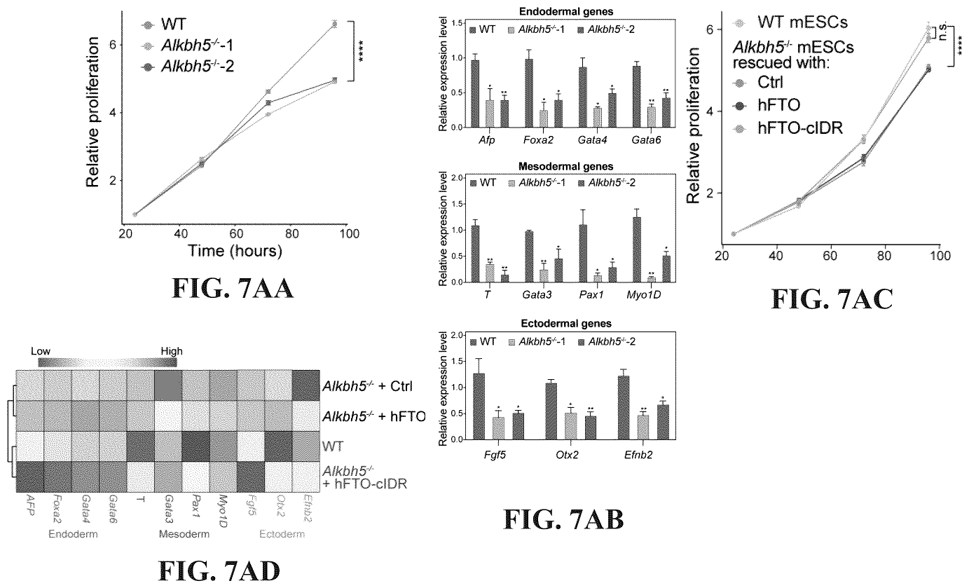

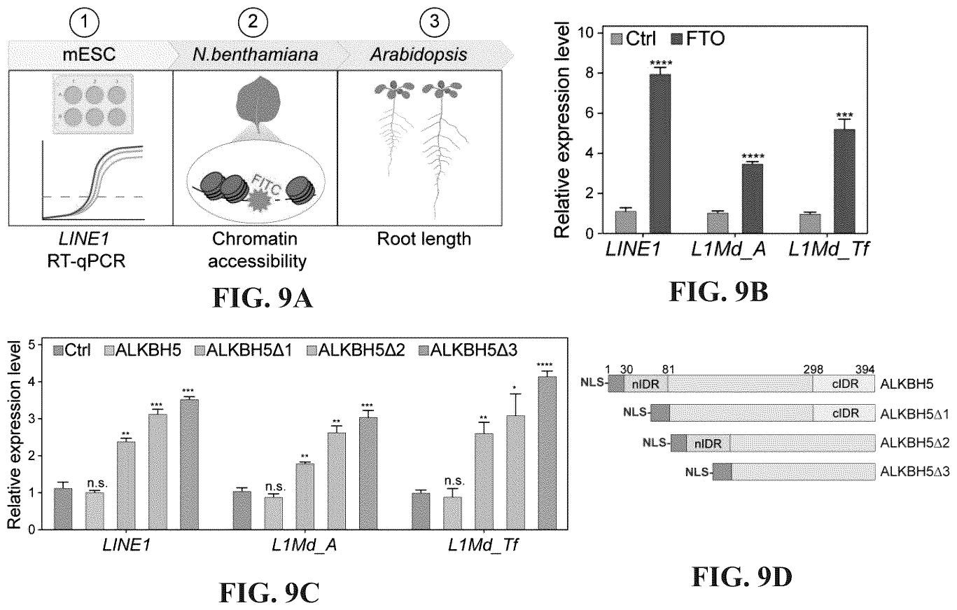

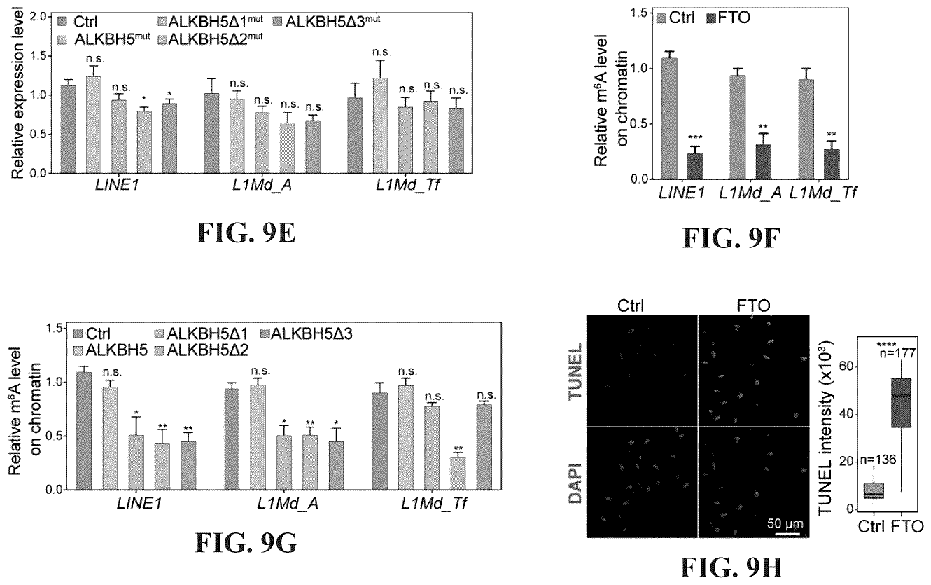

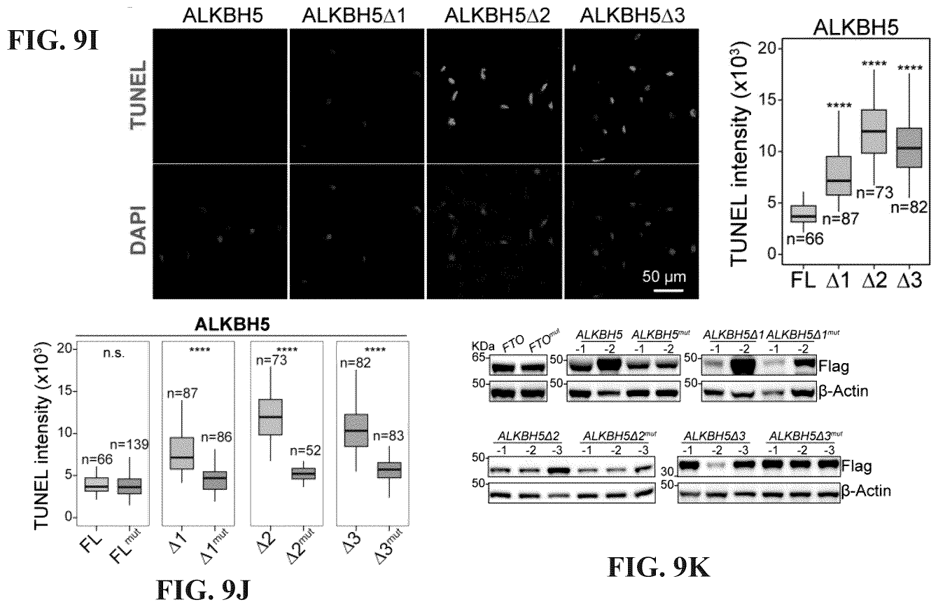

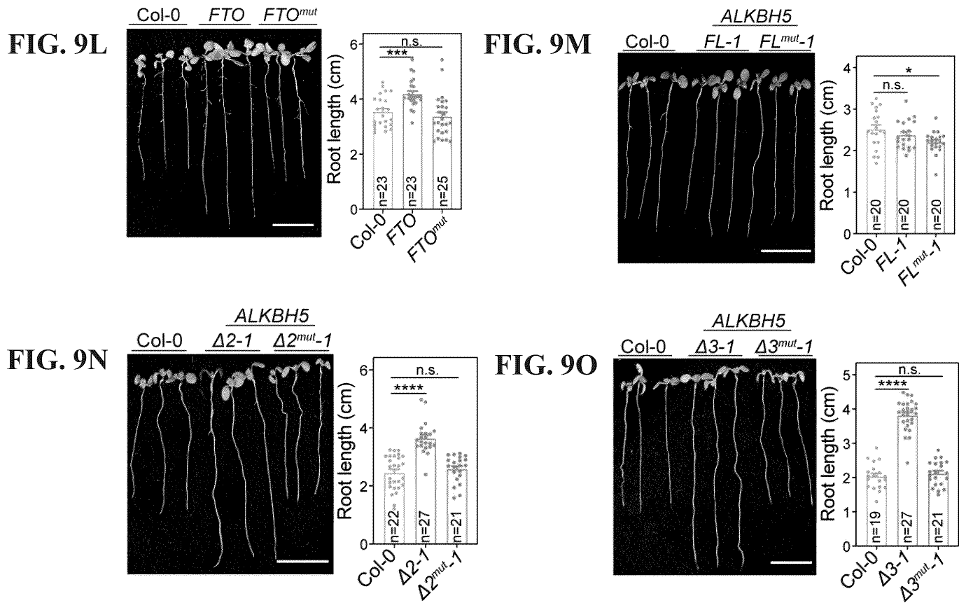

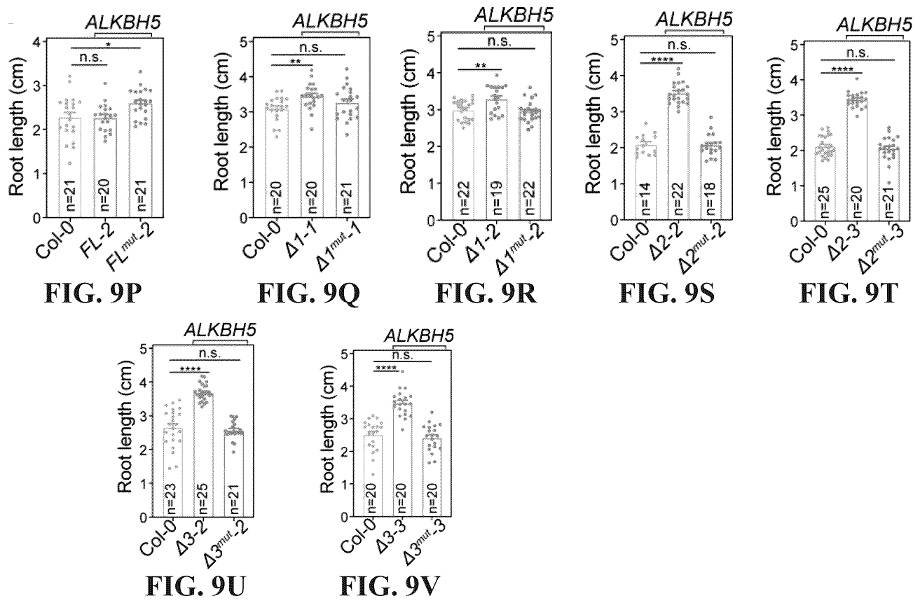

To test this hypothesis, a workflow was developed to functionally test constructs of engineered ALKBH5 homologs (FIG. 9A). These constructs were designed to include nuclear localization signals (NLS) to ensure nuclear entry—a prerequisite for chromatin targeting—and IDR deletions to enable broader m⁶A demethylation. mESCs were used as the initial screening platform because the chromatin regulatory functions of FTO and ALKBH5-ΔcIDR have been established in this system. In mESCs, LINE1 RNAs serve as the major substrates of FTO and ALKBH5-ΔcIDR within the chromatin-associated fraction (Wei et al., 2022) (FIGS. 7A-7AD), making their expression levels robust and reliable readouts for evaluating chromatin regulatory activity of the engineered demethylase constructs. Constructs that can significantly elevate LINE1 RNA levels would be selected for further testing in plants. To rapidly evaluate the selected constructs in a plant system, tobacco (Nicotiana benthamiana) was utilized as a transient expression model due to its high transformation efficiency and robust protein expression (Sparkes et al., 2006). The engineered demethylases could be transiently expressed in tobacco leaves via agroinfiltration, followed by DNase I-treated TUNEL assays to determine whether they induced global chromatin state change in plants. Constructs that could promote chromatin opening in tobacco leaves would then be stably transformed into Arabidopsis for growth evaluation. Given that transgenic expression of mammalian FTO in rice greatly enhanced root growth (Yu et al., 2021) 57 and that Arabidopsis is a faster-growing model plant, root growth in Arabidopsis was selected as a functional readout to assess the engineered demethylase constructs in promoting plant growth.

### Example 2a: Validation of Human FTO Overexpression in mESCs and Tobacco Leaves

This example shows the validation of human FTO overexpression in the pipeline.

### Methods

### Plasmid Construction

For human FTO overexpression in mESCs as an example, pPB-CAG-IRES-Pac was restriction digested with Bglii and Xhol. The human FTO cDNA was PCR amplified with PPB-FTO-F (SEQ ID NO: 64; GTTCCAGATTACGCTAGATCTAAGCGCACCCCGACTGCCGAG) and PPB-FTO-R (SEQ ID NO: 65; TTAGGGAGAGGGGCGCTCGAGTCAGGGTTTTGCTTCCAGAAG) oligonucleotides using human cDNA made by oligo-dT-priming HEK-293T total RNA. The resulting fragment was combined with digested pPB-CAG-IRES-Pac backbone with NEBuilder® HiFi DNA Assembly Master Mix (NEB). For human FTO overexpression in plant, the CDS of human FTO was cloned into XmaI/BamHI-digested pACT2::Flag-GFP vector, generating the plasmid 35S::Flag-NLS-FTO.

### RT-qPCR

ESCs were disrupted in TRIzoI™ Reagent (Invitrogen), and total RNA was extracted using a combination of chloroform extraction and the RNA Clean & Concentrator™ kit (Zymo research), following the manufacturer's instructions. To eliminate any residual DNA contamination, RNA was treated on-column with DNase I. cDNA was then synthesized using PrimeScript™ RT Master Mix (Takara). Quantitative real-time PCR (qPCR) was performed using FastStart Essential DNA Green Master (Roche) on a LightCycler® 96 system (Roche). Relative changes in gene expression were calculated using the ΔΔCₜ method. Primers used for RT-qPCR are listed in Table 2.

TABLE 2
Primer/Oligo Sequences

| | | SEQ ID |
|---|---|---|
| Oligo name | Oligo Sequence (5′ to 3′) | NO |
| PPB-FTO-F | GTTCCAGATTACGCTAGATCTAAGCGCACCCCGACTGC | 64 |
| | CGAG | |
| PPB-FTO-R | TTAGGGAGAGGGGCGCTCGAGTCAGGGTTTTGCTTCCA | 65 |
| | GAAG | |
| H2A-F | ACATGGCGGCGGTGCTGGAGTA | 66 |
| H2A-R | CGGGATGATGCGCGTCTTCTTGTT | 67 |
| mus-LINE1 RNA-F | GAGACATAACAACAGATCCTGA | 68 |
| mus-LINE1 RNA-R | AACTTTGGTACCTGGTATCTG | 69 |
| mus-L1MdA-F | GGATTCCACACGTGATCCTAA | 70 |
| mus-L1MdA-R | TCCTCTATGAGCAGACCTGGA | 71 |
| mus-L1MdTf-F | CAGCGGTCGCCATCTTG | 72 |
| mus-L1MdTf-R | CACCCTCTCACCTGTTCAGACTAA | 73 |
| Oligos for plasmids construction: | | |
| PPB-Flag-HA-humanALKBH5, | | |
| PPB-Flag-HA-humanALKBH5Δ1, | | |
| PPB-Flag-HA-humanALKBH5Δ2, | | |
| PPB-Flag-HA-humanALKBH5Δ3 | | |
| PPB-huBH5WT-F | gttCCAGATTACGCTAGATCTgcggccgccagcggctacac | 75 |
| PPB-huBH5WT-R | ttaGGGAGAGGGGCGCTCGAGtcagtgccgccgcatcttcac | 76 |
| PPB-huBH5de1-F | cagccgggaggcgcgcaaggtgaagagcgg | 77 |
| PPB-huBH5de1-R | ccttgcgcgcctcccggctgcccgccttatag | 78 |
| PPB-huBH5de2-R | TTAGGGAGAGGGGCGCTCGAGtcacagggactttgtttccaacc | 79 |
| PPB-Flag-HA-humanFTO | | |
| PPB-FTOh-F | gttCCAGATTACGCTAGATCTaagcgcaccccgactgccgag | 80 |
| PPB-FTOh-R | ttaGGGAGAGGGGCGCTCGAGTCAgggttttgcttccagaag | 81 |
| PPB-Flag-HA-NLS- | | |
| humanALKBH5, PPB-Flag-HA- | | |
| NLS-humanALKBH5Δ1, PPB- | | |
| Flag-HA-NLS-humanALKBH5Δ2, | | |
| PPB-Flag-HA-NLS- | | |
| humanALKBH5Δ3 | | |
| NLS-hALKBH5-F | CTAGATCTCCAAAAAAGAAGAGAAAGGTAGATCCAAA | 82 |
| | AAAGAAGAGAAAGGTAGATCCAAAAAAGAAGAGAAA | |
| | GGTAggatccgcggccgccagcggctac | |
| PPB-Flag-HA-GFP- | | |
| araALKBH9B-NLS, PPB-Flag- | | |
| HA-GFP-araALKBH9BΔ1-NLS, | | |
| PPB-Flag-HA-GFP- | | |
| araALKBH9BΔ2-NLS, PPB-Flag- | | |
| HA-GFP-araALKBH9BΔ3-NLS, | | |
| PPB-Flag-HA-NLS-araALKBH9B, | | |
| PPB-Flag-HA-NLS- | | |
| araALKBH9BΔ1, PPB-Flag-HA- | | |
| NLS-araALKBH9BΔ2, PPB-Flag- | | |
| HA-NLS-araALKBH9BΔ3 | | |
| ppbfh-bglii-gfp-F | ACGATGTTCCAGATTACGCTAGATCTgtgagcaagggcgaggag | 83 |
| | C | |
| ara9B-GFP-R | tttcgctgccgctgccggacttgtacagctcgtcc | 84 |
| gfp-ara9BWT-F | gtccggcagcggcagcgaaaacgatccatttctccg | 85 |
| ara9BWT-F | AGAAGAGAAAGGTAggttccagatctgaaaacgatccatttctcc | 86 |
| NLS-ara9BWT-R | tggggctgccgctgccaccgtagtttcttctactag | 87 |
| ara9BWT-nls-F | cggtggcagcggcagccccaagaagaagaggaagg | 88 |
| NLS-R | TGCACCTGAGGAGTGAATTCCTCGAGtcacaccttcctcttcttcttg | 89 |
| | g | |
| PPB-FH-ara9BNLS-F | CAAAAAAGAAGAGAAAGGTAggatccgaaaatgaccccttcctgag | 90 |
| NLS9B-L1-R | agctgcccagcatgttctcgaccacgctac | 91 |
| NLS9B-L1-F | cgagaacatgctgggcagctggaagggcag | 92 |
| PPB-FH-ara9B-R | TAACGTTAGGGAGAGGGGCGCTCGAGttagccgtagttccgccgg | 93 |
| | ctag | |
| NLS9B-L2-F | tgatatggcccagcgcgagaatctgagac | 94 |
| NLS9B-L2-R | tctcgcgctgggccatatcagcccaccgc | 95 |
| NLS9B-L3-R | TAACGTTAGGGAGAGGGGCGCTCGAGttagatgccttggagatcag | 96 |
| | gc | |
| PPB-Flag-HA-NLS- | | |
| araALKBH10B, PPB-Flag-HA- | | |
| NLS-araALKBH10BΔ1, PPB-Flag- | | |
| HA-NLS-araALKBH10BΔ2 | | |
| PPBFH-NLS-ara10b-F | TGACCGGTCCAAAAAAGAAGAGAAAGGTAGATCCAAA | 97 |
| | AAAGAAGAGAAAGGTAGATCCAAAAAAGAAGAGAAA | |
| | GGTAggatccACCATCGCCGCTGCTCC | |
| PPBFH-ara10b-R | TTAGGGAGAGGGGCGCTCGAGTCAGCCCACGGTGATCA | 98 |
| | CAG | |
| PPB-Flag-HA-NLS-Os9B, PPB- | | |
| Flag-HA-NLS-Os9BΔ1, PPB-Flag- | | |
| HA-NLS-Os9BΔ2 | | |
| ppbfh-os9B-F | CAAAAAAGAAGAGAAAGGTAggatccgacgcctcctcctccgcctc | 99 |
| ppbfh-os9B-R | TAACGTTAGGGAGAGGGGCGCTCGAGttactcatccaggtttaccct | 100 |
| | gg | |
| ppbfh-os9BΔ1-R | TGCACCTGAGGAGTGAATTCCTCGAGtcattattcttgtacagctgttt | 101 |
| | c | |
| nls-os9BL1-frag2-F | cgtctcctccagggagcagcgggagctgag | 102 |
| nls-os9BL1-frag1-R | cgctgctccctggaggagacgcggctgcgg | 103 |
| PPB-Flag-HA-NLS-Os10B, PPB- | | |
| Flag-HA-NLS-Os10BΔ1, PPB- | | |
| Flag-HA-NLS-Os10BΔ2 | | |
| ppbfh-os10B-F | AGAAGAGAAAGGTAggatccgccgcgccggggccggggtg | 104 |
| ppbfh-os10B-R | TAACGTTAGGGAGAGGGGCGCTCGAGctactttgcgacaacattct | 105 |
| | gc | |
| ppbfh-os10BΔ1-R | TGCACCTGAGGAGTGAATTCCTCGAGtcactaagcagcttctgcgat | 106 |
| | cca | |
| PPBFH-NLS-OS10BΔ2-F | caaaaAAGAAGAGAAAGGTAggatccgacggcctcgtcgcgtggc | 107 |
| nls-os10BΔ2-frag1-R | caatcgcagcgtgaagcgcgagggcgacc | 108 |
| nls-os10BΔ2-frag2-F | cgcgcttcacgctgcgattgccagtggagg | 109 |
| PPBFH-NLS-OS10BΔ2-R | TAACGTTAGGGAGAGGGGCGCTCGAGctacctgggagcagcttct | 110 |
| | gcg | |
| 35S-Flag-NLS | | |
| xmaI-NLS-F | gcgtcccgggCCAAAAAAGAAGAGAAAGGTAggttccagatctggta | 111 |
| | ccggatccaag | |
| bamhi-NLS-R | cttggatccggtaccagatctggaaccTACCTTTCTCTTCTTTTTTGG | 112 |
| | cccgggacgc | |
| 35S-Flag-NLS-humanALKBH5, | | |
| 35S-Flag-NLS-humanALKBH5Δ1, | | |
| 35S-Flag-NLS-humanALKBH5Δ2, | | |
| 35S-Flag-NLS-humanALKBH5Δ3 | | |
| 35s-nls-BH5WT-F | GAAGAGAAAGGTAggttccagatctgcggccgccagcggctacac | 113 |
| 35s-nls-BH5WT-R | cattattatggagaaagcttggatcctcagtgccgccgcatcttca | 114 |
| 35s-nls-BH5Δ2-R | cattattatggagaaagcttggatcctcacagggactttgtttcc | 115 |
| 35S-Flag-GFP-araALKBH9B- | | |
| NLS, 35S-Flag-GFP- | | |
| araALKBH9BΔ1-NLS, 35S-Flag- | | |
| GFP-araALKBH9BΔ2-NLS, 35S- | | |
| Flag-GFP-araALKBH9BΔ3-NLS, | | |
| 35S-Flag-NLS-araALKBH9B, 35S- | | |
| Flag-NLS-araALKBH9BΔ1, 35S- | | |
| Flag-NLS-araALKBH9BΔ2, 35S- | | |
| Flag-NLS-araALKBH9BΔ3 | | |
| 35s-nls-ara9BWT-F | AGAAGAGAAAGGTAggttccagatctgaaaacgatccatttctcc | 116 |
| 35s-nls-ara9BWT-R | cattattatggagaaagcttggatccttaaccgtagtttcttctactag | 117 |
| 35s-nls-ara9BΔ3-R | cattattatggagaaagcttggatccttaaatcccttgcaaatcaggc | 118 |
| 35s-gfpara9BWT-F | AGAAGAGAAAGGTAggttccagatctgtgagcaagggcgaggagc | 119 |
| 35s-nls-gfpara9BWT-R | cattattatggagaaagcttggatccttatcacaccttcctcttcttc | 120 |
| | ttggg | |
| 35S-Flag-NLS-Os9B, 35S-Flag- | | |
| NLS-Os9BΔ1, 35S-Flag-NLS- | | |
| Os9BΔ2 | | |
| 35SNLS-os9BWT-F | AGAAGAGAAAGGTAggttccagatctgacgcctcctcctccgcctc | 121 |
| 35SNLS-os9BWT-R | cattattatggagaaagcttggatccttactcatccaggtttaccctgg | 122 |
| 35s-os9BΔ1-R | cattattatggagaaagcttggatccttattcttgtacagctgtttcta | 123 |
| 35S-Flag-NLS-Os10B, 35S-Flag- | | |
| NLS-Os10BΔ1, 35S-Flag-NLS- | | |
| Os10BΔ2 | | |
| 35SNLS-OS10BWT-F | AGAAGAGAAAGGTAggttccagatctgccgcgccggggccggggtg | 124 |
| 35SNLS-OS10BWT-R | cattattatggagaaagcttggatccctactttgcgacaacattct | 125 |
| 35s-os10BΔ1-R | cattattatggagaaagcttggatccctaagcagcttctgcgatccatg | 126 |
| H204A mutation for ALKBH5 | | |
| BH5-H204A-F | tcgtgtctgccgtggaccccatccacatc | 127 |
| BH5-H204A-R | ggggtccacggcagacacgatgcagccgccgg | 128 |
| H335A/D337A for ALKBH9B | | |
| ALKBH9B-H335A/D337A-F | cccccgccatcgccaaccacgacttcctgcggc | 129 |
| ALKBH9B-H335A/D337A-R | gtggttggcgatggcgggggggatacagtcgccttc | 130 |
| H324A/D326A for Os9B | | |
| Os9B-H324A/D326A-F | caccagccattgccagtcatgattttgttcggcc | 131 |
| Os9B-H324A/D326A-R | atgactggcaatggctggtgggatgcaatctccag | 132 |
| H308A/H310A for Os10B | | |
| Os10B-H308A/H310A-F | accctgcccatgcccctccttggtatggtagacc | 133 |
| Os10B-H308A/H310A-R | aggaggggcatgggcagggtgagaataatctccc | 134 |
| PPB-Flag-HA-FTO-cIDR | | |
| PPB-FH-FTO-CIDR-F | agcaagatccagcagctccgtgttaccacc | 135 |
| PPB-FH-FTO-CIDR-R | cggagctgctggatcttgcttccagcagc | 136 |
| PPB-Flag-HA-KDM4A, PPB-Flag- | | |
| HA-KDM4ΔMIDR | | |
| ppbfh-KDM4AWT-F | acgatgttccagattacgctagatctatggcttctgagtctgaaac | 137 |
| ppbfh-KDM4AWT-R | tgcacctgaggagtgaattcctcgagctactccatgatggcccgg | 138 |
| ppbfh-KDM4AΔIDR-F | gggttctggatccggttctgaggcctgggccaagcctc | 139 |
| ppbfh-KDM4AMIDR-R | cagaaccggatccagaaccctcagctgcttctggcgtgg | 140 |
| PPB-Flag-HA-KDM4B, PPB-Flag- | | |
| HA-KDM4BΔIDR | | |
| ppbfh-KDM4BWT-F | acgatgttccagattacgctagatctatggggtctgaggaccacgg | 141 |
| ppbfh-KDM4BWT-R | tgcacctgaggagtgaattcctcgagctagaagggggctccgggcc | 142 |
| ppbfh-KDM4BΔIDR-F | cggttctggatccggttctttgaggccgctgctgtctc | 143 |
| ppbfh-KDM4BΔIDR-R | aagaaccggatccagaaccggtgagcgccgtgggccgcg | 144 |
| PPB-Flag-HA-KDM6A, PPB-Flag- | | |
| HA-KDM6AΔIDR | | |
| ppbfh-KDM6AWT-F | acgatgttccagattacgctagatctatgaaatcctgcggagtgtc | 145 |
| ppbfh-KDM6AWT-R | tgcacctgaggagtgaattcctcgagtcaagatgaggcggatgg | 146 |
| ppbfh-KDM6AMIDR-F | gaggttctggatccggttctagctcagcagaagttctg | 147 |
| ppbfh-KDM6AΔIDR-R | agaaccggatccagaacctctcatttggtgttgttgc | 148 |
| PPB-Flag-HA-KDM6B, PPB-Flag- | | |
| HA-KDM6BΔIDR | | |
| ppbfh-KDM6BWT-F | acgatgttccagattacgctagatctatgcatcgggcagtggatcc | 149 |
| ppbfh-KDM6BWT-R | tgcacctgaggagtgaattcctcgagtcatcgcgacgtgctggctg | 150 |
| | gggccagcgtgaaggcgtcgtag | |
| ppbfh-KDM6BΔIDR-F | acgatgttccagattacgctagatctccccgggaaaaactcaaccc | 151 |
| ppbfh-KDM6BΔIDR-R | tgcacctgaggagtgaattcctcgagtcatcgcgacgtgctggctg | 152 |
| For Alkbh5 KO: | | |
| mALKBH5-sg1-F | CACCGacaggttccgacacccggat | 153 |
| mALKBH5-sg1-R | AAACatccgggtgtcggaacctgtC | 154 |
| mALKBH5-sg2-F | CACCGGGCGAGGGCTACACGTACG | 155 |
| mALKBH5-sg2-R | AAACCGTACGTGTAGCCCTCGCCC | 156 |
| mALKBH5-sg3-F | CACCGccccgagcgcagcgactacg | 157 |
| mALKBH5-sg3-R | AAACcgtagtcgctgcgctcggggC | 158 |
| type-mALKBH5sg1-R | agcttcctatggtttgcacg | 159 |
| type-mALKBH5sg1-F | ttcggctgcaagttccagttc | 160 |
| type-mALKBH5sg2/3-R | AGATGTGGATGGGGTCAACG | 161 |
| type-mALKBH5sg2/3-F | gggacaactacaaggcggg | 162 |
| For Alkbh5 knockin before cIDR: | | |
| Donor1 ssDNA template: | cctttacaagatgcccttttgagactccatgtgtgtttcc | 163 |
| | tttgcaggacaagattagatgcaccgcgaCTTgaaacaTA | |
| | CCCATACGATGTTCCAGATTACGCTtgatgaaaatccctg | |
| | agcagctccacattgccacccagctatgcttcagatcgcc | |
| | tgtcaggaaacaccagagaccctgcgctgaaacccaaaag | |
| Donor2 ssDNA template: | gtgtttcctgacaggcgatctgaagcatagctgggtggca | 164 |
| | atgtggagctgctcagggattttcatcaAGCGTAATCTGG | |
| | AACATCGTATGGGTAtgtttcAAGtcgcggtgcatctaat | |
| | cttgtcctgcaaaggaaacacacatggagtctcaaaaggg | |
| | catcttgtaaaggcataaaccctatcaggacagatctgtc | |
| gALKBH5-F1 | tctctggaagtcactaggcgt | 165 |
| Donor 1 crRNA | rArA rGrArU rUrArG rArUrG rCrArC rCrGrC rGrArU | 166 |
| | rGrUrU rUrUrA rGrArG rCrUrA rUrGrC rU | |
| Donor 2 crRNA | rArG rGrGrA rUrUrU rUrGrU rUrUrC rCrArA rUrCrG | 167 |
| | rGrUrU rUrUrA rGrArG rCrUrA rUrGrC rU | |
| gALKBH5-R1 | tgacaggcgatctgaagcat | 168 |
| For LINE1 qPCR: | | |
| H2A-F | ACATGGCGGCGGTGCTGGAGTA | 169 |
| H2A-R | CGGGATGATGCGCGTCTTCTTGTT | 170 |
| mus-LINE1 RNA-F | GAGACATAACAACAGATCCTGA | 171 |
| mus-LINE1 RNA-R | AACTTTGGTACCTGGTATCTG | 172 |
| mus-L1MdA-F | GGATTCCACACGTGATCCTAA | 173 |
| mus-L1MdA-R | TCCTCTATGAGCAGACCTGGA | 174 |
| mus-L1MdTf-F | CAGCGGTCGCCATCTTG | 175 |
| mus-L1MdTf-R | CACCCTCTCACCTGTTCAGACTAA | 176 |
| For eCLIP-seq: | | |
| 3′ RNA adaptor | 5′rApp-NN NNN ATC ACG AG ATC GGA AGA GCA CAC | 177 |
| | GTC T/3SpC3/ | |
| SR-RT primer | AGA CGT GTG CTC TTC CGA TCT | 178 |
| 3′ cDNA adaptor | 5′-/Phcs/NNN NNG ATC GTC GGA CTG TAG AAC TCT | 179 |
| | GAA C/3SpC3 | |
| For qPCR in rice: | | |
| qBasta-F | GAAGTCCAGCTGCCAGAAAC | 180 |
| qBasta-R | CTCTACACCCACCTGCTGAA | 181 |
| qBH5-F | ATCGTGTCCGTGTCCTTCTT | 182 |
| qBH5-R | AGGCTTGAACTGGAACTTGC | 183 |
| Actin-F | CACATTCCAGCAGATGTGGA | 184 |
| Actin-R | GCGATAACAGCTCCTCTTGG | 185 |

### MeRIP-RT-qPCR

m⁶A-IP was conducted with 1 μg of non-ribosomal RNA isolated from the chromatin-associated fraction of mESCs, using EpiMark® N⁶-Methyladenosine Enrichment Kit (NEB) following the manufacturer's protocols. To ensure accurate normalization, 1 μL of 1:1000 diluted m⁶A and non-m⁶A spike-in from this kit was added to the RNA sample. A 5% aliquot of this mixture was reserved as input control. Following immunoprecipitation, RNA was extracted separately from both the IP and input fractions using TRIzoI™ Reagent (Invitrogen), and subsequently subjected to reverse transcription quantitative PCR (RT-qPCR). Relative changes in gene expression were determined using the ΔΔCₜ method, with the same primers utilized for RT-qPCR above.

### Dnase I-Treated TUNEL Assay

Paraffin section of Tobacco leaves was deparaffinized by immersion in Histo-Clear® and rehydrated through a series of ethanol solutions. The slides were then fixed with 4% paraformaldehyde (PFA). Following two washes with phosphate-buffered saline (PBS), the slides were treated with 1 U/μl DNase I (Thermo scientific) at 37° C. for 5 minutes. Subsequently, the TUNEL assay was performed using the DeadEnd™ Fluorometric TUNEL System (Promega) following the manufacturer's instructions. Nuclear areas were defined based on DAPI staining. Imaging was performed using Leica SP8 confocal microscope, and the intensity of nuclear TUNEL signal was quantified using Fiji software.

### Results

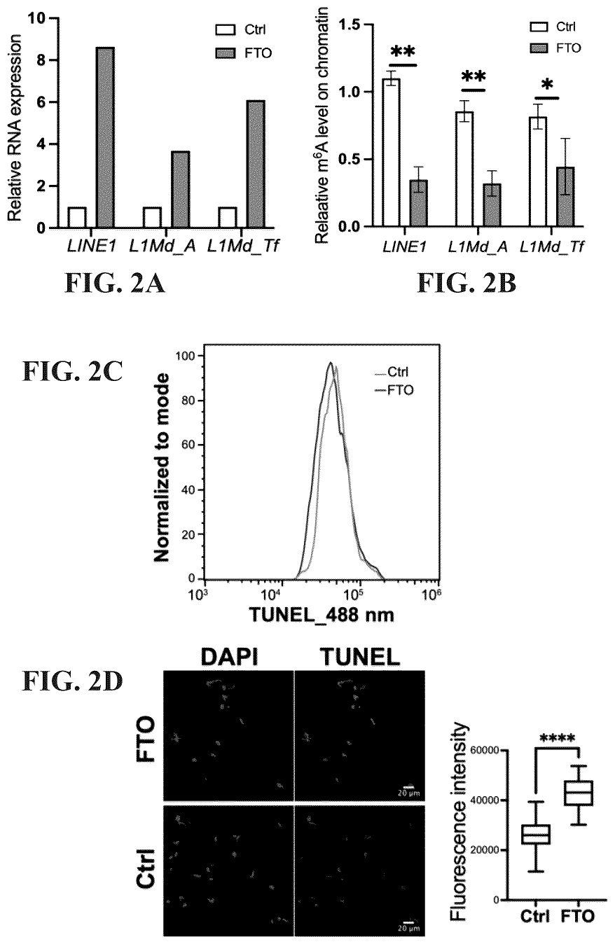

The pipeline was first tested in mESCs and Tobacco leaves. Human FTO was overexpressed in mESCs and whole-cell LINE1 RNA expression level examined. The quantitative PCR (qPCR) results indicated elevated LINE1 RNA level with FTO OE (FIG. 2A). Subsequent isolation of the chromatin-associated fraction enabled Methylated RNA Immunoprecipitation (MeRIP)-qPCR (FIG. 2B), demonstrating a consistent decrease in the m⁶A levels of LINE1 RNA due to FTO overexpression.

Next it was determined whether FTO overexpression in mESCs could induce global chromatin state change. A deoxyribonuclease (DNase) I-treated terminal deoxynucleotidyl transferase-mediated deoxyuridine triphosphate nick end labeling (TUNEL) assay was performed, but did not result in obvious difference (FIG. 2C). This may have been due to the presence of endogenous FTO that could limit effects of further FTO OE on the overall chromatin state in mESCs.

FTO overexpression was next tested in planta, in which human FTO has been reported to be able to promote chromatin openness, thus increase biomass and yield. Tobacco is a powerful model plant for transient protein expression since one can perform highly efficient transformation (PMID: 17487191). Agrobacterium infiltration was performed to transiently express human FTO in leaves of N. benthamiana and significantly increased chromatin accessibility was observed using the DNase I-treated TUNEL assay (FIG. 2D). As a result, whole-cell LINE1 RNA abundance and chromatin-associated LINE1 RNA m⁶A level were set as reliable indicators for evaluating the effects of overexpressing m⁶A demethylase in mESCs, as was chromatin accessibility in tobacco leaves.

### Example 2a: Validation of the Workflow Via Human FTO, ALKBH5, and IDR-Deleted ALKBH5 Overexpression in mESCs and Tobacco Leaves

### Methods

Methods are provided after Example 6.

### Results

To validate the workflow established in Example 1A, human FTO and ALKBH5 were used as benchmarks. As expected, ectopic expression of human FTO or IDR-deleted ALKBH5 variants (ALKBH5_{Δ30-81}, ALKBH5_{Δ298-394}, and ALKBH5_{Δ30-81 and 298-394}) in mESCs led to elevated whole-cell LINE1 RNA levels (FIGS. 2A, 3A, 9B-9D), whereas full-length ALKBH5 and catalytically inactive mutants (H204A) had no effect (FIG. 9C, 9E). MeRIP-qPCR analysis of caRNA further confirmed reduced m⁶A levels on LINE1 RNAs in mESCs expressing human FTO or IDR-deleted ALKBH5 variants (FIGS. 9F-9G). Next, these variants were transiently expressed in tobacco leaves via agrobacterium-mediated infiltration and DNase I-treated TUNEL assays were performed to evaluate chromatin accessibility changes. Tobacco leaves expressing human FTO or IDR-deleted ALKBH5 variants (ALKBH5_{Δ30-81}, ALKBH5_{Δ298-394}, and ALKBH5_{Δ30-81 and 298-394}) exhibited notably increased chromatin accessibility, while full-length or catalytically inactive ALKBH5 variants showed almost no effect (FIGS. 9H-9J).

Next, transgenic Arabidopsis lines expressing these variants were generated (FIG. 9K). Consistent with observations in FTO-transgenic rice, 10-day-old FTO-transgenic Arabidopsis displayed notable longer root growth (FIG. 9L). Importantly, while full-length ALKBH5 failed to promote Arabidopsis root growth, expression of IDR-deleted ALKBH5 variants (ALKBH5_{Δ298-394}, and ALKBH5_{Δ30-81 and 298-394}) increased root length similar to that of FTO when compared to the Col-0 control, whereas their respective inactive mutants had no effect (FIGS. 9M-9V). Again, the cIDR deletion in ALKBH5_{Δ298-394}, and ALKBH5_{Δ30-81 and 298-394} appeared to be key to the root growth phenotype because expression of ALKBH5_{Δ30-81} had limited effect (FIGS. 9Q-9R).

In summary, this screening workflow effectively identified engineered ALKBH5 constructs with cIDR deletions as capable of enhancing chromatin accessibility and plant root growth, providing an effective platform for rapidly testing candidate constructs in model plants.

### Example 3a: The Low Complexity Regions (LCRs) of ALKBH5 Restrict its Access to Chromatin Sites and Limits its Activity on Chromatin-Associated RNAs

This example shows the identification of low complexity regions (LCRs) in ALKBH5, and functional analysis of the LCRs.

### Methods

### Immunofluorescence

mESCs were seeded onto Matrigel®-coated 8-well chambers, fixed with 4% PFA for 15 minutes, and permeabilized with 0.3% Triton™ X-100 in PBS for 15 minutes. Following permeabilization, cells were blocked in IF buffer containing 3% BSA in PBST for 1 hour. Subsequently, cells were incubated overnight at 4° C. with a DYKDDDDK tag (SEQ ID NO: 74) monoclonal antibody conjugated to Alexa Fluor™ 488 (Invitrogen MA1-142-A488, diluted 1:100 in IF buffer, SEQ ID NO: 74). The following day, cells were washed three times with PBS, and nuclei were counterstained with DAPI (Thermo Scientific™). Fluorescent images were acquired using Leica SP8 confocal microscope.

### Low Complexity Domain Prediction

Low complexity domain prediction was performed as in Example 1.

### Results

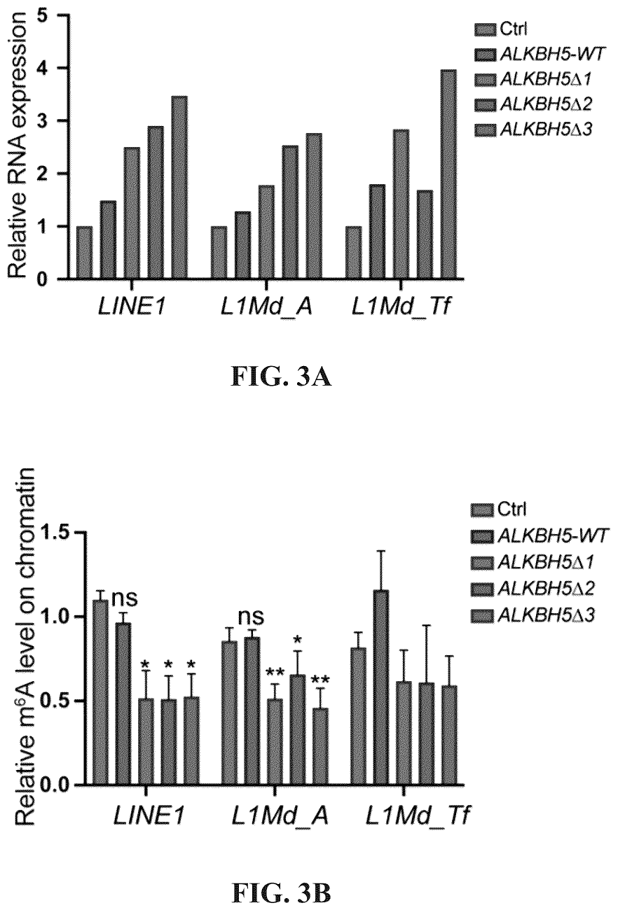

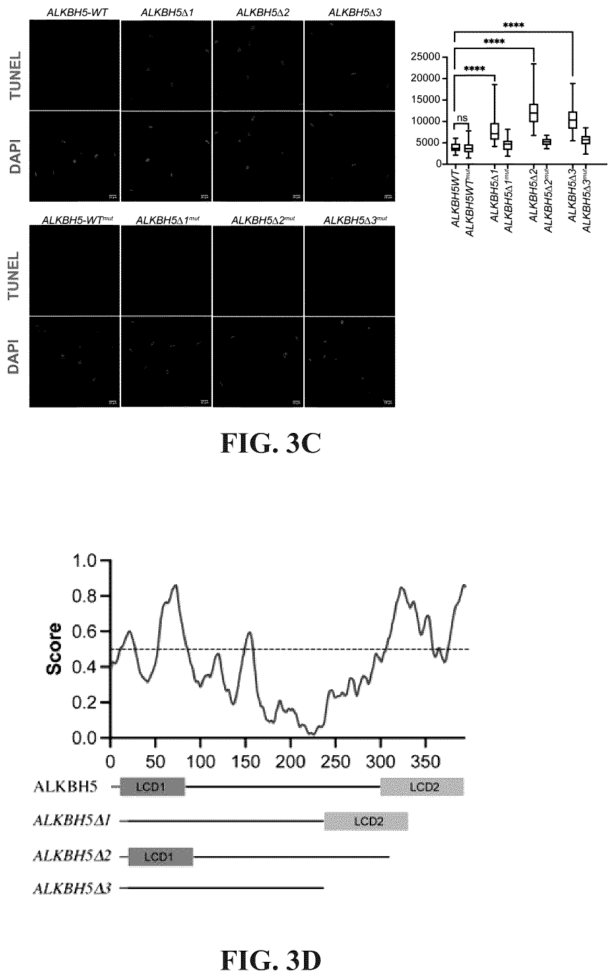

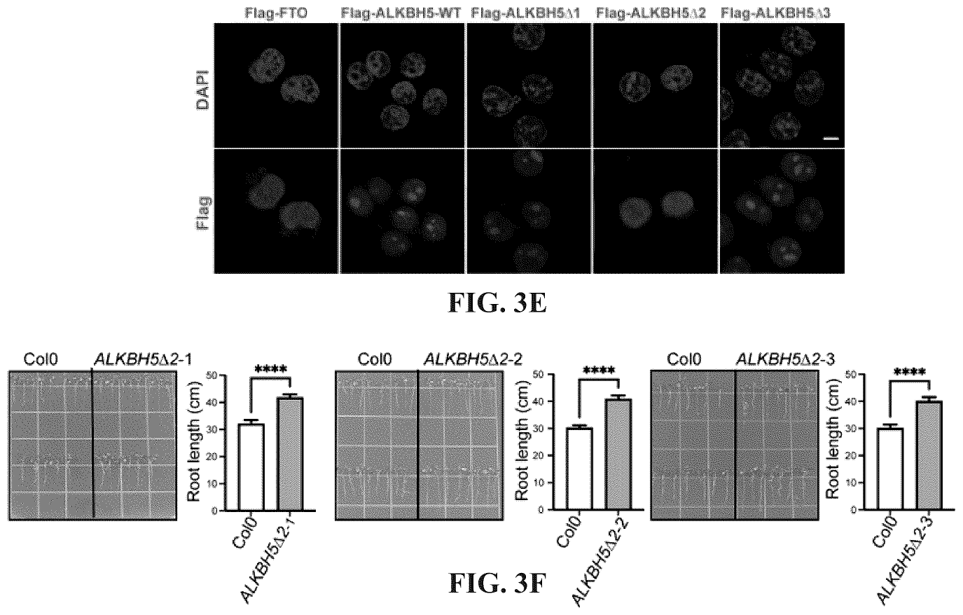

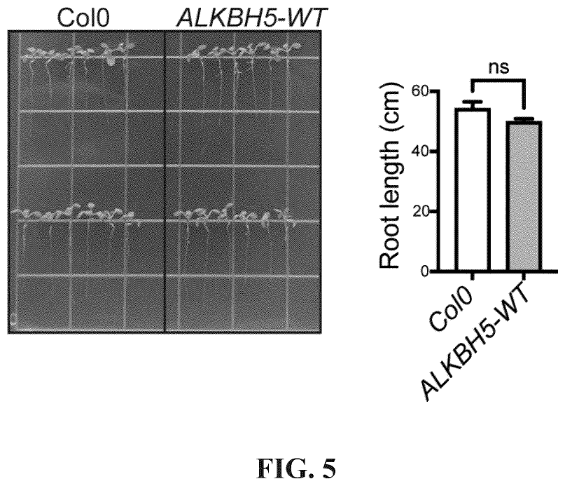

Upon overexpression of the full-length human ALKBH5 in mESCs, neither whole-cell LINE1 RNA level changes nor chromatin-associated LINE1 RNA m⁶A level changes were observed (FIGS. 3A-3B). After Agrobacterium infiltration of the full-length human ALKBH5 in leaves of N. benthamiana, noticeable changes were also absent in DNase I-treated TUNEL signal when compared with catalytically inactive mutant ALKBH5_{H204A} (FIG. 3C). This is consistent with the results in which human FTO could promote plant growth but human ALKBH5 could not (PMID: 34294912; FIG. 5). Distinct features of human FTO compared to ALKBH5 were then investigated to determine the cause of the observed functional differences. A comprehensive sequence and structural analysis revealed that human ALKBH5 possessed additional domains at its N and C terminals, extending beyond the catalytic core in comparison to FTO. Utilizing the Prediction of Intrinsically Unstructured Proteins (IUPred3), these regions were identified as low complexity regions (LCRs) (FIG. 3D). LCRs are known to form reversible biomolecular condensates through liquid-liquid phase separation (LLPS). Immunofluorescence (IF) assays demonstrated that ALKBH5 formed granules in the nucleus, while FTO did not (FIG. 3E). This led to the hypothesis that these LCRs may mediate LLPS, restricting ALKBH5 to specific locations rather than exhibiting a diffuse pattern throughout chromatin like FTO. Consequently, FTO could access and act on a range of different caRNAs to affect chromatin state, while ALKBH5 was restricted to specific locations.

To validate this hypothesis, the two LCRs of ALKBH5 were selectively deleted, and the truncated forms overexpressed individually in mESCs. Overexpression of human ALKBH5 constructs with truncation of the N- or C-terminal LCRs of ALKBH5 (ALKBH5_{Δ30-81} and ALKBH5_{Δ298-394}) elevated whole-cell LINE1 RNA level in mESCs, respectively (FIG. 3A). Simultaneous truncation of both LCRs (ALKBH5_{Δ30-81 and 298-394}) led to further increased whole-cell LINE1 RNA level in mESCs upon overexpression (FIG. 3A). In addition, overexpression of these ALKBH5 truncated forms (ALKBH5_{Δ30-81}, ALKBH5_{Δ298-394} and ALKBH5_{Δ30-81 and 298-394}) effectively decreased m⁶A level of chromatin-associated LINE1 RNA, respectively, while overexpression of catalytically inactive mutants (ALKBH5_{Δ30-81/H204A}, ALKBH5_{Δ298-394/H204A} and ALKBH5_{Δ30-81 and 298-394/H204A}) did not (FIG. 3B).

Next, transient Agrobacterium infiltration of ALKBH5 constructs was performed in leaves of N. benthamiana. Expression of all forms of ALKBH5 truncated versions, but not inactive mutants, notably opened chromatin, with ALKBH5_{Δ298-394}, the C-terminal truncated variant, exhibiting the most significant effect (FIG. 3C). The catalytic domain of human ALKBH5 (amino acids 74 to 294) has been well characterized in previous studies. While researchers have confirmed that the catalytic domain of human ALKBH5 (amino acids 74-294) is active for m⁶A demethylation (PMID: 24778178), recent studies also found that the C terminus (amino acids 294-394) of ALKBH5 promotes its phase separation in vitro (PMID: 37474102). Consistently, we observed ALKBH5_{Δ298-394} did not form nuclear condensates in mESCs (FIG. 3E), confirming the hypothesis that this C-terminal LCR may be responsible for restriction of full length ALKBH5 from accessing different chromatin sites.

Transgenic Arabidopsis and rice plants expressing these variants were then generated. Arabidopsis root growth serves as a relatively fast indicator for evaluating the potential of promoting plant growth by engineered demethylases. The expression of ALKBH5_{Δ298-394} (truncation of the C-terminal LCR) led to significantly increased root growth compared with the wild type control (Col-0) (FIG. 3F). These results confirmed the hypothesis that LCRs in ALKBH5 limits its access to caRNAs m⁶A sites and thus restrict its activity to promote open chromatin.

### Example 3B: The Low Complexity Regions (LCRs) of ALKBH5 Restrict its Access to Chromatin Sites and Limits its Activity on Chromatin-Associated RNAs

### Methods

Methods are provided after Example 6.

### Results

Alkbh5 cIDR Deletion Induces Open Chromatin and Transcription Upregulation in mESCs

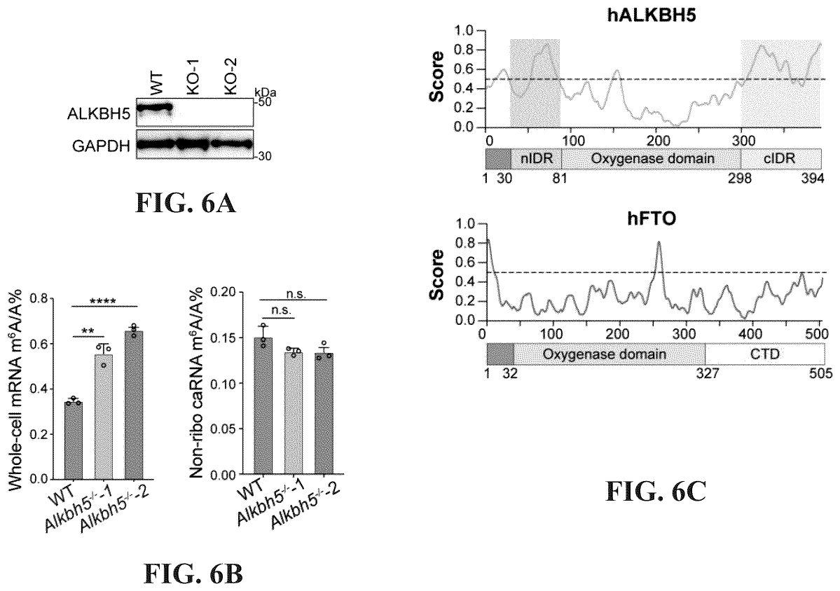

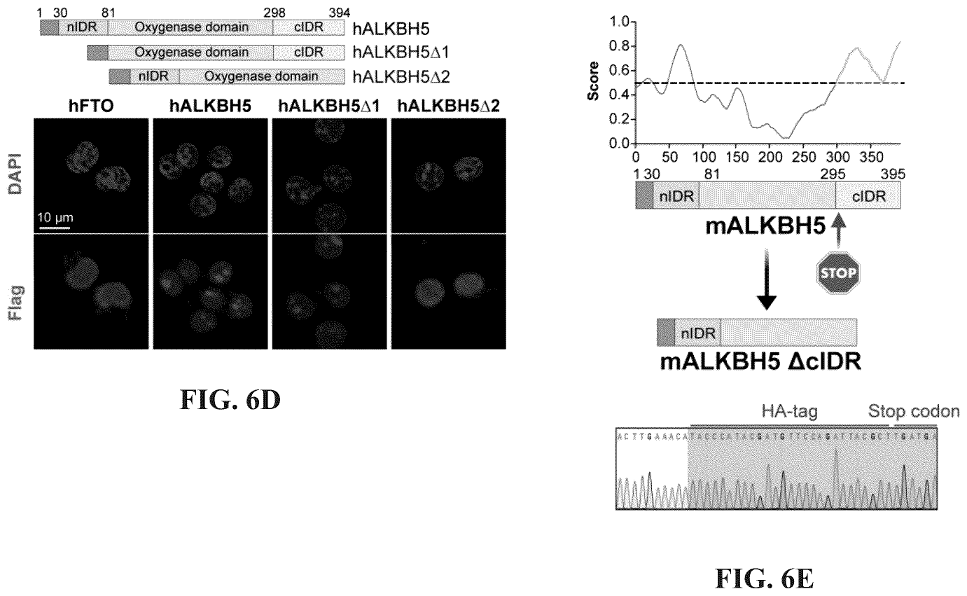

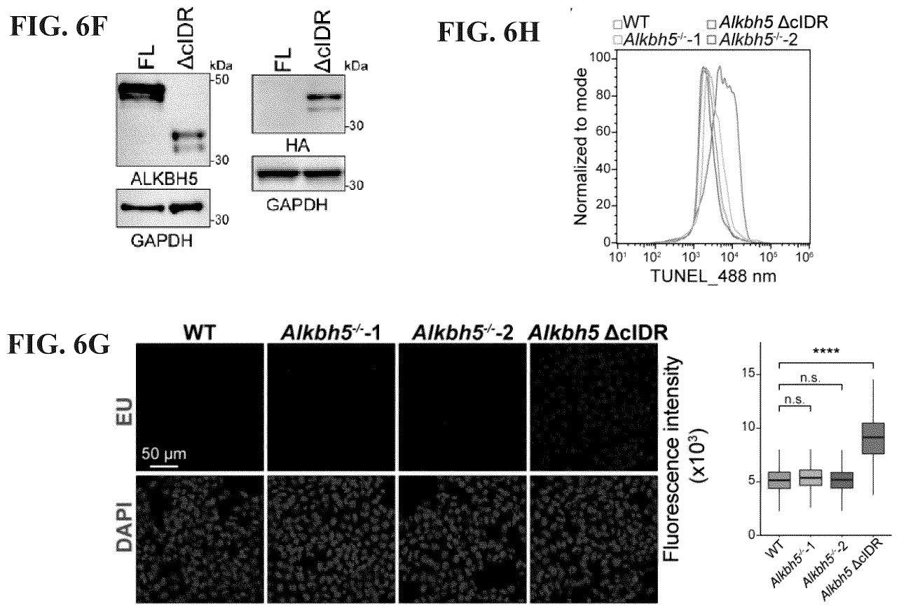

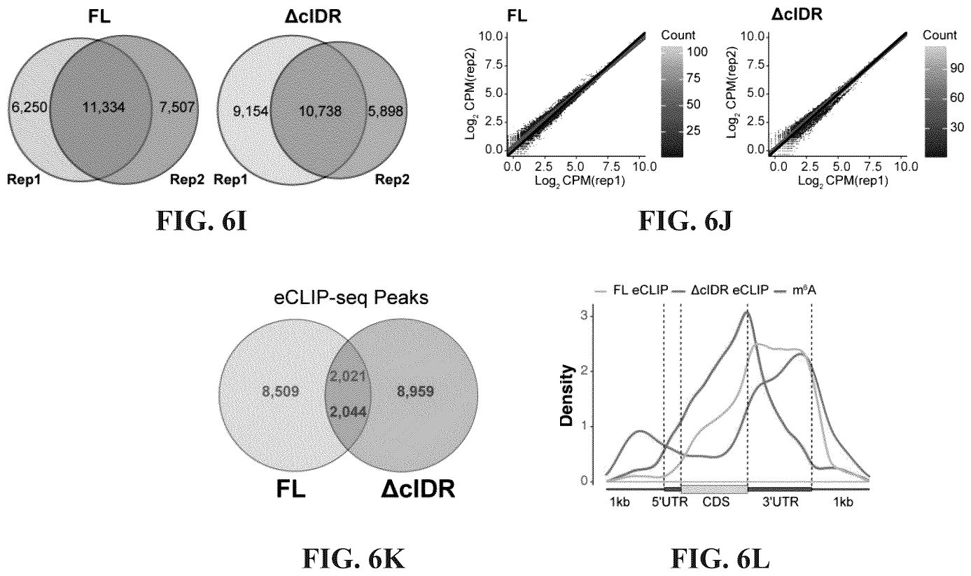

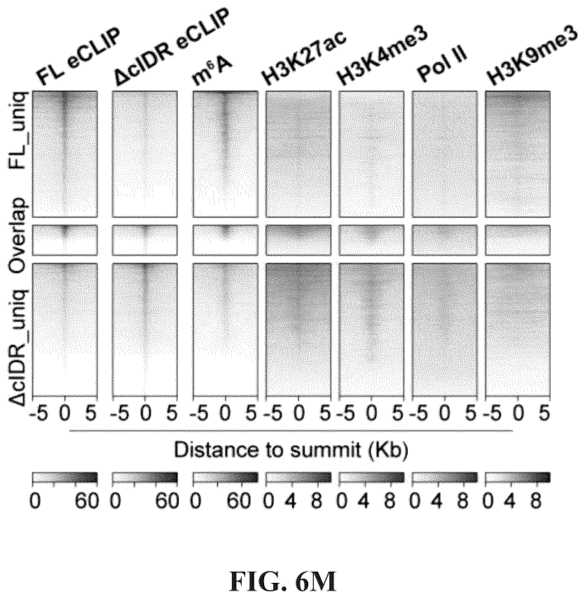

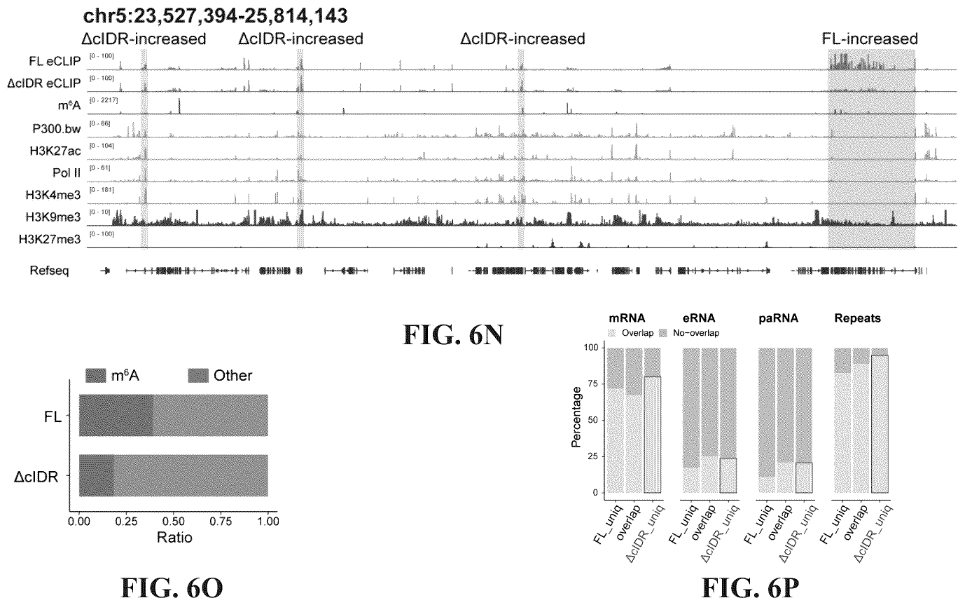

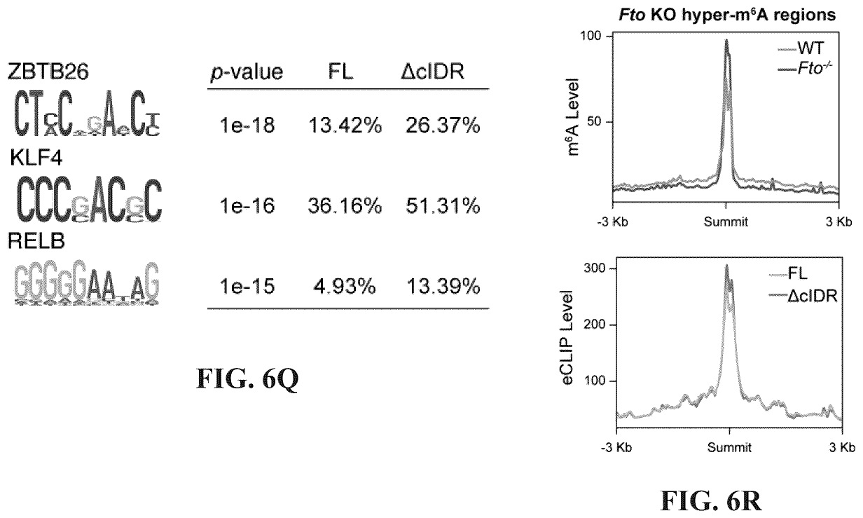

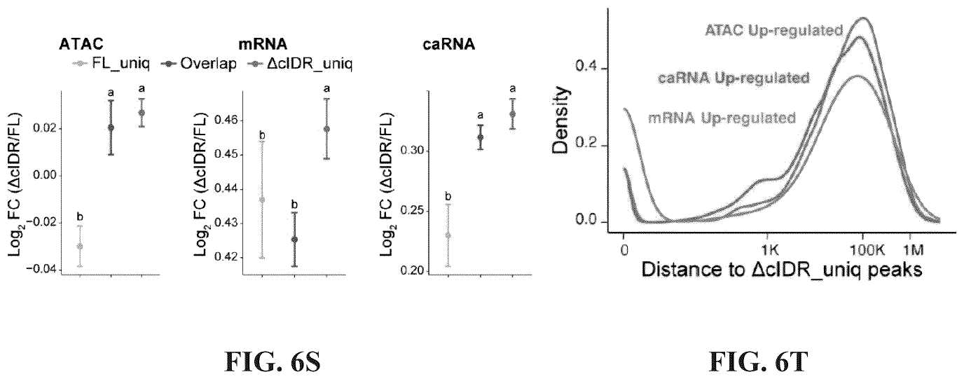

Previous work demonstrated that Fto knockout (KO) in mESCs led to a notable increase in m⁶A levels on caRNA, with minimal impact on mRNA (Wei et al. 2022). Building on these findings, RNA substrate preference of ALKBH5 in mESCs was investigated. Two independent Alkbh5 KO mESC lines were generated (Alkbh5^{-/-}-1 and Alkbh5^{-/-}-2) (FIG. 6A), and whole-cell polyadenylated RNA (polyA+ RNA) as well as non-ribosomal RNA was isolated from the chromatin-associated fraction. Using liquid chromatography-tandem mass spectrometry (LC-MS/MS) to quantify m⁶A/A levels, it was found that Alkbh5 KO resulted in a marked increase in m⁶A levels on mRNA, with only a limited effect on caRNA (FIG. 6B). These observations suggested that ALKBH5, unlike FTO, primarily targets mRNA in mESCs.

To explore the molecular basis for this substrate selectivity, the domain architectures of mammalian ALKBH5 and FTO were compared. Both enzymes contained conserved catalytic domains; however, ALKBH5 also possessed additional intrinsically disorder regions (IDRs) at its N- and C-termini, which were absent in FTO (FIG. 6C). Previous studies also revealed that the catalytic domain of ALKBH5 retained m⁶A demethylation activity comparable to the full-length protein in vitro (Xu et al., 2014; Feng et al., 2014). This suggested that IDRs don't directly affect the catalytic activity of ALKBH5 but may modulate other functional properties. Indeed, IDRs were known to mediate protein-protein interactions, influence RNA-binding activity of RNA-binding proteins, and regulate subcellular localization of various chromatin-modifying proteins (Holchouse and Kragelund, 2024; Bhattarai and Emerson, 2020; Calabretta and Richard, 2015; Wang et al., 2018). Given these roles of IDRs, it was hypothesized that ALKBH5 IDRs might play important roles on its RNA substrate preference compared to FTO.

To test this, human full-length ALKBH5, FTO, and ALKBH5 variants lacking either the N- or C-terminal IDRs were ectopically expressed in mESCs (FIGS. 3E, 6D); human ALKBH5 and FTO were used in all ectopic expression experiments in mESCs and plants unless noted otherwise. FTO exhibited a diffused nuclear localization, while full-length ALKBH5 formed distinct nuclear condensates (FIGS. 3E, 6D). Deletion of the cIDR of ALKBH5 led to a diffused distribution pattern similar to that of FTO (FIGS. 3E, 6D), indicating a crucial role of ALKBH5 cIDR for its condensate formation. This observation aligned with a recent report showing that ALKBH5 undergoes phase separation through its cIDR in vitro (Qin et al., 2023).

To further investigate the function of cIDR, an Alkbh5-ΔcIDR mESC line was generated using CRISPR-Cas9 to introduce an HA-tagged stop codon upstream of the cIDR-encoding region at the endogenous Alkbh5 locus (FIGS. 6E-6F). Deletion of the endogenous cIDR of Alkbh5 resulted in notable increases in global chromatin accessibility and nascent RNA transcription in mESCs (FIGS. 6G-6H). In contrast, Alkbh5 KO had minimal effects on these features (FIGS. 6G-6H). These results indicated that the cIDR of mouse ALKBH5 (mALKBH5) likely suppressed its chromatin activation activity; the deletion of cIDR likely turned mALKBH5 into an FTO-like demethylase that could modulate global chromatin state and transcription (Wei et al., 2022).

To understand how cIDR affected RNA-binding specificity, enhanced cross-linking and immunoprecipitation sequencing (eCLIP-seq) was performed on endogenous full-length mALKBH5 in WT mESCs and on its cIDR-deleted variant (mALKBH5-ΔcIDR) in Alkbh5-ΔcIDR mESC, respectively (FIGS. 61-6J). Only 20% of the eCLIP peaks overlapped between full-length mALKBH5 and mALKBH5-ΔcIDR (FIG. 6K), indicating substantial changes in RNA-binding specificity upon deletion of the cIDR in mALKBH5. Metaplot analysis along mRNA revealed that while both proteins bound near stop codons, mALKBH5-ΔcIDR exhibited increased binding near promoter regions, suggesting a shift in preference towards cis-regulatory elements (CREs) upon cIDR deletion (FIG. 6L).

To further dissect their differences in RNA-binding preferences, eCLIP peaks were catagorized into three groups: (1) unique to full-length mALKBH5 (FL_uniq), (2) overlapping peaks (overlap), and (3) unique to mALKBH5-ΔcIDR (ΔcIDR_uniq). ΔcIDR-unique peaks were enriched for chromatin marks associated with promoters (H3K4me3), enhancers (H3K27ac, EP300), and transcriptionally active regions (Pol II occupancy) (FIGS. 6M-6N). The full-length mALKBH5 centered more around the m⁶A-modified sites on mRNA (FIGS. 6M-60), whereas mALKBH5-ΔcIDR displayed a broader genomic distribution, showing increased binding to various RNA species, including mRNA, as well as promoter-associated RNA (paRNA), enhancer RNA (eRNA), and RNA transcribed from transposable elements (repeat RNA), which are collectively known as chromatin-associated regulatory RNAs (carRNAs) and were previously implicated in chromatin regulation (FIG. 6P) (Wei et al., 2022); Liu et al., 2020). Motif analysis revealed an enrichment of specific transcription factor motifs at ΔcIDR-bound regions (FIG. 6Q), supporting its increased binding to promoter regions. Additionally, mALKBH5-ΔcIDR exhibited higher binding affinity for mouse FTO (mFTO)-sensitive m⁶A sites compared to full-length mALKBH5 (FIG. 6R).

ATAC-seq and transcriptome analyses revealed significantly increased chromatin accessibility and elevated transcription of both mRNA and caRNA at ΔcIDR-unique peaks (FIG. 6S). Notably, these effects are most pronounced at ΔcIDR-bound regions but also extended up to 100 kb away, suggesting their enhancer-like activity (FIG. 6T).

cIDR Deletion Redirects mALKBH5 to Chromatin-Associated Repeat RNAs in mESCs

The extensive changes in RNA-binding profiles observed upon cIDR deletion prompted investigation of their potential impact on the chromatin-associated RNA m⁶A methylome. To address this, m⁶A methylated RNA immunoprecipitation sequencing (MeRIP-seq) was performed on non-ribosomal caRNA isolated from WT, Alkbh5^{-/-} and Alkbh5-ΔcIDR mESCs. The identified RNAs were categorized into camRNA (chromatin-associated, protein-coding mRNA) and carRNAs that include paRNA, eRNA, and repeat RNA.

Differential m⁶A analysis revealed distinct methylation patterns between Alkbh5^{-/-} and Alkbh5-ΔcIDR mESCs when compared to WT mESCs (FIG. 7A). Specifically, Alkbh5 KO resulted in 14,576 hypermethylated peaks (sites demethylated by full-length mALKBH5 in WT mESCs), predominantly mapped to camRNA (FIG. 7B) and distributed across both exons and introns (FIG. 7C-7D). In contrast, Alkbh5-ΔcIDR mESCs displayed 2,409 hypomethylated peaks (sites demethylated by mALKBH5-ΔcIDR), primarily enriched in carRNAs, particularly repeat RNAs (FIG. 7B). The majority of these hypomethylated peaks are located in intergenic regions (FIG. 7C). These differentially methylated sites aligned with the eCLIP-seq findings, where hypermethylated peaks in Alkbh5^{-/-} mESCs were more extensively bound by full-length mALKBH5, and hypomethylated peaks in Alkbh5-ΔcIDR mESCs were preferentially occupied by mALKBH5-ΔcIDR (FIG. 7E), with minimal overlap between the two groups (FIG. 7A).

To evaluate transcriptional impacts of these differential m⁶A patterns, transcripts were categorized into m⁶A-marked and non-m⁶A-marked subgroups. Compared to non-m⁶A-marked transcripts, m⁶A-marked camRNA displayed greater transcriptional down-regulation following Alkbh5 KO (FIG. 7E). Conversely, m⁶A-marked carRNAs, including eRNA, paRNA, and repeat RNA, exhibited greater transcriptional up-regulation upon Alkbh5-ΔcIDR (FIG. 7G). Moreover, transcriptional changes negatively correlated with m⁶A methylation alterations for camRNAs upon Alkbh5 KO, and for carRNAs following Alkbh5-ΔcIDR (FIG. 7H). Collectively, these findings indicated that cIDR deletion shifted mALKBH5 substrate preference from camRNAs to carRNAs.

A closer examination of hypomethylated peaks in Alkbh5-ΔcIDR mESCs revealed that repeat RNAs, particularly LINE1 elements, were the most significantly affected subset. LINE1 elements accounted for the largest proportion of hypomethylated repeat RNAs and exhibited consistently elevated transcription (FIGS. 3A-3B, 71-7J). Gene set enrichment analysis (GSEA) identified evolutionarily young LINE1 subfamilies as notably enriched among hypomethylated repeat RNAs (FIG. 7K). Moreover, m⁶A methylation levels negatively correlated with transcript abundance for these young LINE1 subfamilies (FIG. 7L). Additionally, the elevated abundance was specific to young LINE1s, not old LINE1s (FIG. 7M). LINE1 RNAs constitute approximately 20% of mammalian genomes, with recent studies highlighting the regulatory role of evolutionarily young LINE1s, which remain retrotranscriptionally active, in chromatin states and transcription (Wei et al., 2022; Liu et al., 2020; Liu et al., 2021; Jachowicz et al., 2017; Percharde et al., 2018; Marasca et al., 2022; Li et al., 2024). Together, these results suggested that mALKBH5-ΔcIDR predominantly targeted young LINE1s, resulting in reduced m⁶A methylation and upregulated transcription. This activation of young LINE1s likely contributed to elevated chromatin accessibility and transcription observed in Alkbh5-ΔcIDR mESCs.

These findings partially overlap with earlier observations in Fto^{-/-} mESCs, where mouse FTO (mFTO) demethylates m⁶A on chromatin-associated repeat RNAs, particularly LINE1, thereby regulating chromatin states and transcription (Wei et al., 2022). To explore this overlap, m⁶A peaks located on repeat elements that were elevated upon Fto KO were defined as “mFTO regulatory regions”, based on published caRNA MeRIP-seq data (Wei et al., 2022). When profiling m⁶A and transcription levels across mFTO regulatory regions, reduced m⁶A levels accompanied by elevated transcription were observed in Alkbh5-ΔcIDR mESCs compared to WT mESCs (FIG. 7N), supporting a partial overlap of their m⁶A demethylation profile on repeat RNAs.

It was also previously shown that a nuclear m⁶A-binding protein YTHDC1 can mediate the degradation of the methylated repeat RNA (Liu et al., 2020). In Fto KO mESCs, elevated YTHDC1 binding accelerates LINE1 RNAs decay, leading to reduced transcription rates (Wei et al., 2022). Similarly, the present results showed that Alkbh5-ΔcIDR led to reduced LINE1 RNA decay along with decreased YTHDC1 binding (FIGS. 70-7P). YTHDC1 binding also recruits histone modifiers that install repressive histone marks to suppress local transcription (Wei et al., 2022; Liu et al., 2021; Xu et al., 2021). Consistently, spike-in-calibrated EU-labeled nascent RNA sequencing further revealed a markedly increased transcription rate of repeat RNA upon Alkbh5-ΔcIDR, whereas Alkbh5 KO showed only a modest effect (FIGS. 7Q-7R). Notably, this increased transcription rate was restricted to hypomethylated regions, with LINE1 RNA showing the most pronounced enhancement (FIG. 7S). These results underscored the role of mALKBH5-ΔcIDR-mediated m⁶A demethylation in promoting nascent repeat RNA synthesis and maintain its stability on chromatin.

Despite functional overlaps between mFTO and mALKBH5-ΔcIDR, notable differences existed in their regulatory targets. In Fto^{-/-} mESCs, hypermethylated peaks were predominantly intronic (FIG. 7C), consistent with the established role of mFTO in modulating intragenic LINE1 RNA in mESCs. These LINE1-containing genes are involved in differentiation and development (Wei et al., 2022). In contrast, mALKBH5-ΔcIDR exhibited a more pronounced regulatory effect on intergenic LINE1 (FIG. 7T), suggesting a broader influence of mALKBH5-ΔcIDR on chromatin architecture.

In summary, these findings demonstrated distinct regulatory functions for ALKBH5, FTO and ALKBH5-ΔcIDR within the chromatin-associated fraction in mESCs (FIG. 7U). mALKBH5 primarily demethylated m⁶A on exonic and intronic regions of camRNA, influencing pre-mRNA abundances. mFTO mainly targeted intragenic repeat RNAs, whereas mALKBH5-ΔcIDR preferentially engaged intergenic repeat RNAs. Both mFTO and mALKBH5-ΔcIDR could notably change chromatin organization and transcription activation.

cIDR Enables ALKBH5 Interaction with EJC for mRNA Targeting

Given the substantial changes induced by cIDR deletion for ALKBH5, proteins that directed the full-length ALKBH5 to mRNA in a cIDR-dependent manner were sought. Protcomic analysis identified components of the exon junction complex (EJC) as highly enriched interaction partners of ALKBH5 (Yang et al., 2022; Covelo-Molares et al., 2021). Co-immunoprecipitation results showed that cIDR deletion abolished mALKBH5 interaction with EJC components (FIG. 7V) in mESCs. These observations supported a model wherein the cIDR of ALKBH5 promoted nuclear condensate formation and enabled binding with EJC, directing ALKBH5 for mRNA m⁶A demethylation. Upon cIDR deletion, ALKBH5 was released from condensates and dispersed throughout the nucleus, gaining increased access to chromatin. In this state, ALKBH5 engaged in m⁶A demethylation of chromatin-associated repeat RNA, thereby promoting chromatin openness and activating transcription.

cIDR Mediates Functional Divergence Between FTO and ALKBH5

Previous work demonstrated that depletion of Fto in mESCs leads to decreased proliferation and abnormal differentiation (Wei et al., 2022). While mALKBH5-ΔcIDR and mFTO shared overlapping roles in regulating LINE1 elements, it was hypothesized that ALKBH5-ΔcIDR could compensate for FTO loss in mESCs. Indeed, ectopic expression of human ALKBH5-ΔcIDR partially restored both proliferation and differentiation of Fto--mESCs (FIGS. 7W-7X), suggesting functional similarities between FTO and ALKBH5-ΔcIDR.

To further probe whether ALKBH5 cIDR mediated functional differences between FTO and WT ALKBH5, an FTO-cIDR variant was generated by fusing the cIDR of human ALKBH5 to the C-terminus of human FTO (FIG. 7Y). Ectopic expression of FTO-cIDR in mESCs led to the formation of nuclear condensates (FIG. 7Y), resembling those formed by WT ALKBH5. Similar to WT ALKBH5, FTO-cIDR expression reduced m⁶A levels on mRNA but had limited effects on caRNA, as measured by LC-MS/MS (FIG. 7Z). Furthermore, FTO-cIDR expression in Alkbh5^{-/-} mESCs also rescued proliferation and differentiation defects (FIGS. 7AA-7AD). Together, these findings demonstrated the critical role of ALKBH5 cIDR in shaping the distinct functional profiles between ALKBH5 and FTO.

m⁶A-Hypomethylated LINE1 Acts as Enhancer-Like Element to Modulate Chromatin State

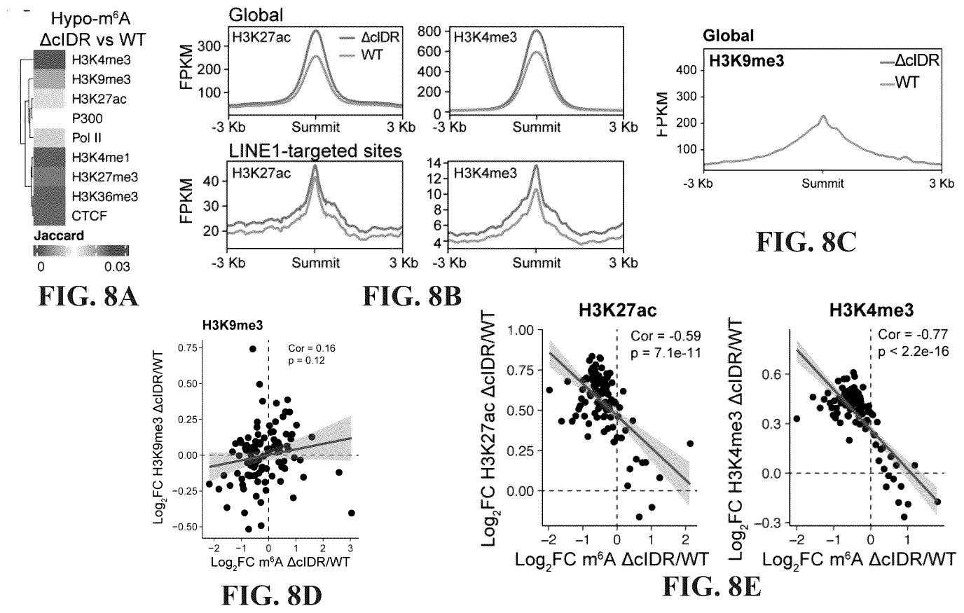

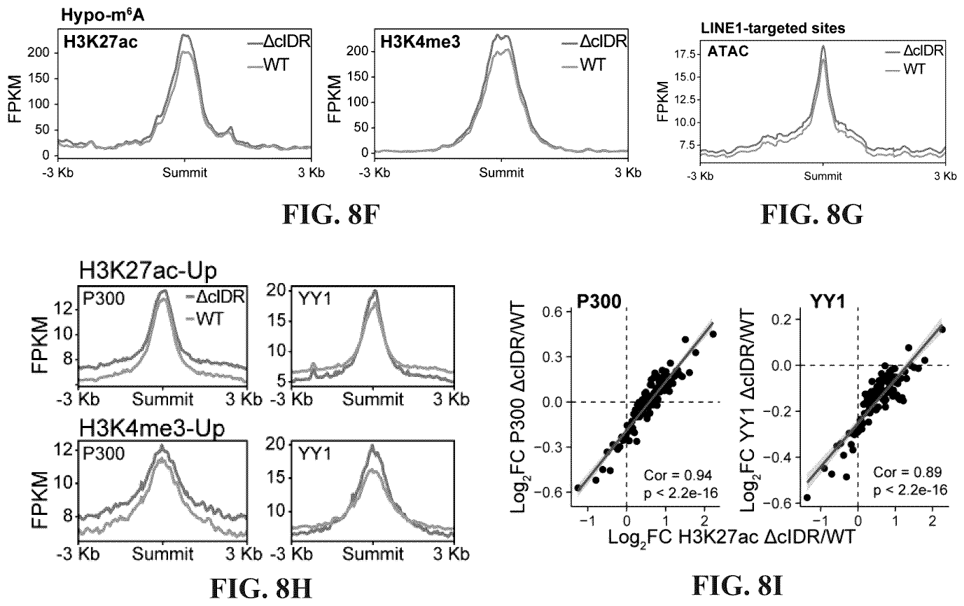

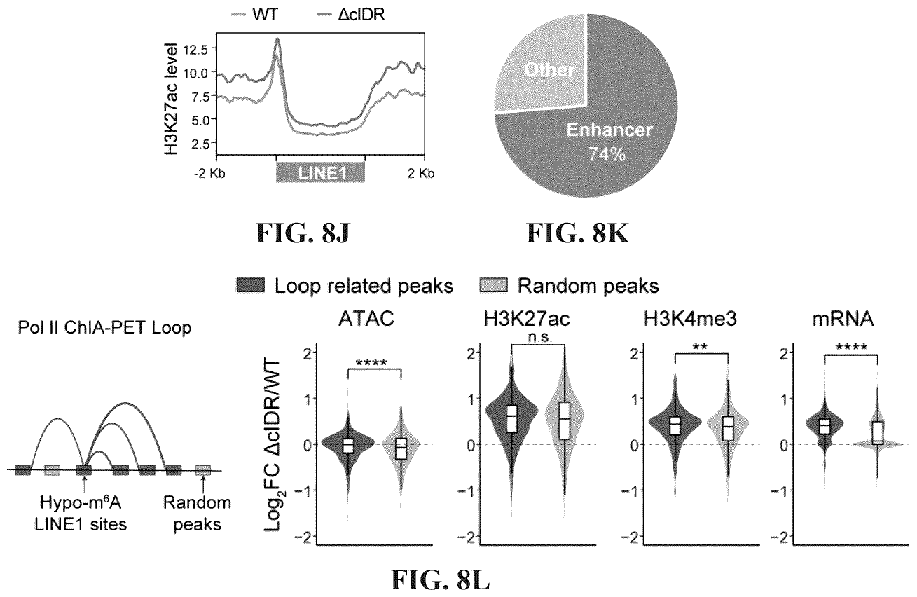

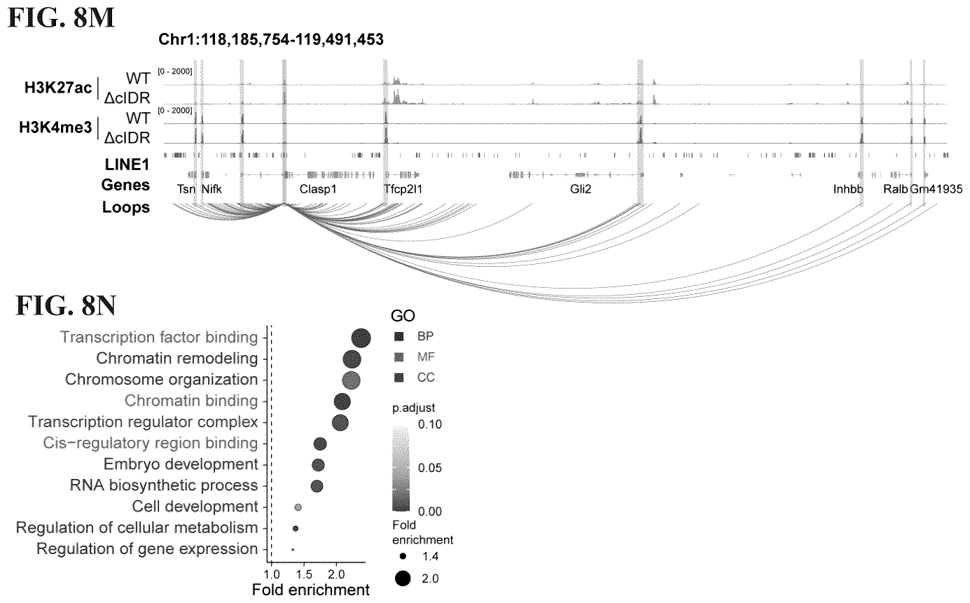

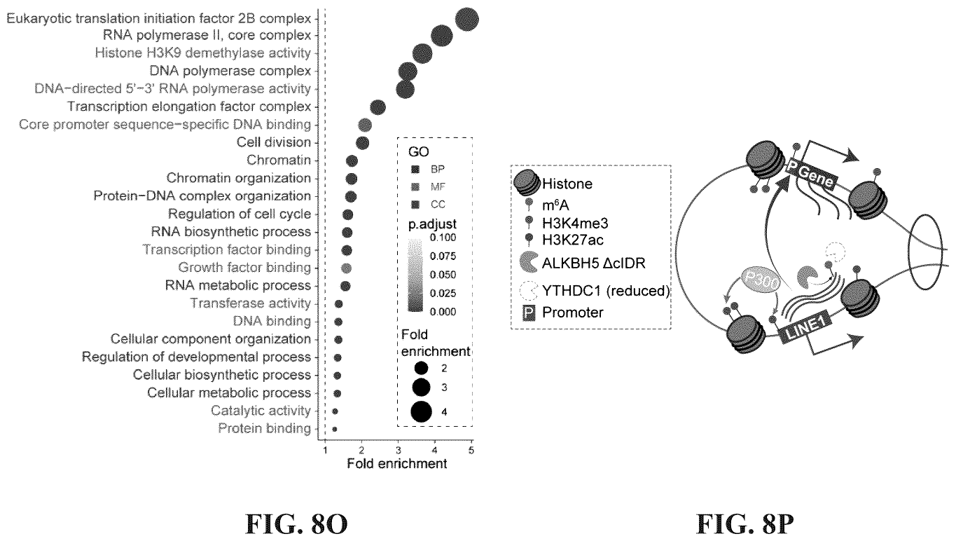

To investigate how mALKBH5-ΔcIDR regulated chromatin state in mESCs, the association of m⁶A-hypomethylated regions with chromatin regulators was examined, and strong overlaps were observed with regions marked by H3K4me3, H3K9me3 and H3K27ac (FIG. 8A). CUT&Tag profiling of H3K4me3 and H3K27ac, and CUT&RUN profiling of H3K9me3, revealed global increases in H3K4me3 and H3K27ac levels following Alkbh5-ΔcIDR (FIG. 8B), while H3K9me3 levels remained largely unchanged (FIGS. 8C-8D). Moreover, increased levels of H3K4me3 and H3K27ac correlated well with reduced m⁶A levels on caRNA upon Alkbh5-ΔcIDR (FIGS. 8E-8F).

LINE1 RNA, identified as the major substrate of mALKBH5-ΔcIDR in mESCs (FIGS. 7A-78), was known to regulate chromatin state (Wei et al., 2022; Liu et al., 2020; Liu et al., 2021; Jachowicz et al., 2017; Percharde et al., 2018; Marasca et al., 2022; Li et al., 2024). To determine whether these histone modification changes were associated with LINE1 RNA, their occupancies were analyzed across LINE1 RNA-targeted genomic sites identified using published LINE1 RNA ChIRP-seq data (Liu et al., 2021). These regions showed elevated H3K4me3 and H3K27ac levels (FIG. 8B), accompanied by increased chromatin accessibility (FIG. 8G). Additionally, chromatin regulators CBP/P300 and YY1, which interacted with caRNAs and promoted chromatin activation (Sigova et al., 2015; Bose et al., 2017), displayed notable increased occupancy at regions with elevated H3K4me3 and H3K27ac levels following Alkbh5-ΔcIDR (FIG. 8H). Importantly, P300 and YY1 binding positively correlated with H3K27ac deposition upon Alkbh5-ΔcIDR (FIG. 8I), suggesting their roles in reinforcing active chromatin state and facilitating transcriptional activation.

LINE1 elements, highly enriched with H3K27ac at their 5′UTR, can function as enhancers to regulate distal gene transcription through enhancer-promoter looping (Li et la., 2024). Consistent with this, elevated H3K27ac was detected at m⁶A-marked LINE1 elements following Alkbh5-ΔcIDR (FIG. 8J). To investigate whether m⁶A-hypomethylated LINE1 RNA acts as an enhancer to regulate transcription through looping interactions, RNA polymerase II-associated chromatin interaction data (Pol II ChIA-PET) from ENCODE was integrated, which provided a comprehensive view of long-range loops between promoters and enhancers associated with active transcription. A specific focus was placed on chromatin loops where one of the two anchor sites overlapped with m⁶A-hypomethylated LINE1 regions following Alkbh5-ΔcIDR, and these were defined as “m⁶A-hypomethylated LINE1-related loops”. This analysis revealed that 74% of m⁶A-hypomethylated LINE1-associated anchors overlapped with enhancer regions (FIG. 8K), supporting their enhancer-like roles.

To further examine chromatin state and transcription changes associated with m⁶A-hypomethylated LINE1-related loops, ATAC-seq, H3K27ac and H3K4me3 CUT&Tag, and transcriptome data were overlaid. Regions overlapping with loop anchors were designated as ‘loop-related peaks’, while randomly selected non-interacting regions served as ‘random peaks’ for comparison (FIG. 8L). Comparative analysis revealed that loop-related peaks exhibited increased chromatin accessibility, elevated H3K4me3 intensity, and enhanced transcription of protein-coding genes (FIGS. 8L-8M). These findings suggested that elevated H3K27ac on LINE1 regions strengthened enhancer-promoter looping, activated transcription, and increased chromatin accessibility at targeted promoters, which were further enhanced with elevated H3K4me3.

Gene ontology (GO) analysis revealed that genes activated through m⁶A-hypomethylated LINE1-related loops were significantly enriched in processes related to transcription and chromatin regulation (FIG. 8N), potentially amplifying the downstream regulatory effects of hypomethylated LINE1. At the global level, genes activated in Alkbh5-ΔcIDR mESCs were enriched in translation, transcription, and cell cycle regulation, reflecting broader impacts on cellular growth and development (FIG. 8O).

In summary, these findings revealed that cIDR deletion in ALKBH5 released the demethylase to induce m⁶A demethylation of LINE1 RNA. These m⁶A-hypomethylated LINE1 RNAs, stabilized against the YTHDC1-mediated degradation, promoted chromatin opening, enhanced H3K27ac and H3K4me3 deposition, and recruited transcriptional activators P300 and YY1. Consequently, LINE1 elements marked by increased H3K27ac functioned like enhancers, regulating distal gene transcription through enhancer-promoter looping. This process may help establish a self-reinforcing transcriptional network, where activated genes and active chromatin states collaboratively drive broader chromatin activation (FIG. 8P).

### Example 3.5: Effects of Truncated Forms of ALKBH5 on Chromatin Regulation in Arabidopsis

### Methods

Methods are provided after Example 6.

### Results

The significant enhancement of root growth observed in IDR-deleted ALKBH5-transgenic Arabidopsis prompted further investigation to probe the underlying mechanism. As in mESCs, human ALKBH5_{Δ298-394}-transgenic Arabidopsis (herein referred to as “ALKBH5-ΔcIDR”) was examined, with full-length ALKBH5-transgenic and catalytically inactive mutant lines serving as controls.

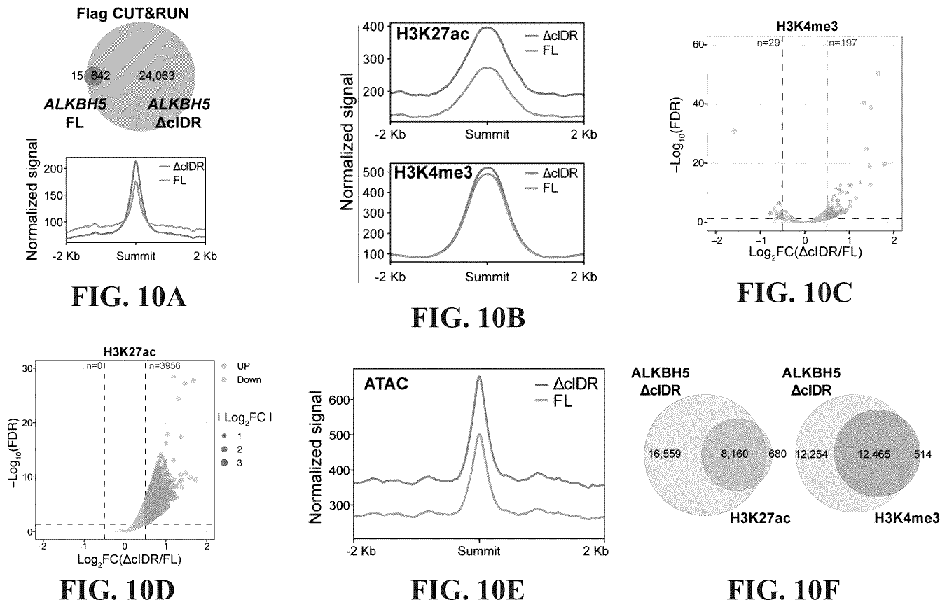

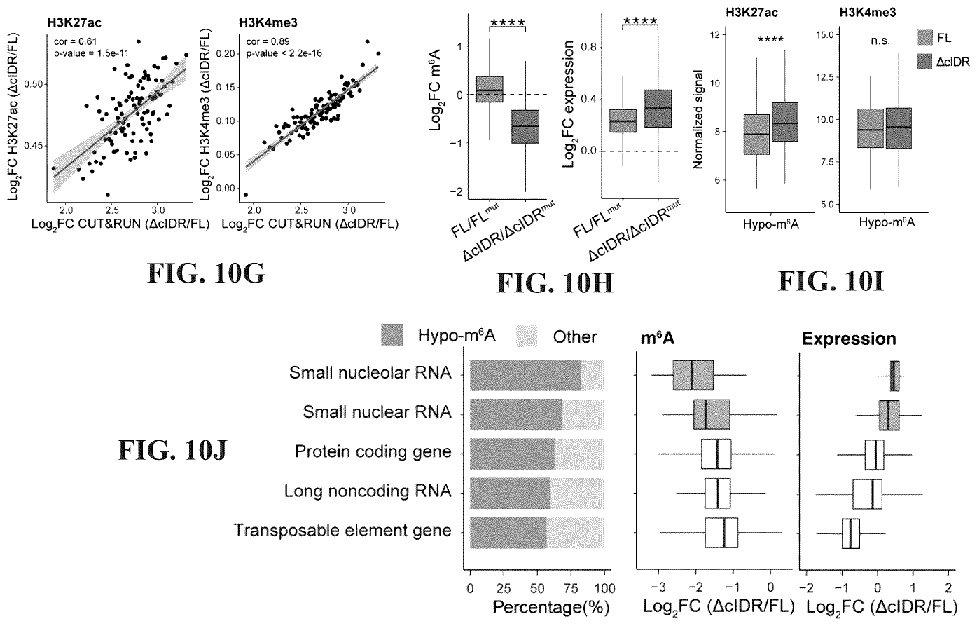

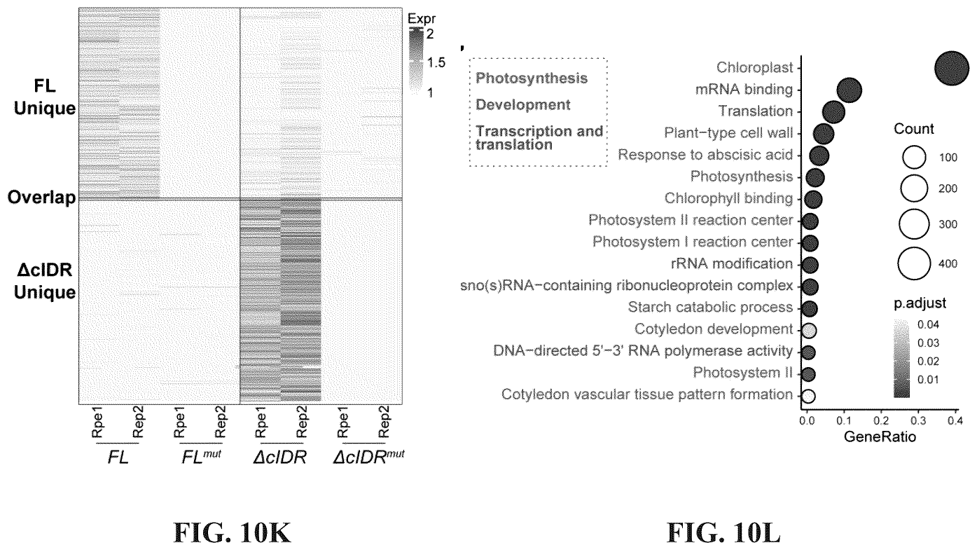

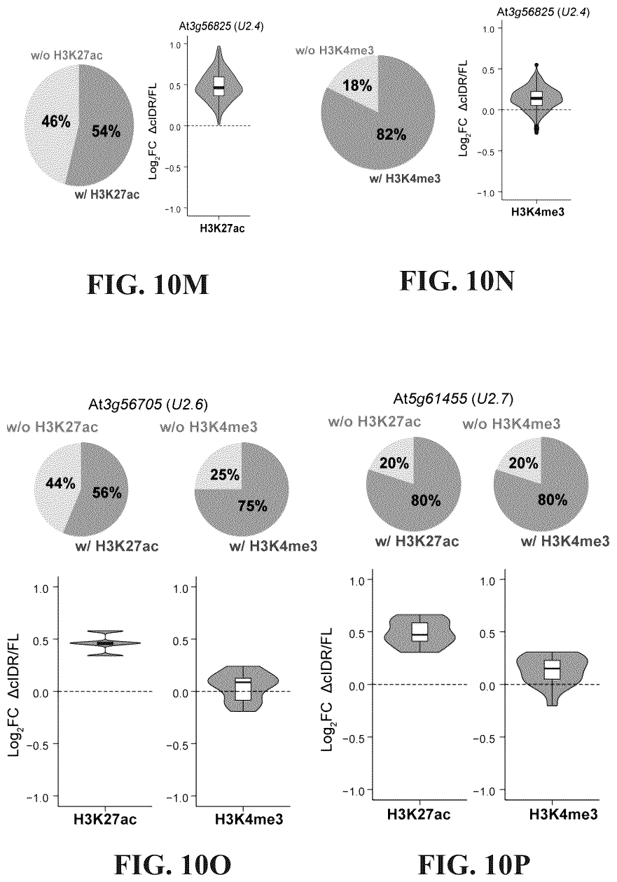

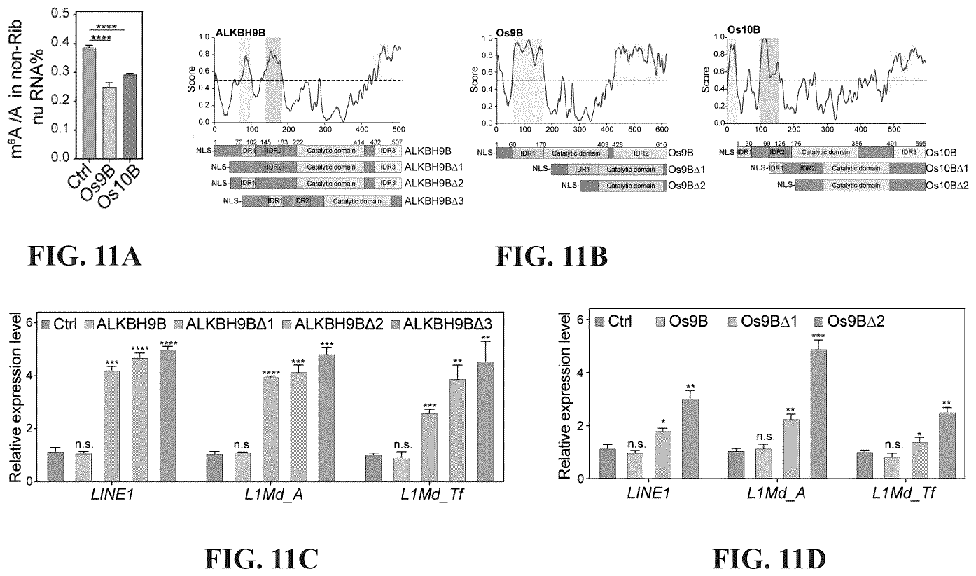

To assess chromatin localization, Flag-tag CUT&RUN assays were performed on transgenic Arabidopsis expressing Flag-tagged full-length ALKBH5 and ALKBH5-ΔcIDR. Notably, ALKBH5-ΔcIDR exhibited dramatically enhanced chromatin binding compared to full-length ALKBH5 in Arabidopsis (24,705 binding sites for ALKBH5-ΔcIDR versus 657 binding sites for ALKBH5) (FIG. 10A), supporting the hypothesis that IDRs acted as restraints limiting chromatin engagement of full-length ALKBH5. Building on the prior observations in mESCs, where Alkbh5-ΔcIDR induced elevated H3K4me3 and H3K27ac levels, it was next investigated whether similar chromatin changes occurred in ALKBH5-ΔcIDR-Arabidopsis. CUT&Tag profiling revealed a global increase in H3K4me3 and H3K27ac levels in ALKBH5-ΔcIDR-Arabidopsis compared to full-length ALKBH5-Arabidopsis (FIGS. 10B-10D), accompanied with enhanced chromatin accessibility as demonstrated by ATAC-seq (FIG. 10E). Importantly, elevated H3K4me3 and H3K27ac signals correlated well with the increased chromatin binding of ALKBH5-ΔcIDR (FIGS. 10F-10G), supporting that the more extensive chromatin engagement of ALKBH5-ΔcIDR contributes to the establishment of active chromatin state.

Given the interplay between chromatin-associated RNA (caRNA) m⁶A methylation and chromatin regulation, it was next investigated whether the elevated chromatin binding and enhanced chromatin activity observed for ALKBH5-ΔcIDR were accompanied by changes in the m⁶A methylome on caRNAs. To this end, non-ribosomal caRNAs were isolated from ALKBH5- and ALKBH5-ΔcIDR-Arabidopsis, along with their respective inactive mutant lines, and MeRIP-seq performed. Analysis revealed significantly greater m⁶A demethylation on caRNAs in ALKBH5-ΔcIDR-Arabidopsis compared to full-length ALKBH5-Arabidopsis, accompanied by increased transcription of m⁶A-marked transcripts (FIG. 10H). These m⁶A-hypomethylated regions in ALKBH5-ΔcIDR-Arabidopsis exhibited increased H3K27ac signals (FIG. 10i). Notably, non-coding RNAs, including small nucleolar RNA (snoRNAs) and small nuclear RNA (snRNAs), displayed significantly reduced m⁶A levels alongside elevated transcription in ALKBH5-ΔcIDR-Arabidopsis (FIG. 10J). The Arabidopsis genome contains limited TEs and previous studies have indicated cis-regulatory roles of non-coding snoRNAs and ncRNAs on chromatin state (Zhang et al., 2024; Li et al., 2021). Consistently, these results suggested that reduced m⁶A levels on these chromatin-associated snoRNAs and ncRNAs elevated their levels on the chromatin, which induced a more open chromatin state (FIGS. 10E, 10I, 10J).

While both transgenic lines showed elevated transcript levels, distinct transcriptional profiles were observed in the ALKBH5-ΔcIDR-mediated m⁶A demethylation (FIG. 10K). GO analysis revealed that genes activated in ALKBH5-ΔcIDR-Arabidopsis were enriched in pathways related to photosynthesis, plant development, and transcriptional and translational regulation (FIG. 10L). Furthermore, the activation of genes involved in transcription and translation suggested a potential amplifying effect, aligning with the findings in mESCs.

To further explore the role of increased caRNAs in chromatin regulation, a subset of top-ranked m⁶A-hypomethylated RNAs (e.g., At3g56825 (U2.4), At3g56705 (U2.6), At5g61455 (U2.7)) in ALKBH5-ΔcIDR-Arabidopsis were selected and the local chromatin state of their previously identified interacting sites (Li et al., 2021) examined. This analysis revealed that over half of these chromatin interaction sites were marked by H3K4me3 and H3K27ac, with both marks showing increased levels in ALKBH5-ΔcIDR-Arabidopsis compared to ALKBH5-Arabidopsis (FIGS. 10M-10P). This aligns with recent reports in Arabidopsis demonstrating that non-coding RNAs may play important roles on regulating chromatin state (Zhang et al., 2024; Li et al., 2021).

Collectively, these results uncovered that m⁶A demethylation by ALKBH5-ΔcIDR elevated the transcript levels of a group of caRNAs. These caRNAs may function as CRE-like regulators, modulating chromatin states and activating transcription in Arabidopsis. This regulation may establish a positive-feedback loop that amplifies transcriptional activity, ultimately promoting plant growth.

### Example 4: Effects of Truncated Forms of Plant ALKBH5 Family Proteins (ALKBH9B and ALKBH10B) on Chromatin Regulation in mESCs and Plant Systems

This example shows the effects of truncated forms of plant ALKBH5 family proteins (ALKBH9B and ALKBH10B) on chromatin regulation in mESCs and plant systems.

### Methods

### Arabidopsis Transformation

Transgenic Arabidopsis seedlings were generated via Agrobacterium-mediated transformation of A. thaliana using the standard floral dip method (PMID: 17406292) in the wild-type Col-0 background. Transgenic T1 populations were screened on compound soil watered with BASTA solution.

### Root Growth Assay

All seeds were sterilized by immersion in 10% sodium hypochlorite solution for 15 minutes, followed by five washes with deionized water. Subsequently, seeds were plated on Murashige and Skoog medium (PhytoTech Labs) supplemented with 0.8% agar and 1.5% sucrose. To synchronize germination, plates were cold-stratified at 4° C. in the dark for 72 hour. Then plates were positioned vertically at 90-degree angle and transferred to normal condition (16-h-light/8-h-dark photoperiod) at 22° C. for another 10 days before plates were imaged and roots were measured.

### Quantitative Analysis of m⁶A Level Using UHPLC-QQQ-MS/MS

50 ng non-ribosomal RNA from the soluble fraction of mESCs was digested with 1 μl of Nuclease P1 (NEB) in 20 μl of 1× Nuclease P1 buffer (NEB) for 2 hour at 37° C. Subsequently, FastAP (1U, Thermo Scientific™) and 2.5 μl of FastAP buffer (Thermo Scientific™) were added, and the solution was further incubated at 37° C. for 4 hours. The resulting solution was then filtered through a 0.22 μm syringe filter and diluted to a final volume of 60 μl. Subsequently, 10 μl of the solution was injected into an LC-MS/MS system. Nucleosides were separated using reverse-phase ultra-performance liquid chromatography on a C18 column, coupled with online mass spectrometry detection using an Agilent 6410 Triple Quadrupole LC mass spectrometer in positive electrospray ionization mode. The nucleosides were quantified using the nucleoside to base ion mass transitions of 282 to 150 (m⁶A), and 268 to 136 (A). Quantification was performed by comparison with the standard curve obtained from pure nucleoside standards running at the same batch of samples. The ratio of m⁶A to A was calculated based on the calculated concentrations.

Additional methods are provided after Example 6.

### Results

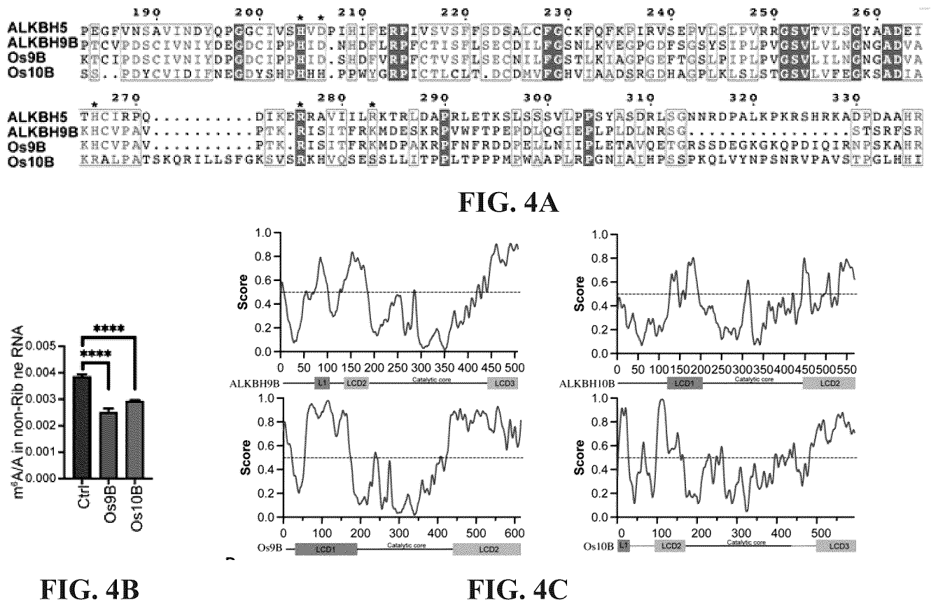

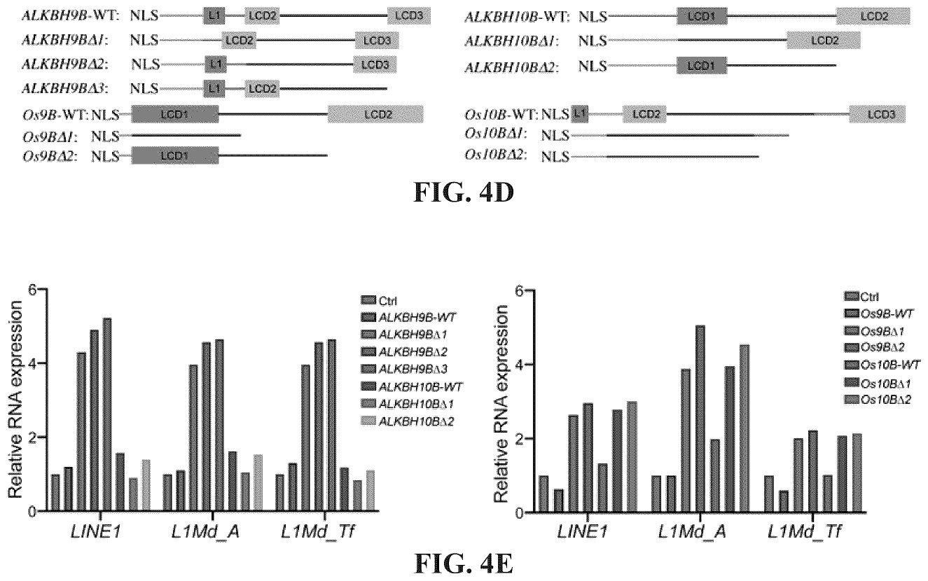

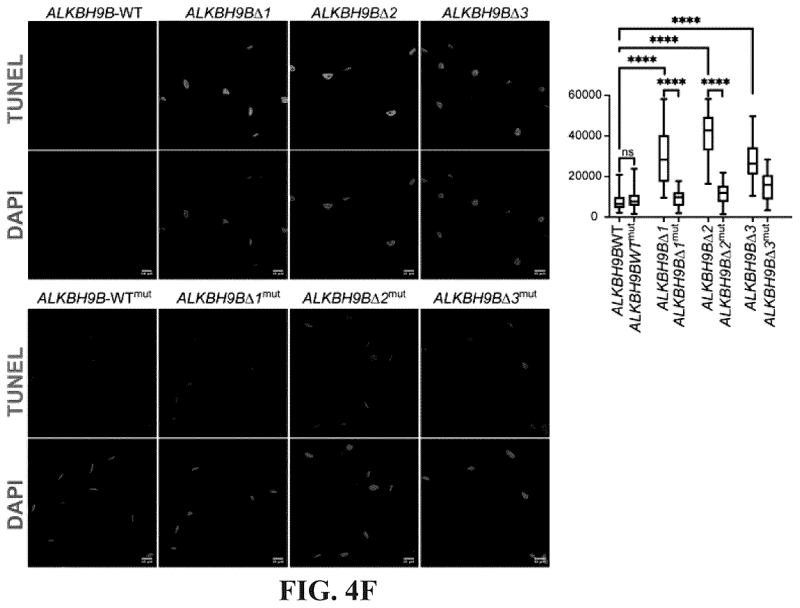

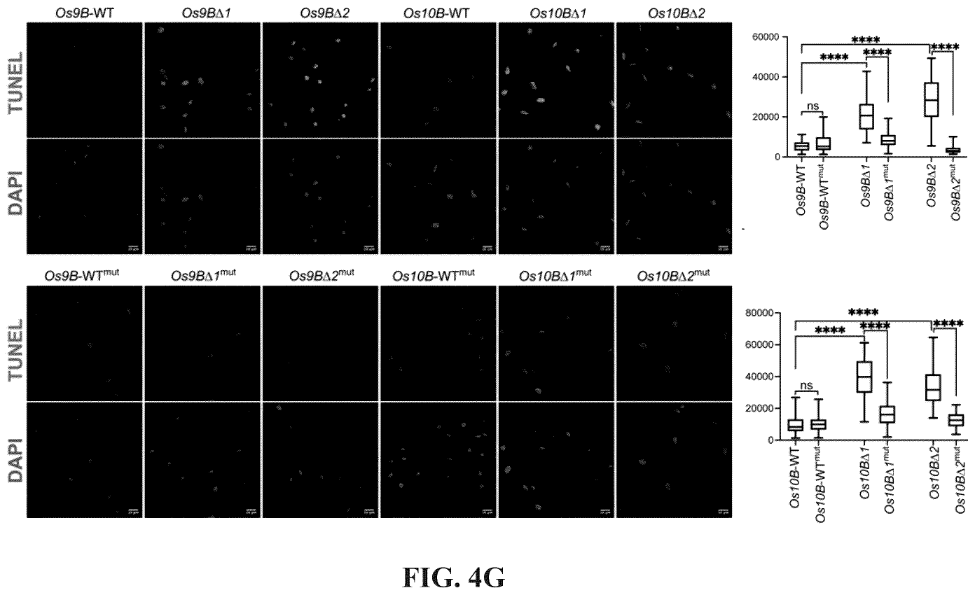

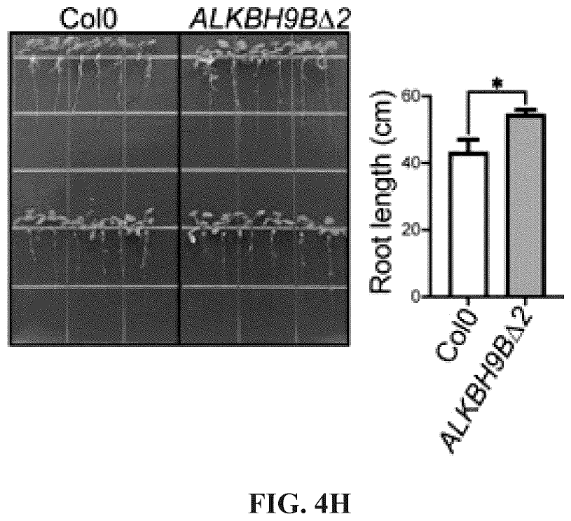

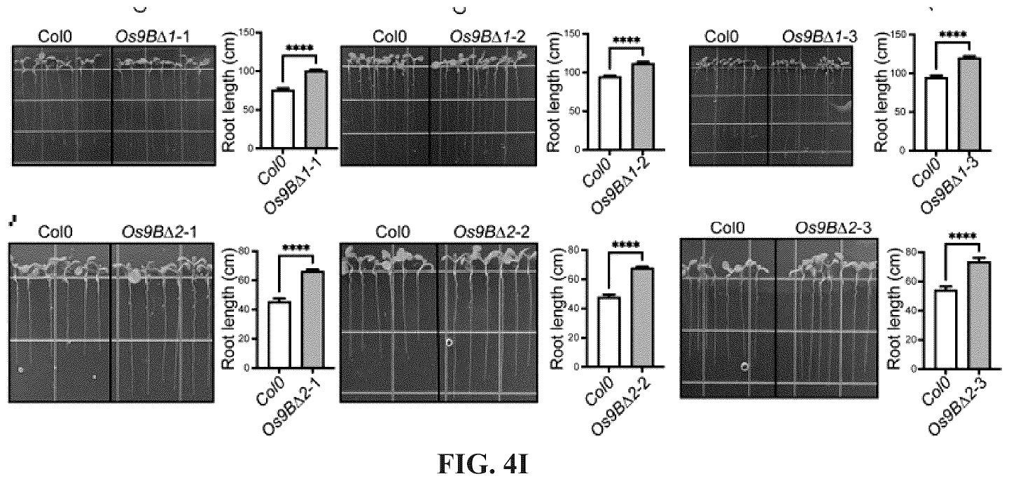

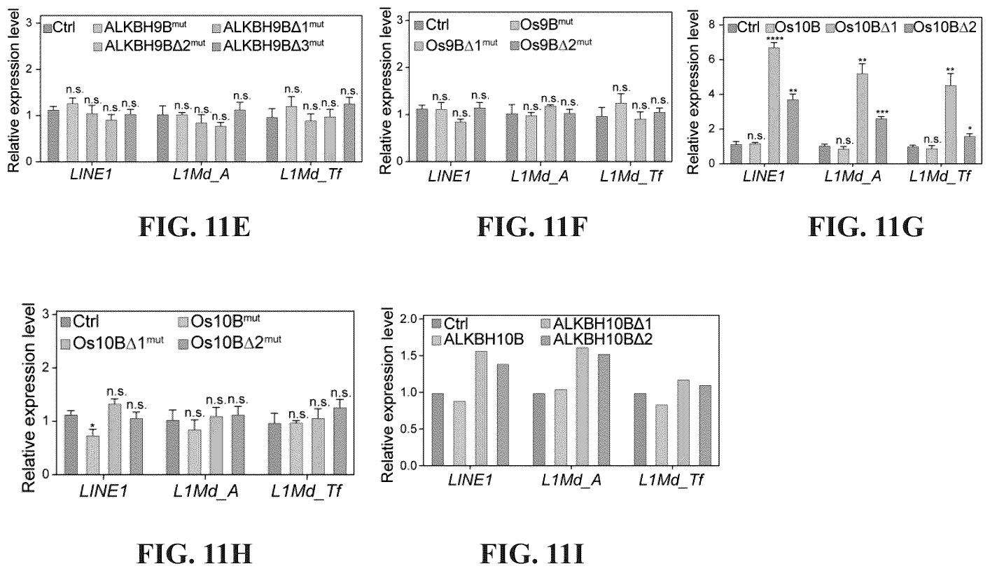

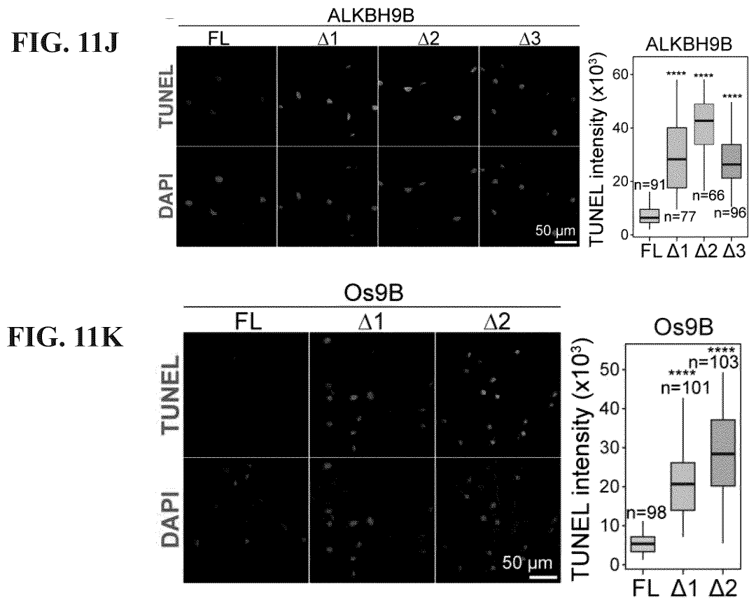

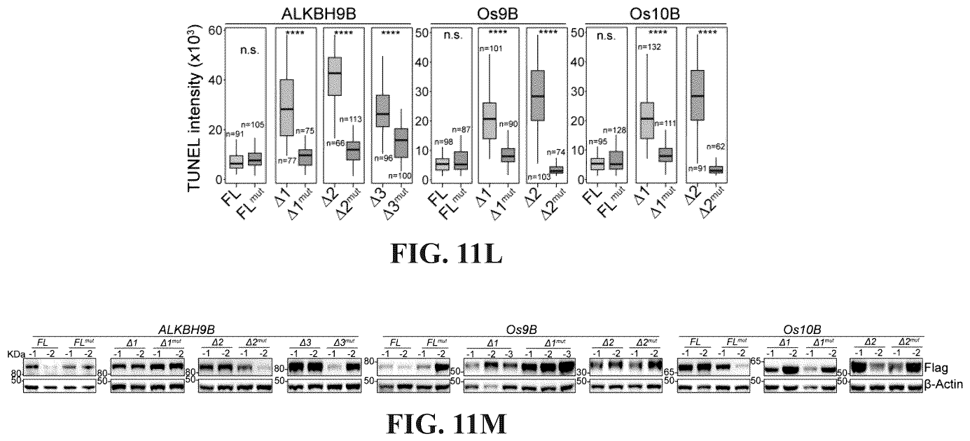

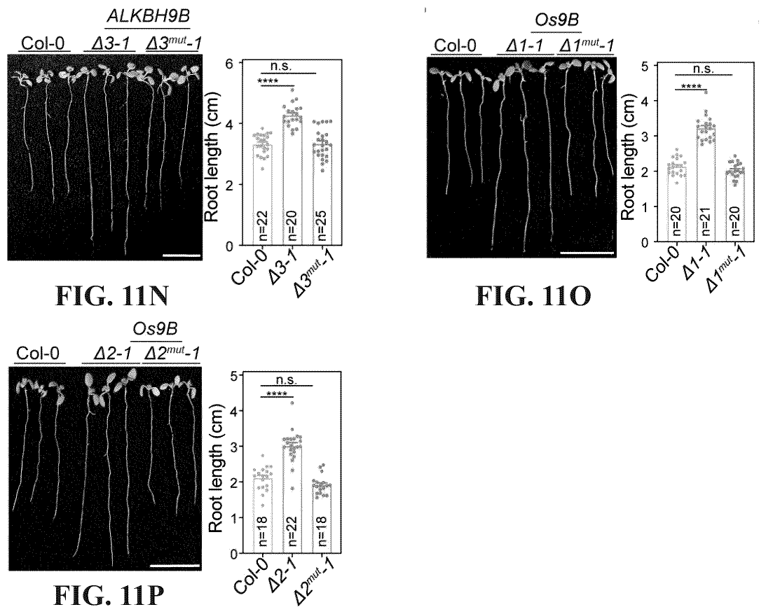

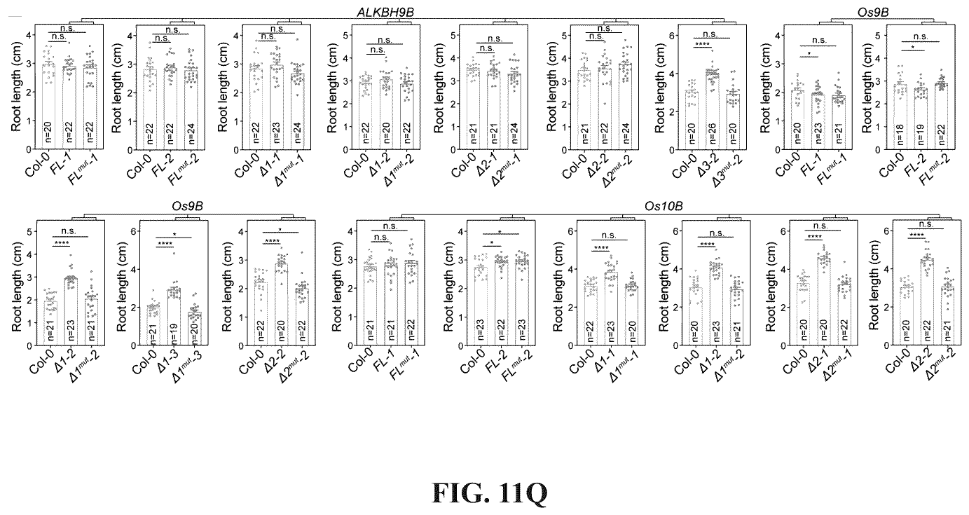

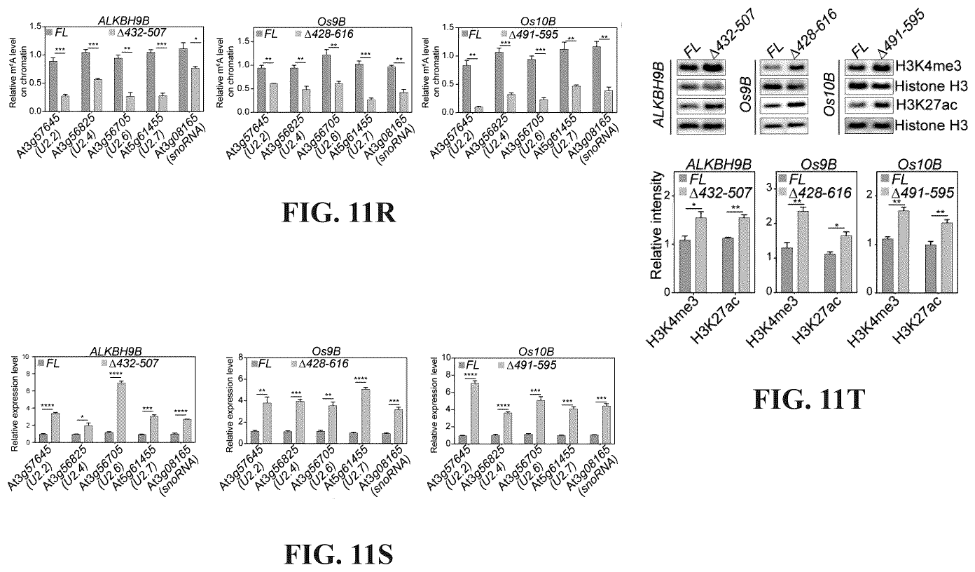

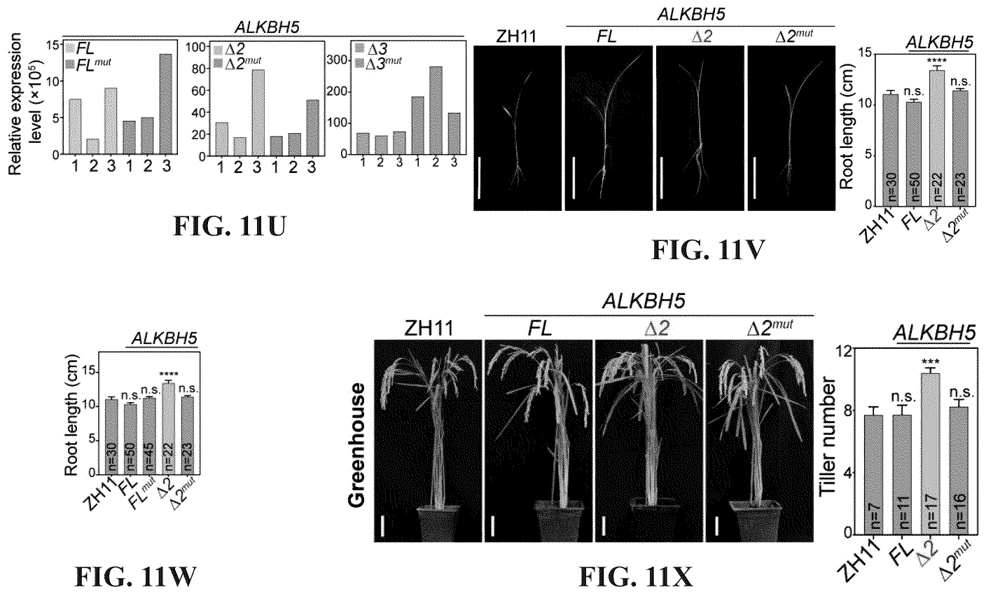

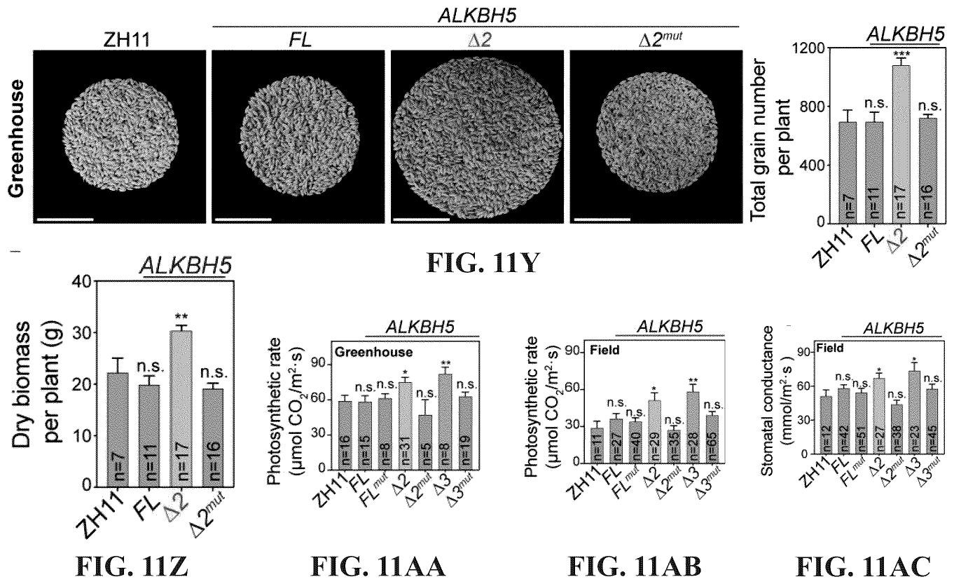

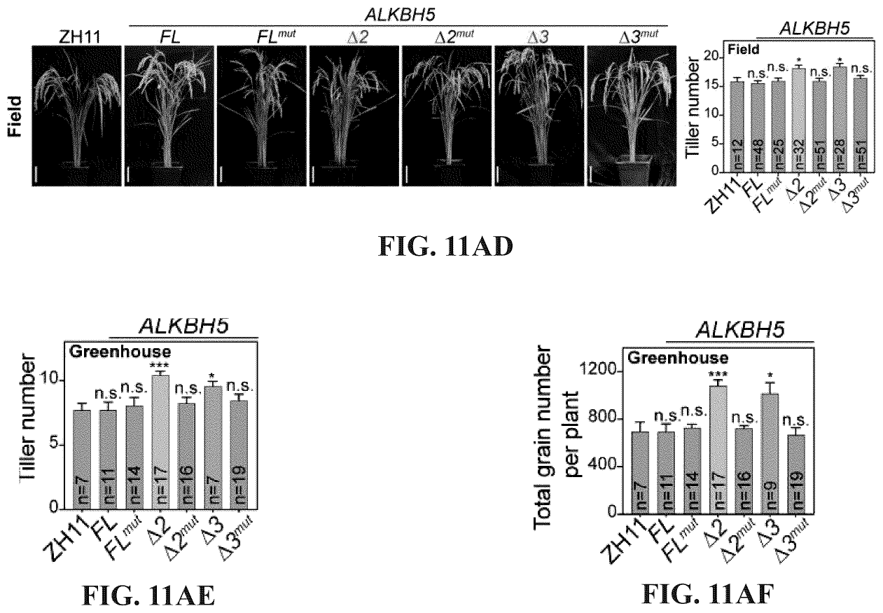

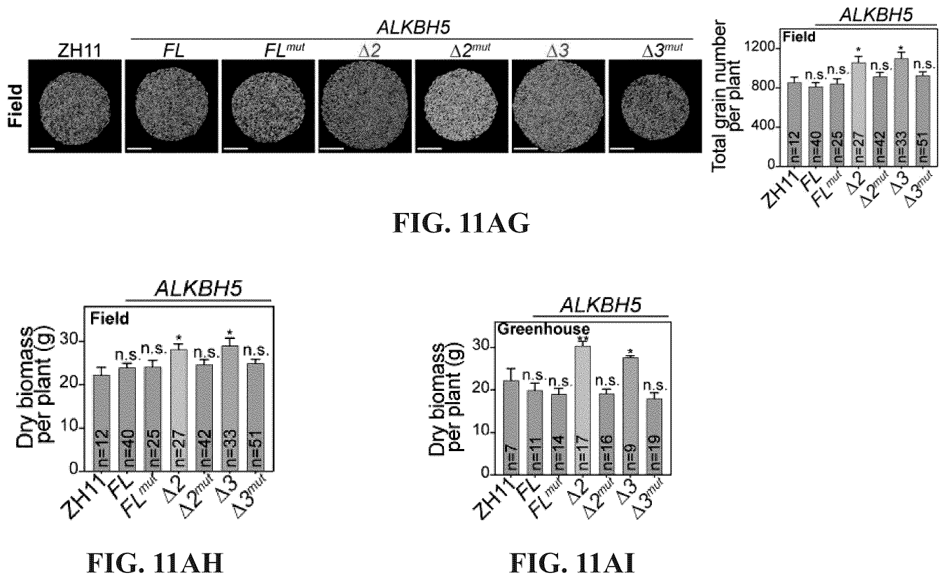

Encouraged by the promising outcomes observed with engineered human ALKBH5-ΔcIDR in Arabidopsis, it was next investigated whether comparable enhancements could be achieved by manipulating plant ALKBH5 orthologs. In Arabidopsis, two m⁶A demethylases, ALKBH9B and ALKBH10B, have been identified, both sharing catalytic domains highly conserved with that of mammalian ALKBH5 (Martinez-Perez et al., 2017; Duan et al., 2017). BLAST analysis further predicted two potential orthologs of human ALKBH5 in rice: LOC_Os06g04660 (herein referred to as “Os9B”) and LOC_Os10g02760 (herein referred to as “Os10B”) (FIG. 4A). In vivo biochemistry assays indicated that both OS9B and Os10B possessed RNA m⁶A demethylation activity (FIGS. 4B, 11A), consistent with recent studies reporting Os9B as an m⁶A demethylase in rice (Xue et al., 2024; Tang et al., 2024). Similar to ALKBH5, these orthologs also contain IDRs outside their catalytic domains (FIGS. 4C, 11B).

Building on insights leaned from human ALKBH5, constructs for Arabidopsis ALKBH9B, rice Os9B, and rice Os10B were engineered with N-terminal nuclear localization signal (NLS) (DNA: SEQ ID NO: 40; amino acid: SEQ ID NO: 41) and IDR deletion to test their chromatin-modulatory activity (FIGS. 4D, 11B (bottom)). The addition of an SV40 NLS (DNA: SEQ ID NO: 40; amino acid: SEQ ID NO: 41) facilitated nuclear localization of the engineered demethylases to ensure their potential access to caRNAs. In mESCs, expression of truncated variants of ALKBH9B (ALKBH9B_{Δ76-102}, ALKBH9B_{Δ145-183} and ALKBH9B_{Δ432-507}), Os9B (Os9B_{Δ60-170 and 428-616} and Os9B_{Δ428-616}), and Os10B (Os10B_{Δ2-30, 99-126 and 491-595} and Os10B_{Δ491-595}) markedly elevated whole-cell LINE1 RNA level in m⁶A-dependent manners, whereas their full-length counterparts had no effect (FIGS. 4E, 11C-11H). Among the two Arabidopsis m⁶A demethylases, ALKBH9B was prioritized due to its higher activity to upregulate LINE1 RNA abundance compared to ALKBH10B in mESCs (FIGS. 4E, 11C-11I).

Next, the engineered constructs were evaluated in plants. Transient overexpression of these variants in tobacco leaves revealed that the IDR-deleted forms of ALKBH9B (ALKBH9B_{Δ76-102}, ALKBH9B_{Δ145-183} and ALKBH9B_{Δ432-507}), Os9B (Os9B_{Δ428-616} and Os9B_{Δ60-170 and 428-616}), and Os10B (Os10B_{Δ491-595} and Os10B_{Δ2-30, 99-126 and 491-595}) significantly increased chromatin accessibility compared to their full length and catalytically inactive counterparts (FIGS. 4F-4G, 11J-11L)). Transgenic Arabidopsis lines stably expressing these variants were then generated (FIG. 11M). IDR-deleted variants—ALKBH9B_{Δ432-507}, Os9B_{Δ428-616}, Os9B_{Δ360-170 and 428-616}, Os10B_{Δ491-595}, and Os10B_{Δ2-30, 99-126 and 491-595}—markedly promoted root growth in Arabidopsis compared to the Col-0 control, while full-length and catalytically inactive variants showed limited effects (FIGS. 4H-41, 11N-11Q).

Next, MeRIP-qPCR was performed on top-ranked m⁶A-hypomethylated loci identified in ALKBH5-ΔcIDR-Arabidopsis, focusing on transgenic Arabidopsis lines expressing ALKBH9B_{Δ432-507}, Os9B_{Δ428-616}, and Os10B_{Δ491-595}. These CIDR-deleted lines exhibited markedly reduced m⁶A levels on caRNAs at these selected loci compared to their full-length counterparts (FIG. 11R). These reductions were accompanied by elevated transcript levels and increased H3K27ac and H3K4me3 signals, indicating more active chromatin state upon cIDR deletion (FIG. 11S-11T).

Together, these findings demonstrate that IDR deletion enhanced the chromatin-modulatory activity of plant ALKBH5 orthologs through a conserved mechanism, aligning with the effects observed for human ALKBH5-ΔcIDR in Arabidopsis. This engineering workflow provided a robust platform for reprogramming plant m⁶A demethylases to promote plant growth.

### Example 5a: Further Effects of Truncated Forms of Plant ALKBH5 Family Proteins (ALKBH9B and ALKBH10B) on Chromatin Regulation in mESCs and Plant Systems

This example shows further validation of effects of truncated forms of plant ALKBH5 family proteins (ALKBH9B and ALKBH10B) on chromatin regulation in mESCs and plant systems.

Inactive mutants of Arabidopsis ALBKH9B and ALKBH10B were generated. Truncations of the inactive mutants, in which the LCRs are disrupted or deleted, were also generated. Overexpression of these mutants were tested in Arabidopsis seedlings to assess root growth phenotype, in comparison to active ALKBH9B, ALKBH10B, and truncations thereof. This allowed for the assessment and comparison of root growth phenotypes between Arabidopsis lines expressing active truncations and those expressing inactive truncations. Inactive truncations did not show an effect on root growth (FIGS. 11N-11Q).

### Example 5B: Transgenic Expression of IDR-Deleted ALKBH5 Increases Rice Yield

This example shows further validation of effects of truncated forms of ALKBH5 family proteins on plant growth in rice.

Given the success of FTO-transgenic approach in increasing rice yield (Yu et al., 2021), it was sought to address the limitations associated with utilizing animal-derived genes in agriculture, by investigating whether IDR-deleted ALKBH5 could deliver agronomic benefits in rice similar to those of FTO. Using human ALKBH5 as a benchmark, transgenic rice lines were generated in the Zhonghuall (ZH11) background, expressing full-length or IDR-deleted ALKBH5 (ALKBH5_{Δ298-394}, and ALKBH5_{Δ30-81 and 298-394}), alongside catalytically inactive mutants as controls (FIG. 11U). Consistent with the observations in Arabidopsis, ALKBH5_{Δ298-394}-transgenic rice seedlings grown under hydroponic culture conditions exhibited significantly longer roots compared to WT (ZH11) and ALKBH5_{Δ298-394}^{mut}-transgenic rice (FIG. 11V-11W). To further evaluate agronomic impacts of these engineered ALKBH5 variants, key productivity traits—including photosynthesis rates, tiller numbers and grain yields-were examined under both greenhouse and field conditions (FIGS. 11Y-11AI). At the filling stage, ALKBH5_{Δ298-394}- and ALKBH5_{Δ30-81 and 298-394}-transgenic rice displayed significantly higher photosynthesis rates, compared to WT and their respective inactive mutant lines, across both greenhouse and field conditions (FIGS. 11AA-11AC). Moreover, ALKBH5_{Δ298-394}-transgenic rice and ALKBH5_{Δ30-81 and 298-394}-transgenic rice displayed a marked increase in tiller numbers, a key contributor to grain yield, compared to WT and their respective inactive mutant rice (FIGS. 11X, 11AD-11AE). Most notably, these improvements culminated in significantly higher grain yields (45-55% increases in greenhouse, 25-30% increases in the field) and biomass (25-40% increases in greenhouse, 25-30% increases in the field) under both greenhouse and field conditions (FIGS. 11A-11B, 11AF-11AI). In contrast, full-length ALKBH5 expression in rice showed no significant impact on any measured trait, underscoring the functional constraints imposed by the IDR domain in limiting its demethylation potential.

### DISCUSSION

IDRs are widely recognized as facilitators of chromatin regulation, enabling dynamic interactions with chromatin-associated factors and promoting flexible molecular assemblies. In addition to this well-established role, it was identified that IDRs on gene-activating demethylases can also act as regulatory restraints to limit chromatin engagement and safeguard genomic stability.

Through comparative analysis of ALKBH5 and FTO in mESCs, it was determined that the cIDR of ALKBH5 anchors its activity primarily to mRNA through binding with exon junction complex (EJC) components, thereby restricting its access to other chromatin regions. Deletion of the cIDR disrupts this restraint, shifting ALKBH5 activity towards caRNAs and driving chromatin opening and transcriptional activation. While both FTO and ALKBH5-ΔcIDR target chromatin-associated LINE1 RNAs in mESCs, they exhibit different genomic preferences. FTO, directed by its binding proteins (Song et al., 2020), predominantly demethylates intragenic LINE1 RNAs within genes governing development and differentiation (Wei et al., 2022), fine-tuning chromatin accessibility and transcription during critical developmental transitions. In contrast, ALKBH5 possesses cIDR that restricts it to mRNA substrates in mammals. ALKBH5-ΔcIDR, with cIDR deletion, preferentially engages intergenic LINE1 RNAs, driving enhancer-like activity and widespread chromatin modulation. The pronounced chromatin regulatory activity of ALKBH5-ΔcIDR underscores the necessity of IDR in ALKBH5 for restraining chromatin engagement and preventing widespread transcriptional activation.

The chromatin regulation insights learned from ALKBH5 and FTO in mESCs extended to plant systems. While human FTO-transgenic rice has shown the promise of manipulating m⁶A demethylation to improve crop yield and biomass (Yu et al., 2021), its reliance on human/animal genes may limit broader applicability and cause compliance concerns. To address this limitation, mechanistic insights learned from ALKBH5 regulation in mESCs were leveraged to engineer both human ALKBH5 and plant ALKBH5 homologs to regulate chromatin state and transcription in plants for the first time.

Specifically, these findings revealed that IDR-deleted ALKBH5 induced widespread m⁶A demethylation on caRNAs in mESCs and Arabidopsis, which promoted chromatin opening and transcription. These caRNAs can act as regulatory elements akin to CREs in modulating chromatin dynamics. Importantly, these insights pointed towards an IDR deletion strategy to engineer plant ALKBH5 homologs for achieving chromatin activation and plant growth promotion. By constructing IDR-deleted ALKBH5 homologs in Arabidopsis and rice, the adaptability of this approach was validated across plant species. These engineered ALKBH5 homologs promoted chromatin accessibility and Arabidopsis growth. Further, significant photosynthesis increase and yield improvement were confirmed in rice with transgenic expression of IDR-deleted human ALKBH5, further supporting the utility of IDR deletion strategies for crop enhancement. Given that all known plant m⁶A demethylases are ALKBH5 homologs (Martinez-Perez et al., 2017; Xue et al., 2024; Tang et al., 2024; Duan et al., 2017), this approach opened a new avenue for reprogramming chromatin dynamics to boost agricultural productivity using engineered plant proteins.

In summary, this study not only provided foundational insights into IDRs that play regulatory restrain roles in RNA to preserve chromatin state but also outlined a practical framework for engineering ALKBH5 family demethylases for agricultural applications. By integrating mechanistic understanding with innovative engineering strategy, the potential of IDR-deleted ALKBH5 and its plant homologs to enhance plant growth and crop yield were demonstrated. These findings have implications for future chromatin reprogramming in plant systems using engineered demethylases.

### Example 6: Genome Editing of Endogenous ALKBH9B or ALKBH10B

This example shows that editing of endogenous ALKBH9B or ALKBH10B results in increased plant growth.

Using genome editing, a stop codon is introduced upstream of the C-terminal LCR of endogenous ALKBH9B and/or ALKBH10B in Arabidopsis. Root growth assays are performed as in Example 4, and the edited plant has improved root growth.

### Additional Methods

### Cell Culture

E14TG2a mES cells (ATCC) were cultured in DMEM (Gibco) supplemented with 15% FBS (Gibco), 1% EmbryoMax nucleosides (Sigma), 1% GlutaMAX supplement (Gibco), 1% MEM non-essential amino acids solution (Gibco), 1% Penicillin-Streptomycin (Gibco), 0.1 mM 2-Mercaptoethanol (Gibco), 1000 U/ml LIF (Sigma), 1 μM PD0325901 (Stemcell) and 3 μM CHIR99021 (Stemcell) at 37° C. in a humidified atmosphere with 5% CO₂. HEK293T cells (ATCC) were maintained in DMEM (Gibco) supplemented with 10% FBS (Gibco) and 1% Penicillin-Streptomycin (Gibco) at 37° C. with 5% CO₂.

### Plasmid Construction

All constructs expressed in mESCs were generated using the PPB-CAG-IRES-Pac backbone, which includes puromycin resistance, as well as HA and Flag tags. For engineering ALKBH5 homologs, a triple SV40 NLS sequence was incorporated downstream of the Flag-HA sequence in the backbone. Coding sequences for human FTO and ALKBH5 were amplified from HEK293T cDNA. Arabidopsis ALKBH9B coding sequences were synthesized, while rice Os9B and Os10B coding sequences were amplified from ORF clones. Coding sequences for KDM4A/4B/6A/6B were amplified from plasmids #101051, #24181, #24168, #24167 (Addgene) respectively. These fragments were inserted into BglII/Xhol-linearized backbones using NEBBuilder HiFi DNA assembly master mix (NEB).

Constructs intended for plant expression were generated using a 35S::Flag-NLS backbone. Fragments were amplified from corresponding plasmids tested in mESCs and assembled into BglII/BamHI-linearized plant backbones via Gibson assembly.

All plasmids were verified through either whole-plasmid or Sanger sequencing. A complete list of oligonucleotides used for cloning is provided in Table 2.

### Construction of Stable Cell Lines

For ectopic expression cell lines, individual piggy Bac plasmids were co-transfected with the pCMV-PBase plasmid encoding a transposase gene at a 1:1 ratio into mESCs using Lipofectamine 3000 (Thermo Scientific) according to the manufacturer's protocol. 48h post-transfection, mESCs were selected with 2 μg/ml puromycin (Gibco). After 5-7 days of continuous selection, surviving cells were pooled for subsequent experiments.

For Alkbh5 KO in mESCs, sgRNAs were clones into the pSpCas9 (BB)-2A-Puro plasmid (Addgene #62988) and transfected into mESCs using Lipofectamine 3000 (Thermo Scientific), following the manufacturer's instructions. 48h post-transfection, mESCs were selected with 2 μg/ml puromycin (Gibco). Following 48h of selection, single-cell clones were isolated and verified by western blotting and Sanger sequencing.

For the generation of HA-tagged stop codon knock-in in mESCs, CRISPR-Cas9 mediated homology-directed repair was performed as previously described (Ludwik et al., 2023; Dewari et al., 2018) with modifications. Briefly, crRNA, tracrRNA, ssDNA donor, Alt-R Cas9 enzyme and Alt-R HDR enhancer V2 were obtained from IDT. Active ribonucleoprotein complexes were assembled and transfected into 6×10⁴ mESCs along with 0.5 μl of 100 μM donor using the P3 Primary Cell 4D-Nucleofector™ X Kit S (Lonza) according to the manufacturer's protocol. 48h post-nucleofection, single-cell clones were isolated and verified by western blotting and Sanger sequencing.

Details of sgRNAs, ssDNA donors, and genotyping primers are provided in Table 2.

### Embryoid Body (EB) Differentiation

mESCs were dissociated using TrypLE and cultured in hanging drops on the lid of a Petri dish for 2 days in mESC medium lacking LIF and 2i. The resulting EBs were collected and transferred to ultra-low attachment plates (Corning) for an additional 5-7 days. Afterward, EBs were harvested for gene expression analysis.

### Cell Proliferation Assay

mESCs were dissociated using TrypLE and seeded into 96-well plates at a density of 2,500 or 5,000 cells per well. Proliferation assays were conducted using the CellTiter 96® AQueous One Solution Reagent (Promega) according to the manufacturer's instructions. Absorbance was measured at 24-, 48-, 72-, and 96-hours post-plating. All absorbance values were normalized to those measured 24 hours post-plating.

### Immunofluorescence Staining

mESCs were seeded onto Matrigel-coated 8-well chambers (Ibidi), fixed with 4% PFA (Thermo Scientific) for 15 min, and permeabilized with 0.3% Triton X-100 (Thermo Scientific) in PBS for 15 min. Following permeabilization, cells were blocked in IF buffer (3% BSA in PBST) for 1 h. Cells were then incubated overnight at 4° C. with primary antibody diluted in IF buffer. The next day, cells were washed three times with PBST, and incubated with secondary antibody diluted in IF buffer for 1 h at room temperature. After incubation, cells were washed three times with PBST, and nuclei were counterstained with DAPI (Thermo Scientific). Fluorescent images were acquired using Leica SP8 confocal microscope. Details of all antibodies used are listed in Table 3.

### Quantitative Analysis of m⁶A Level Using LC-MS/MS

50 ng of non-ribosomal RNA were digested with 1 μl of Nuclease P1 (NEB) in 20 μl of 1× Nuclease P1 buffer (NEB) at 37° C. for 2 h. Following Nuclease P1 digestion, 1 μl of FastAP (Thermo Scientific) and 2.5 μl of FastAP buffer (Thermo Scientific) were added, and the mixture was incubated at 37° C. for an additional 4 hours. The resulting solution was filtered through a 0.22 μm syringe filter and diluted to a final volume of 60 μl. Subsequently, 10 μl of the prepared solution was injected into an LC-MS/MS system for analysis. Nucleosides were separated using reverse-phase ultra-performance liquid chromatography on a C18 column and detected via mass spectrometry using an Agilent 6410 QQQ triple-quadruple LC-MS system in positive electrospray ionization mode. The nucleosides were quantified based on the ion mass transitions of 282 to 150 (m⁶A), and 268 to 136 (A). Quantification was performed by comparing the sample results to a standard curve generated using pure nucleoside standards analyzed within the same batch. The ratio of m⁶A to A was calculated from the determined concentrations.

### Cell Fractionation

mESCs were fractionated as previously described (Wuarin and Schibler, 1994) with modifications. Briefly, cells were collected and washed once with PBS containing 1 mM EDTA. Then cells were transferred to a 2 ml tube and centrifuged at 500×g for 3 min at 4° C. to collect the cell pellet. The pellet was resuspended in 200 μl of ice-cold lysis buffer (10 mM Tris-HCl, pH=7.5, 0.05% NP40, 150 mM NaCl, 1× protease inhibitor, 0.2 U/μl SUPERase·In RNase inhibitor) and incubated on ice for 5 min. Next, 2.5 volumes of ice-cold sucrose cushion (24% RNase-free sucrose in lysis buffer) were gently layered beneath the lysis mixture and centrifuged at 5,000×g for 10 minutes at 4° C. The resulting supernatant was collected as the cytoplasmic fraction. The nuclei pellet was washed twice with 1 ml of ice-cold PBS containing 1 mM EDTA. The nuclei pellet was then resuspended in 200 μl of ice-cold glycerol buffer (20 mM Tris-HCl, pH=7.9, 75 mM NaCl, 0.5 mM EDTA, 0.5 mM DTT, 50% glycerol, 1× protease inhibitor, 0.2 U/μl SUPERase·In RNase inhibitor) with gentle flicking. Two volumes of cold nuclei lysis buffer (10 mM HEPES, pH=7.6, 1 mM DTT, 7.5 mM MgCl₂, 0.2 mM EDTA, 0.3 M NaCl, 1 M UREA, 1% NP-40, 1× protease inhibitor, 0.2 U/μl SUPERase·In RNase inhibitor) was added, followed by vigorously vortexing for 5 seconds twice. The nuclei lysate was incubated on ice for 2 min and centrifuged at 15,000 g for 2 min at 4° C. The supernatant was collected as the soluble nuclear fraction (nucleoplasm). The remaining pellet was gently rinsed twice with cold PBS containing 1 mM EDTA, and was collected as the chromosome-associated fraction.

### RT-qPCR and MeRIP-qPCR

Whole-cell or specific fractions of mESCs were lysed in TRIzoI Reagent (Invitrogen), and RNA was extracted using a combination of chloroform extraction and the RNA clean & Concentrate kit (Zymo research), following the manufacturer's instructions. To eliminate residual DNA contamination, RNA was treated on-column with DNase I. cDNA synthesis was carried out using the PrimeScript RT Master Mix (Takara). Quantitative real-time PCR was performed using FastStart Essential DNA Green Master (Roche) on a LightCycler 96 system (Roche). m⁶A-IP of non-ribosomal RNA from the chromatin-associated fraction of mESCs or Arabidopsis was performed using the EpiMark N⁶-Methyladenosine Enrichment Kit (NEB) following the manufacturer's instructions. m⁶A and non-m⁶A spike-ins from this kit were used as the normalization controls for m⁶A level analysis in MeRIP-RT-qPCR. Relative gene expression changes were calculated using the ΔΔCₜ method. Primer sequences used for RT-qPCR are provided in Table 2.

### Western Blotting

Cells were washed with PBS and lysed with 1×NuPAGE™ LDS Sample Buffer (Invitrogen) containing 1 mM DTT to generate whole cell lysate. For co-immunoprecipitation experiments, proteins were eluted from beads by boiling in 2×NuPAGE™ LDS Sample Buffer (Invitrogen) containing 1 mM DTT at 95° C. for 10 min. For plant samples, tissues were frozen in liquid nitrogen, disrupted using Tissuelyser II (Qiagen). Then the resulting powders were mixed with 2×NuPAGE™ LDS Sample Buffer (Invitrogen) containing 1 mM DTT and heated at 95° C. for 15 min.

Protein samples were separated on NuPAGE™ 4 to 12% Bis-Tris gels or NuPAGE™ 3-8% Tris-Acetate Protein Gels (Invitrogen), and subsequently transferred onto 0.45 μm PVDF membranes using semidry or wet electroblotting (Bio-Rad). Membranes were blocked in TBST containing 5% milk powder at room temperature (RT) for 1h. Subsequently, membranes were incubated overnight at 4° C. with primary antibody diluted in TBST containing 3% BSA. The next day, membranes were washed three times with TBST, and incubated with secondary antibody diluted in TBST containing 3% milk powder for 1 h at RT. Following incubation, membranes were washed three times with TBST, and imaged using iBright™ CL1500 Imaging System (Invitrogen™). Details of all antibodies used are listed in the Table 3.

### Co-immunoprecipitation

Cells were harvested, and nuclei were isolated following the cell fractionation protocol. The isolated nuclei were resuspended in Nuclei Lysis Buffer C (20 mM Tris-CI pH 7.5, 10% glycerol, 420 mM NaCl, 4 mM MgCl₂, 0.2 mM EDTA, 0.5 mM DTT, and 1× protease inhibitor) and incubated on ice for 30 minutes. The soluble nuclear extract (nuclear extract A) was obtained by centrifugation at 15,000 g for 15 minutes at 4° C. The remaining insoluble chromatin fraction was resuspended in Buffer A (10 mM Tris-Cl, pH 7.5, 150 mM NaCl, 1.5 mM MgCl₂, 0.5 mM DTT, and 1× protease inhibitor) at two volumes of Nuclei Lysis Buffer C, followed by DNase I treatment. After a 30-minute incubation at 37° C., the soluble chromatin fraction (nuclear extract B) was collected via centrifugation at 15,000 g for 15 minutes at 4° C. Nuclear extracts A and B were combined and incubated with the appropriate antibody or IgG control pre-bound to Protein A/G beads at room temperature for 2 hours. The beads were washed four times with wash buffer, prepared by mixing Buffer A and Buffer C in a 2:1 ratio. Finally, 2×NuPAGE™ LDS Sample Buffer (Invitrogen) containing 1 mM DTT was added directly to the beads, and the samples were boiled at 95° C. for 10 minutes before further analysis.

### Nascent RNA Synthesis Assay

mESCs were seeded onto Matrigel-coated 8-well chambers (Ibidi) one day prior to 5-ethynyluridine (EU) labeling. The nascent RNA synthesis assay was conducted the following day using the Click-iT™ RNA Alexa Fluor™ 594 Imaging Kit (Invitrogen) according to the manufacturer's instructions. Cell nuclei were counterstained with DAPI (Thermo Scientific). Fluorescent images were captured using a Leica SP8 confocal microscope, and the intensity of the EU signal was quantified using Fiji software.

### Dnase I-Treated TUNEL Assays

For mESCs, the DNase I-treated TUNEL assay was conducted using the DeadEnd™ Fluorometric TUNEL System (Promega) according to the manufacturer's instructions. Prior to rTdT labeling, cells were treated with 1 U/μl DNase I (Thermo Scientific) at 37° C. for 5 min. Flow cytometry was performed using a BD Fortessa™ (BD), and data were analyzed with FlowJo™ software.

For paraffin section of tobacco leaves, the samples were deparaffinized by immersion in Histo-Clear (National Diagnostics) and rehydrated through a graded ethanol series. Slides were then fixed with 4% PFA (Thermo Scientific) and washed twice with PBS. Subsequently, slides were treated 1 U/μl DNase I (Thermo scientific) at 37° C. for 5 min. The TUNEL assay was then performed using the DeadEnd™ Fluorometric TUNEL System (Promega) following the manufacturer's instructions. Slides were mounted with ProLong™ Diamond Antifade Mountant with DAPI (Invitrogen) and cover glasses (Fisherbrand). Imaging was performed using a Leica SP8 confocal microscope, and the intensity of the TUNEL signal was quantified using Fiji software.

eCLIP-seq

eCLIP-seq in mESCs was performed as previously described (Blue et al., 2022) with modifications. mESCs were UV crosslinked at 400 mJ/cm² (254 nm) once and nuclei were isolated following the cell fractionation protocol. Nuclei were lysed in eCLIP lysis buffer (50 mM Tris-HCl pH 7.5, 100 mM NaCl, 1% NP-40, 0.1% SDS, 0.5% sodium deoxycholate, 1× protease inhibitor, 0.5 U/μl Murine Rnase inhibitor) and sonicated using Bioruptor to solubilize the chromatin. The supernatant was partially digested with Rnase I at 37° C. for 5 min and quenched by adding SUPERase·In RNase inhibitor. A 10% aliquot of the mixture was set aside for sized-matched input. The remaining mixture was incubated with ALKBH5 or HA antibody pre-bound to Dynabeads Protein A/G (Thermo Scientific) at room temperature for 2 hours. The beads were sequentially washed with high-salt wash buffer (50 mM Tris-HCl pH 7.5, 1M NaCl, 1 mM EDTA, 1% NP-40, 0.1% SDS, 0.5% sodium deoxycholate) and low salt wash buffer (20 mM Tris-HCl pH 7.5, 5 mM NaCl, 10 mM MgCl₂, 0.2% Tween-20). RNA-protein complexes were dephosphorylated with FastAP (Thermo Scientific) and T4 PNK (NEB). After ligation with the 3′ RNA adaptor, excessive adaptor was removed with additional washes using high salt wash buffer and low salt wash buffer. Subsequently, IP and Input samples were separated using NuPAGE™ 4-12% Bis-Tris gels and wet-transferred to a nitrocellulose membrane at 30 V overnight. A region of the membrane corresponding to the expected protein size plus ˜75 kDa was excised. RNA fragments were released using Proteinase K (NEB) treatment at 37° C. for 30 min and purified using the RNA Clean & Concentrator-5 Kit (Zymo). RNA was reverse transcribed with SR-RT primer using SuperScript III (Thermo Scientific), ligated with a 3′ cDNA adaptor, and PCR amplified using LongAmp Taq 2X Master Mix (NEB). Fragments between 160 and 307 bp were isolated from a 3.5% low-melt agarose gel. Sequencing was performed on an Illumina NovaSeq X platform in paired-end mode (150 bp per read) at Azenta company. Details of adaptors and primers sequences are provided in Table 2.

Arabidopsis Chromatin-Associated RNA (caRNA) and Nuclei Isolation

Arabidopsis nuclei fractionation was performed as described previously (Long et al., 2021) with modifications. Briefly, 3-5 g of Arabidopsis seedlings were frozen in liquid nitrogen and ground into powder using a mortar and pestle. The resulting powder was transferred to 20 ml of Honda buffer (0.44 M sucrose, 1.25% Ficoll, 2.5% Dextran T40, 20 mM Tris-HCl pH 7.5, 10 mM MgCl₂, 1 mM DTT, 1×protease inhibitor) and incubated on ice for 10 minutes. The mixture was filtered through a 40 μm cell strainer. Following centrifugation at 600×g for 5 minutes, the pellet was resuspended in 15 ml of Honda buffer containing 0.5% Triton X-100 and incubated on ice for another 5 minutes. The suspension was filtered through two layers of Miracloth (Sigma) and centrifuged at 600×g for 10 minutes. The resulting pellet was washed twice with Honda buffer containing 0.1% Triton X-100 and designated as the nuclei fraction for subsequent applications such as ATAC-seq, CUT&Tag, and CUT&RUN. The pellet was resuspended in 300-500 μl of Honda buffer, and a 10 μl aliquot was stained with DAPI to assess the nuclei count under a fluorescence microscope.

For caRNA extraction, the nuclei pellet was washed with cold PBS containing 1 mM EDTA. The nuclei were then resuspended in 200 μl of ice-cold glycerol buffer (20 mM Tris-HCl, pH 7.9, 75 mM NaCl, 0.5 mM EDTA, 0.85 mM DTT, 50% glycerol, 1× protease inhibitor, 0.2 U/μl SUPERase·In RNase inhibitor) with gentle mixing. Two volumes of cold nuclei lysis buffer (10 mM HEPES, pH 7.6, 1 mM DTT, 7.5 mM MgCl₂, 0.2 mM EDTA, 0.3 M NaCl, 1 M urea, 1% NP-40, 1× protease inhibitor, 0.2 U/μl SUPERase·In RNase inhibitor) were added, and the mixture was vortexed vigorously for 5 seconds twice. The lysate was incubated on ice for 2 minutes, and the pellet was collected by centrifugation at 15,000×g for 2 minutes at 4° C. The pellet was gently rinsed twice with cold PBS containing 1 mM EDTA and collected as the chromosome-associated fraction. For caRNA extraction, the pellet was resuspended in 200 μl of Turbo DNase I mixture (5 μl Turbo DNase I, 1× Turbo DNase I buffer, 0.2 U/μl SUPERase·In RNase inhibitor) and incubated at 37° C. for 15 minutes. Subsequently, three volumes of TRIzoI LS reagent were added for RNA extraction.

### Spike-In Calibrated ATAC-Seq

Spike-in calibrated ATAC-seq was performed using the ATAC-Seq Kit (Active Motif) according to the manufacturer's instructions. For mESCs, 5×10⁴ cells were collected, mixed with 10 μl of ATAC-Seq Spike-In Nuclei (Active Motif), and washed once with ice-cold PBS. The cell pellet was resuspended in 100 μl of ice-cold ATAC Lysis Buffer and centrifuged at 500×g for 10 minutes. For Arabidopsis nuclei, 5×10⁴ nuclei were collected, mixed with 10 μl of ATAC-Seq Spike-In Nuclei (Active Motif), and washed once with ice-cold PBS. The tagmentation reaction was performed following the ATAC-Seq Kit protocol. Immediately after the tagmentation reaction, DNA fragments were purified and subjected to PCR amplification using Q5 High-Fidelity 2X Master Mix (NEB). The PCR products were purified with AMPure XP Beads (Beckman Coulter). Sequencing was carried out on an Illumina NovaSeq X platform in paired-end mode (150 bp per read).

### CUT&Tag

CUT&Tag was performed using the CUT&Tag-IT® Core Assay Kit (Active Motif) according to the manufacturer's instructions. For mESCs, 2×10⁵ cells were collected, mixed with 10 μl of CUT&Tag-IT Spike-In Nuclei (Active Motif), and washed twice with 1× Wash Buffer. For Arabidopsis, 2.5×10⁵ isolated nuclei were mixed with 10 μl of CUT&Tag-IT Spike-In Nuclei (Active Motif) and washed twice with 1× Wash Buffer. For each sample, 20 μl of Concanavalin A bead slurry was used. During cells washing, Concanavalin A beads were activated by incubation in 1× Binding Buffer. mESCs or Arabidopsis nuclei were then bound to the activated Concanavalin A beads by incubation in 1× Binding Buffer for 10 minutes. Bound mESCs or Arabidopsis nuclei were resuspended in 50 u L of ice-cold Antibody Buffer containing specific primary antibodies and spike-in antibody, followed by overnight incubation at 4° C. After removing the primary antibodies, the samples were incubated with Guinea Pig Anti-Rabbit secondary antibody (1:100 dilution) in Dig-Wash Buffer at room temperature for 60 minutes. Following three washes with Dig-Wash Buffer, the samples were incubated with CUT&Tag-IT™ Assembled pA-Tn5 Transposases at room temperature for 60 minutes. The tagmentation reaction was initiated by adding 125 μL of Tagmentation Buffer and incubating at 37° C. for 60 minutes. The reaction was stopped, and DNA fragments were released by adding 4.2 μl of 0.5 M EDTA, 1.25 μl of 10% SDS, and 1.1 μl of Proteinase K (10 mg/ml), followed by incubation at 55° C. for 60 minutes. The DNA fragments were purified and subjected to PCR amplification using Q5 High-Fidelity 2X Master Mix (NEB). PCR products were purified with AMPure XP Beads (Beckman Coulter). Sequencing was carried out on an Illumina NovaSeq X platform in paired-end mode (150 bp per read).

### CUT&RUN

CUT&RUN was performed using the ChIC/CUT&RUN Assay Kit (Active Motif) according to the manufacturer's instructions. For histone modifications, YY1, or EP300, CUT&RUN Spike-In Control (Active Motif) was used as the spike-in. For HA or Flag-tagged proteins, the SNAP-CUTANA™ HA Tag Panel or SNAP-CUTANA™ DYKDDDDK Tag Panel (Epicypher), respectively, was used as the spike-in.

For mESCs, 2.5×10⁵ cells were collected and mixed with 10 μl of CUT&RUN Spike-In Nuclei (Active Motif). mESC nuclei were isolated by incubation with Complete Nuclei Isolation Buffer on ice for 10 minutes. After incubation, the CUT&RUN Spike-In Nuclei were added to mESCs or Arabidopsis nuclei and washed twice with Complete Dig-Wash Buffer. During cells washing, Concanavalin A beads were activated by incubation in 1× Binding Buffer. mESC or Arabidopsis nuclei were then bound to the activated Concanavalin A beads by incubation in Complete Dig-Wash Buffer for 10 minutes.

The bound nuclei were resuspended in 50 μl of ice-cold Antibody Buffer containing specific primary antibodies and spike-in antibody, followed by overnight incubation at 4° C. For samples using the SNAP-CUTANA™ HA Tag Panel or SNAP-CUTANA™ DYKDDDDK Tag Panel, 1 μl of the corresponding panel was added instead of the spike-in antibody during this step.

The following day, the primary antibody mixture was removed, and the beads were washed twice with Cell Permeabilization Buffer. 50 μl of Cell Permeabilization Buffer containing 2.5 μl of ChIC/CUT&RUN pAG-MNase was added to each sample, followed by a 10-minute incubation at room temperature. After two washes with Cell Permeabilization Buffer, targeted chromatin digestion was activated by the addition of 1 μl of 0.1 M CaCl₂) and incubation at 4° C. for 2 hours. The reaction was stopped by adding 40 μL of Stop Solution.

The released DNA fragments were purified and used for library preparation with the NEBNext® Ultra™ II DNA Library Prep Kit for Illumina (NEB) according to the manufacturer's instructions. Sequencing was performed on an Illumina NovaSeq X platform in paired-end mode (150 bp per read).

### Nascent RNA-seq

mESCs were seeded into eight 6-cm dishes at equal cell densities one day before EU labeling. The following day, cells were incubated in media containing 1 mM EU for 10, 20, 40, or 60 minutes. After incubation, cells were lysed with TRIzoI (Invitrogen), and total RNA was extracted. An equal amount of RNA extracted from EU-labeled Drosophila cells was added to each sample as a spike-in for further normalization.

Nascent RNA was captured using the Click-iT™ Nascent RNA Capture Kit (Invitrogen) according to the manufacturer's instructions. The captured RNA was eluted from the beads using elution buffer (95% v/v formamide, 10 mM EDTA pH 8.0, 1.5 mM biotin) by heating at 65° C. for 5 minutes followed by 90° C. for another 5 minutes. The eluted RNA was purified by ethanol precipitation with glycogen (Invitrogen) as a carrier and dissolved in nuclease-free water (Thermo Scientific).

RNA libraries were constructed using the SMARTer Stranded Total RNA-seq Kit v2 (Takara) according to the manufacturer's protocol. Sequencing was performed on an Illumina NovaSeq X platform in paired-end mode (150 bp per read).\

Chromatin-Associated RNA (caRNA) MeRIP-seq

For mESCs, 1 μl of 1:1000 diluted m⁶A and non-m⁶A spike-in controls from the EpiMark N⁶-Methyladenosine Enrichment Kit (NEB) was added as a spike-in to 1 μg of non-ribosomal RNA isolated from the chromatin-associated fraction. For Arabidopsis, 1 μl of 1:100 diluted m⁶A and non-m⁶A spike-in controls from the same kit was added as a spike-in to 200 ng of non-ribosomal RNA isolated from the chromatin-associated fraction. RNA was fragmented using RNA Fragmentation Reagents (Invitrogen) by incubating at 70° C. for 6 minutes, followed by purification with the Oligo Clean & Concentrator Kit (Zymo Research). 5% of the fragmented RNA was saved as input. m⁶A immunoprecipitation was performed using the EpiMark N⁶-Methyladenosine Enrichment Kit (NEB) according to the manufacturer's instructions. Library preparation was carried out using the SMARTer Stranded Total RNA-seq Kit v2 (Takara) following the manufacturer's protocol. Sequencing was performed in paired-end mode (150 bp per read) on an Illumina NovaSeq X platform.

### Measurement of Nuclear RNA Lifetime by RT-qPCR

WT and Alkbh5-ΔcIDR mESCs were seeded into eight 6-cm dishes at equal cell densities one day prior to the assay. The following day, cells were treated with media containing 5 μg/mL actinomycin D for 0, 3, and 6 hours. After incubation, cells were collected, and nuclear fractions were isolated following the cell fractionation protocol. Nuclear RNA was extracted using TRIzoI Reagent (Thermo Scientific). To normalize RNA quantities, 1 μl of 1:100 diluted m⁶A and non-m⁶A spike-in controls from the EpiMark N⁶-Methyladenosine Enrichment Kit (NEB) was added proportionally to the total nuclear RNA. RNA levels were quantified by RT-qPCR. The half-life (t_{1/2}) of nuclear RNA was calculated using a one-phase decay model in GraphPad Prism.

### CLIP-qPCR of YTHDC1

CLIP was performed as described in the eCLIP-seq section, with the following modifications: RNA was partially digested with 1 U/μl RNase T1 (Thermo Scientific) at 25° C. for 5 minutes, followed by chilling on ice for 5 minutes. After immunoprecipitation, the beads were washed sequentially with high-salt and low-salt wash buffers, followed by Proteinase K digestion. RNA was then extracted using TRIzoI Reagent (Thermo Scientific) and subjected to RT-qPCR analysis.

### Plant Materials and Growth Conditions

All wild-type (WT) and transgenic Arabidopsis lines used in this study were in the Arabidopsis thaliana Columbia (Col-0) background. Seeds were surface sterilized in 10% sodium hypochlorite solution for 10 minutes, washed five times with sterile water, and grown on Murashige and Skoog (MS) medium (PhytoTech Labs) supplemented with 1.5% sucrose and 0.8% agar (pH 5.8). Seedlings were grown under controlled conditions in either a growth chamber or a growth room at 21° C. with a 16-hour light/8-hour dark photoperiod.

All wild-type (WT) and transgenic rice lines used in this study were in the Zhonghuall (ZH11) background. Rice plants were cultivated in greenhouse and experimental fields. For field cultivation, rice plants were grown in standard paddy conditions during normal rice planting season in experimental fields located in Guangzhou, China (N 23°19′53″, E 113°35′06″). Plants were spaced at 20×20 cm (row×plant distance) and managed following standard agricultural practices, including irrigation, fertilization, and disease control, in accordance with normal agricultural guidelines.

### Plant Transformation

Transgenic Arabidopsis lines were generated via Agrobacterium-mediated floral dipping (Zhang et al., 2006) in the wild-type Col-0 background. Transformed T1 seeds were screened on compound soil watered with BASTA solution. Transgenic lines with successful gene insertion were confirmed by western blotting and basta resistance. T3 homozygous lines were selected and used for all subsequent analyses.

For transgenic rice lines, transformants were selected on medium supplemented with BASTA. Twenty independent TO lines were generated to obtain T1 progeny. T-DNA copy number (CN) in T1 plants was determined by quantitative PCR (qPCR) using primers specific for the BASTA resistance gene and the internal reference gene Actin, following established protocols (Yang et al., 2005). At least three independent lines with a single-copy insertion (CN=1) of the target gene were selected for further analysis and advanced to the T2 generation. Lines with multiple T-DNA insertions were excluded. Homozygous lines in the T2 generation were identified by qPCR-based T-DNA copy number analysis. Primer sequences are provided in Table 2.

### Root Length Assay

Seeds were surface sterilized by immersion in 10% sodium hypochlorite solution for 15 minutes, followed by five washes with deionized water. Sterilized seeds were plated on Murashige and Skoog (MS) medium (PhytoTech Labs) supplemented with 0.8% agar and 1.5% sucrose. To synchronize germination, the plates were kept in the dark at 4° C. for 72 hours. Plates were then positioned vertically at a 90° angle and transferred to a growth chamber with a 16-hour light/8-hour dark photoperiod at 21° C. After 10 days of growth, root length was quantified by scanning the seedlings and analyzing the images using Fiji software.

### Measurements of Photosynthesis Parameters

Photosynthetic parameters of WT and transgenic rice lines at the grain-filling stage were measured using a portable photosynthesis measurement CI-340 system (Zealquest Scientific Technology Co., Ltd), following the manufacturer's instructions. Measurements were conducted on plants grown in both field and greenhouse conditions.

### Agrobacterium-Mediated Transient Expression in Tobacco Leaves

Agrobacterium-mediated transient expression in Nicotiana benthamiana leaves was performed as described (Li et al., 2009). Briefly, Agrobacterium tumefaciens strain GV3101 carrying the indicated constructs was cultured overnight at 28° C. in LB medium. The bacterial cells were pelleted and resuspended in washing solution containing 10 mM MgCl₂ and 100 μM acetosyringone. The bacterial suspension was diluted to an OD₆₀₀ of 0.5 using infiltration solution (¼ MS medium supplemented with 1% sucrose, 100 μM acetosyringone, and 0.005% Silwet L-77). Young, fully expanded Nicotiana benthamiana leaves were infiltrated with the bacterial suspension using a needleless syringe. Following infiltration, plants were incubated in the dark for 12-24 hours and then transferred to normal growth conditions for a 2-days recovery period. Leaves were then collected for paraffin embedding.

### Paraffin Section Preparation

Paraffin sections were prepared as previously described (Javelle et al., 2011). Tobacco leaves were fixed in freshly prepared 4% PFA containing 0.1% Tween-20 and 0.1% Triton X-100 at 4° C. overnight. To facilitate thorough fixative penetration, tissues were subjected to vacuum infiltration for 15-20 minutes. Following fixation, tissues were dehydrated through a graded ethanol series (30%, 40%, 50%, 60%, 70%, 85%, and 95%), followed by two washes in 100% ethanol to ensure complete dehydration. Samples were then cleared in Histoclear through a stepwise gradient (25%, 50%, 75%, and 100%) and infiltrated with molten paraffin wax at 60° C. Tissues were embedded in paraffin blocks using peel-away molds and allowed to solidify at room temperature. Sections of 7-10 μm thickness were cut using a Leica rotary microtome and floated on sterile water at 42° C. for flattening. Slides were dried overnight at 42° C. to ensure proper tissue adhesion and stored at room temperature for downstream analyses.

Chromosome-Associated RNA m⁶A-seq and RNA-seq Analysis

Raw reads quality control (QC) was performed using fastp (Chen et al., 2018). Reads were aligned to either the mouse genome and transcriptome (GRCm39) or the Arabidopsis genome and transcriptome (TAIR 10), along with spike-in genomes that included unmodified control RNA and m⁶A-modified control RNA (EpiMark N⁶-Methyladenosine Enrichment Kit, NEB), using HISAT with the parameter “-rna-strandness RF” (Kim et al., 2015).

Post alignment, low-quality mapped reads were filtered, and PCR duplicates were removed using Picard. Strand-specific separation of mapped reads was performed using samtools (Li et al., 2009b). m⁶A peaks for each strand were identified using MACS2 (Zhang et al., 2008) with the parameters—nomodel and—keep-dup all. Peaks with fewer than 10 reads in the input sample were excluded to minimize bias from expression abundance. Peaks identified in at least two biological replicates were merged using bedtools (Quinlan and Hall, 2010) for downstream analysis.

For comparison analysis, union peaks across samples were generated using bedtools, and reads under these peaks were counted using featureCounts (Liao et al., 2014) for both IP and input samples. Raw counts were normalized to Counts Per Million (CPM). IP enrichment was calculated as CPM_{IP}/CPM_{Input}. A scaling factor (SF) was computed based on CPM_{IP m6A control RNA}/CPM_{input m6A control RNA}. The final m⁶A quantification for each sample was calculated using IP enrichment/SF. Pearson correlation coefficients for m⁶A levels across union peaks were calculated to assess reproducibility among biological replicates. Differential m⁶A regions were identified using edgeR (Robinson et al., 2010), and m⁶A peaks were assigned to genes using ChIPseeker (Yu et al., 2015).

Gene and repeats expression levels were quantified by dividing read counts under genes by read counts of the m⁶A control RNA in input samples. Differentially expressed genes (DEGs) were identified using edgeR (Robinson et al., 2010).

Analyses for different RNA categories, GO enrichment, GSEA, and repeat subfamilies, were performed using clusterProfiler (Yu et al., 2012).

eCLIP-seq Data Analysis

Raw reads were trimmed using cutadapt with the parameters “-e 0.1-n 1-O 6-q 20--nextseq-trim=20-m 21:27-a NNNNNATCACGAGATCGGAAGAGC-A NNNNNGATCGTCGGACTG”. UMI information was extracted using umi_tools (https://github.com/CGATOxford/UMI-tools?tab=readme-ov-file) with the parameter “extract--random-seed 1--bc-pattern=NNNNN--bc-pattern2=NNNNNNNNNNN”.

After QC, clean reads were aligned to the mouse genome (GRCm39) using STAR (Dobin et al., 2013). Duplicate reads were filtered based on UMI information using umi_tools. Reads mapped to rRNA were removed. Peaks were called using MACS2 (Zhang et al., 2008), and peak distances to genes were calculated using ChIPseeker (Yu et al., 2015). For profile visualization and IGV visualization, bigWig files were generated using deepTools (Ramirez et al., 2014) with RPKM normalization.

### Transcription Rate Data Analysis

Data QC and alignment were performed using fastp (Chen et al., 2018) and HISAT2 (Kim et al., 2015), respectively. The counts under genes and repeats were calculated using featureCounts (Liao et al., 2014). For each gene, the expression level was normalized by dividing the raw counts under genes by the raw counts under ERCC (Thermo Scientific).

For each time point, the relative transcription rates were normalized to the 10-minute sample. Log₂ fold changes (log₂FC) relative to the 10-minute sample were calculated for each time point using edgeR (Robinson et al., 2010). These log₂FC values were used to represent the transcription rates for subsequent data analysis.

### Histone Modification CUT&Tag, CUT&RUN, and ATAC-seq Analysis

For CUT&Tag, CUT&RUN, and ATAC-seq reads, QC was performed using fastp (Chen et al., 2018). Reads were aligned to the mouse or Arabidopsis genome and the spike-in sequence using BWA (Li and Durbin, 2009). Low-quality mapped reads were filtered with samtools⁹⁷, and PCR duplicates were removed using Picard. Peaks were called using MACS2. Reads under peaks were calculated using featureCounts (Liao et al., 2014).

Normalized signals were calculated as reads under peaks/reads in spike-in sequence. For sample comparisons, edgeR (Robinson et al., 2010) was used to identify differential peaks based on normalized signals. For profile and IGV visualization, bigWig files were generated using deepTools with the parameters “--scaleFactor SF-bs 10--normalizeUsing None”, where SF represents 1e6/readsₛₚᵢₖₑ₋ᵢₙ.

### RNA Categories Definition

The eRNAs were defined by the union peaks of H3K27ac, H3K4me1, and EP300 in mESCs. The paRNAs were defined by H3K4me3 peaks in mESCs. The Arabidopsis RNA categories information was downloaded from the TAIR website

Public Data Used in this Study

ChIP-seq data of CREs in mESCs (Shen et al., 2012): GSE29218; Pol II ChIA-PET data in mESCs (Consortium, E. P., 2012): ENCSR309NAP and ENCSR067GFU; caRNA MeRIP-seq in Fto and WT mESCs (Wei et al., 2022): GSE133600; LINE1 ChIRP-seq in mESC (Liu et al., 2021): GSE146467; RNA-chromatin interaction data in Arabidopsis (Li et al., 2021): GSE163845.

### Quantification and Statistical Analysis

All statistical details and methods are indicated in the figures and figure legends, including the exact values of n and experimental measures. Statistical analysis and plots were generated using the GraphPad Prism or R software.

TABLE 3
Antibodies.

| | | |
|---|---|---|
| REAGENT or RESOURCE | SOURCE | IDENTIFIER |

Antibodies

| | | |
|---|---|---|
| DYKDDDDK Tag Monoclonal Antibody (L5), Alexa | Invitrogen | Cat# MA1-142-A488 |
| Fluor ™ 488 | | RRID: AB_2610653 |
| Goat anti-Rabbit IgG (H + L) Cross-Adsorbed Secondary | Invitrogen | Cat# A-11008 |
| Antibody, Alexa Fluor ™ 488 | | RRID: AB_143165 |
| Goat anti-Mouse IgG (H + L) Cross-Adsorbed Secondary | Invitrogen | Cat# A-11001 |
| Antibody, Alexa Fluor ™ 488 | | RRID: AB_2534069 |
| Goat anti-Rabbit IgG (H + L) Cross-Adsorbed Secondary | Invitrogen | Cat# A-11012 |
| Antibody, Alexa Fluor ™ 594 | | RRID: AB_2534079 |
| Goat anti-Mouse IgG (H + L) Cross-Adsorbed Secondary | Invitrogen | Cat# A-11005 |
| Antibody, Alexa Fluor ™ 594 | | RRID: AB_2534073 |
| HA-Tag (C29F4) Rabbit mAb | CST | Cat# 3724 |
| | | RRID: AB_1549585 |
| Anti-Histone H3 (tri methyl K9) antibody - ChIP Grade | Abcam | Cat# ab8898 |
| | | RRID: AB_306848 |
| Tri-Methyl-Histone H3 (Lys27) (C36B11) Rabbit mAb | CST | Cat# 9733 |
| | | RRID: AB_2616029 |
| Anti-HA tag antibody | Abcam | Cat# ab9110 |
| | | RRID: AB_307019 |
| Anti-ALKBH5 antibody [EPR18958] | Abcam | Cat# ab195377 |
| | | RRID: AB_2827986 |
| Anti-FTO antibody [5-2H10] | Abcam | Cat# ab92821 |
| | | RRID: AB_10565042 |
| Anti-eIF4A3 antibody (ab32485) | Abcam | Cat# ab32485 |
| | | RRID: AB_732124 |
| Recombinant Anti-Mago nashi homolog 2 antibody | Abcam | Cat# ab186431 |
| [EPR14037-43] | | RRID: N/A |
| Anti-Aly/Ref antibody [EPR17942] | Abcam | Cat# ab202894 |
| | | RRID: AB_3094572 |
| Anti-Y14 antibody [4C4] | Abcam | Cat# ab5828 |
| | | RRID: AB_2269494 |
| Monoclonal ANTI-FLAG ® M2-Peroxidase (HRP) antibody | Sigma | Cat# A8592 |
| produced in mouse | | RRID: AB_439702 |
| Anti-mouse IgG, HRP-linked Antibody | CST | Cat# 7076S |
| | | RRID: AB_330924 |
| Anti-rabbit IgG, HRP-linked Antibody | CST | Cat# 7074S |
| | | RRID: AB_2099233 |
| GAPDH (D16H11) Rabbit mAb (HRP Conjugate) | CST | Cat# 8884S |
| | | RRID: AB_11129865 |
| Mouse monoclonal Anti-Rabbit IgG light chain (HRP) | Abcam | Cat# ab99697 |
| | | RRID: AB_10673897 |
| Anti-ACT \| Actin (polyclonal) | AgriSera | Cat# AS13 2640 |
| | | RRID: AB_2722610 |
| Histone H3K27ac antibody (pAb) | Active Motif | Cat# 39034 |
| | | RRID: N/A |
| Histone H3K9me3 antibody (pAb) | Active Motif | Cat# 39062 |
| | | RRID: N/A |
| Histone H3K4me3 antibody (pAb) | Active Motif | Cat# 39060 |
| | | RRID: N/A |
| Anti-YTHDC1 antibody | Abcam | Cat# ab122340 |
| | | RRID: AB_11128253 |
| YY1 (D5D9Z) Rabbit mAb | CST | Cat# 46395 |
| | | RRID: AB_2799302 |
| p300 (D8Z4E) Rabbit mAb | CST | Cat# 86377 |
| | | RRID: AB_2800077 |

### REFERENCES

Bhattarai, A., and Emerson, I. A. (2020). Dynamic conformational flexibility and molecular interactions of intrinsically disordered proteins. J Biosciences 45. ARTN 29 10.1007/s12038-020-0010-4.
Blue, S. M., Yee, B. A., Pratt, G. A., Mueller, J. R., Park, S. S., Shishkin, A. A., Starner, A. C., Van Nostrand, E. L., and Yeo, G. W. (2022). Transcriptome-wide identification of RNA-binding protein binding sites using seCLIP-seq. Nat Protoc 17, 1223-1265. 10.1038/s41596-022-00680-z.
Bose, D. A., Donahue, G., Reinberg, D., Shiekhattar, R., Bonasio, R., and Berger, S. L. (2017). RNA Binding to CBP Stimulates Histone Acetylation and Transcription. Cell 168, 135-149 e122. 10.1016/j.cell.2016.12.020.
Calabretta, S., and Richard, S. (2015). Emerging Roles of Disordered Sequences in RNA-Binding Proteins. Trends Biochem Sci 40, 662-672. 10.1016/j.tibs.2015.08.012.
Chen, S., Zhou, Y., Chen, Y., and Gu, J. (2018). fastp: an ultra-fast all-in-one FASTQ preprocessor. Bioinformatics 34, 1884-1890. 10.1093/bioinformatics/bty560.
Consortium, E. P. (2012). An integrated encyclopedia of DNA elements in the human genome. Nature 489, 57-74. 10.1038/nature11247.
Covelo-Molares, H., Obrdlik, A., Postulkova, I., Dohnalkova, M., Gregorova, P., Ganji, R., Potesil, D., Gawriyski, L., Varjosalo, M., and Vanacova, S. (2021). The comprehensive interactomes of human adenosine RNA methyltransferases and demethylases reveal distinct functional and regulatory features. Nucleic Acids Res 49, 10895-10910. 10.1093/nar/gkab900.
Dewari, P. S., Southgate, B., McCarten, K., Monogarov, G., O'Duibhir, E., Quinn, N., Tyrer, A., Leitner, M. C., Plumb, C., Kalantzaki, M., et al. (2018). An efficient and scalable pipeline for epitope tagging in mammalian stem cells using Cas9 ribonucleoprotein. Elife 7. 10.7554/eLife.35069.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
Duan, H. C., Wei, L. H., Zhang, C., Wang, Y., Chen, L., Lu, Z., Chen, P. R., He, C., and Jia, G. (2017). ALKBH10B Is an RNA N (6)-Methyladenosine Demethylase Affecting Arabidopsis Floral Transition. Plant Cell 29, 2995-3011. 10.1105/tpc. 16.00912.
Feng, C., Liu, Y., Wang, G., Deng, Z., Zhang, Q., Wu, W., Tong, Y., Cheng, C., and Chen, Z. (2014). Crystal structures of the human RNA demethylase Alkbh5 reveal basis for substrate recognition. J Biol Chem 289, 11571-11583. 10.1074/jbc.M113.546168.
Holehouse, A. S., and Kragelund, B. B. (2024). The molecular basis for cellular function of intrinsically disordered protein regions. Nat Rev Mol Cell Biol 25, 187-211. 10.1038/s41580-023-00673-0.
Jachowicz, J. W., Bing, X., Pontabry, J., Boskovic, A., Rando, O. J., and Torres-Padilla, M. E. (2017). LINE-1 activation after fertilization regulates global chromatin accessibility in the early mouse embryo. Nat Genet 49, 1502-1510. 10.1038/ng.3945.
Javelle, M., Marco, C. F., and Timmermans, M. (2011). In situ hybridization for the precise localization of transcripts in plants. J Vis Exp, e3328. 10.3791/3328.
Jumper, J., Evans, R., Pritzel, A. et al. (2021) Highly accurate protein structure prediction with AlphaFold. Nature 596, 583-589. 10.1038/s41586-021-03819-2.
Kim, D., Langmead, B., and Salzberg, S. L. (2015). HISAT: a fast spliced aligner with low memory requirements. Nat Methods 12, 357-360. 10.1038/nmeth.3317.
Li, X., Bie, L., Wang, Y., Hong, Y., Zhou, Z., Fan, Y., Yan, X., Tao, Y., Huang, C., Zhang, Y., et al. (2024). LINE-1 transcription activates long-range gene expression. Nat Genet 56, 1494-1502. 10.1038/s41588-024-01789-5.
Li, H., and Durbin, R. (2009). Fast and accurate short read alignment with Burrows-Wheeler transform. Bioinformatics 25, 1754-1760. 10.1093/bioinformatics/btp324.
Li, H., Handsaker, B., Wysoker, A., Fennell, T., Ruan, J., Homer, N., Marth, G., Abecasis, G., Durbin, R., and Genome Project Data Processing, S. (2009b). The Sequence Alignment/Map format and SAMtools. Bioinformatics 25, 2078-2079. 10.1093/bioinformatics/btp352.
Li, L., Luo, H., Lim, D. H., Han, L., Li, Y., Fu, X. D., and Qi, Y. (2021). Global profiling of RNA-chromatin interactions reveals co-regulatory gene expression networks in Arabidopsis. Nat Plants 7, 1364-1378. 10.1038/s41477-021-01004-x.
Li, J. F., Park, E., von Arnim, A. G., and Nebenfuhr, A. (2009). The FAST technique: a simplified Agrobacterium-based transformation method for transient gene expression analysis in seedlings of Arabidopsis and other plant species. Plant Methods 5, 6. 10.1186/1746-4811-5-6.
Liao, Y., Smyth, G. K., and Shi, W. (2014). featureCounts: an efficient general purpose program for assigning sequence reads to genomic features. Bioinformatics 30, 923-930. 10.1093/bioinformatics/btt656.
Liu, J., Dou, X., Chen, C., Chen, C., Liu, C., Xu, M. M., Zhao, S., Shen, B., Gao, Y., Han, D., and He, C. (2020). N (6)-methyladenosine of chromosome-associated regulatory RNA regulates chromatin state and transcription. Science 367, 580-586. 10.1126/science.aay6018.
Liu, J., Gao, M., He, J., Wu, K., Lin, S., Jin, L., Chen, Y., Liu, H., Shi, J., Wang, X., et al. (2021). The RNA m (6) A reader YTHDC1 silences retrotransposons and guards ES cell identity. Nature 591, 322-326. 10.1038/s41586-021-03313-9.
Long, Y., Jia, J., Mo, W., Jin, X., and Zhai, J. (2021). FLEP-seq: simultaneous detection of RNA polymerase II position, splicing status, polyadenylation site and poly(A) tail length at genome-wide scale by single-molecule nascent RNA sequencing. Nat Protoc 16, 4355-4381. 10.1038/s41596-021-00581-7.
Ludwik, K. A., Telugu, N., Schommer, S., Stachelscheid, H., and Diecke, S. (2023). ASSURED-optimized CRISPR protocol for knockout/SNP knockin in hiPSCs. STAR Protoc 4, 102406. 10.1016/j.xpro.2023.102406.
Marasca, F., Sinha, S., Vadala, R., Polimeni, B., Ranzani, V., Paraboschi, E. M., Burattin, F. V., Ghilotti, M., Crosti, M., Negri, M. L., et al. (2022). LINE1 are spliced in non-canonical transcript variants to regulate T cell quiescence and exhaustion. Nat Genet 54, 180-193. 10.1038/s41588-021-00989-7.
Martinez-Perez, M., Aparicio, F., Lopez-Gresa, M. P., Belles, J. M., Sanchez-Navarro, J. A., and Pallas, V. (2017). Arabidopsis m (6) A demethylase activity modulates viral infection of a plant virus and the m (6) A abundance in its genomic RNAs. Proc Natl Acad Sci USA 114, 10755-10760. 10.1073/pnas. 1703139114.
Percharde, M., Lin, C. J., Yin, Y., Guan, J., Peixoto, G. A., Bulut-Karslioglu, A., Biechele, S., Huang, B., Shen, X., and Ramalho-Santos, M. (2018). A LINE1-Nucleolin Partnership Regulates Early Development and ESC Identity. Cell 174, 391-405 e319. 10.1016/j.cell.2018.05.043.
Qin, X., Long, Y., Bai, X., Cao, L., Yan, H., Zhang, K., Wang, B., and Wu, X. (2023). The disordered C terminus of ALKBH5 promotes phase separation and paraspeckles assembly. J Biol Chem 299, 105071. 10.1016/j.jbc.2023.105071.
Quinlan, A. R., and Hall, I. M. (2010). BEDTools: a flexible suite of utilities for comparing genomic features. Bioinformatics 26, 841-842. 10.1093/bioinformatics/btq033.
Ramirez, F., Dundar, F., Diehl, S., Gruning, B. A., and Manke, T. (2014). deepTools: a flexible platform for exploring deep-sequencing data. Nucleic Acids Res 42, W187-191. 10.1093/nar/gku365.
Robinson, M. D., McCarthy, D. J., and Smyth, G. K. (2010). edgeR: a Bioconductor package for differential expression analysis of digital gene expression data. Bioinformatics 26, 139-140. 10.1093/bioinfor Yu, G., Wang. L. G., and He, Q. Y. (2015). ChIPseeker: an R/Bioconductor package for ChIP peak annotation, comparison and visualization. Bioinformatics 31, 2382-2383. 10.1093/bioinformatics/btv145.matics/btp616.
Shen, Y., Yue, F., McCleary, D. F., Ye, Z., Edsall, L., Kuan, S., Wagner, U., Dixon, J., Lee, L., Lobanenkov, V. V., and Ren, B. (2012). A map of the cis-regulatory sequences in the mouse genome. Nature 488, 116-120. 10.1038/nature11243.
Sigova, A. A., Abraham, B. J., Ji, X., Molinie, B., Hannett, N. M., Guo, Y. E., Jangi, M., Giallourakis, C. C., Sharp, P. A., and Young, R. A. (2015). Transcription factor trapping by RNA in gene regulatory elements. Science 350, 978-981. 10.1126/science.aad3346.
Song, H., Wang, Y., Wang, R., Zhang, X., Liu, Y., Jia, G., and Chen, P. R. (2020). SFPQ Is an FTO-Binding Protein that Facilitates the Demethylation Substrate Preference. Cell Chem Biol 27, 283-291 e286. 10.1016/j.chembiol.2020.01.002.
Sparkes, I. A., Runions, J., Kearns, A., and Hawes, C. (2006). Rapid, transient expression of fluorescent fusion proteins in tobacco plants and generation of stably transformed plants. Nat Protoc 1, 2019-2025. 10.1038/nprot.2006.286.
Tang, J., Lei, D., Yang, J., Chen, S., Wang, X., Huang, X., Zhang, S., Cai, Z., Zhu, S., Wan, J., and Jia, G. (2024). OsALKBH9-mediated m (6) A demethylation regulates tapetal PCD and pollen exine accumulation in rice. Plant Biotechnol J 22, 2410-2423. 10.1111/pbi. 14354.
Wang, J., Choi, J. M., Holehouse, A. S., Lee, H. O., Zhang, X., Jahnel, M., Maharana, S., Lemaitre, R., Pozniakovsky, A., Drechsel, D., et al. (2018). A Molecular Grammar Governing the Driving Forces for Phase Separation of Prion-like RNA Binding Proteins. Cell 174, 688-699 c616. 10.1016/j.cell.2018.06.006.
Wei, J., Yu, X., Yang, L., Liu, X., Gao, B., Huang, B., Dou, X., Liu, J., Zou, Z., Cui, X. L., et al. (2022). FTO mediates LINE1 m (6) A demethylation and chromatin regulation in mESCs and mouse development. Science 376, 968-973. 10.1126/science.abe9582.
Wuarin, J., and Schibler, U. (1994). Physical isolation of nascent RNA chains transcribed by RNA polymerase II: evidence for cotranscriptional splicing. Mol Cell Biol 14, 7219-7225. 10.1128/mcb.14.11.7219-7225.1994.
Xu, W., Li, J., He, C., Wen, J., Ma, H., Rong, B., Diao, J., Wang, L., Wang, J., Wu, F., et al. (2021). METTL3 regulates heterochromatin in mouse embryonic stem cells. Nature 597, 317-321. 10.1038/s41586-021-03210-1.
Xu, C., Liu, K., Tempel, W., Demetriades, M., Aik, W., Schofield, C. J., and Min, J. (2014). Structures of human ALKBH5 demethylase reveal a unique binding mode for specific single-stranded N⁶-methyladenosine RNA demethylation. J Biol Chem 289, 17299-17311. 10.1074/jbc.M114.550350.
Xuc, F., Zhang, J., Wu, D., Sun, S., Fu, M., Wang, J., Searle, I., Gao, H., and Liang, W. (2024). m (6) A demethylase OsALKBH5 is required for double-strand break formation and repair by affecting mRNA stability in rice meiosis. New Phytol 244, 2326-2342. 10.1111/nph. 19976.
Yang, L., Ding, J., Zhang, C., Jia, J., Weng, H., Liu, W., and Zhang, D. (2005). Estimating the copy number of transgenes in transformed rice by real-time quantitative PCR. Plant Cell Rep 23, 759-763. 10.1007/s00299-004-0881-0.
Yang, X., Triboulet, R., Liu, Q., Sendinc, E., and Gregory, R. I. (2022). Exon junction complex shapes the m (6) A epitranscriptome. Nat Commun 13, 7904. 10.1038/s41467-022-35643-1.
Yu, Q., Liu, S., Yu, L., Xiao, Y., Zhang, S., Wang, X., Xu, Y., Yu, H., Li, Y., Yang, J., et al. (2021). RNA demethylation increases the yield and biomass of rice and potato plants in field trials. Nat Biotechnol 39, 1581-1588. 10.1038/s41587-021-00982-9.
Yu, G., Wang, L. G., Han, Y., and He, Q. Y. (2012). clusterProfiler: an R package for comparing biological themes among gene clusters. OMICS 16, 284-287. 10.1089/omi.2011.0118.
Yu, G., Wang, L. G., and He, Q. Y. (2015). ChIPseeker: an R/Bioconductor package for ChIP peak annotation, comparison and visualization. Bioinformatics 31, 2382-2383. 10.1093/bioinformatics/btv145.
Zhang, Y., Dong, Q., Wang, Z., Liu, Q., Yu, H., Sun, W., Cheema, J., You, Q., Ding, L., Cao, X., et al. (2024). A fine-scale Arabidopsis chromatin landscape reveals chromatin conformation-associated transcriptional dynamics. Nat Commun 15, 3253. 10.1038/s41467-024-47678-7.
Zhang, X., Henriques, R., Lin, S. S., Niu, Q. W., and Chua, N. H. (2006). Agrobacterium-mediated transformation of Arabidopsis thaliana using the floral dip method. Nat Protoc 1, 641-646. 10.1038/nprot.2006.97.
Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-Seq (MACS). Genome Biol 9, R137. 10.1186/gb-2008-9-9-r137.

What is claimed is:

1. A recombinant DNA encoding an engineered RNA m⁶A demethylase, wherein the engineered RNA m⁶A demethylase comprises at least one disrupted endogenous region, wherein the region is a low complexity region (LCR) and/or intrinsically disordered region (IDR).

2. The recombinant DNA of claim 1, wherein the at least one disrupted endogenous region is a low complexity region (LCR).

3. The recombinant DNA of claim 1, wherein the RNA m⁶A demethylase comprises a disrupted endogenous N-terminal region and/or a disrupted C-terminal region.

4. The recombinant DNA of claim 1, wherein the disruption of the endogenous region is selected from the group consisting of a partial truncation, a full truncation, a deletion, and a replacement.

5. The recombinant DNA of claim 1, wherein the RNA m⁶A demethylase comprises a deletion of at least about 10%, 20%, 30%, 40%, 50%, 60%, 70%, 80%, 90%, or 100% of an endogenous region, wherein the region is an LCR or IDR.

6. The recombinant DNA of claim 1, wherein the RNA m⁶A demethylase is an engineered ALKBH5 or an engineered ALKBH5 homolog.

7. The recombinant DNA of claim 1, wherein the RNA m⁶A demethylase comprises an amino acid sequence with at least about 70%, 75%, 80%, 85%, 90%, 91%, 92%, 93%, 94%, 95%, 96%, 97%, 98%, 99%, or 100% sequence identity to a sequence selected from the group consisting of SEQ ID NOs: 42-44.

8. The recombinant DNA of claim 1, wherein the endogenous region leads to assembly of the endogenous RNA m⁶A demethylase in foci or condensates within the cell.

9. The recombinant DNA of claim 6, wherein the engineered RNA m⁶A demethylase is an engineered ALKBH5 comprising a deletion of at least about 10%, 20%, 30%, 40%, 50%, 60%, 70%, 80%, 90%, or 100% of:
(i) amino acids 30-81 and/or
(ii) amino acids 298-394
of endogenous ALKBH5 (SEQ ID NO: 1).

10. The recombinant DNA of claim 9, wherein the RNA m⁶A demethylase is selected from the group consisting of ALKBH5_{Δ30-81}, ALKBH5_{Δ298-394}, and ALKBH5_{Δ30-81 and 298-394}.

11. The recombinant DNA of claim 6, wherein the RNA m⁶A demethylase is an engineered plant ALKBH5 homolog.

12. The recombinant DNA of claim 11, wherein the RNA m⁶A demethylase is Arabidopsis ALKBH9B, optionally wherein the ALKBH9B comprises a deletion of at least about 10%, 20%, 30%, 40%, 50%, 60%, 70%, 80%, 90%, or 100% of:
(i) amino acids 76-102,
(ii) amino acids 145-183, and/or
(iii) amino acids 432-507
of endogenous ALKBH9B (SEQ ID NO: 3).

13. The recombinant DNA of claim 11, wherein the RNA m⁶A demethylase is Arabidopsis ALKBH10B, optionally wherein the ALKBH10B comprises a deletion of at least about 10%, 20%, 30%, 40%, 50%, 60%, 70%, 80%, 90%, or 100% of:
(i) amino acids 131-190 and/or
(ii) amino acids 501-569
of endogenous ALKBH10B (SEQ ID NO: 5).

14. The recombinant DNA of claim 11, wherein the RNA m⁶A demethylase is Os9B, optionally wherein the Os9B comprises a deletion of at least about 10%, 20%, 30%, 40%, 50%, 60%, 70%, 80%, 90%, or 100% of:
(i) amino acids 60-170 and/or
(ii) amino acids 428-616
of endogenous Os9B (SEQ ID NO: 6).

15. The recombinant DNA of claim 11, wherein the RNA m⁶A demethylase is Os10B, optionally wherein the Os10B comprises a deletion of at least about 10%, 20%, 30%, 40%, 50%, 60%, 70%, 80%, 90%, or 100% of:
(i) amino acids 2-30,
(ii) amino acids 99-126, and/or
(iii) amino acids 491-595
of endogenous Os10B (SEQ ID NO: 8).

16. The recombinant DNA of claim 11, wherein the RNA m⁶A demethylase is rapeseed ALKBH9B, tobacco ALKBH9B, tobacco ALKBH10B, alfalfa ALKBH9B, sorghum ALKBH9B, maize ALKBH9B, maize ALKBH10B, wheat ALKBH9B, or wheat ALKBH10B.

17. The recombinant DNA of claim 1, wherein the RNA m⁶A demethylase is operably linked to at least one nuclear localization signal (NLS).

18. The recombinant DNA of claim 1, wherein the engineered RNA m⁶A demethylase is operably linked to a promoter for expression in a plant.

19. An expression vector comprising the recombinant DNA of claim 1.

20. A plant or plant cell comprising the recombinant DNA of claim 1, wherein the plant or a plant comprising the plant cell has improved growth compared to a control plant.

21. The plant or plant cell of claim 20, wherein the plant or plant cell is a plant selected from the group consisting of Arabidopsis, rice, corn, soybean, potato, cassava, cowpea, cotton, turf grass, strawberry, blueberry, blackberry, raspberry, and wheat, or a plant cell therefrom.

22. The plant of claim 20, wherein the plant is improved in a characteristic selected from the group consisting of biomass, yield, root growth, root expansion, stem cell expansion of the root, stem cell expansion of the shoot, drought resistance, and pest resistance.

23. A plant or plant cell comprising a modified endogenous ALKBH5 homolog gene, wherein the modified endogenous ALKBH5 homolog gene encodes a protein comprising at least one disrupted endogenous region, wherein the region is a low complexity region (LCR) and/or intrinsically disordered region (IDR).

24. The plant or plant cell of claim 23, wherein the plant or a plant comprising the plant cell has improved growth compared to a control plant.

25. A method of improving growth of a plant, comprising:
a) engineering a plant to comprise the recombinant DNA of claim 1, and
b) growing the plant,
wherein the plant has improved growth compared to a control plant.

26. A method of improving growth of a plant, comprising:
a) genetically modifying the plant by transforming the plant with one or more gene editing components that target an endogenous ALKBH5 homolog gene, wherein the endogenous ALKBH5 homolog gene is modified to encode a protein comprising at least one disrupted endogenous region, wherein the region is a low complexity region (LCR) or an intrinsically disordered region (IDR), and
b) growing the plant,
wherein the plant has improved growth compared to a control plant.

27. A plant produced by the method of claim 25.

28. A plant produced by the method of claim 26.

29. A method of identifying an intrinsically disordered region (IDR) or intrinsically disordered domain (IDD) in an ALKBH5 gene or ALKBH5 homolog, the method comprising:
(i) providing a polypeptide that encodes an ALKBH5 gene or ALKBH5 homolog, and
(ii) identifying a region of the polypeptide for which
(a) the IUPred score of the region exceeds 0.5,
(b) in an AlphaFold-predicted structure of the polypeptide the region has a pLDDT of less than 70, and
(c) the region has less than 70%, less than 65%, less than 60%, less than 55%, less than 50%, less than 45%, less than 40%, less than 35%, less than 30%, less than 25%, less than 20%, less than 15%, less than 10%, or less than 5% identity with the catalytic domain of ALKBH5,
wherein the region is therefore identified as an IDR or IDD.


SEQUENCE LISTING (192 sequences)

SEQ ID NO: 1 - protein, 394 aa, Homo sapiens
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHRPRILEMDKEENRRSVLLPTHRRRGSFSSENYWRKSYESSEDCSEAAGSPARKVKMRRH

SEQ ID NO: 2 - DNA, 1185 bp, Homo sapiens
  source 1..1185: mol_type = unassigned DNA
atggcggccgccagcggctacacggacctgcgtgagaagctcaagtccatgacgtcccgggacaactataaggcgggcagccgggaggccgccgccgctgccgcagccgccgtagccgccgcagccgcagccgccgctgccgccgaaccttaccctgtgtccggggccaagcgcaagtatcaggaggactcggaccccgagcgcagcgactatgaggagcagcagctgcagaaggaggaggaggcgcgcaaggtgaagagcggcatccgccagatgcgcctcttcagccaggacgagtgcgccaagatcgaggcccgcattgacgaggtggtgtcccgcgctgagaagggcctgtacaacgagcacacggtggaccgggccccactgcgcaacaagtacttcttcggcgaaggctacacttacggcgcccagctgcagaagcgcgggcccggccaggagcgcctctacccgccgggcgacgtggacgagatccccgagtgggtgcaccagctggtgatccaaaagctggtggagcaccgcgtcatccccgagggcttcgtcaacagcgccgtcatcaacgactaccagcccggcggctgcatcgtgtctcacgtggaccccatccacatcttcgagcgccccatcgtgtccgtgtccttctttagcgactctgcgctgtgcttcggctgcaagttccagttcaagcctattcgggtgtcggaaccagtgctttccctgccggtgcgcaggggaagcgtgactgtgctcagtggatatgctgctgatgaaatcactcactgcatacggcctcaggacatcaaggagcgccgagcagtcatcatcctcaggaagacaagattagatgcaccccggttggaaacaaagtccctgagcagctccgtgttaccacccagctatgcttcagatcgcctgtcaggaaacaacagggaccctgctctgaaacccaagcggtcccaccgcaaggcagaccctgatgctgcccacaggccacggatcctggagatggacaaggaagagaaccggcgctcggtgctgctgcccacacaccggcggaggggtagcttcagctctgagaactactggcgcaagtcatacgagtcctcagaggactgctctgaggcagcaggcagccctgcccgaaaggtgaagatgcggcggcactga

SEQ ID NO: 3 - protein, 507 aa, Arabidopsis thaliana
MENDPFLRQYQPSELKIASEFLTNWLPFLSKDLCKDCNHLLSNRIRSLDPAHCSNNTDKVDGECKTGSCSVVENMGSERASNNVDDNYDEKSENGEDCDNHSLGSWKGSEIVFGSFPEDFSSVLQSRPAVVETASPRMRWADMAQEDEFDEEEEEEEEERDSSRKGFDASSMKTPEKPKLSRDQRENLRLINVKRKKDFICLERVKGKIVNVLDGLELHTGVFSAVEQKRIVDQVYQLQEKGRRGELKKRTFTAPHKWMRGKGRETIQFGCCYNYAPDRAGNPPGILQREEVDPLPHLFKVIIRKLIKWHVLPPTCVPDSCIVNIYDEGDCIPPHIDNHDFLRPFCTISFLSECDILFGSNLKVEGPGDFSGSYSIPLPVGSVLVLNGNGADVAKHCVPAVPTKRISITFRKMDESKRPVWFTPEPDLQGIEPLPLDLNRSGSTSRFSRLNNHNGTNQRGHGRRGGGNGYDSRGYYNPERSSEHNDSGDWPSSQRRGMPRPSRRNYG

SEQ ID NO: 4 - DNA, 1521 bp, Arabidopsis thaliana
  source 1..1521: mol_type = unassigned DNA
atggaaaacgatccatttctccggcagtaccagccgtcggagcttaagatcgcttcggagtttctgacgaattggcttccttttttatctaaagatctatgcaaggattgtaatcaccttctctccaatcgaatccgttctcttgacccagcacattgtagtaacaacactgacaaagttgatggagagtgtaaaactggttcttgttcagtggtggaaaatatgggatcagaaagagctagtaacaatgtggatgataattatgatgagaagtcggaaaatggcgaagactgtgataatcattcgttaggtagttggaagggaagtgaaattgtatttggttcctttcctgaagattttagtagtgtattgcaatctagaccagctgttgttgaaacagcaagccctcgtatgcgttgggctgatatggcacaagaggatgagttcgatgaggaggaggaggaggaggaagaggagagggattcgagtaggaaaggttttgatgcgagttcgatgaagactccagagaagcctaagttgtctagagatcagagagagaatctcaggctgattaatgtgaagagaaagaaagactttatttgcttggagagagttaagggaaagattgttaacgtgcttgatggacttgagttgcacactggtgtttttagcgcagtggagcagaaacggattgttgatcaagtgtatcaacttcaagagaagggacgaagaggagaacttaaaaagcgtacatttactgctccacataagtggatgagaggaaaaggacgtgagactatacaatttggctgttgctacaactacgctccagacagagctggaaacccaccgggtatccttcaacgtgaagaagtggatccactacctcatcttttcaaagtaatcattagaaagttgattaaatggcatgtactgcctccaacttgtgtgcccgatagctgcattgtgaacatctatgatgaaggtgactgcatacctcctcacatcgacaaccacgactttctccgtcccttctgtaccatctctttcctcagtgaatgcgatatcctctttggctcaaacctgaaagttgagggtcctggtgacttctcgggttcatactcaataccgcttcctgtgggatcagttctggtgttaaatggaaatggagctgatgtagctaagcactgtgtacctgcagttcctacaaaaaggatatcaattacatttaggaaaatggatgagtcgaaacgcccggtttggttcaccccagagcctgatttgcaagggattgagccattgccattggacctgaaccggtctggttctacttcaaggttctcaagattaaacaaccataacggtactaaccaaagaggacatggacgtagaggaggaggcaatggttatgactcaagaggttactataatcctgagagaagcagtgaacacaatgactctggagactggccatcaagccagagaagaggaatgccacgtcctagtagaagaaactacggt

SEQ ID NO: 5 - protein, 569 aa, Arabidopsis thaliana
MTIAAAPARQTDRSATGFNPAYVTTAKAVSVPVQVPPATVVSEGLGKDALISWFRGEFAAANAIIDAMCSHLRIAEEAVSGSEYEAVFAAIHRRRLNWIPVLQMQKYHSIAEVAIELQKVAAKKAEDLKQKKTEEEAEEDLKEVVATEEEEVKKECFNGEKVTENDVNGDVEDVEDDSPTSDITDSGSHQDVHQTVVADTAHQIICHSHEDCDARSCEIKPIKGFQAKEQVKGHTVNVVKGLKLYEELLKEDEISKLLDFVAELREAGINGKLAGESFILFNKQIKGNKRELIQLGVPIFGHVKADENSNDTNNSVNIEPIPPLLESVIDHFVTWRLIPEYKRPNGCVINFFEEGEYSQPFLKPPHLEQPISTLVLSESTMAYGRILSSDNEGNFRGPLTLSLKQGSLLVMRGNSADMARHVMCPSQNKRVSITFFRIRPDTYHNHSQPNSPRNDGVMTMWQPYQMTPTPFLNGYDHSIDMMPKLGVLRPPMVMMAPPPVQPMILPSPNVMGTGGGTGVFLPWASVNSSRKHVKHLPPRAQKKRLLPLPPAASSSPAGGSTSEPVITVG

SEQ ID NO: 6 - protein, 616 aa, Oryza sativa
MDASSSASAGGEADAMALVQGYNADELAIAGEFLTTWLPFLSAGLCASCADSLRSRVSSLLPPQAEESPSSPPPRIDQIEPSGWESDPATAHPQHLPFEPSGWDSDPPQLPPEQEQQKQKPQPAEKPRKSWADMAQEDELAAAAEEDAAAAAADDGEEGSEAGRPGVQLTREQRELRRFRNVVRRKDFICFERVNGRLVNILAGLELHCGVFSAAEQKRIVDYVYDLQEMGKHGELGDRTYTEPQRWMRGKGRVTIQFGCCYNYATDKNGNPPGIIRTIASDPMPSLFKIMIKRLVRWHVLPKTCIPDSCIVNIYDPGDCIPPHIDSHDFVRPFCTVSFLSECNILFGSTLKIAGPGEFTGSLPIPLPVGSVLILNGNGADVAKHCVPAVPTKRISITFRKMDPAKRPFNFRDDPELLNIIPLETAVQETGRSSDEGKGKQPDIQIRNPSKAHRNKKSKVRTSPGKGGRGGILGDGPPQYAQAQVTGISSQQNFHGQPTISGSSAERERRPVGPLRESRYQQDAPGMQSNMDGIRERANWLAQERMHGNSMNSIDDGTESQERRQRMEHRQILMINRTINDDMDSLSIGSHESDQTRVSVRTLYNKPRRTRVNLDE

SEQ ID NO: 7 - DNA, 1851 bp, Oryza sativa
  source 1..1851: mol_type = unassigned DNA
atggacgcctcctcctccgcctccgccggcggcgaggccgacgcgatggcgctcgtgcaggggtacaacgccgacgagctcgccatcgccggggagttcctcaccacgtggctccccttcctctccgcgggcctctgcgcctcctgcgccgactccctccgcagccgcgtctcctccctgctgcccccgcaagccgaggaatccccgtcgtcgccgccgccgcggatcgatcagattgagccctcggggtgggagtccgatccggctacggctcacccgcagcatctgccgttcgagccgagcggatgggactcggatccgccgcagctgccaccggagcaggagcagcagaagcagaagccgcagccggcggagaagccgcggaagtcgtgggcagacatggctcaggaggacgagctcgccgccgcggcagaggaggacgcggcggcagccgcggctgatgacggcgaggaagggagcgaggctggtaggccgggtgtgcagctgacgagggagcagcgggagctgaggcggttccggaacgtggtgcggaggaaggatttcatctgttttgagagggtcaatggtcggctcgtcaatatcctcgctggcctggaacttcattgcggtgtgtttagcgcggccgaacagaagcgtattgtcgactacgtatacgatcttcaggagatgggcaagcacggggagcttggagatcgtacgtatacggaaccccaaaggtggatgcgtggtaaagggcgagtaacaatccaatttggatgctgttataattatgccacggacaagaatggaaacccaccaggcattattcgaactattgcttctgatccgatgcctagcttatttaagatcatgatcaagagattggtacggtggcatgttctaccaaagacttgcataccagacagttgtattgtcaatatatatgaccctggagattgcatcccaccacatattgatagtcatgattttgttcggccattctgtactgtttcattcctcagtgagtgcaacattctttttggatctactctgaaaattgctggccctggagagttcactggttcacttccaattcctctgcctgttgggtcggtgcttatcctaaatggcaatggtgctgatgtagcaaagcattgtgttcctgcagttccgaccaaaaggatatccattaccttcagaaagatggatccagcgaagcgtccgttcaatttccgagatgatccagaattgcttaacataattcctctagaaacagctgtacaagaaactggcagatcatcagatgaaggtaagggcaagcagcctgacatacagatcagaaatccgagcaaagcacacaggaacaagaaatctaaagtaagaacatctcctggaaagggtggacggggcggcattctcggagatggacctcctcaatatgcacaagctcaagtcactggcatttcgtcacagcaaaacttccatggccagcctaccatctctggttcaagtgctgagagagaaagacgccctgttggtccattgagagagtcaagatatcagcaggatgcacctggcatgcaatctaacatggatggtatcagagagcgggcaaattggttggctcaagagaggatgcatggtaacagcatgaattcgattgacgatggcacggaatctcaggaaaggaggcagcgaatggagcacagacagatattgatgatcaaccgcacgatcaacgatgatatggattccctttcaatcgggagccatgaatctgaccagactcgtgtaagcgtacgaacactatacaacaagccaaggaggaccagggtaaacctggatgagtaa

SEQ ID NO: 8 - protein, 595 aa, Oryza sativa
MAAPGPGWVVPPPPQPQPQQQQQAVVVDGRDGLVAWLRGEFAAANAIIDLLLAHARDAADPAGFDAVAAAVQRRRHHWAPVLHLQHYFPVTEVALALHHAAARQGPPPPPPPPRPPSGSAGAEGDDAAIASGGVKEVETSAEATQNSQLVSHISHATEAQPQKGLHVISNVVPVPTCFVVNEVIDGRMVNVLEGLKLYKGYVDLTEIGKVLSFVNEAKTMRRKPGLEGQTVVVAKRPMKGHGREIIQLGLPITEGPPEDEHLREVKVDPIPGVLQNLFDSLVHQKVVPSSPDYCVIDIFNEGDYSHPHHHPPWYGRPICTLCLTDCDMVFGHVIAADSRGDHAGPLKLSLSTGSVLVFEGKSADIAKRALPATSKQRILLSFGKSVSRKHVQSESSLLITPPLTPPPMPWAAPLRPGNIAIHPSSPKQLVYNPSNRVPAVSTPGLHHIPSNGIQTVFVAPLPITPKAVPFASAVTLPNSTAAWIAEAAPRPASPRLPLQGTGVFLPPGSGNPPPAQKLGVKHADAKPFFPQESSASSSGVSARAHKANGSVSSKPTRKDDMAEAKPKCNGSSDGGSSVAHAKATGGLEEQNVVAK

SEQ ID NO: 9 - DNA, 1788 bp, Oryza sativa
  source 1..1788: mol_type = unassigned DNA
atggccgcgccggggccggggtgggtggtgccgccgccgccgcagccgcagccgcagcagcagcagcaggcggtggtggtggacggccgggacggcctcgtcgcgtggctgcgcggcgagttcgcggcggccaacgccatcatcgacctcctcctcgcccacgcccgcgacgccgccgatcccgccgggttcgacgccgttgccgccgccgtccagcgcaggcggcaccactgggcccccgtcctccacctccagcattacttccccgtcaccgaggtcgccctcgcgcttcaccacgccgccgcgcgccaggggccgccgcctcctcctccgccgccgcgccccccgagcggtagcgccggtgccgagggagatgatgctgcgattgccagtggaggtgtgaaggaagtggagacatcggctgaagcaactcagaattctcagttggtttctcatatttcacatgctacagaagcccaacctcagaaaggacttcatgtaatcagtaatgtggttccagtcccaacttgctttgtggttaatgaagtaattgatggcaggatggtgaatgttctagaggggcttaagttgtataaagggtatgtagatctaactgagataggaaaggttctttcttttgtgaatgaagctaagactatgcggcgtaaaccaggactggaagggcaaacagttgtagttgccaaaaggccaatgaagggtcatggaagagaaattattcagcttggtcttcccatcacagaaggccctcctgaagatgaacatttaagagaggtaaaggtggatcctattcctggtgtgcttcaaaatctgtttgatagcttggttcatcagaaagttgtcccttccagtccagattactgtgttatcgatatcttcaatgagggagattattctcaccctcatcatcatcctccttggtatggtagacccatttgtacactctgcctgactgactgcgacatggtatttggccatgttattgcagcagatagccgaggcgatcatgcaggtccactgaaactctcactctctacagggtctgttctagtttttgagggaaagagcgctgatatagcaaaacgggcactccctgctacaagtaagcagcgtatcttactaagttttgggaagtctgtgtcaaggaagcacgttcagtcggaaagcagcttgcttatcactcccccattgacacctcctccgatgccttgggctgcaccattgaggccagggaacatagcaattcatccctcaagtcctaaacagcttgtctacaaccctagcaatagagtacctgcagtttcaacccctggacttcaccatattccatccaatggcatccagacagttttcgttgcaccacttcctatcactccaaaagctgttccatttgcatcagctgtcacattgccaaacagtacagccgcatggatcgcagaagctgctcccaggcccgcttcacctcgtttacctcttcagggtacaggggtattccttcctcctggatcaggcaatccacccccagctcagaagttgggcgtaaaacatgctgatgctaaaccattttttccacaagaatcatctgcctcttcatctggtgtaagtgctcgtgcacataaggctaatggtagtgtttcttccaaaccaacaaggaaagatgatatggctgaagctaagccaaagtgcaatgggagttcagatggtggtagctctgttgcccatgccaaggcaactggaggtttagaggagcagaatgttgtcgcaaagtag

SEQ ID NO: 10 - protein, 467 aa, Brassica napus
MEDDPFLRKFQPSELKIASEFLTNCLPFLSRDLCKDCVNVLSDRIRSLDPEHCSKVGGEGNSKAGLESEGTSKIETEISFGTLSEVLGSVLPSRQAAETASPRMSWADMSQEDEFEEEDEEEESRKAIDASPMKTPEKPKLSREQREDLRLKNVKRKKDFICLERFKGKLVNVVDGLELHTGVFSAVEQKRIVDKVYELEERGRKGELRERTFTAPQKWMRGKGRVTIQFGCCYNYATDRAGNPPGILQREEVDPLPSLFKVIIRRLIRWHVLPPTCVPDSCIVNIYDEGDCIPPHIDNHDFLRPFCTISFLSECNILFGSNLKIEGPGEFSGSFSVPLPVGSVLVLNGNGADVAKHCVPAVPTKRISITFRKMDESKRPVWFTPEPDLQGIEPLPLDLNRSDSAARSAGSSSNHNGSNRRGGYGRRGGNNYETRGYYTPERSSEQYESREWSSSQRRGRPRPGRTS

SEQ ID NO: 11 - DNA, 1404 bp, Brassica napus
  source 1..1404: mol_type = unassigned DNA
atggaagacgaccctttcctccggaagttccagccatcggaactcaagatcgcgtcggagttcttgacgaattgccttcctttcttgtctagagatctctgcaaagactgcgtcaacgttctctccgatcgaatccgctctcttgacccagaacattgtagtaaagttggtggagaaggtaatagcaaagctggtttggaatcagaaggaactagtaagatagagactgaaatttcatttggaaccttgtctgaagttcttggtagtgtactgccatctcgacaagctgctgaaacagcaagccctagaatgtcttgggctgatatgagtcaagaagatgagtttgaggaggaggacgaagaggaggagagtaggaaagctattgatgcgagtccgatgaagactccagagaagcctaagttatcaagagagcaaagagaggatctcaggctgaagaacgtgaagagaaagaaagatttcatttgcttggagagatttaagggaaagctcgttaacgtagttgatgggcttgagttgcacactggtgtgttcagcgcagtggagcagaaaaggattgttgataaagtgtatgaacttgaagagagaggacgcaaaggagaacttagagagcgtacgtttactgctccacagaagtggatgagaggcaaaggacgtgtgactattcaattcggctgttgttacaactacgctacagatagagctggaaacccaccgggtatccttcaacgcgaagaagtggatccattaccttctcttttcaaagtgatcatcagaaggttgattagatggcatgtcctgcctccgacttgtgtgcccgatagctgcattgtcaacatctacgatgaaggtgactgtatacctccccacatcgacaaccacgactttctccgtcccttctgcaccatctcgttcctcagtgaatgcaacatcctctttggctcaaaccttaaaatagaaggtcctggtgaattctccggttcattctcagtaccacttcctgtaggatcagttctggtgttaaatggaaatggagctgatgtagctaaacactgtgtacctgcagttcccacaaaaaggatatcaatcacgtttaggaaaatggatgagtctaaacgcccggtttggttcactccagaacctgatttgcaagggatcgagccactgccgttggacctgaaccggtctgattctgctgcaaggtccgcaggatcaagcagcaaccacaacggcagtaacagaagaggaggatatggacgtagaggaggaaacaattacgagacgagaggttactatactcccgagagaagcagtgaacagtatgagtctagagaatggtcatcaagccagagaagaggaaggccacgtccgggtagaacctcctaa

SEQ ID NO: 12 - protein, 540 aa, Nicotiana benthamiana
MSESDQQRTQKADPFLLNYNPEELRIASEFLSNWLPFLSRDLCHSCTHTLSDRIRSLNFKVGGDAECLKQPKNISVLTPERCDSDGCNGSQENCDNNSLGSWNDCGDLNDNADTNSLGSWKDGTSGGEPFEEPSGDRHSSDSCIDGADSLSRPVGEASTSEAFSSSLPVTTPKMKMSWADMAQEDELQAEEVSESIALSSQVNGVSEEEITTPESKPKTELSREQREYIRFCNVKRKKDFICLERVNGKIVNILDGLELHKGVFSAAEQIRIVKYVEKLDQMGKNGELKERTYTAPAKWMRGKGRVTIQFGCCYNYAPDKSGNPPGILKSETVDPLPDLLKVMIRRLVRWHVMPPTCVPDSCIINIYEEGDCIPPHIDNHDFVRPFCTVSFLSECNIVFGSNLKIVGPGEFAGAIAIPLPVGSVLVLKGNGADVAKHCVPAVPTKRISITFRRMDESRRPVGYVPEQDLQGLQPLSYEADVQKKSKSSKPRYSARKQSALREESMESVKMPIRRHSEPRYTGRYRQGPANRQRFRVNTEN

SEQ ID NO: 13 - DNA, 1623 bp, Nicotiana benthamiana
  source 1..1623: mol_type = unassigned DNA
atgtccgagtccgaccagcagcgaactcagaaggccgatccattcctccttaattacaatcccgaagagctccgaatcgcatcagagtttctgtccaattggctgccctttctctcccgtgacctctgccactcctgcacccacaccctctccgatcgcatccgatccctcaattttaaagttggtggtgatgcagaatgcttgaagcaaccaaagaatatcagtgttttgactccggaacgatgtgactcggatggatgcaatgggagtcaagagaattgtgacaacaattcacttggaagttggaatgattgtggtgacttgaatgataatgcagatacaaactcattggggagttggaaagacgggactagtgggggggagccatttgaggagccttccggggacaggcattcatctgacagttgtatagatggcgcagattccctttctcggcctgttggagaggcatctacaagtgaagcattcagctcatccttacctgtgactaccccgaaaatgaagatgtcgtgggcagatatggctcaggaggatgagcttcaagctgaggaagtgagtgagtcaattgccttgagtagtcaggtgaatggtgtgtctgaagaggaaattactactccggagagtaagccaaaaacagaattgtcaagggagcaacgagagtacattagattctgtaatgtgaagaggaaaaaggatttcatttgtttggagagggtgaatgggaaaatcgtgaacatacttgatggattggagctacataagggtgtctttagcgcagctgagcaaattagaatagttaaatatgtggagaagcttgatcagatggggaagaatggggaattgaaagagaggacttatactgcaccagcaaagtggatgaggggcaaaggacgtgtgacaatccaatttggttgttgttacaactatgcaccagacaagagtgggaatcccccagggatcctcaaaagtgaaactgttgatccattgcctgatcttttgaaggtcatgattagaaggcttgtaaggtggcatgtgatgcctccaacttgtgttccagacagctgtattatcaacatttatgaagagggggattgcataccaccgcatattgacaatcacgatttcgtgcgtccattttgtaccgtttcattccttagcgagtgcaatattgtgttcggttccaacttgaaaatagttggtcccggtgagtttgctggtgcaattgcaataccgttgccagtgggatctgtactggtcttgaaaggaaatggagctgatgtagctaaacactgtgtgccagctgtgcctactaaaaggatttcaatcacatttagaagaatggacgaatctagaaggccagttggatatgttcctgagcaagatttacaagggctgcagcccttgtcatatgaagcagacgtacagaagaaatcaaaatcctcaaaaccaaggtattctgcaagaaaacagtcagcattacgggaagagagcatggaaagtgtaaagatgccaataagaaggcattcagagccccgttacacaggtcgatatcgacaaggacctgcaaacaggcagaggtttagggtgaatacagaaaattga

SEQ ID NO: 14 - protein, 638 aa, Nicotiana benthamiana
MQSGNATLVPEKMHGGGGGEAVEPPPHQQQWFMGDERDGFISWLRGEFAAANAIIDALCHHLRLVGEQPGEYDGVIGCVQQRRGNWSTVLHMQQYFSVAEVIYALHQVEWRKQQKGGFNNKRNSGGSRGGGGGGWRSEGHNFTMDANSKEFYSKSNGVEKIDVIEKEIDVKQGEKKELVGNPEGDSSMKSSVCIEAADSQSEMDKTDHKRDSNSDGSCKAENESRSSEVPNEKQNVTVVPKTFVATEIYDGKPVNVVDGMKLYEELLSSSEVSKLVTLVNDLRASGRRGQLSAQTFIVSKRPMKGHGREMIQLGLPIADAPPEDEAAFATFKERKMEVIPGLFQDVIERLSAMQVLTAKPDACTIDIFNEGDHSQPHMWPYWYGRPVAMLFLTECEMTFGKMIGVDHPGDYRGSLKLSFAPGSVLVMQGKSTDFARHAIPSIRKQRILVTFTKVQPRRFKSGDSQRFSSSAGGAAPQWVPPPSRSPNHIRHPFGPKHYGSMPTTGVLPVPAIRSQLAPPNGIQPIFVPAAVAPPMAFPAPVALPPASGGWAAPPPRHPPPRLPLPGTGVFLPPGSGASATGNNPAENPTSSEKDSAGSLRDTTASLKDNVDSEVETQEGNGKIDDSGAETAVANEERH

SEQ ID NO: 15 - DNA, 1917 bp, Nicotiana benthamiana
  source 1..1917: mol_type = unassigned DNA
atgcaatcaggaaatgccacgttagttccggagaaaatgcacggcggtggtggaggagaggcggtggaaccaccgccacatcagcagcagtggtttatgggtgatgagagggatgggtttatatcatggttaagaggtgaatttgctgcagcaaatgctataattgatgctttgtgtcatcatttaaggttagtaggtgagcagcctggtgagtatgatggtgtaattggatgtgtacaacaaaggagagggaattggagcactgtgttgcatatgcagcagtacttttcagtagctgaagtgatctatgcattgcatcaggttgagtggaggaagcagcaaaaagggggttttaataataagaggaatagtgggggttctagaggaggaggaggagggggttggagaagtgagggtcataattttactatggatgctaatagtaaagagttttatagtaaatctaacggggtcgaaaagattgatgttatagaaaaagaaatagatgtgaagcaaggggaaaagaaagaattggttggaaatccagaaggagatagctcaatgaagagttcagtttgcatagaagctgctgactcacaaagtgaaatggacaaaaccgatcataaacgtgattcaaattctgatgggtcttgtaaggcggagaatgaatctcgatccagcgaagtaccaaatgagaagcagaatgttactgttgtaccaaaaacttttgttgctacagagatatacgatggaaaaccggttaatgttgttgatggcatgaaactgtacgaggaactgctaagcagttcagaagtttcaaaacttgttactttggtaaatgatttgagagcttctgggagaaggggacagctttctgcacagacatttatagtctcaaagagaccgatgaaggggcacggaagggagatgatccagctgggcctacccattgcagatgcacctcctgaagatgaagctgcctttgcaacatttaaagagcggaaaatggaagtgattcctggcttgttccaagatgttattgaacgactgagtgctatgcaagttttaactgcgaagccagatgcttgcactattgacatcttcaatgagggggaccactcacaacctcatatgtggccttattggtatgggcggcctgtagctatgttgttcttgacagaatgtgaaatgactttcggtaagatgattggtgtggaccatccgggcgattatcgaggctctctcaaactctcttttgcaccaggatccgtacttgtaatgcagggaaaatcaaccgattttgctagacatgctattccatccatccgtaagcagcgtatacttgtgacctttacaaaggtgcagccaagaagattcaagtctggtgatagtcagcgtttctcttcatcagcaggaggtgctgccccccagtgggtcccccctcctagcaggtcaccaaatcatatccgtcatccttttgggcccaagcattatggttccatgccaacaaccggcgtattacccgttccagctattcgttcacagttagctcctccaaatggtatccagccaatttttgttcccgctgcggttgcaccgcctatggcttttcctgcaccagttgctcttccacctgcctcaggtggatgggcagcaccccctcctaggcatcccccaccacgacttccactccctggcactggcgttttccttcctccaggctctggggcttcagccactggcaacaatcctgctgaaaatccaacttcttcagaaaaggacagtgcaggttctcttagagataccactgcttctctgaaagataacgtggatagtgaagtagaaacacaagagggcaacggaaagatagatgatagtggtgcagagacagcagtggctaatgaagaacgacactaa

SEQ ID NO: 16 - protein, 508 aa, Medicago sativa
MELLSSLTKEEILEVLSHGLCHQCETLGHDRIRRTLHKRKRGETWYHGEGWEDIGDEIYMSMNGNGSNSESLNTRVRKKGKSPINSSLKHDSTLDENVGKHGENLYPGGDLKDIGGAIHMNGNASLAKSLNKRIRIVYNPRNSLQKHDSTLTVKDNKPNFSKQSDTELLLNDGTVDVSSSKNELSEEQKEHIRYNSEIQCKKDFTFIERINGRDINLLQGLELHTDVFNATEQDEIVEYIYGLQRRGQQGRLRDRTYSKPRKWMRGKGRETLQFGCCYNYAVDKYGNPPGICRTEEVDPLPDVFKQMIKRMVRWNIIPPTCVPDSCIVNIYDVGDCIPPHIDHHDFVRPFYSVSFLNEAKILFGSNLKEIQPGEFSGPASISLPLGSVFVLNGNGADIAKHCIPSVSSKRISITFRKMDERKLPYKYPPDPELVGIKSLSDSHLNKPDRNSETQVKKAGSVQSKPNPVLKTRHESSFHIKDEYPQTFYGNFSNFRKLKDSEFERTERH

SEQ ID NO: 17 - DNA, 1527 bp, Medicago sativa
  source 1..1527: mol_type = unassigned DNA
atggaacttctgagttcattgacgaaagaagagattttagaagttctatctcatggtttatgtcatcagtgtgaaaccctagggcatgatcgtatcagaagaacccttcataaaagaaagcgcggtgagacttggtatcatggtgaaggatgggaagatattggagatgaaatatacatgtctatgaatggtaacggttcaaattcagaatctttgaatacaagagttagaaaaaaggggaagagtccaataaactcttcactaaaacatgattctactctcgatgagaatgtaggaaaacacggtgagaatttgtatcccggtggagacttgaaagatattggaggagcaatacacatgaatggcaatgcttcacttgcaaaatctttgaataaaagaattagaatagtgtacaatccaaggaactctttacaaaaacatgattctactctcacagtgaaggataacaaacccaattttagcaagcagtctgatactgaattattgttaaatgatggaactgtggatgtttcttcatctaaaaatgagttatcggaagagcaaaaggagcatatccgttataattctgaaattcaatgcaaaaaagattttaccttcattgaaagaatcaatgggagagacataaatttgctgcagggtcttgaacttcacaccgatgttttcaatgctacggagcaggatgaaattgtcgagtatatatacggattgcaaaggagaggacaacaaggaagacttagagatagaacatactcgaaacctagaaagtggatgcgaggtaaaggacgtgaaaccctgcaatttggctgctgctacaattatgcagtggacaaatatggtaaccctcctggtatatgtagaactgaagaggttgatcctttgccggacgtgttcaagcaaatgataaagagaatggttagatggaacataattcctccgacgtgtgttcctgacagttgcattgtcaacatatatgatgtaggggactgcattcctcctcacattgaccatcatgactttgtcaggccgttttattccgtctcattcttgaacgaagctaaaatattatttggttcaaatctgaaagaaattcaacccggagagttttcgggtcctgcttctatttctttgcctctagggtcagtattcgttttgaatggtaatggagcagatattgctaaacattgtattccatctgtgtcgtcaaaaaggatttctattacatttagaaagatggatgagagaaaattaccatacaagtatccacctgatcctgagctagtgggaatcaagtcactttccgattcccatctaaataaaccggataggaattcagaaacacaagtaaagaaggctggaagtgttcaatccaaacctaatccagttttaaagactagacatgaatcgtcgtttcacatcaaagacgagtaccctcagactttttatggtaacttcagtaactttagaaaattgaaggacagcgaattcgaacgcacagaaagacattaa

SEQ ID NO: 18 - protein, 611 aa, Sorghum bicolor
MAMAPSSSSSGGGGGDSAGAADPMALVQGYTAEELAIAGEFLTTWLPFLSAGLCPSCVSSLRGRVDSLLPRAEESPPPPSPRQPVDQIEPSGWDSDPALQQNLPFEPSGWDSDPPPPAPAPAPAEKPRMSWADMAQEDELAAAAEDDAAAAAAEDGDEGAEAGRPKVQPTREQREQRRLSSIVRNKDFRCYERVHGQLVNILAGLELHAGVFSAAEQRRIVQCVYDLQARGQRGELGERTYTEPQKWMRGKGRVTIQFGCCYNYATDKNGNPPGIIRTIVSDPMPDLFKTMIKRLVRWQVLPTTCVPDSCIVNIYEPGDCIPPHIDSHDFVRPFCTVSFLSECNILFGSNLKASAPGEFTGSIATPLPVGSVLVLNGNGADVAKHCVPAVPTKRISITFRKMDPAKRPFDFKDDPELLNLAPLGTAVQETGISSDEGKGKLPDAQITNLSKVSRGKRSKGRTTPGKYGSGILGEQPPRHAQAPAVEVLSLQSPHGQRPVSASSAERERNSAWRSRELRYQSSSPGVQSQADNSREWPRRSVHERLSSVEDGVESAERRPWMEHRQISLINRTLNNDMNSLSLTSRESGDQSRASARTLYNKPRRTRVILED

SEQ ID NO: 19 - DNA, 1836 bp, Sorghum bicolor
  source 1..1836: mol_type = unassigned DNA
atggccatggcgccctcctcctcttcctccggaggcggcggcggcgacagcgccggagccgccgacccgatggcgctcgtgcaggggtacacggccgaggagctcgccatcgcgggggagttcctcaccacctggctccccttcctctccgccggcctctgcccttcctgcgtctcctcgctccgcggccgcgtcgactccctgctcccccgagcagaggagtcgccgccgccgccgtcgccgcggcagccggtcgatcagattgagccgtcagggtgggactcggatccggcgctccagcagaaccttcctttcgagccgagcggttgggattcggatccgcccccacccgcgccggcgccggcgccggctgagaagccgaggatgtcgtgggcggacatggcgcaggaggacgagctcgccgcggcggcggaggacgacgcggcggccgcggctgccgaggatggggacgagggggccgaggcggggaggcccaaggtgcagcccacgagggagcagcgggaacagaggaggctcagtagcatcgtgcgcaacaaggatttcaggtgctacgagagagttcatggccagctcgtcaacatcctcgctggccttgagcttcatgccggcgtgttcagcgcagctgagcagcggcggattgtccaatgcgtgtatgacctccaggcgagagggcagcgtggggagctcggagagcgcacatatacagaaccccaaaaatggatgcgtggtaaaggccgggtaacaatccaatttggatgctgttacaattatgctacggataagaatggaaatccaccaggcatcatacggaccattgtttctgatccaatgcctgacctgtttaagactatgattaagagattggtacgatggcaagttctgcctacaacttgtgtaccagacagctgcattgtaaatatatatgaacctggagattgcatcccaccacatatagatagccatgattttgttaggccattttgcactgtttctttcctcagcgaatgcaatattctttttggatctaacctgaaagcttctgcccctggagaattcaccggttcaattgcgacccctctccctgtcggatctgtgcttgttctgaatggcaatggtgctgatgtagcaaagcattgtgttccagcagtccctaccaaaaggatatccattacattcagaaaaatggatccagcaaagcgtccattcgattttaaagatgacccagaactgcttaaccttgcccctctagggacagctgtacaagaaactggcatatcatcagatgaaggcaagggcaagctacctgacgcccaaataacaaatctgagcaaagtatccagaggcaagaggtctaaaggaagaacaactcctgggaaatatggaagcggcattcttggagagcagcctcctaggcatgcacaagcccctgcagtcgaagtgctgtcgcttcaaagtccgcatggccagcgccctgtctctgcctcaagtgctgagagggaaaggaattctgcttggaggtcaagggaattgagatatcaatctagttcacctggcgtgcaatctcaagctgacaacagcagggagtggccacgtcggtcggtgcacgagaggttgagttccgttgaagatggtgtggaatctgcggaaaggagaccttggatggagcataggcagatatcgttgataaaccgaacactaaacaatgacatgaactctctttcgctcacaagccgtgaatcaggcgatcagtctcgtgctagcgcgcgaaccttgtataacaagcccaggaggaccagagtaatcctggaagattga

SEQ ID NO: 20 - protein, 615 aa, Zea mays
MAMAPSSSSSGGGGGDGANAADAMALVQGYTAEELAIAGEFLTTWLPFLSAGLCPSCASSLRGRVDSLITRGRAAEESPPVDQIEPSGWDSDPADPQNLPFEPSGWDSDPPPPPPPAPAPAPVEKPRMSWADMAQEDELAAAAEDDAAAAAADEGEDGAEAGRPKVPLTREQREQRRLSSLVRNKEFRCYERVRGQLVNILAGLELHAGVFSAAEQRRIVQCVYDLQARGQRGELGERTYTEPQKWMRGKGRVTIQFGCCYNYATDKKGNPPGIIRTIVSDPMPDLFKTMIKRLVRWQVLPATCVPDSCIVNIYEPGDCIPPHIDSHDFVRPFCTVSFLSECNILFGPNLKVAAPGEFIGSIAIPLPVGSVLVLNGNGADVAKHCVPAVPTKRISITFRKMDPAKRPFGFEDDPELLNLAPLGTVVQETGISSDEGKGKLPDVQITNMGKASRGKRSKGRTAAGKYGSGILGEQSPGHAQAPAVEVQSLRNPHGQRPVSASSAERERNSAGRSRESRHQSSTLVVHSQVDNSRQWPRRSAQERWHSNGMSFSEDGVGSAERRPRVEHRQISLINQTISDDMDSLSLSGRESGDQPRASVRTVYNKPRRTRLSLDD

SEQ ID NO: 21 - DNA, 1848 bp, Zea mays
  source 1..1848: mol_type = unassigned DNA
atggccatggcgccgtcctcctcttcctccggtggcggcggcggcgacggcgccaacgccgccgacgcgatggctctcgtgcaggggtacacggccgaggagctcgccatcgcgggggagttcctcaccacctggctccccttcctctctgccggcctctgtccctcctgcgcctcctcgctccgcggccgcgtcgactccctgatcacgcgaggccgcgcagctgaggaatcgccgccggtcgatcagattgagccgtcagggtgggactcggatccagcggacccgcagaaccttcctttcgagccgagtggttgggattcggatccgcccccacccccgccgccggcgccggcgccggcgccggtggagaaaccgaggatgtcgtgggcggacatggcgcaggaggacgagctcgccgcggcggcggaggatgacgcggcggccgcagcggccgacgagggggaggacggggccgaggcggggaggcccaaggtgccgctcacgagggagcagcgggaacagaggaggctcagtagcttggtgcgcaacaaggagttcaggtgctacgagagagttcgtggccagctcgtcaacatcctcgctggccttgagcttcatgccggcgtgttcagcgcagctgagcagcggcgaattgtccaatgtgtgtatgacctccaggcaagagggcagcgtggggagctcggagagcgcacatatacagaaccccaaaaatggatgcgtggtaaaggccgggtaacaatccaatttggatgctgttacaattacgctacggataagaaaggaaatccaccaggcattatacggaccattgtttctgatccaatgcctgacctctttaagactatgattaagagattggtacgatggcaagttctgcctgcaacttgtgtaccggacagctgcattgtaaatatatatgaacctggagattgcatcccaccacatatagatagccatgattttgttaggccattttgcactgtttctttcctcagcgaatgcaacattctttttggacccaacttgaaagttgctgcccctggagaattcatcggttcaattgcgatccctctccctgtcggatctgtgctagtcctgaatggcaatggtgctgatgtagcaaaacattgtgtcccagcagtccctaccaaaaggatatccattacattcagaaaaatggacccagcaaagcgtccatttggctttgaagatgacccggaactacttaatcttgctcctctagggacagttgtacaagaaactggcatatcatcagatgaaggcaagggcaagctacctgacgtccaaataacaaatatgggcaaagcatccagaggcaagaggtctaaaggaagaacagctgctgggaaatatgggagcggcattcttggagaacagtctcctgggcatgcccaagcccctgcagtggaagtgcagtcactacggaatccgcatggccagcgtcctgtctccgcttcaagtgctgagagggaaaggaattctgctgggaggtcaagggaatcgagacatcaatctagtacgcttgtggtgcactcacaagtggacaatagcaggcagtggccacgtcggtcggcgcaagagaggtggcatagtaatggcatgagtttctctgaagatggtgtgggatccgcggaaaggagaccacgggtggagcatagacagatttcattgataaaccaaactataagcgacgacatggattccctgtcgctctcaggccgtgaatcaggcgaccagcctcgtgctagtgtgcggaccgtgtataacaagcccaggaggaccagattaagcctagatgattga

SEQ ID NO: 22 - protein, 573 aa, Zea mays
MTTAAGMPGLPVGSAAAAGVPAAPEVAARDAVIGWFRGEFAAANAMIDALCGHLAQIGGGGPEYDPVFAAIHRRRANWFPVLHMQKFYPVADVAAELRRVADARAAAGSCCYSEEAASTVIHEPMEEAEAEAEAEPEPEQEHEQDPAPAAELADGAVEYHEPDVEVDSSGDSSERKPPSTEDDTVADGHHTDQGSQGEHSLPESYPICSDHEECIARPERIKIQKGFVAKESVKGHMVNVVKGLKIYEDAFTTSEIMKVADFINEIRQAGRNGELSGETFIFFNKQIKGNKREIIQLGVPLFQPTTEDANCHTEPIPLVLQAVIDHLVLWRLIPESRKPNSVVINFFDEDEHSQPYFKPPHLDNPICTLLLSETTMAFGRSLVTDSNGNYKGPLTLSLKQGSLLVMRGNSADMARHVVCPSSNRRVSITFARVRPSTPVDMSPLPSPTKAMTPWQPQPVTASQVPSPVSMTQKPPVSGAIIGYAPTPQAVLAPAAWGMAMRAPVMMVAAAPARPMVMASSGTGGGNISKRMGRSGTGVFLPWTVGPKRYNKHLPPRIQKRRFSAMMSPIEAQG

SEQ ID NO: 23 - DNA, 1722 bp, Zea mays
  source 1..1722: mol_type = unassigned DNA
atgacgacggcggccggcatgccggggttgccggtgggctcggccgccgcggctggggttcccgcggcgcccgaggtggcggccagggacgcggtgatcggctggttccgcggcgagttcgcggccgcgaacgccatgattgacgcgctgtgcggccacctggcgcagatcggcggcggcgggcccgagtacgatcccgtcttcgccgcgatccaccgccgccgcgccaactggttccccgtcctccacatgcagaagttctaccccgtcgcggacgtcgccgccgagctgcgccgagttgcggacgcccgcgccgccgcagggtcctgctgctactccgaggaggccgcgtccacggtgatccacgagcccatggaagaggctgaggctgaggctgaggctgagcccgaacccgagcaggagcacgagcaggatccggcaccggcggccgagctggctgacggcgccgtggagtaccacgagccggacgtggaagttgattcatcgggagattcgtcggagcggaagcccccctccaccgaagatgacaccgtcgcagacggacatcatactgatcaaggatctcaaggggaacacagcctaccagagagctatcccatctgctctgaccacgaggagtgcattgcccgtcctgagaggatcaagatccagaaaggtttcgtggccaaggaatccgtgaaggggcacatggttaatgtcgtgaaaggcttgaagatatatgaagatgcgtttacgacatcagagatcatgaaagttgcagatttcatcaacgaaattcgtcaggctggcagaaatggagaactttcaggtgagactttcatattcttcaacaagcagatcaaaggaaacaagagggagatcatacagcttggtgtaccgttatttcaacctaccacagaggatgctaattgtcacacagaaccaatcccccttgtcctgcaggctgtcattgaccaccttgttctttggcgtttaataccagaaagcaggaaaccaaatagtgtcgtcatcaacttcttcgatgaggatgaacattcgcagccctacttcaagcctccccatcttgacaaccccatttgcactcttctgctgtctgaaaccacgatggcatttggcaggtcacttgtcactgacagcaacggtaactacaaaggacctctcacactttcactaaagcaagggtccttactagtgatgcgtgggaacagtgccgacatggcgcgacatgtggtgtgcccgtcgtccaaccggcgcgtgagcatcacgttcgcaagggtgaggccatcgacgccggtggacatgagcccactcccgtcccccaccaaggccatgacaccctggcagccgcagccggtcacggcctcccaggtgccgtcgcccgtgagcatgacgcagaagcccccggtcagcggtgccataatcggttatgccccgaccccgcaggccgtgctggcgcccgcagcctggggcatggccatgcgcgcgcccgtcatgatggtggctgcagcgcctgcgaggcccatggtgatggcctcatcgggcaccggcggcggcaacatcagcaagcggatgggccgcagcggcaccggcgtgttcctgccttggacggtcggcccgaagcggtacaacaagcaccttccaccgcgcatccagaagcgccggttctcggcgatgatgtcgcccatcgaggcgcagggctga

SEQ ID NO: 24 - protein, 612 aa, Triticum aestivum
MAAGDGADPMELVRGQYDADELAIAGEFLTTWLPFLSAGLCPSCVGSLRGRVASLLPPPPPPTPRAEEPAPPPPVERVVATGWDSDPAAPRHLPFEPSGWDSDPPPPPPQRQQAEAEKPRMSWADMAQEDELAAAAAAADDDGEEGEEEEAGGAGRPRARLTREQRELHRFRNVLRKDDFICLERVKGQLVNILAGLELHAGVFSTAEQKRIVDCVYGLQEMGKRGELGDRTYTEPEKWMRGKGRVTIQFGCCYNYATDRKGNPPGIIRTFVSDPIPELFKVMIKRLVRWHILPTDCVPDSCIVNMYDPGDCIPPHIDSHDFVRPFCTVSFLSECNILFGSSLKIAGPGEFTGSFAIPLPVGSVLVINGNGADVAKHCVPAVPSKRISITFRKMDPAKRPFSFKDDPELLNITPLEAPAAPETSRSSDEGKGKQLGVQTRDPGSRSSRSRKSKGRTTPAGKAGWRGILGDQPPQHPQSPISSVSSERERDSIGRSREPRYPPGSDRERDSVERPREPRQPHDTPSHGEDLRDRLNRPPHERTPGSGVFFVNNGADSQARGQRMEHRQLQMINRTINDDMDSLSVGSHESSDQPRVSVRTIHNRPRTRINLGW

SEQ ID NO: 25 - DNA, 1839 bp, Triticum aestivum
  source 1..1839: mol_type = unassigned DNA
atggccgccggcgacggcgccgacccgatggagctcgtgcgggggcagtacgacgccgacgagctcgccatcgccggggagttcctcaccacctggctccccttcctctccgcgggcctctgcccctcctgcgtcggctcgctccgcggccgcgtcgcctccctcctccccccgccgccgcccccgacgccgcgagcggaggagccggcgccaccgccgcccgtcgagagggtcgttgccacggggtgggactcggatccggccgccccgcggcatctcccgttcgagcccagcgggtgggactcggatccgcccccgccgcccccgcagcggcagcaggcggaggcggagaagccccggatgtcgtgggcggacatggctcaggaggacgagctcgccgccgccgccgcggccgccgacgacgacggggaggagggggaggaggaggaggccggcggggcggggaggccgagggcgaggctgaccagggagcagcgggagctgcatcggttccggaacgtgctgcggaaggacgacttcatatgcttggagagggtcaagggccagctcgtcaacatcctcgccggcctggagctccacgccggcgtgttcagcaccgccgagcagaagcgcatcgtcgactgcgtctacggcctgcaggagatgggcaagcgcggggagcttggagatcgtacatatacagaacctgagaaatggatgcgtggtaaagggcgggtaacaattcaatttggatgctgttataactatgctacggacaggaaaggaaatccaccaggcatcattcgaacttttgtttctgatccgatccctgaactatttaaggtcatgatcaagagattggtaaggtggcatattctgccaacggattgtgtaccagacagttgcattgtcaacatgtatgaccctggagattgcatcccaccacatatagacagccatgattttgttcgaccattttgtactgtttcattcctcagtgaatgcaacatactttttgggtctagtctgaaaatcgctggtcctggagaatttacgggctcatttgcaattcctctgcccgttgggtctgtgctcgtcataaatggcaatggtgctgatgttgcaaagcattgtgttccagcggtaccatccaaaaggatatccattaccttcagaaaaatggatcctgcaaagcgtccattcagtttcaaggatgatcctgaactgctgaacattactcctctcgaagcaccagctgcacccgagactagcagatcctcggatgaaggcaaggggaagcagcttggcgtacaaactcgggatccaggcagcagatcatccagaagcaggaaatccaaaggaagaacgacccctgctggaaaggctggatggaggggcattcttggagaccaacctccacagcacccacagagccccatctccagtgtgagctctgagagagagagggactccattgggaggtcgagagagccaaggtatccaccgggttctgacagagaaagggactccgttgagaggccgagagagccaagacaaccgcatgacacgccatctcatggcgaggacctcagagaccggctgaacaggccgcctcatgagaggacgcctggcagcggcgtgttcttcgtcaacaatggcgcggactcccaggccagggggcaacggatggagcacaggcagctacagatgatcaaccgcaccatcaacgacgacatggactccctctccgtcgggagccacgagtcgtctgaccagccccgtgtcagcgtgcggaccatacacaacagaccaaggaccagaataaacctgggctggtag

SEQ ID NO: 26 - protein, 566 aa, Triticum aestivum
MTTPAAGAPGSPVAAPDQVAARDAVIGWYRGEFAAANAVIDALCGHLAQIGGADYDAVFAALHRRRLNWFPVIHMQKFYSVADVAAELRRVSEARAAAAAAYSEEEAASTVIHEPMDELVVTVAAEPDPEPEREHEPIPEAQPDVSVNPVVAVHPVAADHEPEADGEDSSGDSSERKAASTEDDAAHDGPDNTDQGSQGGHSLPESYPICSDHDECIARPERIKIQKGFMAKESVKGHMVNVVKGLKIYEDVFTTMELMKVADFINEIRQAGRNGELSGETFIFFNKQIKGNKREIIQLGVPLFQHTTEETNCHIEPIPVVLQAVIDHLVLWRLIPESRKPNSVIINFFDEDEHSQPYFKPPHLDNPISTLLLSETSMAFGRSLVTDSNGNYKGPLTLSLKQGSLLVMRGNSADMARHVVCPSSNRRVSITFVRVRPSTPVDLSPLPSPTKAMTLWQPPPTAATAGMQKPPHGSNGAIIGYCPAPQAMLAPAWGMAVRAAPVMMVAAPARPMVMAPSSNINKRMGRGGTGVFLPWTVGPKRYNKHLPPRIQKRRFSAMMSPIESQG

SEQ ID NO: 27 - DNA, 1701 bp, Triticum aestivum
  source 1..1701: mol_type = unassigned DNA
atgacgacaccggcggcgggggccccggggtcaccggtggcggcgccggatcaggtggcggccagagacgccgtgatcgggtggtaccgcggcgagttcgcggccgccaacgccgtgatcgacgcgctctgcggccacctcgcgcagatcggcggggccgactacgacgccgtcttcgccgcgctccaccgccgccgcctcaactggttccccgtcatccacatgcagaagttctactccgtcgccgacgtcgccgccgagctccgccgcgtctccgaggcccgcgccgccgcggccgccgcctactccgaggaggaggccgcgtccacggtgatccacgagcccatggacgagctcgtcgtcaccgttgccgcggagccggaccccgagcccgagcgcgagcacgagccgataccggaggcgcagcccgacgtctccgtcaaccccgtcgtcgccgtccaccccgtcgcggcggaccacgagccggaggcggacggcgaagattcctcgggagattcgtcggagcggaaggcagcctccacggaagacgacgccgcccacgacggaccagataacaccgaccaaggatctcaaggcgggcacagcctaccagagagctaccctatctgctctgaccacgacgagtgcatcgcccgcccagagaggatcaagatccagaaaggtttcatggcgaaggagtccgtgaaggggcacatggttaacgttgtgaaaggcctgaagatatatgaagatgtgttcacaacaatggagctcatgaaggttgctgatttcatcaatgaaattcgccaggctggtagaaatggggagctttcaggtgagaccttcatattcttcaacaagcagatcaaagggaataagagagagatcattcagctcggtgtcccactatttcaacataccacagaggaaaccaattgtcatatagaaccaatcccagttgtcctgcaggctgtcatcgaccaccttgttctttggcgcttaataccagaaagcaggaaaccaaacagtgtcatcatcaatttcttcgatgaggacgagcactcgcagccctacttcaagcctccccacctggacaaccccatttccactctcctgctgtctgaaacctcaatggcatttggaaggtcacttgtcactgacagcaacggcaactacaagggacccctcacactctcactgaagcaagggtcacttctggtgatgcgcgggaacagcgcggacatggcgcgccacgtcgtgtgcccgtcgtccaaccgccgcgtgagcatcacgttcgtgagggtgaggccatcaactccggtggacctcagcccgctcccgtcgcccaccaaggccatgacgctctggcagcctcccccgactgcggcaacggccggcatgcagaaaccgcctcacggcagcaacggcgccatcatcggctactgcccggcgccacaggccatgctcgccccggcttggggcatggccgtgcgcgcggcacccgtcatgatggtcgccgcgccggcgaggccaatggtgatggcgccctccagcaacatcaacaagaggatggggcgcggcggcaccggcgtgttcctgccgtggacggtggggcccaagaggtacaacaagcacctccccccgcgcatccagaagcgccggttctcggcgatgatgtcgcccatagagtcgcagggctga

SEQ ID NO: 28 - protein, 331 aa, Homo sapiens
YLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFE

SEQ ID NO: 29 - protein, 215 aa, Homo sapiens
KRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQ

SEQ ID NO: 30 - protein, 292 aa, Arabidopsis thaliana
GSEIVFGSFPEDFSSVLQSRPAVVETASPRMRWADMAQEDEFDEEEEEEEEERDSSRKGFDASSMKTPEKPKLSRDQRENLRLINVKRKKDFICLERVKGKIVNVLDGLELHTGVFSAVEQKRIVDQVYQLQEKGRRGELKKRTFTAPHKWMRGKGRETIQFGCCYNYAPDRAGNPPGILQREEVDPLPHLFKVIIRKLIKWHVLPPTCVPDSCIVNIYDEGDCIPPHIDNHDFLRPFCTISFLSECDILFGSNLKVEGPGDFSGSYSIPLPVGSVLVLNGNGADVAKHCVP

SEQ ID NO: 31 - protein, 296 aa, Arabidopsis thaliana
KQKKTEEEAEEDLKEVVATEEEEVKKECFNGEKVTENDVNGDVEDVEDDSPTSDITDSGSHQDVHQTVVADTAHQIICHSHEDCDARSCEIKPIKGFQAKEQVKGHTVNVVKGLKLYEELLKEDEISKLLDFVAELREAGINGKLAGESFILFNKQIKGNKRELIQLGVPIFGHVKADENSNDTNNSVNIEPIPPLLESVIDHFVTWRLIPEYKRPNGCVINFFEEGEYSQPFLKPPHLEQPISTLVLSESTMAYGRILSSDNEGNFRGPLTLSLKQGSLLVMRGNSADMARHVMC

SEQ ID NO: 32 - protein, 300 aa, Micromonas commoda
VPTGAYITPEHDEYDACVARSYVGFVVDPPDILPDELHADVAAAFKTMAKRGLFTRDVLAAGKTVSPTFVSRTLVGERGMTYHYQKLRIFALPWSDEDTPEGSPLRVVRRLNDAMKRRSRALLTRHRDARVPGDVTGSCEFNVTLINLMEPERKESVALKDEGQFGMGKASVSWHSDSSLQDTSTVAVYHQTSGGIDGDDTSWHVALKCLDGVTPALRVPLRSHSAYYIMRDFNLHHHHAVLNGDTRRFSSTHRVAVTAKDTFDYVRGRCERALAVLPSLKWRAGRWPKGTKPPRDASST

SEQ ID NO: 33 - protein, 287 aa, Ostreococcus tauri
QSRARPHCASPKMPDVLGPGDGDAYERVRSFHYDGFVVDDPEILPEALHDDVARAFDSLLREGEFTHDVLSAGNKVSRTFVTRTLLGDRGTTYHYQKLRLFAQPWDGEKRWEYATARRLNEALTRRTREILRGTRHGESECEYNVTLINRMDTREESEVALKDEALFDLGTTSVSWHSDSSLRENSTVAVYSTVDDPERADWSVALRALGKQCPALRVPLKDKATYYMCGDFNATHHHAVLTGSSARYSSTHRVGVVEKDTFQYIKRRCLEALALIPDLENKSASLD

SEQ ID NO: 34 - protein, 308 aa, Micromonas pusilla
GTSGSDDDDDDAAPSFLTPSHAEYERCMKTSYLGFHVDDPSALPEKTHREIARAFERMASDGIFHRDVLAAGKSVSPTFVSRTLLGERGMTYHYQRLRIFAHPWDEETQPDASHPFRAVKRLNDALVSRSTAVLRASSDPRLGDGVDGSCDFNVTLINLMEPASRCAEVPLKDEALFGMGKARSTSVSWHSDSSLQDVSTVAVYHQTDDDDDDSWAVALKTLDGVTPAMRTPLRSREMYYMLRDFNAHHHHAVITGESRRFSSTHRVAVVAKDTFEHIKTRCERALRLLPGIKKIAQVGPGPGDDLAA

SEQ ID NO: 35 - protein, 275 aa, Ostreococcus lucimarinus
SSSVLEPEDGEPFARVHRAHYRGFVVDAPSVLPASLHDDVERAFDDMRSRGEFTHDVVSAGNKVSTTYVRRCLLGEDGMTYHYQKLRLFAQPWRGREAYEVVRRLNETLTRSARERCEKLGGAFAESECEYNVTLINYMETEGESEIELRNEEKFDLGTTSVSWHSDSSLRENSTVAVYHTYEAPERKDWRVALRALNAECEVLCVPLEDKATYYMCGEFNATHHHAVLTGSSARYSSTHRVAVVAKDTFQYIKRRCIDALAIVPDLERENKPLD

SEQ ID NO: 36 - protein, 154 aa, Homo sapiens
PEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHR

SEQ ID NO: 37 - protein, 136 aa, Arabidopsis thaliana
PTCVPDSCIVNIYDEGDCIPPHIDNHDFLRPFCTISFLSECDILFGSNLKVEGPGDFSGSYSIPLPVGSVLVLNGNGADVAKHCVPAVPTKRISITFRKMDESKRPVWFTPEPDLQGIEPLPLDLNRSGSTSRFSR

SEQ ID NO: 38 - protein, 152 aa, Oryza sativa
KTCIPDSCIVNIYDPGDCIPPHIDSHDFVRPFCTVSFLSECNILFGSTLKIAGPGEFTGSLPIPLPVGSVLILNGNGADVAKHCVPAVPTKRISITFRKMDPAKRPFNFRDDPELLNIIPLETAVQETGRSSDEGKGKQPDIQIRNPSKAHR

SEQ ID NO: 39 - protein, 160 aa, Oryza sativa
SSPDYCVIDIFNEGDYSHPHHHPPWYGRPICTLCLTDCDMVFGHVIAADSRGDHAGPLKLSLSTGSVLVFEGKSADIAKRALPATSKQRILLSFGKSVSRKHVQSESSLLITPPLTPPPMPWAAPLRPGNIAIHPSSPKQLVYNPSNRVPAVSTPGLHHI

SEQ ID NO: 40 - DNA, 21 bp, synthetic construct
  source 1..21: mol_type = other DNA
ccaaaaaagaagagaaaggta

SEQ ID NO: 41 - protein, 7 aa, synthetic construct
PKKKRKV

SEQ ID NO: 42 - protein, 221 aa, Homo sapiens
QQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLET

SEQ ID NO: 43 - protein, 124 aa, Arabidopsis thaliana
PHLFKVIIRKLIKWHVLPPTCVPDSCIVNIYDEGDCIPPHIDNHDFLRPFCTISFLSECDILFGSNLKVEGPGDFSGSYSIPLPVGSVLVLNGNGADVAKHCVPAVPTKRISITFRKMDESKRP

SEQ ID NO: 44 - protein, 127 aa, Arabidopsis thaliana
PLLESVIDHFVTWRLIPEYKRPNGCVINFFEEGEYSQPFLKPPHLEQPISTLVLSESTMAYGRILSSDNEGNFRGPLTLSLKQGSLLVMRGNSADMARHVMCPSQNKRVSITFFRIRPDTYHNHSQP

SEQ ID NO: 45 - protein, 612 aa, Triticum aestivum; identical to SEQ ID NO: 24

SEQ ID NO: 46 - DNA, 1839 bp, Triticum aestivum; identical to SEQ ID NO: 25
  source 1..1839: mol_type = unassigned DNA

SEQ ID NO: 47 - protein, 531 aa, Triticum aestivum
MTTPAAGAPGSPVAAPDQVAARDAVIGWYRGEFAAANAVIDALCGHLAQIGGADYDAVFAALHRRRLNWFPVIHMQKFYSVADVAAELRRVSEARAAAAAAYSEEEAASTVIHEPMDELVVTVAAEPDPEPEREHEPIPEAQPDVSVNPVVAVHPVAADHEPEADGEDSSGDSSERKAASTEDDAAHDGHNTDQGSQGGHSLPESYPICSDHDECIARPERIKIQKGFMAKESVKGHMVNVVKGLKIYEDVFTTMELMKVADFINEIRQAGRNGELSGHIEPIPVVLQAVIDHLVLWRLIPESRKPNSVIINFFDEDEHSQPYFKPPHLDNPISTLLLSETSMAFGRSLVTDSNGNYKGPLTLSLKQGSLLVMRGNSADMARHVVCPSSNRRVSITFVRVRPSTPVDLSPLPSPTKAMTLWQPPPTAATAGMQKPPHGSNGAIIGYCPAPQAMLAPAWGMAVRAAPVMMVAAPARPMVMAPSSNINKRMGRGGTGVFLPWTVGPKRYNKHLPPRIQKRRFSAMMSPIESQG

SEQ ID NO: 48 - DNA, 1683 bp, Triticum aestivum
  source 1..1683: mol_type = unassigned DNA
atgacgacaccggcggcgggggccccggggtcaccggtggcggcgccggatcaggtggcggccagggacgccgtgatcgggtggtaccgcggcgagttcgcggccgccaacgccgtgatcgacgcgctgtgcggccacctcgcgcagatcggcggggccgactacgacgccgtcttcgccgcgctccaccgccgccgcctcaactggttccccgtcctccacatgcagaagttctactccgtcgccgacgtcgccgccgagctgcgtcgcgtctccggcgcccgcgccgccgcggccgccgcctactccgaggaggaggccgcgtccacggtgatccacgagcccatggacgagctcgtcgtcaccgtcgccgcggagccggaccccgagcccgagcgcgagcacgagccgataccggaggcgcagcccgatgtctccgtccaccccgtcgacgcggaccgcgagccggaggcggacggcgaagattcctcgggagattcgtcggagcggaaggcggcctccacggaagacgacgccgcccacgacggaccagataacaccgaccaaggatctcaaggcgggcacagcctaccagagagctaccctatctgctctgaccacgacgagtgcatcgcccgcccagagaggatcaagatccagaaaggtttcatggcgaaggagtccgtgaaggggcacatggttaacgttgtgaaaggcctgaagatatatgaagacgtgttcacaacaatggagctcatgaaggttgctgatttcatcaatgaaattcgccaggctggtagaaatggggaactttcaggtgaaaccttcatattcttcaacaagcagatcaaaggaaacaagagagagatcattcagctgggtgtcccactatttcaacataccacagaggaaaccaattgtcatatagaaccaatcccagttgtcctgcaggctgtcatcgaccaccttgttctttggcgcctaataccagaaagcaggaaaccaaacagtgtcatcatcaatttcttcgatgaggacgagcactcgcagccctacttcaagcctccccacctggacaaccccatttccactctcctgctgtctgaaacctcgatggcatttggaaggtcacttgtcactgacagcaacggcaactacaagggaccgctcacactctcactgaagcaagggtcacttctggtgatgcgcgggaacagcgcggacatggcgcgccatgtcgtgtgcccgtcgtccaaccgccgcgtgagcatcacgttcgtgagggtgaggccatcgactccggtggacctcagcccgctcccgtcgcccaccaaggccatgacgctctggcagcctcccccgactgcggcaacggccggcatgcagaaaccaccccatggcagcaacggcgcaatcattggctactgccccgcgccgcaggccatgctcgccccggcttggggcatggccgtgcgcgcggcgcctgtcatgatggtcgccgcgccggcgaggccaatggtgatggcgccctccagcaacatcaacaagaggatggggcgcggaggcaccggcgtgttcctgccgtggacggttgggcccaagaggtacaacaagcacctccccccgcgcatccagaagcgccggttctcggcgatgatgtcgcccatagagtcgcagggctga

SEQ ID NO: 49 - protein, 493 aa, Manihot esculenta
MMEGDMSRKSPEARRGEDFIELLKTMDHEEVLQVLSGGFCQHCQALLETRIHNLINAKVNKMSLSNGGTEVSKEFQSADAISINSSNGNLESLSPGKASLTSSSNQESESYSARQKGVTANSSSASVNSHQKSIDHPGFEILLNDGLADVSSVVDNGLSEEQKEQFRFSRVGRKKDFVCSERINGKPTNILKGLELHTKVFNPEEQKKIVECVYNLQRMGRKGQLRERTYSEPTKWMRGKGRITMQFGCCYNYAMDKNGKAPGIVQDEVVDPLPPTFKQMIKRMVRWHVLPATCIPNSCIVNIYDEGDCIPPHIDHHDFLRPFCTVSFLTECNIVFGSNLKILSPGEFSGPVSIPLPVGSVLVLNGNGADIAKHCIPGVPAKRISITFRKMDDSKLPYQFSPDPELLGIKPLIYSPLTKSPYQQSQQQKPFGYPSVVKSLVQQSNHQNGNLTATKSRSGNTNGTSFFINKDDFPPLGSSKSSGPSRANRVGSK

SEQ ID NO: 50 - DNA, 1482 bp, Manihot esculenta
  source 1..1482: mol_type = unassigned DNA
atgatggaaggagatatgtcccgaaagtcgccagaagcccgtcgtggtgaggatttcatagaacttctcaagacaatggaccatgaagaggttttgcaggttttgtctggaggattttgccagcactgtcaggctcttttagaaactcgaattcacaatctcatcaatgcaaaagttaataagatgtctttgtccaatggtggcaccgaagtttcaaaagaatttcagtctgctgatgcaataagcatcaattcatcaaatggtaatttggaatctttgtctcctgggaaagcaagcctaacttcgagctctaaccaagaatctgaatcttactcagcgaggcaaaaaggggtcactgccaattcttcatctgcatcagtgaacagccatcagaaatcaattgatcatccaggttttgaaatactgttgaatgatggacttgcagacgtgtcttctgtggtggacaatggcttatcagaggagcaaaaggagcagtttaggttttcaagagttggcaggaaaaaggattttgtgtgttctgagagaatcaatgggaagccaaccaatattcttaaagggcttgaacttcataccaaggttttcaatcctgaagagcagaagaaaatagttgaatgtgtctacaatctacagcgcatgggcaggaaggggcaactcagagaacgcacatattcagaacctacaaaatggatgcgtggtaaaggacgtatcacaatgcaatttggatgctgttacaattatgcaatggacaaaaatggaaaggccccaggaatagttcaagacgaagttgttgatcctctaccacccactttcaagcaaatgatcaagagaatggttaggtggcatgtgttgcctgccacatgcattcctaacagctgcatcgtgaatatatatgatgaaggagattgcattcctccccatattgatcatcatgactttcttcggccattttgtactgtgtcattcttgactgagtgcaacatagtcttcggttcaaatttgaagattcttagtccaggagagttctctgggcctgtctctatacctttgcctgtcggatcggttcttgttctaaatggtaatggagcagacattgctaagcactgtattccaggagtcccggcaaaaaggatttccataacattcaggaaaatggatgacagcaaactaccataccagttctcaccagatcctgagctgttgggaattaagcctctgatatattcgcccttgacgaagtcgccataccaacaaagccagcagcaaaaaccttttggttatccttcagttgttaagtcgctggtccaacagagcaaccaccagaatggcaacctcactgcaacgaaatctagaagtggcaacactaatggcacctcatttttcatcaataaagatgattttcctccacttggcagctcaaagtcttctggtccatccagggcgaatagagttggatcaaaatga

SEQ ID NO: 51 - protein, 690 aa, Manihot esculenta
MAMPTGNVVISDKMQFPVAGGGSGGGVGTEIHQQHHHRHQWFPMDERDGFISWLRGEFAAANAIIDSICHHLRSVGEPGEYDLVIGCIQQRRCNWNPVLHMQQYFSVGEVIVALQQVALRKQQQQQQRYYYDQAKVGGKEFKRSWNAGFNKGQKGGGEVVKEAVNSSVESHSFDWNSSGNGGSEKFEEIKSSGDGGKVEDKSLALGEDKKDAVAKPHIDNHLKGSENSEGTLSENDAESEAVDEQSSSKENDERSSQNNGVKRSLPITPKTFVGSEIVDGKMANVVDGLKLYEHLLDNVEVSELVSLVNDLRASGRRGQFQGHTYVVSKRPMKGHGREMIQLGLPIADAPVEDDAAGTSKDRRIESIPTLLQDVIDQFVSKQIITVKPDSCIIDIYNEGDHSQPHMWPPWFGKPISVLFLTECDLTFGRVITADHPGDYRGSLKLPLAPGSLLVMQGKSTDFAKHAIPAIRKQRILVTFTKSQPKKFAQSDGQRLPSSAGAPSHHWGPPPSRSPNHIRHPVPKHYPVIPSSGVLSAPPIRPQIPPPNGVQPLFVTAPVAAPMPFPAPVPIPPVSTGWPAAPPRHPPNRLPVPVPGTGVFLPPPGSGNASSPQISAATTEINFPAETSSVLDKENGIGKANLVSCASPKEESDGKSQRQDCNGISDGRSLSKEEQQQNADHTVVNKSTGTV

SEQ ID NO: 52 - DNA, 2073 bp, Manihot esculenta
  source 1..2073: mol_type = unassigned DNA
atggcaatgccaacgggaaatgtggtcatatctgataaaatgcagtttcccgttgctggcggtggtagtggtggcggagttgggactgagatccaccagcagcatcaccatcgccaccagtggttccccatggatgagcgtgacgggtttatctcgtggctgaggggggagtttgcggccgccaacgctatcattgactccatttgccaccacttgcgctctgttggggaacccggtgagtatgacttagttattgggtgtatacagcagaggaggtgcaattggaaccctgtgctgcatatgcagcagtatttctcggtgggggaggtgattgttgcgttgcagcaggttgcactaaggaagcaacagcagcagcaacagagatattactatgatcaggcgaaagttggggggaaggagtttaagagatcgtggaatgctgggtttaacaaaggacagaaaggtggtggtgaggtggtgaaagaagctgttaattccagtgtcgaaagtcatagttttgattggaattcttcagggaatggagggagtgagaaattcgaggaaatcaaatcaagtggtgatggtggaaaagtggaggataagagtctagcacttggtgaggataagaaagatgctgttgcaaagccacatatagataaccacttgaagggttcagaaaattcagaaggaaccttgtctgagaatgatgctgagtctgaggctgtggatgagcaatccagctctaaagagaatgatgaaaggtccagccagaataacggtgtgaagcggagtctccctatcactccaaaaacatttgttggttcagagatagttgatggaaagatggctaatgtggttgatgggctcaaattatatgaacacttgcttgataatgtggaagtttcagagcttgtttcgttggttaatgatttgagagcttcaggtagaagaggacaatttcaaggtcatacatatgtggtctccaagagacctatgaagggacatggtagagagatgattcaattgggtcttcccatagctgatgcacctgtagaagatgatgctgctggaacttctaaagaccgcagaatagaatccatccctaccttgctgcaagatgtcattgatcaatttgtaagcaagcagataattaccgtgaagccagactcttgcataattgacatatataatgagggggatcactcgcagcctcatatgtggcctccgtggtttgggaagcctatttctgtgcttttcttgacagaatgtgacctgactttcgggagagtaatcacggcagaccatcctggggattacagagggtctctcaagctgcctcttgcacctggatccctccttgtgatgcaaggcaaatccacagattttgctaaacatgcaattcctgcaatccgaaaacaacgcatacttgttaccttcaccaaatctcaacccaaaaaatttgcacaaagtgatggccaacggctaccttcctccgctggagccccatctcaccactggggtcccccgccaagtagatcaccaaatcacattcgccatcctgtgcccaagcattatccagtaattccatcatctggtgtattgtcagctccacccatcaggccacaaattccccctccaaatggtgttcaaccactctttgtcactgcccccgttgcagctccaatgcctttccctgcaccagttcctatcccacctgtgtcaactggatggccagcagctcccccaaggcatcctccaaatcgtcttcctgtccctgtccctggcactggtgttttccttcctccaccaggttctggtaacgcatcgtctccacagatatcagcggctacaactgaaataaactttcctgcggagacatcatcagttctagataaggaaaatggaatagggaaggctaatcttgtttcttgtgcttctccaaaagaggaatcagatggaaagtctcaaagacaagattgtaatggaatttcagatggaagatctctgtcaaaggaagaacaacagcaaaatgctgatcatacagtggtgaacaagtcaactggaacagtttag

SEQ ID NO: 53 - protein, 473 aa, Vigna unguiculata
MEKQHHQQRKPNNINASDTDPFLLNYTPSDLRTASEFLATWLPFLSQDLCARCTQSLSDRIQSIDPGEVPQNDSPNDQNDAEDNCDTHSLGSWKDGAEVNSCVETPSQRVCWADMAQEDDEFGEEENNNNVVNVVMGNSNDASGVGAAVAEKPTLPPERREYLRFTSVRRKKDFACFERVNGKLVNILGGLELHTGIFSAAEQRRIVNYVASLQDMGKRGELKERTFSAPQKWMRGKGRQTIQFGCCYNYAVDRDGNPPGILPHSMVDPLPDLFKVIIRRLIKWHVLPPTCVPDSCIVNIYEEGDCIPPHIDNHDFVRPFCTVSFLSECNIVFGANLKIVGPGEFDGSIAIPLPVGSVLVLNGNGADVAKHCVPAVPAKRISITFRRMDESKRPFGYVPEPDLQGIQPLTYKAEDEKKSGGHRSSHHVRRQRDRRGGRNDAMGFASRNDRFSERDSNHNTPRSGNRWSRNLDG

SEQ ID NO: 54 - DNA, 1422 bp, Vigna unguiculata
  source 1..1422: mol_type = unassigned DNA
atggagaaacagcaccaccaacaacgtaaacccaacaacatcaacgcttccgacaccgaccctttcctcttaaactacacaccttcggatctccgaaccgcttcggagtttctcgccacgtggctccccttcctctcccaagatctctgcgcgcgctgcactcaatctctctccgatcgtattcaatccatcgacccaggtgaagtgccccaaaacgacagccccaatgaccaaaacgacgctgaagataactgcgacacgcactcgctggggagttggaaagatggggcggaggtgaattcctgtgtggagactcctagccagagagtttgttgggctgacatggcccaagaggatgatgagtttggcgaagaagaaaacaacaacaatgtggttaatgttgttatgggtaattcgaatgatgcttctggtgtgggtgcggctgtggctgagaagcctactttgcctccggagcggagggagtaccttaggttcacgagtgtgaggcgcaagaaagactttgcctgctttgagagggttaatgggaagcttgtcaacattcttggagggcttgaactccacactgggattttcagtgctgctgaacagagaagaattgtcaattatgttgcttcattacaggacatggggaaaagaggagaattgaaagagcgaacattttctgctccccaaaagtggatgaggggcaagggacgacaaactatccaatttgggtgctgttacaattatgcagtggatagagatggtaacccacctggtattcttccacattcaatggttgatcctttacctgatcttttcaaagtcatcatccgacgactgatcaagtggcatgtactacctcctacttgtgtgccagacagttgcattgttaatatatatgaagaaggggactgtatacctccacacatagataaccatgactttgttcgaccattttgcactgtctcatttcttagcgagtgcaatatagtttttggagcaaacttaaagattgtaggccccggtgaatttgatggttcgattgctattccattacctgtgggatctgtacttgtcttgaatggaaatggagctgatgtagctaagcactgtgtgccagcagttcctgcaaaaaggatatcaatcacatttagaagaatggacgagtcaaagaggccttttgggtatgttcctgaacctgatcttcagggaatccaaccattgacctataaagctgaggacgaaaagaaatcaggtggacacagatctagtcatcatgtgagaagacaaagagaccggagaggtggcaggaatgatgcaatgggatttgcatctagaaatgataggttctcagagcgtgattcgaatcataatacacccagatctggaaataggtggagtagaaatcttgatggttga

SEQ ID NO: 55 - protein, 508 aa, Vigna unguiculata
MAAGPTSPSDRPTMAPPPMLLSDSVAKDAILAWFRGEFAAANAIIDALCAHLATFTHDYDAAFTAIHRRRLNWIPVLQMQKYHSIADITLELRRVSDRNHNADDQPKSDDKTTSPESVVGNGAADEHEEYESPESEITDSGSQEMQASPMNVNLCSNHELCEGRPSQFKLTKGFTAKESVKGHMVNVVKGLKLYEDIFTDLELCKLTDFVNEIHAAGQNGELCGETFILFNKQMKGNKRELIQLGVPIFGQIKEDVKSNIEPIPALLQGVIDHMIQWQLLPEYKRPNGCIINFFEEGEFSQPFLKPPHLDQPVSTLLLSESTMAFGRVLMSENDGNYKGPLMLSLKQGSLLVMRGNSADMARHVMCPSPNRRVSITFFRVRPDSNQYQSPTPAMTSAMTLWQPGIASPYALPNGALSSYEGMDMMPKWGVLRGPMVMLTPMRPVALTPRKLAGGGTGVFLPWNVPSRKPARHLPPRAQKGRLLTLPSPVVEPQMGESTSEPSSICVEG

SEQ ID NO: 56 - DNA, 1527 bp, Vigna unguiculata
  source 1..1527: mol_type = unassigned DNA
atggtggcaggcccaacctcaccctccgatcgacccaccatggcccctcccccaatgctcctctccgactcggtcgccaaggatgccatactggcttggtttcgcggcgagttcgctgcagccaacgccatcatcgacgccctctgcgctcacctcgccacctctacccacgactatgatgctgccttcaccgccatccaccgccgtaggctcaactggatccccgtcctccagatgcagaagtaccactccattgccgacatcacccttgagctccgcagggtctccgacagaaaccacaacgctgacgatcaacccaaatccgacgacaaaacgacgtcgccagagagtgtcgtcgaaaatggtgcggctgatgaacacgaggagtatgaatcgcctgagagcgagattactgattcaggatctcaagaaatgcaggctagttccatgaacgttaatttatgttccaatcatgaactttgtgaaggacgtccttcacagtttaagttgactaaaggttttactgcaaaagagtctgtcaagggacatatggtgaatgtagtaaaaggattgaagttatatgaagacattttcactgatttagaactatgcaagttgacagactttgtgaatgagattcatgctgctggtcagaatggagaactgtgtggtgaaacttttatattattcaacaagcagatgaagggaaacaagagagaactgattcagctgggtgttccaatatttggacagataaaggaggacgtaaaaagcaatatagagccaattcctgcacttctccaaggtgtcattgatcacatgattcagtggcaattacttcccgagtacaaaagaccaaatggttgtattatcaacttttttgaagagggggagttttctcaaccattcctcaaaccaccgcatctggatcaacctgtgtccactcttcttctctctgaatcaacaatggctttcggacgtgtacttatgagtgaaaatgatggaaactacaaaggacctctcatgctctcattgaagcaggggtctcttttagtgatgagaggaaacagtgctgacatggcaaggcacgtaatgtgtccatctcctaacagaagggtcagcatcaccttcttcagagtgaggccagattccaatcaataccagtcaccaactcctgctatgacaagtgcgatgactctgtggcaacctggcattgctagtccatatgctttgccaaatggtgctctaagtagctatgagggtatggacatgatgccaaaatggggagtgcttcgtggtccaatggtgatgctaactcctgtgcgtcctgtagcattgactcctcgcaaacttgctggtggtggaactggggtgtttttgccatggaatgttccatctagaaaacctgccaggcatcttcccccacgtgcccaaaaagggaggcttctcacattaccttcacccgttgttgaaccccaaatgggagaatccacttctgaaccaagcagcatttgtgttgaaggataa

SEQ ID NO: 57 - protein, 516 aa, Gossypium sp.
MNDLERVKKEDPFLVKYQPSELRIASEFLTTWLPFLSRDLCRRCTQILSRRIRSLDPGDAELANNDLNAIHSTHNNTESEDLNENCRDNCAVNSIGSSKDEDTNSLGSWKDEGNGFSEPIGEASTSSGVACASPLFQTPVPRQSWADMAQEDELEEVEEEEEEEEEQLVSESSKRIVSLTASTGELRISKVVEKPKLSRDQRESIRFMNVKRKKDFICLERVNGKFVNILEGLELHEGIFSAAEQKRIVDHVYMLQEMGRKGELKERTYTAPHKWMRGKGRITIQFGCCYNYAMDKNGNPPGILQNEVVDPIPDLFKVIIRRLVRCHVLPPTCVPDSCIVNIYEEGDCIPPHLDTHDFVRPFCTVSFLSECNVVFGSNLKVVDAGEFSGPIAIPLPVGSVLVLNGNGADVAKHCVPAVPTKRISITFRRMDELKRPIGYAPESDLQGIEPLSYDAERPKGFNSPKSERPVRQPFRREDKREAKGLTENNEPSEWRSSTRPRRGPSYRRRFTVNQSR

SEQ ID NO: 58 - DNA, 1677 bp, Gossypium sp.
  source 1..1677: mol_type = unassigned DNA
atgaacgatctcgagcgagtcaagaaggaagacccgtttctcgtcaagtatcagccctccgaactcagaatcgcctctgagttcttaaccacgtggcttcctttcctttccagggatctctgccgtcgatgcactcaaatcctctcccgacgaatccgctctctagacccaggagatgctgagctggccaacaatgatctgaatgcaatccattctacccacaataatactgaatcagaagacttaaatgagaattgtcgtgacaattgtgctgtgaactcaattgggagttcaaaagatgaagataccaactcattgggtagttggaaagatgagggaaatggattttctgaacccatcggagaagcatccactagtagtggagttgcatgcgcatcacctctgtttcaaacccctgttccccgtcaatcatgggcagacatggcacaggaagatgagcttgaggaagtagaagaagaagaagaagaagaagaagagcaactggtgtcagagtcaagcaagcggattgttagtttaactgcttctacaggggagttgaggatatctaaggttgtcgagaagcctaaattgtctagggatcagagagagtccattaggtttatgaatgtgaagaggaagaaagattttatatgtttggagagagttaatggtaagtttgtaaatattctcgaggggcttgaactccatgagggaattttcagtgctgcagaacagaagaggatagttgaccatgtttacatgcttcaggagatggggagaaaaggggagttgaaagaacgcacatatacagcacctcacaaatggatgaggggaaaagggcgcataaccatccaatttggttgttgttacaattatgcaatggacaaaaatggtaacccacctggtattcttcaaaatgaggttgtggatcctatacctgatctcttcaaggtaataattagaaggctagtcagatggcatgtgcttcctccgacctgtgtgcctgacagttgtattgttaatatatatgaagaaggtgactgcatacctcctcacattgacaaccatgattttgttagaccattctgcactgtatcatttcttagcgagtgcaatgtagtttttgggtcaaacttgaaggttgtagatgctggtgagttttccgggccaattgcaattcccctacctgtcgggtcagttctagttttaaatggaaatggagcagatgttgctaagcattgtgtgcctgcagttcctacgaagaggatatcgatcacatttaggagaatggatgaattgaaacggcctataggatatgctccagaatcagacttgcagggcattgaaccattgtcatatgatgcagagaggcccaagggatttaattcaccaaaatctgaacgccctgtaaggcaaccgtttagaagagaagataaaagggaagccaagggcttaacagagaataatgagccctcagaatggcgttcctcaacccggcctcgaagaggcccttcatataggagaaggttcactgtgaatcagagcagacttgaggttggcttgacccggtggaagctgctggaatttgatagattggattgttgctataattgcttggttctactttcagctacaagaaagacgagattctgtactttctttccttttctcaattga

SEQ ID NO: 59 - protein, 662 aa, Gossypium sp.
MAVPSGNAVLSDKMQFPAPSAAVAGAGGGDAGAVGGAGGGGGGGAEFNQHHHRNWFPDERDGFIYWLRGEFAAANAMIDSLCHHLREVGEVGEYEAVIACIQQRRCHWNPVLHMQQYFSIAEVSYALQQVAWRCRQRHCDHGKVGGKDFKRSGFGFKGHRVEVAKEIQNSVLDTNGNSTVTAVSERNERGSEKYEELKLGGELGKVEDKGSVVTEEKSSLSCLARNSWNRLEHDSHPAQNQNENQTLALHPKTFVGNEMFDGNMVNVVDGLKLYEKLFDEKEVSDLVSLINDLRAAGKRGHFQVQTYVASKKPMKGHGREMIQLGLPIADAPLDGETAARTSKVDRRIEAIPSLLQDTIERLVDLQVITAKPDSCIIDVYNEGDHSPPRMWPPWFGNPTFVLFLTECDITFGRVIGVDHPGDFKGSLKLSLSPGSLLVMEGKSADFAKHALPSVRKHRMLVIFTKYQPKKSVADNQRPPSPSVSQSSQWGPPSSRSLNRFRHSAGTKHFAAIPTNGVLPAAAIQQQIPPANVVQPLFVPAPAIPFPAPVPIPPGSTGWSAAAGQRHFPPHLPIPGTGVFLPPPGSNSSCQLLSTIATELNVPMETISPLENSNGSGKLTHHTTSSRGKCDGPSPKQGCNGSVDETGEGRAAMCLTSKPANAV

SEQ ID NO: 60 - DNA, 1989 bp, Gossypium sp.
  source 1..1989: mol_type = unassigned DNA
atggcagtgccatcgggaaatgcagttttatcagataaaatgcagtttcctgctccttcagctgcagtagctggcgccggaggtggtgatgctggagccgttggcggtgcaggaggaggaggtggaggaggtgcagagttcaaccagcaccatcatcggaattggtttcccgatgagcgtgatggctttatctattggttgcggggtgaattcgcagcagccaatgctatgattgactccctttgtcatcatttgcgtgaggtcggggaagttggggagtatgaagccgtcatagcttgcattcagcaaaggagatgtcactggaaccctgtgcttcatatgcagcagtacttttcgattgctgaggtttcctatgctcttcaacaggtggcctggagatgtaggcaaaggcattgtgatcatgggaaagttgggggaaaggattttaaaaggtctggatttggctttaaggggcatagggtagaggtggccaaagaaatacagaattctgtgctggatactaatgggaattctactgttaccgctgtttcagagagaaatgagagagggagtgagaaatatgaggaacttaagttaggtggtgagttagggaaggtggaagataaaggctcagttgtcacagaggaaaaatcgtctctttcctgtttagctaggaacagctggaatagattggaacatgattcacatcctgcacaaaatcagaatgaaaaccagactctggcattgcatccaaaaacttttgttggtaatgagatgtttgacggaaacatggtcaatgtggtggatgggctaaaattgtatgagaaactgtttgatgagaaggaagtttctgaccttgtctccttgataaatgatttaagggctgctgggaaaagaggacattttcaagttcagacatatgtagcttcaaaaaaaccgatgaagggacatggcagagagatgattcaattgggccttccaattgctgatgcccctctggatggtgaaactgctgccaggacttccaaagtagatcggagaatagaagccattccttccttgttgcaagatactattgaacgtttggtcgacttgcaagttattactgcaaagccagactcttgcatcattgatgtctataatgagggggatcattcaccgcctcgaatgtggccaccttggtttggaaaccctactttcgtcttgttcttgactgagtgtgacattacttttggaagggttattggggttgatcatccgggtgatttcaaaggctctctaaagctttctctttcacctggatcgctcctcgtaatggaaggaaaatcggctgattttgccaaacatgcactaccttctgtccgaaagcaccgcatgcttgttatatttaccaagtatcaaccaaagaaatctgtggctgacaaccagaggcctccttcaccttcggtttctcagtcttcacaatggggtccaccatctagtcgatcactcaaccgttttcgccattctgctggcaccaagcattttgcagcaataccaacaaatggtgtactgccagctgcagcaattcagcaacaaattccacccgcaaatgttgttcaaccattatttgtgcctgcacctgctattccatttcctgctccagttcccatcccgcctggttcgactgggtggtcagccgctgctggtcaaaggcattttccacctcatctacccattcctggcactggagttttcttgcctccaccaggctcaaattcatcgtgtcaactattgtcaactattgcaaccgaactcaatgttcccatggagacaatttcccctttggaaaacagtaatggttcagggaaactcactcaccatacaacttcatctagagggaagtgtgatggaccatcaccaaaacaaggctgcaatggaagtgtagatgaaaccggtgaaggaagagcagcaatgtgcttgacaagcaaacctgctaatgcagtttag

SEQ ID NO: 61 - DNA, 1710 bp, Arabidopsis thaliana
  source 1..1710: mol_type = unassigned DNA
atgacgattgcggcagcgccagcacgccagacagatcgatcagccactggtttcaaccctgcttacgtcacgacggcgaaggcggtgagcgttccggttcaagttcctccggcgactgtggtttccgaaggattaggtaaagatgcgttgatctcatggttcagaggagaattcgctgcggcaaacgcgataatcgacgcgatgtgctcgcatctaaggatagctgaagaagcggtatctggatcggagtacgaagctgtattcgcggcgatccatcgccgtcgtctgaattggatccctgttcttcagatgcagaagtatcattccatcgctgaagtagcgatcgagcttcagaaagtggcggctaagaaagcggaggatctgaaacagaagaaaacagaggaggaggcggaggaagatctgaaagaggtggtggcgacggaggaagaggaagtgaagaaagagtgttttaacggcgagaaagtaacggagaatgacgtaaacggagatgtagaagacgttgaagatgattcaccaactagtgatatcactgattcaggttctcatcaggatgttcaccaaactgttgttgcggatactgcacatcagattatatgccatagccatgaagattgtgatgcacgttcatgtgagatcaaacctatcaaaggtttccaagctaaagaacaagtcaaaggccacactgtgaatgttgtgaaaggactaaagctgtatgaagagttacttaaagaagatgagatatctaaattgttagactttgtggcagaactacgagaagctggcataaacgggaagcttgcaggtgagagctttatactgttcaacaaacagattaaagggaacaagagagagctgattcagcttggtgtccccatctttggtcatgtcaaggcggatgaaaactctaacgacaccaacaactctgttaacatcgagccaattccaccacttcttgagagtgtcattgatcactttgtcacatggagactcatcccagaatacaagagacctaacggctgtgtcatcaacttctttgaagagggtgaatactcacagcctttcctcaaaccacctcacttagaacaaccaatctccactcttgtcctctctgaatcaacaatggcctatggacgcattctctcaagtgacaacgaaggcaacttcagaggacctttgacactctctctcaaacaaggatctttgttggtgatgagagggaacagtgcagacatggcaagacatgtaatgtgtccatcacaaaacaaaagagtaagcatcacattctttcgtattcgacctgacacatatcataaccattcacaacccaacagtcctcgcaacgacggtgtcatgacaatgtggcaaccctaccaaatgacaccaactccattcctcaatggttatgatcattcaattgacatgatgccaaaacttggagtcctacgtcctccaatggtcatgatggcaccaccaccggttcaaccaatgatattaccaagtcccaatgtgatgggaaccggtggtggtaccggtgttttcttaccatgggcctctgttaatagctcaagaaaacatgtgaagcatttgcctccacgcgcgcagaagaagcgattacttccgcttcctcctgctgcttcttcttctccagctggaggatccacctctgagcctgtgatcactgtaggttaa

SEQ ID NO: 62 - DNA, 1707 bp, synthetic construct
  source 1..1707: mol_type = other DNA
acgattgcggcagcgccagcacgccagacagatcgatcagccactggtttcaaccctgcttacgtcacgacggcgaaggcggtgagcgttccggttcaagttcctccggcgactgtggtttccgaaggattaggtaaagatgcgttgatctcatggttcagaggagaattcgctgcggcaaacgcgataatcgacgcgatgtgctcgcatctaaggatagctgaagaagcggtatctggatcggagtacgaagctgtattcgcggcgatccatcgccgtcgtctgaattggatccctgttcttcagatgcagaagtatcattccatcgctgaagtagcgatcgagcttcagaaagtggcggctaagaaagcggaggatctgaaacagaagaaaacagaggaggaggcggaggaagatctgaaagaggtggtggcgacggaggaagaggaagtgaagaaagagtgttttaacggcgagaaagtaacggagaatgacgtaaacggagatgtagaagacgttgaagatgattcaccaactagtgatatcactgattcaggttctcatcaggatgttcaccaaactgttgttgcggatactgcacatcagattatatgccatagccatgaagattgtgatgcacgttcatgtgagatcaaacctatcaaaggtttccaagctaaagaacaagtcaaaggccacactgtgaatgttgtgaaaggactaaagctgtatgaagagttacttaaagaagatgagatatctaaattgttagactttgtggcagaactacgagaagctggcataaacgggaagcttgcaggtgagagctttatactgttcaacaaacagattaaagggaacaagagagagctgattcagcttggtgtccccatctttggtcatgtcaaggcggatgaaaactctaacgacaccaacaactctgttaacatcgagccaattccaccacttcttgagagtgtcattgatcactttgtcacatggagactcatcccagaatacaagagacctaacggctgtgtcatcaacttctttgaagagggtgaatactcacagcctttcctcaaaccacctcacttagaacaaccaatctccactcttgtcctctctgaatcaacaatggcctatggacgcattctctcaagtgacaacgaaggcaacttcagaggacctttgacactctctctcaaacaaggatctttgttggtgatgagagggaacagtgcagacatggcaagacatgtaatgtgtccatcacaaaacaaaagagtaagcatcacattctttcgtattcgacctgacacatatcataaccattcacaacccaacagtcctcgcaacgacggtgtcatgacaatgtggcaaccctaccaaatgacaccaactccattcctcaatggttatgatcattcaattgacatgatgccaaaacttggagtcctacgtcctccaatggtcatgatggcaccaccaccggttcaaccaatgatattaccaagtcccaatgtgatgggaaccggtggtggtaccggtgttttcttaccatgggcctctgttaatagctcaagaaaacatgtgaagcatttgcctccacgcgcgcagaagaagcgattacttccgcttcctcctgctgcttcttcttctccagctggaggatccacctctgagcctgtgatcactgtaggttaa

SEQ ID NO: 63 - RNA, 33 nt, synthetic construct
  source 1..33: mol_type = other RNA
  modified_base 26: mod_base = OTHER; note = m6A modified nucleotide
cgcttgtgtgtgctgctggacttggactcgcgc

SEQ ID NO: 64 - DNA, 42 bp, synthetic construct
  source 1..42: mol_type = other DNA
gttccagattacgctagatctaagcgcaccccgactgccgag

SEQ ID NO: 65 - DNA, 42 bp, synthetic construct
  source 1..42: mol_type = other DNA
ttagggagaggggcgctcgagtcagggttttgcttccagaag

SEQ ID NO: 66 - DNA, 22 bp, synthetic construct
  source 1..22: mol_type = other DNA
acatggcggcggtgctggagta

SEQ ID NO: 67 - DNA, 24 bp, synthetic construct
  source 1..24: mol_type = other DNA
cgggatgatgcgcgtcttcttgtt

SEQ ID NO: 68 - DNA, 22 bp, synthetic construct
  source 1..22: mol_type = other DNA
gagacataacaacagatcctga

SEQ ID NO: 69 - DNA, 21 bp, synthetic construct
  source 1..21: mol_type = other DNA
aactttggtacctggtatctg

SEQ ID NO: 70 - DNA, 21 bp, synthetic construct
  source 1..21: mol_type = other DNA
ggattccacacgtgatcctaa

SEQ ID NO: 71 - DNA, 21 bp, synthetic construct
  source 1..21: mol_type = other DNA
tcctctatgagcagacctgga

SEQ ID NO: 72 - DNA, 17 bp, synthetic construct
  source 1..17: mol_type = other DNA
cagcggtcgccatcttg

SEQ ID NO: 73 - DNA, 24 bp, synthetic construct
  source 1..24: mol_type = other DNA
caccctctcacctgttcagactaa

SEQ ID NO: 74 - protein, 8 aa, synthetic construct
DYKDDDDK

SEQ ID NO: 75 - DNA, 41 bp, synthetic construct
  source 1..41: mol_type = other DNA
gttccagattacgctagatctgcggccgccagcggctacac

SEQ ID NO: 76 - DNA, 42 bp, synthetic construct
  source 1..42: mol_type = other DNA
ttagggagaggggcgctcgagtcagtgccgccgcatcttcac

SEQ ID NO: 77 - DNA, 30 bp, synthetic construct
  source 1..30: mol_type = other DNA
cagccgggaggcgcgcaaggtgaagagcgg

SEQ ID NO: 78 - DNA, 32 bp, synthetic construct
  source 1..32: mol_type = other DNA
ccttgcgcgcctcccggctgcccgccttatag

SEQ ID NO: 79 - DNA, 44 bp, synthetic construct
  source 1..44: mol_type = other DNA
ttagggagaggggcgctcgagtcacagggactttgtttccaacc

SEQ ID NO: 80 - DNA, 42 bp, synthetic construct; identical to SEQ ID NO: 64
  source 1..42: mol_type = other DNA

SEQ ID NO: 81 - DNA, 42 bp, synthetic construct; identical to SEQ ID NO: 65
  source 1..42: mol_type = other DNA

SEQ ID NO: 82 - DNA, 101 bp, synthetic construct
  source 1..101: mol_type = other DNA
ctagatctccaaaaaagaagagaaaggtagatccaaaaaagaagagaaaggtagatccaaaaaagaagagaaaggtaggatccgcggccgccagcggctac

SEQ ID NO: 83 - DNA, 45 bp, synthetic construct
  source 1..45: mol_type = other DNA
acgatgttccagattacgctagatctgtgagcaagggcgaggagc

SEQ ID NO: 84 - DNA, 35 bp, synthetic construct
  source 1..35: mol_type = other DNA
tttcgctgccgctgccggacttgtacagctcgtcc

SEQ ID NO: 85 - DNA, 36 bp, synthetic construct
  source 1..36: mol_type = other DNA
gtccggcagcggcagcgaaaacgatccatttctccg

SEQ ID NO: 86 - DNA, 45 bp, synthetic construct
  source 1..45: mol_type = other DNA
agaagagaaaggtaggttccagatctgaaaacgatccatttctcc

SEQ ID NO: 87 - DNA, 36 bp, synthetic construct
  source 1..36: mol_type = other DNA
tggggctgccgctgccaccgtagtttcttctactag

SEQ ID NO: 88 - DNA, 35 bp, synthetic construct
  source 1..35: mol_type = other DNA
cggtggcagcggcagccccaagaagaagaggaagg

SEQ ID NO: 89 - DNA, 49 bp, synthetic construct
  source 1..49: mol_type = other DNA
tgcacctgaggagtgaattcctcgagtcacaccttcctcttcttcttgg

SEQ ID NO: 90 - DNA, 46 bp, synthetic construct
  source 1..46: mol_type = other DNA
caaaaaagaagagaaaggtaggatccgaaaatgaccccttcctgag

SEQ ID NO: 91 - DNA, 30 bp, synthetic construct
  source 1..30: mol_type = other DNA
agctgcccagcatgttctcgaccacgctac

SEQ ID NO: 92 - DNA, 30 bp, synthetic construct
  source 1..30: mol_type = other DNA
cgagaacatgctgggcagctggaagggcag

SEQ ID NO: 93 - DNA, 49 bp, synthetic construct
  source 1..49: mol_type = other DNA
taacgttagggagaggggcgctcgagttagccgtagttccgccggctag

SEQ ID NO: 94 - DNA, 29 bp, synthetic construct
  source 1..29: mol_type = other DNA
tgatatggcccagcgcgagaatctgagac

SEQ ID NO: 95 - DNA, 29 bp, synthetic construct
  source 1..29: mol_type = other DNA
tctcgcgctgggccatatcagcccaccgc

SEQ ID NO: 96 - DNA, 48 bp, synthetic construct
  source 1..48: mol_type = other DNA
taacgttagggagaggggcgctcgagttagatgccttggagatcaggc

SEQ ID NO: 97 - DNA, 100 bp, synthetic construct
  source 1..100: mol_type = other DNA
tgaccggtccaaaaaagaagagaaaggtagatccaaaaaagaagagaaaggtagatccaaaaaagaagagaaaggtaggatccaccatcgccgctgctcc

SEQ ID NO: 98 - DNA, 41 bp, synthetic construct
  source 1..41: mol_type = other DNA
ttagggagaggggcgctcgagtcagcccacggtgatcacag

SEQ ID NO: 99 - DNA, 46 bp, synthetic construct
  source 1..46: mol_type = other DNA
caaaaaagaagagaaaggtaggatccgacgcctcctcctccgcctc

SEQ ID NO: 100 - DNA, 49 bp, synthetic construct
  source 1..49: mol_type = other DNA
taacgttagggagaggggcgctcgagttactcatccaggtttaccctgg

SEQ ID NO: 101 - DNA, 50 bp, synthetic construct
  source 1..50: mol_type = other DNA
tgcacctgaggagtgaattcctcgagtcattattcttgtacagctgtttc

SEQ ID NO: 102 - DNA, 30 bp, synthetic construct
  source 1..30: mol_type = other DNA
cgtctcctccagggagcagcgggagctgag

SEQ ID NO: 103 - DNA, 30 bp, synthetic construct
  source 1..30: mol_type = other DNA
cgctgctccctggaggagacgcggctgcgg

SEQ ID NO: 104 - DNA, 40 bp, synthetic construct
  source 1..40: mol_type = other DNA
agaagagaaaggtaggatccgccgcgccggggccggggtg

SEQ ID NO: 105 - DNA, 48 bp, synthetic construct
  source 1..48: mol_type = other DNA
taacgttagggagaggggcgctcgagctactttgcgacaacattctgc

SEQ ID NO: 106 - DNA, 50 bp, synthetic construct
  source 1..50: mol_type = other DNA
tgcacctgaggagtgaattcctcgagtcactaagcagcttctgcgatcca

SEQ ID NO: 107 - DNA, 45 bp, synthetic construct
  source 1..45: mol_type = other DNA
caaaaaagaagagaaaggtaggatccgacggcctcgtcgcgtggc

SEQ ID NO: 108 - DNA, 29 bp, synthetic construct
  source 1..29: mol_type = other DNA
caatcgcagcgtgaagcgcgagggcgacc

SEQ ID NO: 109 - DNA, 30 bp, synthetic construct
  source 1..30: mol_type = other DNA
cgcgcttcacgctgcgattgccagtggagg

SEQ ID NO: 110 - DNA, 48 bp, synthetic construct
  source 1..48: mol_type = other DNA
taacgttagggagaggggcgctcgagctacctgggagcagcttctgcg

SEQ ID NO: 111 - DNA, 58 bp, synthetic construct
  source 1..58: mol_type = other DNA
gcgtcccgggccaaaaaagaagagaaaggtaggttccagatctggtaccggatccaag

SEQ ID NO: 112 - DNA, 58 bp, synthetic construct
  source 1..58: mol_type = other DNA
cttggatccggtaccagatctggaacctacctttctcttcttttttggcccgggacgc

SEQ ID NO: 113 - DNA, 45 bp, synthetic construct
  source 1..45: mol_type = other DNA
gaagagaaaggtaggttccagatctgcggccgccagcggctacac

SEQ ID NO: 114 - DNA, 46 bp, synthetic construct
  source 1..46: mol_type = other DNA
cattattatggagaaagcttggatcctcagtgccgccgcatcttca

SEQ ID NO: 115 - DNA, 45 bp, synthetic construct
  source 1..45: mol_type = other DNA
cattattatggagaaagcttggatcctcacagggactttgtttcc

SEQ ID NO: 116 - DNA, 45 bp, synthetic construct; identical to SEQ ID NO: 86
  source 1..45: mol_type = other DNA

SEQ ID NO: 117 - DNA, 49 bp, synthetic construct
  source 1..49: mol_type = other DNA
cattattatggagaaagcttggatccttaaccgtagtttcttctactag

SEQ ID NO: 118 - DNA, 48 bp, synthetic construct
  source 1..48: mol_type = other DNA
cattattatggagaaagcttggatccttaaatcccttgcaaatcaggc

SEQ ID NO: 119 - DNA, 45 bp, synthetic construct
  source 1..45: mol_type = other DNA
agaagagaaaggtaggttccagatctgtgagcaagggcgaggagc

SEQ ID NO: 120 - DNA, 53 bp, synthetic construct
  source 1..53: mol_type = other DNA
cattattatggagaaagcttggatccttatcacaccttcctcttcttcttggg

SEQ ID NO: 121 - DNA, 46 bp, synthetic construct
  source 1..46: mol_type = other DNA
agaagagaaaggtaggttccagatctgacgcctcctcctccgcctc

SEQ ID NO: 122 - DNA, 49 bp, synthetic construct
  source 1..49: mol_type = other DNA
cattattatggagaaagcttggatccttactcatccaggtttaccctgg

SEQ ID NO: 123 - DNA, 49 bp, synthetic construct
  source 1..49: mol_type = other DNA
cattattatggagaaagcttggatccttattcttgtacagctgtttcta

SEQ ID NO: 124 - DNA, 46 bp, synthetic construct
  source 1..46: mol_type = other DNA
agaagagaaaggtaggttccagatctgccgcgccggggccggggtg

SEQ ID NO: 125 - DNA, 46 bp, synthetic construct
  source 1..46: mol_type = other DNA
cattattatggagaaagcttggatccctactttgcgacaacattct

SEQ ID NO: 126 - DNA, 49 bp, synthetic construct
  source 1..49: mol_type = other DNA
cattattatggagaaagcttggatccctaagcagcttctgcgatccatg

SEQ ID NO: 127 - DNA, 29 bp, synthetic construct
  source 1..29: mol_type = other DNA
tcgtgtctgccgtggaccccatccacatc

SEQ ID NO: 128 - DNA, 32 bp, synthetic construct
  source 1..32: mol_type = other DNA
ggggtccacggcagacacgatgcagccgccgg

SEQ ID NO: 129 - DNA, 33 bp, synthetic construct
  source 1..33: mol_type = other DNA
cccccgccatcgccaaccacgacttcctgcggc

SEQ ID NO: 130 - DNA, 36 bp, synthetic construct
  source 1..36: mol_type = other DNA
gtggttggcgatggcgggggggatacagtcgccttc

SEQ ID NO: 131 - DNA, 34 bp, synthetic construct
  source 1..34: mol_type = other DNA
caccagccattgccagtcatgattttgttcggcc

SEQ ID NO: 132 - DNA, 35 bp, synthetic construct
  source 1..35: mol_type = other DNA
atgactggcaatggctggtgggatgcaatctccag

SEQ ID NO: 133 - DNA, 34 bp, synthetic construct
  source 1..34: mol_type = other DNA
accctgcccatgcccctccttggtatggtagacc

SEQ ID NO: 134 - DNA, 34 bp, synthetic construct
  source 1..34: mol_type = other DNA
aggaggggcatgggcagggtgagaataatctccc

SEQ ID NO: 135 - DNA, 30 bp, synthetic construct
  source 1..30: mol_type = other DNA
agcaagatccagcagctccgtgttaccacc

SEQ ID NO: 136 - DNA, 29 bp, synthetic construct
  source 1..29: mol_type = other DNA
cggagctgctggatcttgcttccagcagc

SEQ ID NO: 137 - DNA, 46 bp, synthetic construct
  source 1..46: mol_type = other DNA
acgatgttccagattacgctagatctatggcttctgagtctgaaac

SEQ ID NO: 138 - DNA, 45 bp, synthetic construct
  source 1..45: mol_type = other DNA
tgcacctgaggagtgaattcctcgagctactccatgatggcccgg

SEQ ID NO: 139 - DNA, 38 bp, synthetic construct
  source 1..38: mol_type = other DNA
gggttctggatccggttctgaggcctgggccaagcctc

SEQ ID NO: 140 - DNA, 39 bp, synthetic construct
  source 1..39: mol_type = other DNA
cagaaccggatccagaaccctcagctgcttctggcgtgg

SEQ ID NO: 141 - DNA, 46 bp, synthetic construct
  source 1..46: mol_type = other DNA
acgatgttccagattacgctagatctatggggtctgaggaccacgg

SEQ ID NO: 142 - DNA, 46 bp, synthetic construct
  source 1..46: mol_type = other DNA
tgcacctgaggagtgaattcctcgagctagaagggggctccgggcc

SEQ ID NO: 143 - DNA, 38 bp, synthetic construct
  source 1..38: mol_type = other DNA
cggttctggatccggttctttgaggccgctgctgtctc

SEQ ID NO: 144 - DNA, 39 bp, synthetic construct
  source 1..39: mol_type = other DNA
aagaaccggatccagaaccggtgagcgccgtgggccgcg

SEQ ID NO: 145 - DNA, 46 bp, synthetic construct
  source 1..46: mol_type = other DNA
acgatgttccagattacgctagatctatgaaatcctgcggagtgtc

SEQ ID NO: 146 - DNA, 44 bp, synthetic construct
  source 1..44: mol_type = other DNA
tgcacctgaggagtgaattcctcgagtcaagatgaggcggatgg

SEQ ID NO: 147 - DNA, 38 bp, synthetic construct
  source 1..38: mol_type = other DNA
gaggttctggatccggttctagctcagcagaagttctg

SEQ ID NO: 148 - DNA, 37 bp, synthetic construct
  source 1..37: mol_type = other DNA
agaaccggatccagaacctctcatttggtgttgttgc

SEQ ID NO: 149 - DNA, 46 bp, synthetic construct
  source 1..46: mol_type = other DNA
acgatgttccagattacgctagatctatgcatcgggcagtggatcc

SEQ ID NO: 150 - DNA, 69 bp, synthetic construct
  source 1..69: mol_type = other DNA
tgcacctgaggagtgaattcctcgagtcatcgcgacgtgctggctggggccagcgtgaaggcgtcgtag

SEQ ID NO: 151 - DNA, 46 bp, synthetic construct
  source 1..46: mol_type = other DNA
acgatgttccagattacgctagatctccccgggaaaaactcaaccc

SEQ ID NO: 152 - DNA, 46 bp, synthetic construct
  source 1..46: mol_type = other DNA
tgcacctgaggagtgaattcctcgagtcatcgcgacgtgctggctg

SEQ ID NO: 153 - DNA, 25 bp, synthetic construct
  source 1..25: mol_type = other DNA
caccgacaggttccgacacccggat

SEQ ID NO: 154 - DNA, 25 bp, synthetic construct
  source 1..25: mol_type = other DNA
aaacatccgggtgtcggaacctgtc

SEQ ID NO: 155 - DNA, 24 bp, synthetic construct
  source 1..24: mol_type = other DNA
caccgggcgagggctacacgtacg

SEQ ID NO: 156 - DNA, 24 bp, synthetic construct
  source 1..24: mol_type = other DNA
aaaccgtacgtgtagccctcgccc

SEQ ID NO: 157 - DNA, 25 bp, synthetic construct
  source 1..25: mol_type = other DNA
caccgccccgagcgcagcgactacg

SEQ ID NO: 158 - DNA, 25 bp, synthetic construct
  source 1..25: mol_type = other DNA
aaaccgtagtcgctgcgctcggggc

SEQ ID NO: 159 - DNA, 20 bp, synthetic construct
  source 1..20: mol_type = other DNA
agcttcctatggtttgcacg

SEQ ID NO: 160 - DNA, 21 bp, synthetic construct
  source 1..21: mol_type = other DNA
ttcggctgcaagttccagttc

SEQ ID NO: 161 - DNA, 20 bp, synthetic construct
  source 1..20: mol_type = other DNA
agatgtggatggggtcaacg

SEQ ID NO: 162 - DNA, 19 bp, synthetic construct
  source 1..19: mol_type = other DNA
gggacaactacaaggcggg

SEQ ID NO: 163 - DNA, 200 bp, synthetic construct
  source 1..200: mol_type = other DNA
cctttacaagatgcccttttgagactccatgtgtgtttcctttgcaggacaagattagatgcaccgcgacttgaaacatacccatacgatgttccagattacgcttgatgaaaatccctgagcagctccacattgccacccagctatgcttcagatcgcctgtcaggaaacaccagagaccctgcgctgaaacccaaaag

SEQ ID NO: 164 - DNA, 200 bp, synthetic construct
  source 1..200: mol_type = other DNA
gtgtttcctgacaggcgatctgaagcatagctgggtggcaatgtggagctgctcagggattttcatcaagcgtaatctggaacatcgtatgggtatgtttcaagtcgcggtgcatctaatcttgtcctgcaaaggaaacacacatggagtctcaaaagggcatcttgtaaaggcataaaccctatcaggacagatctgtc

SEQ ID NO: 165 - DNA, 21 bp, synthetic construct
  source 1..21: mol_type = other DNA
tctctggaagtcactaggcgt

SEQ ID NO: 166 - RNA, 36 nt, synthetic construct
  source 1..36: mol_type = other RNA
aagattagatgcaccgcgatgttttagagctatgct

SEQ ID NO: 167 - RNA, 36 nt, synthetic construct
  source 1..36: mol_type = other RNA
agggattttgtttccaatcggttttagagctatgct

SEQ ID NO: 168 - DNA, 20 bp, synthetic construct
  source 1..20: mol_type = other DNA
tgacaggcgatctgaagcat

SEQ ID NO: 169 - DNA, 22 bp, synthetic construct
  source 1..22: mol_type = other DNA
acatggcggcggtgctggagta

SEQ ID NO: 170 - DNA, 24 bp, synthetic construct
  source 1..24: mol_type = other DNA
cgggatgatgcgcgtcttcttgtt

SEQ ID NO: 171 - DNA, 22 bp, synthetic construct
  source 1..22: mol_type = other DNA
gagacataacaacagatcctga

SEQ ID NO: 172 - DNA, 21 bp, synthetic construct
  source 1..21: mol_type = other DNA
aactttggtacctggtatctg

SEQ ID NO: 173 - DNA, 21 bp, synthetic construct
  source 1..21: mol_type = other DNA
ggattccacacgtgatcctaa

SEQ ID NO: 174 - DNA, 21 bp, synthetic construct
  source 1..21: mol_type = other DNA
tcctctatgagcagacctgga

SEQ ID NO: 175 - DNA, 17 bp, synthetic construct
  source 1..17: mol_type = other DNA
cagcggtcgccatcttg

SEQ ID NO: 176 - DNA, 24 bp, synthetic construct
  source 1..24: mol_type = other DNA
caccctctcacctgttcagactaa

SEQ ID NO: 177 - DNA, 32 bp, synthetic construct
  source 1..32: mol_type = other DNA
nnnnnatcacgagatcggaagagcacacgtct

SEQ ID NO: 178 - DNA, 21 bp, synthetic construct
  source 1..21: mol_type = other DNA
agacgtgtgctcttccgatct

SEQ ID NO: 179 - DNA, 31 bp, synthetic construct
  source 1..31: mol_type = other DNA
nnnnngatcgtcggactgtagaactctgaac

SEQ ID NO: 180 - DNA, 20 bp, synthetic construct
  source 1..20: mol_type = other DNA
gaagtccagctgccagaaac

SEQ ID NO: 181 - DNA, 20 bp, synthetic construct
  source 1..20: mol_type = other DNA
ctctacacccacctgctgaa

SEQ ID NO: 182 - DNA, 20 bp, synthetic construct
  source 1..20: mol_type = other DNA
atcgtgtccgtgtccttctt

SEQ ID NO: 183 - DNA, 20 bp, synthetic construct
  source 1..20: mol_type = other DNA
aggcttgaactggaacttgc

SEQ ID NO: 184 - DNA, 20 bp, synthetic construct
  source 1..20: mol_type = other DNA
cacattccagcagatgtgga

SEQ ID NO: 185 - DNA, 20 bp, synthetic construct
  source 1..20: mol_type = other DNA
gcgataacagctcctcttgg

SEQ ID NO: 186 - protein, 150 aa, Glycine max
MEHTNYNDVHCVETIVNVPFSWEHKPGLSKVTTHGHHNGTSMVLQPPPCSSSSSGSGSDHKHKVEEESQILRAVQTSLRISSFRMESQKEEDPFVEAYKKCTRSPFIHQQSNRVQKNIRSRPNIWKYVNFFSCKHSDDVCGNKAVHTYKS

SEQ ID NO: 187 - protein, 666 aa, Glycine max
MASYRPFPPQSSQIPNPIPGGNHQYNNNNNQNQNWGAYGDPSTSSFPQIPPNSNYHQNQHHVPYAPAPSNPHHPPHYPYPPPPPPPPPPEASYQPPPPPPPPAPMYYPSSSQYSNQPPPPPPPLSPPPPPPPVSPPPPPPATQNNNEERRFKEPSKSGRREYEHSNHGIAHKQHKQQQPPLPVKKMNNGPPGRAETDEEKRLRKKREFEKQRQEEKHRQQLKESQNTVLQKTHLLSSGKGHGMIAGSRMGERRSTPLLGAERVENRLKKPTTFLCKLKFRNELPDPSAQPKLMSFKKDKDQYAKYTITSLEKMYKPKLFVEPDLGIPLDLLDLSVYNPPRVRPPLAPEDEELLRDDEAATPIKKDGIKRKERPTDKGVAWLVKTQYISPLSMESTKQSLTEKQAKELREMKGRGILDNLNSRERQIREIQASFEAAKSDPVHATNKDLYPVEVMPLLPDFDRYDDQFVVAAFDNAPTADSEMYAKMNKSVRDAFESKAVMKSYVATGLDPANPEKFLAYMAPAPGELSKDIYDENEDVSYSWIREYHWDVRGDDADDPTTFLVAFDESEARYLPLPTKLVLRKKRAKEGRSGDEVEQCPVPARVTVRRRSSVAAIERKDSGVYTSSKGNSFKRVGLEMDDGLEDQHRGAPHQDNYQSSGAEDYMSD

SEQ ID NO: 188 - protein, 643 aa, Solanum lycopersicum
MLDHHHRSSTTDRFLLDYNADELRIAGEFLSNWFPFLSRDFCSSCTHTLSHRIRSLGRREAVGDAEQLKQQENFVVLTPELPDSNACNGNHDNCYGNSLGSWQDCAVLNGSADTNSLGSWKDGAVVQEPFDDALTCRNKSNSYVGGRSLGSLKGYADLNDTADTNSLGSWKDGADVQEPFDEALAPRNKSDNNVGGAVHPLGSWKDYADLNDTADTNSLGSWKDGADVQEPFDEALAHRNKSDSYIGVADRPIGSWKDCADLNDNVDTNSLGSWKDSADVQEPFEGALTPRIKPDNYLGGAARPVKEASRSKTFRSSRPTAIWRMKMPLADELDAVEISESSICSSQLRNGNGMNKEETSVQGAKPKMELSMEQREHIRFCNVKRKKDFICLERVNGKIVNILDGLELHTGVFSMAEQNRIVKFVEKLEEIGKSGQLKERTYTAPQKWMRGKGRATIQFGCCYNYATDKKGNSPGILKSETVDPLPDLLKSMVRRLVRWHVLPPDCVPDSCIVNIYDVGDCIPPHIDSHDFFRPFCTVSFLSECNVVFGSNLKIVGPGDFAGAIAIPLPMGSVLVLNGNGADVAKHCVPAVPTKRISITFRRMNESRRPIGCAPEQDLLGLQPISHEADRYEKSKTYKPWHSK

SEQ ID NO: 189 - protein, 538 aa, Solanum lycopersicum
MSDHQRTRKDDPFLLNYNSDELRIASEFLSNWLPFLSRDLCRSCTHTLSDRIRSLNSKVSGNAECLKQPKNVSVLTPERCDSDGCNGSQENCDNNSLGSWNDYGDLNDNADTNSLGSWKDGAGGEPFEELSVDRNSSDGCIDGADSQSQSVGEASTSEAFKSSLPVTTPKVKMSWADMAQEDELQAEEVSESIALRSQVNGVAEEEITNPESKQKTELSREKREYIRFCNVKRKKDFICLERVRGKIVNILDGLELHKGVFSAAEQIRIVKYAEKLDQMGKNGELKERTYTTPTKWMRGKGRVTIQFGCCYNYAPDKNGNPPGILKSETVDPLPDLLKVMIRRLIRWHVMPSTCVPDSCIINIYEEGDCIPPHIDNHDFVRPFCTVSFLSECNIVFGSNLKIVGPGEFAGAIAIPLPVGSVLVLNGNGADVAKHCVPAVPTKRISITFRRMDESKRPTGYVPEHDLQGLQPLSYESDSQKKSSSSRPRFSARKQSVRQEEESRERVKMPMRRHSEPRYPGRYRGGPANRQWYGANTEN

SEQ ID NO: 190 - protein, 431 aa, Solanum lycopersicum
MAGDYSRWSPEPRDFKAMLKKLSHEQLLQVLSEGLCSYCEEVIESRVNNLNFIQKSSSDDAISPKRKLNADAYTPFPQVGDSTNSGRSKRFVSALSKPVINKQPASDLSNFEIKLNDGPSRSVVGDDLVEDQKEFIRFSQVGRRKDFVHYENVNEKQINVLKGLELHTNVFNPDEQKEIVELVYSLQRMGQKQQLRARTYSEPRKWMRGKGRITLQFGCCYNYAMDKNGNPPGIVRDEEVDPLPPLFKKMIKRMVRWHVLPTSCVPDSCIVNIYDEGDCIPPHIDHHDFVRPFCTVSFLAECKILFGTTLKIINPGEFSGPFSLPLPLGSVLILNGNGADVAKHCVPSVPAKRISITFRKMDVSKLPYRYTPDPELVGIERLIPSPSLDSSRNRYHGKIDKLPNSENKVFSNEDDFPPLGKSTSSSRRSRR

SEQ ID NO: 191 - protein, 385 aa, Arabidopsis thaliana
MEPNYEEDVFLAKYQSSELKIASEFLTNWLPFLSRDLCNDCAHVLSDRIRSLDPEHCSNGEVKAGSGSMVDNMESEEVDNMESEEVDNKMDNIYDGKLCVSHSLDSLKAGAFVFGSLSDSVSHAMPSSQSTVSEAASAQMSWADMGEEDGLEEEDQKENELGSHGVDVSPSVGDSMKTPEKRKLSREERERYRFMNVKKMKVFSCYEKVRGRSVNILEGLELHTGVFSAVEQKKIVDFVYELQEKGRRGELRERTFTAPHKWMRGKGRVTIQFGCCYNYAPDKAGNPPGILQRGDVDPMPSIFKVIIKRLVGWHVLPPTCVPDSCIVNIYEEDDCIPPHIDNHDFLRPFCTVSFLSECNILFGSNLKVLGPGEFSGSYSIPLPVG

SEQ ID NO: 192 - protein, 327 aa, Arabidopsis thaliana
MDNYSFIRQSELEIASKVNLLSKRIRSQEQDFSHMTLEDELSEEEDHKDSSREAFGSSLENHKLSRKQRTHIRAINVKRKRDFVCLEKVNGELVNILEGLELHTEVFNAAEQRRIVDKVCELQEKVQKGELKRAFTAQGKGRSTIQFGCCFNYRTSKTGNLAGILKHETVDPLPHLFKVIIRRLVKWHVLPPTCVPDCCVVNIYDEGDCIPPHIDHHDFLRPFCTVSFLSECNILFGSNLKVEETGEYSGGSYSLPLPVGSVLVLNGNGADVAKHCVPEVPTKRISITFRKMDESKWPVWYTPEPYLQGIQPLTYELKSCGSSDHVF